SEQUENCE LISTING (174 sequences)

SEQ ID NO: 1 - DNA, 15 bp, Homo sapiens
  source 1..15: mol_type = genomic DNA
gcactcctgcatgta

SEQ ID NO: 2 - DNA, 15 bp, Homo sapiens
  source 1..15: mol_type = genomic DNA
ctcattgtaactcct

SEQ ID NO: 3 - DNA, 15 bp, Homo sapiens
  source 1..15: mol_type = genomic DNA
ctccgaatcatgttg

SEQ ID NO: 4 - DNA, 15 bp, Homo sapiens
  source 1..15: mol_type = genomic DNA
tgtacccgcttgtga

SEQ ID NO: 5 - DNA, 15 bp, Homo sapiens
  source 1..15: mol_type = genomic DNA
tctcagtgtttgtgg

SEQ ID NO: 6 - DNA, 15 bp, Homo sapiens
  source 1..15: mol_type = genomic DNA
gtgtgttgtcctatg

SEQ ID NO: 7 - DNA, 15 bp, Homo sapiens
  source 1..15: mol_type = genomic DNA
agttcagtcaaccga

SEQ ID NO: 8 - DNA, 15 bp, Homo sapiens
  source 1..15: mol_type = genomic DNA
tttgtcctgtacgcc

SEQ ID NO: 9 - DNA, 15 bp, Homo sapiens
  source 1..15: mol_type = genomic DNA
cgcgcacccattaaa

SEQ ID NO: 10 - DNA, 15 bp, Homo sapiens
  source 1..15: mol_type = genomic DNA
acagcgccgtattta

SEQ ID NO: 11 - DNA, 15 bp, Homo sapiens
  source 1..15: mol_type = genomic DNA
tgttcccgctcaact

SEQ ID NO: 12 - DNA, 15 bp, Homo sapiens
  source 1..15: mol_type = genomic DNA
tcaatccttccgctt

SEQ ID NO: 13 - DNA, 15 bp, Homo sapiens
  source 1..15: mol_type = genomic DNA
gctgcgctttccatt

SEQ ID NO: 14 - DNA, 15 bp, Homo sapiens
  source 1..15: mol_type = genomic DNA
ttgcttaccgccaga

SEQ ID NO: 15 - DNA, 15 bp, Homo sapiens
  source 1..15: mol_type = genomic DNA
tgtcctacccaactt

SEQ ID NO: 16 - DNA, 15 bp, Homo sapiens
  source 1..15: mol_type = genomic DNA
gcgatggtagattat

SEQ ID NO: 17 - DNA, 15 bp, Homo sapiens
  source 1..15: mol_type = genomic DNA
aatagcgagcaagta

SEQ ID NO: 18 - DNA, 15 bp, Homo sapiens
  source 1..15: mol_type = genomic DNA
agcttacctgcacga

SEQ ID NO: 19 - DNA, 15 bp, Homo sapiens
  source 1..15: mol_type = genomic DNA
gatccctttgtcact

SEQ ID NO: 20 - DNA, 15 bp, Homo sapiens
  source 1..15: mol_type = genomic DNA
aattcaaccgtcgcc

SEQ ID NO: 21 - DNA, 15 bp, Homo sapiens
  source 1..15: mol_type = genomic DNA
gttcctatggacgac

SEQ ID NO: 22 - DNA, 15 bp, Homo sapiens
  source 1..15: mol_type = genomic DNA
gtacgcagtccttct

SEQ ID NO: 23 - DNA, 15 bp, Homo sapiens
  source 1..15: mol_type = genomic DNA
gtcaactctttagcg

SEQ ID NO: 24 - DNA, 15 bp, Homo sapiens
  source 1..15: mol_type = genomic DNA
tgatggcctattggg

SEQ ID NO: 25 - DNA, 15 bp, Homo sapiens
  source 1..15: mol_type = genomic DNA
ttccgcctctctttg

SEQ ID NO: 26 - DNA, 15 bp, Homo sapiens
  source 1..15: mol_type = genomic DNA
agtaagttcagcgta

SEQ ID NO: 27 - DNA, 15 bp, Homo sapiens
  source 1..15: mol_type = genomic DNA
ctgggcaattactcg

SEQ ID NO: 28 - DNA, 15 bp, Homo sapiens
  source 1..15: mol_type = genomic DNA
ttctgggtccctaga

SEQ ID NO: 29 - DNA, 15 bp, Homo sapiens
  source 1..15: mol_type = genomic DNA
agattccttcgtgtt

SEQ ID NO: 30 - DNA, 15 bp, Homo sapiens
  source 1..15: mol_type = genomic DNA
tagcgagcccgtata

SEQ ID NO: 31 - DNA, 15 bp, Homo sapiens
  source 1..15: mol_type = genomic DNA
ctggagcgattagaa

SEQ ID NO: 32 - DNA, 15 bp, Homo sapiens
  source 1..15: mol_type = genomic DNA
cagtaagttcgggac

SEQ ID NO: 33 - DNA, 15 bp, Homo sapiens
  source 1..15: mol_type = genomic DNA
caatcagacctatga

SEQ ID NO: 34 - DNA, 15 bp, Homo sapiens
  source 1..15: mol_type = genomic DNA
aagttcactctttgc

SEQ ID NO: 35 - DNA, 15 bp, Homo sapiens
  source 1..15: mol_type = genomic DNA
cttcactctgtcagg

SEQ ID NO: 36 - DNA, 15 bp, Homo sapiens
  source 1..15: mol_type = genomic DNA
gagctacttcactcg

SEQ ID NO: 37 - DNA, 15 bp, Homo sapiens
  source 1..15: mol_type = genomic DNA
cgtaacgtagagcga

SEQ ID NO: 38 - DNA, 15 bp, Homo sapiens
  source 1..15: mol_type = genomic DNA
tcaggtcctttcaac

SEQ ID NO: 39 - DNA, 15 bp, Homo sapiens
  source 1..15: mol_type = genomic DNA
ccaaagtaagagcca

SEQ ID NO: 40 - DNA, 15 bp, Homo sapiens
  source 1..15: mol_type = genomic DNA
tcagtcagatggtat

SEQ ID NO: 41 - DNA, 24 bp, synthetic construct
  source 1..24: mol_type = other DNA
accgcagtcatatgtgctacccca

SEQ ID NO: 42 - DNA, 24 bp, synthetic construct
  source 1..24: mol_type = other DNA
aaactggggtagcacatatgactg

SEQ ID NO: 43 - DNA, 24 bp, synthetic construct
  source 1..24: mol_type = other DNA
accgggtcacatgaaatccagggg

SEQ ID NO: 44 - DNA, 24 bp, synthetic construct
  source 1..24: mol_type = other DNA
aaaccccctggatttcatgtgacc

SEQ ID NO: 45 - DNA, 24 bp, synthetic construct
  source 1..24: mol_type = other DNA
accggcagacgaatcagactgggt

SEQ ID NO: 46 - DNA, 24 bp, synthetic construct
  source 1..24: mol_type = other DNA
aaacacccagtctgattcgtctgc

SEQ ID NO: 47 - DNA, 24 bp, synthetic construct
  source 1..24: mol_type = other DNA
accgtgaatttgtaaggttagaga

SEQ ID NO: 48 - DNA, 24 bp, synthetic construct
  source 1..24: mol_type = other DNA
aaactctctaaccttacaaattca

SEQ ID NO: 49 - DNA, 24 bp, synthetic construct
  source 1..24: mol_type = other DNA
accggagatggcaagagctacttc

SEQ ID NO: 50 - DNA, 24 bp, synthetic construct
  source 1..24: mol_type = other DNA
aaacgaagtagctcttgccatctc

SEQ ID NO: 51 - DNA, 24 bp, synthetic construct
  source 1..24: mol_type = other DNA
accggtgctgctttatagcttact

SEQ ID NO: 52 - DNA, 24 bp, synthetic construct
  source 1..24: mol_type = other DNA
aaacagtaagctataaagcagcac

SEQ ID NO: 53 - DNA, 24 bp, synthetic construct
  source 1..24: mol_type = other DNA
accgcacaagctcgtggaagacag

SEQ ID NO: 54 - DNA, 24 bp, synthetic construct
  source 1..24: mol_type = other DNA
aaacctgtcttccacgagcttgtg

SEQ ID NO: 55 - DNA, 24 bp, synthetic construct
  source 1..24: mol_type = other DNA
accgccttgaacacaaaaaacctg

SEQ ID NO: 56 - DNA, 24 bp, synthetic construct
  source 1..24: mol_type = other DNA
aaaccaggttttttgtgttcaagg

SEQ ID NO: 57 - DNA, 24 bp, synthetic construct
  source 1..24: mol_type = other DNA
accgaagcaggtgatatcctccag

SEQ ID NO: 58 - DNA, 24 bp, synthetic construct
  source 1..24: mol_type = other DNA
aaacctggaggatatcacctgctt

SEQ ID NO: 59 - DNA, 24 bp, synthetic construct
  source 1..24: mol_type = other DNA
accgttgtaaaggactactcacag

SEQ ID NO: 60 - DNA, 24 bp, synthetic construct
  source 1..24: mol_type = other DNA
aaacctgtgagtagtcctttacaa

SEQ ID NO: 61 - DNA, 24 bp, synthetic construct
  source 1..24: mol_type = other DNA
accgtgtgtgatgcatccagtctg

SEQ ID NO: 62 - DNA, 24 bp, synthetic construct
  source 1..24: mol_type = other DNA
aaaccagactggatgcatcacaca

SEQ ID NO: 63 - DNA, 24 bp, synthetic construct
  source 1..24: mol_type = other DNA
accgcagatcttagaacctacagc

SEQ ID NO: 64 - DNA, 24 bp, synthetic construct
  source 1..24: mol_type = other DNA
aaacgctgtaggttctaagatctg

SEQ ID NO: 65 - DNA, 24 bp, synthetic construct
  source 1..24: mol_type = other DNA
accgtcacatcagaccacatccag

SEQ ID NO: 66 - DNA, 24 bp, synthetic construct
  source 1..24: mol_type = other DNA
aaacctggatgtggtctgatgtga

SEQ ID NO: 67 - DNA, 24 bp, synthetic construct
  source 1..24: mol_type = other DNA
accgggagcagagaacttgtgttg

SEQ ID NO: 68 - DNA, 24 bp, synthetic construct
  source 1..24: mol_type = other DNA
aaaccaacacaagttctctgctcc

SEQ ID NO: 69 - DNA, 24 bp, synthetic construct
  source 1..24: mol_type = other DNA
accgtggtgggattcctgctcaca

SEQ ID NO: 70 - DNA, 24 bp, synthetic construct
  source 1..24: mol_type = other DNA
aaactgtgagcaggaatcccacca

SEQ ID NO: 71 - DNA, 24 bp, synthetic construct
  source 1..24: mol_type = other DNA
accgtgagtggatgtattcccaga

SEQ ID NO: 72 - DNA, 24 bp, synthetic construct
  source 1..24: mol_type = other DNA
aaactctgggaatacatccactca

SEQ ID NO: 73 - DNA, 24 bp, synthetic construct
  source 1..24: mol_type = other DNA
accgtattcccagacggatgcggg

SEQ ID NO: 74 - DNA, 24 bp, synthetic construct
  source 1..24: mol_type = other DNA
aaaccccgcatccgtctgggaata

SEQ ID NO: 75 - DNA, 24 bp, synthetic construct
  source 1..24: mol_type = other DNA
accgtacccgagagtaccctctgg

SEQ ID NO: 76 - DNA, 24 bp, synthetic construct
  source 1..24: mol_type = other DNA
aaacccagagggtactctcgggta

SEQ ID NO: 77 - DNA, 24 bp, synthetic construct
  source 1..24: mol_type = other DNA
accgcatgagacttgttcttcttt

SEQ ID NO: 78 - DNA, 24 bp, synthetic construct
  source 1..24: mol_type = other DNA
aaacaaagaagaacaagtctcatg

SEQ ID NO: 79 - DNA, 24 bp, synthetic construct
  source 1..24: mol_type = other DNA
accgacccactgttaggggctggg

SEQ ID NO: 80 - DNA, 24 bp, synthetic construct
  source 1..24: mol_type = other DNA
aaaccccagcccctaacagtgggt

SEQ ID NO: 81 - DNA, 24 bp, synthetic construct
  source 1..24: mol_type = other DNA
accgaagagatctattgtagaaca

SEQ ID NO: 82 - DNA, 24 bp, synthetic construct
  source 1..24: mol_type = other DNA
aaactgttctacaatagatctctt

SEQ ID NO: 83 - DNA, 24 bp, synthetic construct
  source 1..24: mol_type = other DNA
accgtagagcccaagtaagcgttg

SEQ ID NO: 84 - DNA, 24 bp, synthetic construct
  source 1..24: mol_type = other DNA
aaaccaacgcttacttgggctcta

SEQ ID NO: 85 - DNA, 24 bp, synthetic construct
  source 1..24: mol_type = other DNA
accgcgaagagaggaagctctcgg

SEQ ID NO: 86 - DNA, 24 bp, synthetic construct
  source 1..24: mol_type = other DNA
aaacccgagagcttcctctcttcg

SEQ ID NO: 87 - DNA, 24 bp, synthetic construct
  source 1..24: mol_type = other DNA
accgaaaacgtgtgggtacgacct

SEQ ID NO: 88 - DNA, 24 bp, synthetic construct
  source 1..24: mol_type = other DNA
aaacaggtcgtacccacacgtttt

SEQ ID NO: 89 - DNA, 24 bp, synthetic construct
  source 1..24: mol_type = other DNA
accggcgataatgactgataaggg

SEQ ID NO: 90 - DNA, 24 bp, synthetic construct
  source 1..24: mol_type = other DNA
aaaccccttatcagtcattatcgc

SEQ ID NO: 91 - DNA, 24 bp, synthetic construct
  source 1..24: mol_type = other DNA
accgcgataatgactgataagggc

SEQ ID NO: 92 - DNA, 24 bp, synthetic construct
  source 1..24: mol_type = other DNA
aaacgcccttatcagtcattatcg

SEQ ID NO: 93 - DNA, 24 bp, synthetic construct
  source 1..24: mol_type = other DNA
accgtcagtgttgcccttgcaatg

SEQ ID NO: 94 - DNA, 24 bp, synthetic construct
  source 1..24: mol_type = other DNA
aaaccattgcaagggcaacactga

SEQ ID NO: 95 - DNA, 24 bp, synthetic construct
  source 1..24: mol_type = other DNA
accgcccttatcagtcattatcgc

SEQ ID NO: 96 - DNA, 24 bp, synthetic construct
  source 1..24: mol_type = other DNA
aaacgcgataatgactgataaggg

SEQ ID NO: 97 - DNA, 24 bp, synthetic construct
  source 1..24: mol_type = other DNA
accgtcggttgagactgcttgcac

SEQ ID NO: 98 - DNA, 24 bp, synthetic construct
  source 1..24: mol_type = other DNA
aaacgtgcaagcagtctcaaccga

SEQ ID NO: 99 - DNA, 24 bp, synthetic construct
  source 1..24: mol_type = other DNA
accgccctgtatcgtggctgataa

SEQ ID NO: 100 - DNA, 24 bp, synthetic construct
  source 1..24: mol_type = other DNA
aaacttatcagccacgatacaggg

SEQ ID NO: 101 - DNA, 24 bp, synthetic construct
  source 1..24: mol_type = other DNA
accggatggtataagccgagaaag

SEQ ID NO: 102 - DNA, 24 bp, synthetic construct
  source 1..24: mol_type = other DNA
aaacctttctcggcttataccatc

SEQ ID NO: 103 - DNA, 24 bp, synthetic construct
  source 1..24: mol_type = other DNA
accgctgtttatctgcagcagctc

SEQ ID NO: 104 - DNA, 24 bp, synthetic construct
  source 1..24: mol_type = other DNA
aaacgagctgctgcagataaacag

SEQ ID NO: 105 - DNA, 24 bp, synthetic construct
  source 1..24: mol_type = other DNA
accgctgaagtgggtacactcttt

SEQ ID NO: 106 - DNA, 24 bp, synthetic construct
  source 1..24: mol_type = other DNA
aaacaaagagtgtacccacttcag

SEQ ID NO: 107 - DNA, 24 bp, synthetic construct
  source 1..24: mol_type = other DNA
accgatataggtaaagccagaccg

SEQ ID NO: 108 - DNA, 24 bp, synthetic construct
  source 1..24: mol_type = other DNA
aaaccggtctggctttacctatat

SEQ ID NO: 109 - DNA, 24 bp, synthetic construct
  source 1..24: mol_type = other DNA
accggcttctcgattatgctacac

SEQ ID NO: 110 - DNA, 24 bp, synthetic construct
  source 1..24: mol_type = other DNA
aaacgtgtagcataatcgagaagc

SEQ ID NO: 111 - DNA, 24 bp, synthetic construct
  source 1..24: mol_type = other DNA
accgctcttggtgtcactgtggtt

SEQ ID NO: 112 - DNA, 24 bp, synthetic construct
  source 1..24: mol_type = other DNA
aaacaaccacagtgacaccaagag

SEQ ID NO: 113 - DNA, 24 bp, synthetic construct
  source 1..24: mol_type = other DNA
accggcatcatttattgtgtagcg

SEQ ID NO: 114 - DNA, 24 bp, synthetic construct
  source 1..24: mol_type = other DNA
aaaccgctacacaataaatgatgc

SEQ ID NO: 115 - DNA, 24 bp, synthetic construct
  source 1..24: mol_type = other DNA
accgcattgctgctgttcctaaat

SEQ ID NO: 116 - DNA, 24 bp, synthetic construct
  source 1..24: mol_type = other DNA
aaacatttaggaacagcagcaatg

SEQ ID NO: 117 - DNA, 24 bp, synthetic construct
  source 1..24: mol_type = other DNA
accgtggtgaagtgcttaggctgt

SEQ ID NO: 118 - DNA, 24 bp, synthetic construct
  source 1..24: mol_type = other DNA
aaacacagcctaagcacttcacca

SEQ ID NO: 119 - DNA, 24 bp, synthetic construct
  source 1..24: mol_type = other DNA
accggtatctttgcacatcctgcc

SEQ ID NO: 120 - DNA, 24 bp, synthetic construct
  source 1..24: mol_type = other DNA
aaacggcaggatgtgcaaagatac

SEQ ID NO: 121 - DNA, 24 bp, synthetic construct
  source 1..24: mol_type = other DNA
accggcagtcacctcatatgtcag

SEQ ID NO: 122 - DNA, 24 bp, synthetic construct
  source 1..24: mol_type = other DNA
aaacctgacatatgaggtgactgc

SEQ ID NO: 123 - DNA, 24 bp, synthetic construct
  source 1..24: mol_type = other DNA
accgcatatgtcagagggcactgt

SEQ ID NO: 124 - DNA, 24 bp, synthetic construct
  source 1..24: mol_type = other DNA
aaacacagtgccctctgacatatg

SEQ ID NO: 125 - DNA, 24 bp, synthetic construct
  source 1..24: mol_type = other DNA
accgggaaatgccatgaactctag

SEQ ID NO: 126 - DNA, 24 bp, synthetic construct
  source 1..24: mol_type = other DNA
aaacctagagttcatggcatttcc

SEQ ID NO: 127 - DNA, 24 bp, synthetic construct
  source 1..24: mol_type = other DNA
accgagtcatcactgcgtgaatgc

SEQ ID NO: 128 - DNA, 24 bp, synthetic construct
  source 1..24: mol_type = other DNA
aaacgcattcacgcagtgatgact

SEQ ID NO: 129 - DNA, 24 bp, synthetic construct
  source 1..24: mol_type = other DNA
accgctcagcttcctggtgacaga

SEQ ID NO: 130 - DNA, 24 bp, synthetic construct
  source 1..24: mol_type = other DNA
aaactctgtcaccaggaagctgag

SEQ ID NO: 131 - DNA, 24 bp, synthetic construct
  source 1..24: mol_type = other DNA
accgacgttttcctttctgctctg

SEQ ID NO: 132 - DNA, 24 bp, synthetic construct
  source 1..24: mol_type = other DNA
aaaccagagcagaaaggaaaacgt

SEQ ID NO: 133 - DNA, 24 bp, synthetic construct
  source 1..24: mol_type = other DNA
accgtcccaggtgacagtcaacag

SEQ ID NO: 134 - DNA, 24 bp, synthetic construct
  source 1..24: mol_type = other DNA
aaacctgttgactgtcacctggga

SEQ ID NO: 135 - DNA, 24 bp, synthetic construct
  source 1..24: mol_type = other DNA
accgcaaacaccagatgttcagag

SEQ ID NO: 136 - DNA, 24 bp, synthetic construct
  source 1..24: mol_type = other DNA
aaacctctgaacatctggtgtttg

SEQ ID NO: 137 - DNA, 24 bp, synthetic construct
  source 1..24: mol_type = other DNA
accgattacattctgaggactcag

SEQ ID NO: 138 - DNA, 24 bp, synthetic construct
  source 1..24: mol_type = other DNA
aaacctgagtcctcagaatgtaat

SEQ ID NO: 139 - DNA, 24 bp, synthetic construct
  source 1..24: mol_type = other DNA
accgagagtcaagaaacctgacag

SEQ ID NO: 140 - DNA, 24 bp, synthetic construct
  source 1..24: mol_type = other DNA
aaacctgtcaggtttcttgactct

SEQ ID NO: 141 - DNA, 24 bp, synthetic construct
  source 1..24: mol_type = other DNA
accgccattactaggttctccctg

SEQ ID NO: 142 - DNA, 24 bp, synthetic construct
  source 1..24: mol_type = other DNA
aaaccagggagaacctagtaatgg

SEQ ID NO: 143 - DNA, 24 bp, synthetic construct
  source 1..24: mol_type = other DNA
accgctgatttcagtagtgaaact

SEQ ID NO: 144 - DNA, 24 bp, synthetic construct
  source 1..24: mol_type = other DNA
aaacagtttcactactgaaatcag

SEQ ID NO: 145 - DNA, 24 bp, synthetic construct
  source 1..24: mol_type = other DNA
accgcttgtccttcacgaagccgg

SEQ ID NO: 146 - DNA, 24 bp, synthetic construct
  source 1..24: mol_type = other DNA
aaacccggcttcgtgaaggacaag

SEQ ID NO: 147 - DNA, 24 bp, synthetic construct
  source 1..24: mol_type = other DNA
accgtgaatcggctggtagttggt

SEQ ID NO: 148 - DNA, 24 bp, synthetic construct
  source 1..24: mol_type = other DNA
aaacaccaactaccagccgattca

SEQ ID NO: 149 - DNA, 24 bp, synthetic construct
  source 1..24: mol_type = other DNA
accgaatagcattcaggatgtacg

SEQ ID NO: 150 - DNA, 24 bp, synthetic construct
  source 1..24: mol_type = other DNA
aaaccgtacatcctgaatgctatt

SEQ ID NO: 151 - DNA, 24 bp, synthetic construct
  source 1..24: mol_type = other DNA
accgaaagagctaagaaacctctg

SEQ ID NO: 152 - DNA, 24 bp, synthetic construct
  source 1..24: mol_type = other DNA
aaaccagaggtttcttagctcttt

SEQ ID NO: 153 - DNA, 24 bp, synthetic construct
  source 1..24: mol_type = other DNA
accggaaacacaagatatgtgctg

SEQ ID NO: 154 - DNA, 24 bp, synthetic construct
  source 1..24: mol_type = other DNA
aaaccagcacatatcttgtgtttc

SEQ ID NO: 155 - DNA, 24 bp, synthetic construct
  source 1..24: mol_type = other DNA
accgcgcaaggatctggtccacag

SEQ ID NO: 156 - DNA, 24 bp, synthetic construct
  source 1..24: mol_type = other DNA
aaacctgtggaccagatccttgcg

SEQ ID NO: 157 - DNA, 24 bp, synthetic construct
  source 1..24: mol_type = other DNA
accgcagtggttcctaccgaaagt

SEQ ID NO: 158 - DNA, 24 bp, synthetic construct
  source 1..24: mol_type = other DNA
aaacactttcggtaggaaccactg

SEQ ID NO: 159 - DNA, 24 bp, synthetic construct
  source 1..24: mol_type = other DNA
accgaatcacctgacttacaatgg

SEQ ID NO: 160 - DNA, 24 bp, synthetic construct
  source 1..24: mol_type = other DNA
aaacccattgtaagtcaggtgatt

SEQ ID NO: 161 - DNA, 24 bp, synthetic construct
  source 1..24: mol_type = other DNA
accggtagcaaaagtagtactctg

SEQ ID NO: 162 - DNA, 24 bp, synthetic construct
  source 1..24: mol_type = other DNA
aaaccagagtactacttttgctac

SEQ ID NO: 163 - DNA, 24 bp, synthetic construct
  source 1..24: mol_type = other DNA
accgcactcggctgcgcgaagtgg

SEQ ID NO: 164 - DNA, 24 bp, synthetic construct
  source 1..24: mol_type = other DNA
aaacccacttcgcgcagccgagtg

SEQ ID NO: 165 - DNA, 11 bp, synthetic construct
  source 1..11: mol_type = other DNA
tgactcatttc

SEQ ID NO: 166 - DNA, 10 bp, synthetic construct
  source 1..10: mol_type = other DNA
aaaaaaaaaa

SEQ ID NO: 167 - DNA, 13 bp, synthetic construct
  source 1..13: mol_type = other DNA
ttttttttttttt

SEQ ID NO: 168 - DNA, 15 bp, synthetic construct
  source 1..15: mol_type = other DNA
aaaaaaaaaaaaaaa

SEQ ID NO: 169 - DNA, 16 bp, synthetic construct
  source 1..16: mol_type = other DNA
aaaaaaaaaaaaaaaa

SEQ ID NO: 170 - DNA, 12 bp, synthetic construct
  source 1..12: mol_type = other DNA
aaaaaaaaaaaa

SEQ ID NO: 171 - DNA, 34 bp, synthetic construct
  source 1..34: mol_type = other DNA
tgtcccagataaggagagggcagtgccacctacg

SEQ ID NO: 172 - DNA, 1193 bp, Homo sapiens
  source 1..1193: mol_type = genomic DNA
gtgtgagtgacttcatggcaccgacattgctgtttttaaatgaggatacagtaaattgcagtccgaggaaggctaactggaatcaacatacccgtagctttagaaagcagtttccgcaccagcgaagagtacaagagcgatggaaccccatgttcctggaagtttgcacatcagagtaaacaaacttgaaaacccctcttgatagcagaattcacccagccttgttccattttctcttaacaaaacacaccgcaaaagctctcacaagctgctttgatgaagccacatgtatttcccccttcacaatttacaggaagttactcttaaaagaaagtgattctggtgtttaccgcctgtgttaaagggacagagttcctttttatttctgataacgtttgagcgaaatacagaaactatctgtagactagcatagtcggtacgtgagtaaggaaaagcaataacctgctgtccggtgagcacaaaattcctgctacgaacagtgccttactgctgcttggagactgcaagtcgcagatcacactaggtattgactgattgtataaggaaatttcttaaagtctaaagtaaaggtggtacctcctaaaaagaggggaagagagaaaactttgtgtggaaggataaggagtgtgtttatagtttcagtaagagtgtacgttttaatttttcttcttcctctgcctctttgccaagtagcctgagtgcatctgttatccagaagtagtattactctaggacaaacttcaaattcttcattctgcgttgcctttaaggaacaacatactttcttcctgttctttttccaaaaacacacgcctatggctctgtgtgtggtgttttagccagcctcctcccagataaggggttcccttccctcctttgcattgaaaggaaagtgcaagtctggacatgtttatcaagaggaaaagtgacttctcagtaatagactgtcaaattcgggctgctgcccgagtgttcgctttgttatggcaggtgaagttcacctttgccccacccagtgtttccacaaaaaggcaaggttccaagtattcatatgaacaagtgttactttaggacttggagggttgggggtggaggatgtttgcatagttgaagccttgggcgggggtgtaggaaacggcgagtacagaggccatagaaaaagctaagactca

SEQ ID NO: 173 - DNA, 520 bp, Homo sapiens
  source 1..520: mol_type = genomic DNA
agttctgccaagtttgaacgttagcaggagaaacggaatgttacaacttttggggcggggggcggggaaacgtgcgttacatacacaacagcttgaggaccagacagctccactgtattacactagctgcaaaaacaatttaacttgctcttttgaagtaagatttgtgtcttttgtacctggggatttgagctgagaaatcagaaactgtgtaggtaaattttaagtttccttaatttaaggaacgtgcgccccctaattctgccgcgccaggaaggagggcgatctggagtgtttagaatacaatagagcccaagtaagcgttgaggttaagtgccttcaaagggaagtaagaagattccaagtcaatgttgaaatacacatgcgaagagaggaagctctcggcggctgtgctagcaatctgggggaaagccctgggctcggccaggtggtgttggccacgttggccacgcccccacttcgcgcagccgagtggctaaggaaatcttaagcagggagg

SEQ ID NO: 174 - DNA, 398 bp, Homo sapiens
  source 1..398: mol_type = genomic DNA
cacaagccacatgagacttgttcttcttttcttgaataaaagttttatcaaagttatgcaaattagtatagacccactgttaggggctgggaggagaagcagtcaatgtaacatgaaataaaatagactaagatcaaaaacaaactattctgtttagctgactcatttcaaaatgaaatcagtaattttgtaacaaggggtgggggatcagcagatgttttcaaaggatacgaaatttcagttagataagagatgtaagttcaagagatctattgtagaacaaggtgtttatatagttaataacagtgtatttctgtaaattgctaggagagtagattttaagtgttctcaccacaaaaaaagagacatgagataatgcatattgctaattagctcaa

(19) United States
(12) Patent Application Publication
(10) Pub. No.: US 2026/0117178 A1
(43) Pub. Date: Apr. 30, 2026

(54) MEANS TO TREAT AUTOIMMUNITY BY REFINING REGULATORY T CELLS

(71) Applicant: Yale University, New Haven, CT (US)

(72) Inventors: Tomokazu SUMIDA, New Haven, CT (US); David HAFLER, New Haven, CT (US)

(73) Assignee: Yale University, New Haven, CT (US)

(21) Appl. No.: 19/112,073

(22) PCT Filed: Sep. 15, 2023
(86) PCT No.: PCT/US23/74346

§ 371 Date: Mar. 14, 2025

Related U.S. Application Data
(60) Provisional application No. 63/407,293, filed on Sep. 16, 2022.

(51) Int. Cl. C12N 5/0783; A61K 31/7088; A61K 38/00; C07K 16/00; C12N 15/113

(52) U.S. Cl. CPC C12N 5/0637; A61K 31/7088; A61K 38/00; C07K 16/00; C12N 15/113; C12N 2310/14; C12N 2310/16; C12N 2310/20; C12N 2501/40; C12N 2503/02

(57) ABSTRACT

The present disclosure provides methods for restoring regulatory T cell (Treg) function. Methods of treating a disease (e.g., an autoimmune disease or a cancer) in a subject in need thereof are also disclosed. The present disclosure further provides methods of screening for a compound for restoring Treg function.

### CROSS REFERENCE TO RELATED APPLICATIONS

This patent application claims the benefit of U.S. Provisional Application No. 63/407,293, filed Sep. 16, 2022, the disclosure of which is incorporated by reference herein in its entirety for all purposes.

### STATEMENT REGARDING FEDERALLY SPONSORED RESEARCH

This invention was made with government support under AI039671 awarded by National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Sep. 15, 2023, is named 251609_000093_SL.xml and is 156,044 bytes in size.

### FIELD OF THE INVENTION

The present disclosure relates to methods for restoring regulatory T cell (Treg) function. Methods of treating a disease (e.g., an autoimmune disease or a cancer) in a subject in need thereof are also disclosed. The present disclosure further relates to methods of screening for a compound for restoring Treg function.

### BACKGROUND

Human autoimmune diseases constitute a leading cause of death among young adults with an increasing incidence in recent years. Multiple sclerosis (MS) is a canonical, genetically mediated autoimmune disease induced by environmental factors where genetic perturbation of cis-regulatory elements in pathogenic immune cells leads to immune dysregulation and generation of autoreactive T cells and antibodies (4-6). Among immune cells, CD4⁺ (cluster of differentiation 4 positive) T cells play a central role in both mediating and regulating autoimmunity. As CD4⁺ T cells display a large degree of functional diversity, interrogation of total CD4⁺ T cell populations has not to date identified causal transcriptional changes (7, 8). Thus, interrogation of CD4⁺ T cell subpopulations is required to understand the pathophysiological characteristics of CD4⁺ T cells in autoimmune disorders in order to identify a central transcriptional factor associated with loss of immune regulation, perhaps linked to genetic variation associated with disease risk.

Human CD4⁺ Foxp3⁺ (forkhead box P3 positive) regulatory T cells (Tregs) play a central role in the maintenance of immune homeostasis and prevention of autoimmunity (9-11). Tregs from patients with autoimmune disease exhibit a dysfunctional phenotype (12-14) and this has been found among multiple autoimmune disorders. Evidence has shown that environmental factors, such as vitamin D and fatty acids, affect Treg phenotype and function. In addition, high salt intake has been epidemiologically linked with autoimmune diseases (15) and higher physiologic salt concentrations induce proinflammatory T helper 17 cells (Th17) cells mediated by serum- and glucocorticoid-inducible kinase 1 (SGK1) (16) and modulate the stability of Tregs (17, 18), resembling the phenotype observed in autoimmune diseases including MS (19). These environmental factors act, in part, through epigenetic changes that can be identified by examining the histone and methylation landscape of cells (20). An investigation of the genetic architecture of 20 autoimmune diseases and MS showed that causal variants may be enriched in regulatory elements that are active in immune cells (6). Moreover, many of these genetic loci are common among multiple autoimmune diseases, implicating the shared immunomodulatory mechanism in human autoimmunity (21, 22). These findings, together with increases in the incidence of autoimmune diseases over the past three decades that may not be explained by genetic factors alone, point to both genetic and epigenetic factors as mediators of risk for autoimmunity. While these lines of evidence suggest that both genetic and epigenetic alterations might disturb Treg homeostasis, the underlying mechanism in Treg dysfunction in human autoimmune diseases has not been elucidated.

### SUMMARY OF THE INVENTION

As specified in the Background section above, there is a great need in the art for identifying underlying mechanism(s) in Treg dysfunction in disease, e.g., in an autoimmune disease including MS and/or a cancer, in particular, toward development of methods and compositions for the restoration of Treg function, as well as methods for treating subjects in need thereof (e.g., subjects afflicted with an autoimmune disease and/or a cancer) involving such methods and/or compositions. The present application addresses these and other needs.

In the present disclosure, the phenotypic and functional characteristics of human CD4⁺ T cells were interrogated, focusing primarily on memory Treg (mTreg) through comprehensive transcriptomic and epigenetic profiling. Both bulk and single-cell RNA-seq (scRNA-seq) were adopted for transcriptional profiling and Assay for Transposase-Accessible Chromatin using sequencing (ATAC-seq) was adopted for probing epigenetic regulation to understand the molecular mechanisms that drive dysfunctional programs in MS Tregs. Reported herein are findings that PRDM1 (PR/SET Domain 1), which encodes the protein Blimp1 (B lymphocyte-induced maturation protein-1), is upregulated in both mTreg and mTconv from patients with MS with a more significant increase in mTreg. This transcriptional signature of Tregs is shared across different autoimmune diseases, suggesting the common transcriptional signature driving dysfunctional Tregs in human autoimmunity. Specifically, an alternative short isoform of PRDM1 (PRDM1-S), which codes for protein in primates but not in rodents, is primarily elevated in MS mTreg compared to the evolutionary conserved long PRDM1 isoform (PRDM1-L). This short isoform disturbs PRDM1-L-mediated gene regulation and drives a dysfunctional Th17-like program in MS Tregs. Gene overexpression experiments in primary human Tregs, together with bulk and single-cell RNA-seq transcriptional profiling described herein, demonstrate a unique link between the PRDM1-S and SGK1 that can account for Treg dysfunction observed in MS. Moreover, while genome-wide chromatin accessibility in mTreg remains comparable between MS and healthy controls, the transcription factor footprints and motifs within accessible chromatin regions are different; significantly enriched binding of Activator protein 1 (AP-1) and interferon-regulatory factor (IRF) family transcription factors was observed in MS mTreg, suggesting rewiring of regulatory circuits. Given that AP-1 activation can be induced by the CD28 pathway, a positive expression quantitative trait locus (eQTL) effect of the autoimmune risk loci at CD28 region supports a genetic mechanism for enrichment of AP-1 in MS Treg. CRISPR activation (CRISPRa)-based validation of cis-regulatory elements for the PRDM1-S identified an active enhancer element with Activator protein 1 (AP-1) and interferon-regulatory factor (IRF) transcription factor (TF) binding motifs. These findings reveal a regulatory program by which Tregs become dysfunctional in humans that is shared across multiple autoimmune diseases. Multimodal datasets of human CD4⁺ T cells reported herein provide a rich resource for understanding the loss of immune regulation in autoimmune diseases and suggest that the primate specific short PRDM1 isoform is a critical, targetable transcriptional regulator in human autoimmunity.

In one aspect, provided herein is a method of restoring a regulatory T cell (Treg) function comprising contacting the Treg with an effective amount of an agent capable of reducing the expression and/or function of a short isoform of PRDM1 (PRDM1-S).

In some embodiments, the agent inhibits the expression and/or function of the protein (Blimp1-S) encoded by PRDM1-S gene.

In some embodiments, the agent inhibits the expression of the mRNA transcribed from PRDM1-S promoter.

In some embodiments, the agent blocks one or more of a regulatory element(s) that controls the transcription of PRDM1-S mRNA.

In some embodiments, the regulatory element is a promoter, an enhancer or an insulator.

In some embodiments, the agent is a siRNA, a shRNA, a miRNA, or an antisense oligonucleotide.

In some embodiments, the agent is a small molecule, an antibody or antigen-binding fragment thereof, or an aptamer.

In some embodiments, the agent is a CRISPR interference (CRISPRi) or CRISPRoff molecule.

In another aspect, provided herein is a method of restoring a regulatory T cell (Treg) function comprising contacting the Treg with an effective amount of an agent capable of increasing the expression and/or function of a long isoform of PRDM1 (PRDM1-L).

In some embodiments, the agent activates one or more regulatory element(s) that controls the transcription of PRDM1-L mRNA.

In some embodiments, the regulatory element is a promoter, an enhancer or an insulator.

In some embodiments, the agent is a CRISPR activation (CRISPRa) molecule.

In some embodiments, the contacting occurs in vivo.

In some embodiments, the contacting occurs ex vivo.

In some embodiments, the Treg is in a subject and the agent is administered to the subject.

In another aspect, provided herein is a method of treating a disease in a subject in need thereof comprising administering to the subject an effective amount of an agent capable of reducing the expression and/or function of a short isoform of PRDM1 (PRDM1-S).

In some embodiments, the agent inhibits the expression and/or function of the protein (Blimp1-S) encoded by PRDM1-S gene.

In some embodiments, the agent inhibits the expression of the mRNA transcribed from PRDM1-S promoter.

In some embodiments, the agent blocks the one or more regulatory element(s) that controls the transcription of PRDM1-S mRNA.

In some embodiments, the regulatory element is a promoter, an enhancer or an insulator.

In some embodiments, the agent is a siRNA, a shRNA, a miRNA, or an antisense RNA.

In some embodiments, the agent is a small molecule, an antibody or antigen-binding fragment thereof, or an aptamer.

In some embodiments, the agent is a CRISPR interference (CRISPRi) or CRISPRoff molecule.

In another aspect, provided herein is a method of treating a disease in a subject in need thereof comprising administering to the subject an effective amount of an agent capable of increasing the expression and/or function of a long isoform of PRDM1 (PRDM1-L).

In some embodiments, the agent activates one or more regulatory element(s) that controls the expression of PRDM1-L mRNA.

In some embodiments, the regulatory element is a promoter, an enhancer or an insulator.

In some embodiments, the agent is a CRISPR activation (CRISPRa) molecule.

In some embodiments, the disease is an autoimmune disease, an acute viral infection, a chronic viral infection, a cardiovascular disease, a neurodegenerative disease, a metabolic syndrome, or a cancer.

In some embodiments, the autoimmune disease is multiple sclerosis.

In some embodiments, the subject is a dry-nosed mammal.

In some embodiments, the subject is a human.

In another aspect, provided herein is a method of screening for a candidate compound that restores a regulatory T cell (Treg) function, comprising: (a) determining the expression or function of a short isoform of PRDM1 (PRDM1-S) and/or a long form of PRDM1 (PRDM1-L) in the Treg before and after contacting said Treg with a test compound; and (b) selecting a compound that i) reduces the expression and/or function of PRDM1-S in comparison with the expression and/or function of PRDM1-S determined in the absence of the test compound; and/or ii) increases the expression or function of long form of PRDM1 (PRDM1-L) in comparison with the expression and/or function of PRDM1-L determined in the absence of the test compound.

### BRIEF DESCRIPTION OF THE DRAWINGS

FIGS. 1A-1F show deep transcriptomic analysis of memory regulatory T-cells (Treg) and Tconv highlight PRDM1 upregulation in multiple sclerosis (MS). (FIG. 1A) Schematic of study design. Memory Tconv (mTconv) or memory Tregs (mTreg) were fluorescence-activated cell sorting (FACS) sorted from peripheral blood CD4⁺ T cells from patients with MS and healthy control subjects (HC). Bulk RNA-seq was performed on the discovery cohort and differentially expressed genes (DEGs) were identified (HC: n=20, MS: n=26). The selected DEGs were validated by an independent validation cohort (HC: n=23, MS: n=16). (FIG. 1B) Volcano plots showing DEGs for mTreg and mTconv between MS and HC. (FIG. 1C) Overlapped DEGs between mTreg and mTconv. (FIG. 1D) qPCR validation for PRDM1 expression in both discovery and validation cohorts. (FIG. 1E) Protein validation for Blimp1 expression using flow cytometry (HC; n=12, MS; n=9). (FIG. 1F) Heatmap depicting expression patterns of selected six genes in mTreg/Fr2 eTreg across 12 autoimmune diseases. P*<0.05, P**<0.01; Statistical significance computed by unpaired t-test (FIG. 1D, FIG. 1E).

FIGS. 2A-2H demonstrate single-cell dual omics analysis reveals elevated PRDM1 in Th17-like Treg in MS. (FIG. 2A) Surface protein guided CD4⁺ T cells subtype annotation. Four CD4⁺ T cell subtypes were distinguished by CD25, CD127, CD45RO, and CD45RA protein expression. (FIG. 2B) Uniform Manifold Approximation and Projection (UMAP) based on gene expression for CD4⁺ T cells demonstrating decent overlap with protein-based subtype annotation. (FIG. 2C) FOXP3, IKZF2 (IKAROS Family Zinc Finger 2), PRDM1, and BACH2 (BTB Domain And CNC Homolog 2) expressions on UMAP (all cells passed QC [quality control] are plotted). (FIG. 2D) Combined differential analysis for bulk- and scRNA-seq in mTreg. Representative differentially expressed genes with pseudo-bulk analysis with scRNA-seq are shown. Gene expression changes in MS relative to control with indicated genes are computed at the single-cell level (see, e.g., Methods described herein, for details) (2). The size of dots is scaled proportionally to the average number of mRNA reads quantified within each batch and condition; the y-axis (disease effect) shows average gene expression after adjusted by confounding factors by matching; the error bars capture one standard deviation for the disease effects in Bayesian inference. Experimental batches for paired HC and MS samples are color coded (#1-5). (FIG. 2E) Combined differential analysis for bulk- and scRNA-seq in mTconv. (FIG. 2F) Sub-cell type analysis based on CITE-seq. Surface CXCR3 (CD183) and CCR6 (CD196) protein expressions of four subtypes are shown (log 10 scale). (FIG. 2G) Heatmaps showing the marker genes and proteins to define subtypes for each mTconv and mTreg. (FIG. 2H) The changes of PRDM1 expression at subtype-level analysis in mTreg between MS and control subjects.

FIGS. 3A-3H show elevated alternative short PRDM1 isoform in MS mTreg. (FIG. 3A) Schematic of PRDM1 short and long isoforms. PR; PR domain, Pro/Ser; Proline/serine rich region, ZnF; five C2H2 zink fingers. (FIG. 3B) ATAC-seq (mTreg and nTreg), DHS (ENCODE primary Treg, Roadmap primary T cells, ENCODE monocytes, Roadmap Primary B cells), and HiDRA peaks at PRDM1 locus. (FIG. 3C) PRDM1-S and PRDM1-L isoform expression across 9 different immune cell types in peripheral blood assessed by bulk RNA-seq (n=6). (FIG. 3D) Western blot analysis of Blimp1 expression from 8 different immune cell types in peripheral blood. Conventional Blimp1 and alternative Blimp1-S are distinguished by different size. (FIG. 3E) PRDM1-S and PRDM1-L gene expression assessed by bulk RNA-seq in mTreg between MS and HC. (FIG. 3F) Schematic of how PRDM1-L mediated gene regulation can be disrupted by enriched TF binding of RF1/2. (FIG. 3G) Quantile-quantile plot showing the co-expression correlation between PRDM1-L signature genes with PRDM1 at a single-cell level in mTreg for MS and HC. Correlation between PRDM1-L signature genes and PRDM1 expression is stronger in HC compared to MS. Wilcoxon's test p-value as summary values between MS vs HC: p-value=0.005763. (FIG. 3H) Normalized gene expression for PRDM1-S and PRDM1-L on human primary Tregs with and without PRDM1-L specific gene knockdown by lentiviral shRNA transduction. Lentiviral transduced GFP⁺ cells were sorted by FACS at day 5. mRNAs were isolated and bulk RNA-seq was performed.

FIGS. 4A-4E show short PRDM1 induces SGK1 and Treg dysfunction. (FIG. 4A) Volcano plot showing statistical significance and fold change for genes differentially expressed by PRDM1-S overexpression in primary mTreg. (FIG. 4B) SGK1 expression was assessed by qPCR for overexpression of PRDM1-S and PRDM1-L with primary human mTreg (left) and Jurkat T cells (right). P***<0.001, P****<0.0001; Statistical significance computed by one-way ANOVA with Dunn's multiple comparisons tests. (FIG. 4C) Pseudo-bulk analysis of SGK1, PRDM1, RORC, IRF4 and BATF expression in scRNA-seq at each of four major mTreg subtypes. (FIG. 4D) In vitro Treg suppression assay with human primary Tregs. T effector cell proliferation was assessed after 5 days of co-culture with Tregs transduced with GFP (green fluorescent protein) control vector vs PRDM1-S overexpression (OE) vector. P**<0.01; Statistical significance computed by two-way repeated measures ANOVA. (FIG. 4E) Flow cytometry analysis for Foxp3 in primary Treg cells by overexpression of PRDM1-S compared to GFP control (n=10). P**<0.01; Statistical significance computed by paired t test.

FIGS. 5A-5F show AP-1 and IRF TF bindings are enriched in MS mTreg. (FIG. 5A) Schematic of ATAC-seq experiments for mTreg from MS (n=26) and healthy control (n=21). (FIG. 5B) TF motif enrichment analysis in mTreg between MS and HC by Hierarchical Independent Component Analysis (HINT). IRF and AP-1 motifs are significantly enriched in MS mTreg. (FIG. 5C) TF footprint enrichment analysis in mTreg between MS and HC by HINT and Transcription factor Occupancy prediction By Investigation of ATAC-seq Signal (TOBIAS). IRF and AP-1 footprints are significantly enriched in MS mTreg. (FIG. 5D) The lead SNP (rs12614091) of MS genome-wide association studies (GWAS) at CD28 locus is cis-eQTL. (FIG. 5E) Heatmap showing the co-expression analysis between PRDM1 isoforms and AP-1 family TF genes in HC and MS mTreg. (FIG. 5F) Schematic of how MS susceptible locus on CD28 is linked with AP-1 enrichment in MS mTreg.

FIGS. 6A-6C show active enhancer element for short PRDM1 with AP-1 and IRF bindings. (FIG. 6A) Schematic experimental overview. 1. Identification of candidate cis-regulatory elements regulating PRDM1 expression from ATAC-seq peaks. 2. CRISPRa based examination of PRDM1-S specific cis-regulatory elements. (FIG. 6B) CRISPRa validation for top 20 PRDM1 associated regulatory elements. Top: Top 20 accessible chromatin elements that are associated with PRDM1 expression are highlighted. Potential interactions of regulatory elements with PRDM1 gene are analyzed by GeneHancer database and shown on the top. Middle and bottom; CRISPRa-induced expression of short PRDM1 (middle) and long PRDM1 (bottom) were assessed by qPCR. Detailed information for all 20 regions is shown in Table 4. (FIG. 6C) Top: H3K27ac, H3K4me1, and H3K4me3 MINT-ChIP signal on the #2 peak region in mTreg from HC and MS. Four replicates of HC and two replicates of MS are merged into one representative track respectively. Middle: Footprint analysis on #2 peak region with TOBIAS footprint score. Bottom: AP-1 and IRF ChIP-seq signals identified on #2 peak region in ENCODE data are shown. AP-1/IRF composite motif identified in #2 peak region is highlighted. FIG. 6C discloses SEQ ID NO: 165.

FIGS. 7A-7F show transcriptomic analysis in mTreg and mTconv with MS patients, related to FIG. 1. (FIG. 7A) Experimental workflow for isolating four major CD4⁺ T cell subpopulations by FACS. (FIG. 7B) Top individual DEGs expression at subject level. (FIG. 7C) Correlation of PRDM1 expression in mTreg and mTconv. (FIG. 7D) qPCR validation for top four DEGs in mTreg. Data for the discovery cohort (left side) and validation cohort (right side) are shown for each HC and MS. (FIG. 7E) PRDM1 expression in Fr2 eTreg and Fr1 nTreg across 10 and 6 autoimmune diseases are shown respectively. (FIG. 7F) PRDM1 and ID3 (Inhibitor Of DNA Binding 3) expression in Fr2 eTreg from HC, systemic lupus erythematosus (SLE), idiopathic inflammatory myopathy (JIM), and antineutrophil cytoplasmic antibody (ANCA)-associated vasculitis (AAV) are shown respectively P*<0.05, P**<0.01, P***<0.001, P****<0.0001; Statistical significance computed by one-way ANOVA with Dunn's multiple comparisons tests.

FIGS. 8A-8G show single-cell dual omics analysis with CD4⁺ T cells in MS, related to FIG. 2. (FIG. 8A) Experimental workflow for dual omics single-cell analysis of HC and MS CD4⁺ T cells. Age, sex, and ethnicity matched HC and MS subject are processed at the same time as one experimental batch. Total five experimental batches were included in this study. (FIG. 8B) Histograms showing numbers of genes detected per cell (left) and frequency of mitochondrial genes per cell (right). Cells before and after quality control selection are highlighted in dark grey and light gray respectively. (FIG. 8C) Gene expression UMAP of all cells color coded for experimental five batches (#1-5) (top) and disease condition (bottom). (FIG. 8D) Annotated cell numbers of CD4⁺ T cell subpopulation within total CD4⁺ T cells (left), CD4⁺CD25⁺⁺ T cells (middle), and combined total cells (right). Cell numbers for each batch and summary of five batches from HC and MS are shown. (FIG. 8E) Representative gene and protein expressions UMAP. (FIG. 8F) Numbers of upregulated and downregulated DEGs in each CD4⁺ T cell subpopulation are shown. (FIGS. 8G-8H) Representative differential gene analysis in each CD4⁺ T cell subpopulation is depicted (see also, e.g., methods described herein).

FIGS. 9A-9F show S3 single cell dual omics analysis with CD4⁺ T cells sub cell types in MS, related to FIG. 2. (FIG. 9A) Surface protein guided mTreg and mTconv subtype annotation. CD196, CD183, and CD194 expressions were shown in four subtypes for each mTreg and mTconv. (FIG. 9B) Heatmaps showing the marker genes and proteins to define subtypes for each mTconv and mTreg at individual subject level. (FIG. 9C) Key marker expressions (CD196, CD183, CD194, CXCR3 and GATA3) for each subtype in mTreg and mTconv. (FIG. 9D) UMAP based on CITE-seq (Cellular Indexing of Transcriptomes and Epitopes by Sequencing) based protein expressions for the subtypes in mTreg and mTconv. (FIG. 9E) Numbers of upregulated and downregulated DEGs in each subtype for mTreg (top) and mTconv (bottom). (FIG. 9F) Representative differential gene and surface protein analysis in each subtype in mTreg and mTconv are depicted (see also, e.g., methods described herein).

FIGS. 10A-10G show S4 elevated alternative short PRDM1 isoform in MS mTreg, related to FIG. 3. (FIG. 10A) Representative bulk RNA-seq coverage tracks at PRDM1 locus from nine different immune cell types in peripheral blood. Unique exonic regions for PRDM1-L and PRDM1-S are highlighted. (FIG. 10B) qPCR validation of short and long PRDM1 isoform expression between HC and MS from discovery cohort (left box plot) and validation cohort (right box plot). P*<0.05, P**<0.01; Statistical significance computed by unpaired t test. (FIG. 10C) Short and long PRDM1 isoform expression between HC and SLE from ImmuNexUT data. (FIG. 10D) Heatmaps depicting the Spearman's correlation between PRDM1-L expression and PRDM1-L signature genes (top) and Treg signature genes (bottom). (FIG. 10E) Coexpression analysis with bulk RNA-seq data for short and long PRDM1 isoform with curated immune related genes. (FIG. 10F) Western blot analysis of Blimp1 expression from 8 different immune cell types in peripheral blood and Blimp1-S or Blimp-1 overexpressed (OE) 293T cells by anti-Blimp1 Ab (C-7) (top) and anti-Blimp1 Ab (C14A4) (bottom). (FIG. 10G) Ratio of PRDM1-S vs PRDM1-L in bulk RNA-seq and qPCR data.

FIGS. 11A-11E show short PRDM1 induces SGK1 and Treg instability, related to FIG. 4. (FIG. 11A) qPCR validation of total PRDM1 and long PRDM1 expression by short and long PRDM1 isoform overexpression in mTreg. (FIG. 11B) Volcano plot showing statistical significance and fold change for genes differentially expressed by long PRDM1 overexpression in primary mTregs. (FIG. 11C) SGK1 expression assessed by scRNA-seq in four main CD4+ T cell subpopulations (top) and mTreg sub cell-types (bottom). ADE: average disease effect between disease cells and matched healthy cells across all the individuals, both MS and HC. ADC: average disease effect only measured within the healthy control group. ADD: average disease effect only measured within the disease group. (FIG. 11D-11E) SGK1 expression in Fr2 eTreg and Fr1 nTreg across 10 and 6 autoimmune diseases respectively. P*<0.05, P**<0.01, P***<0.001, P****<0.0001; Statistical significance computed by one way ANOVA with Dunn's multiple comparisons.

FIGS. 12A-12B show cis-eQTL effect of CD28 locus and co-expression with AP-1/IRF TFs and PRDM1 isoforms, related to FIG. 5. (FIG. 12A) CD28 cis-eQTL effect with different immune cell types. Data were analyzed with ImmuNexUT (left) and DICE (right). Boxed dots represent cell types with significant cis-eQTL effect on CD28. (FIG. 12B) Heatmap showing the co-expression analysis between PRDM1 isoforms and AP-1/IRF family TF genes in HC and MS mTreg.

FIGS. 13A-13E show identification of active enhancer for short PRDM1 in human T cells, related to FIG. 6. (FIG. 13A) Schematic of CRISPRa experiment for short and long PRDM1 induction with targeting each promoter element (left) and qPCR quantification of short and long PRDM1 expression (right). (FIG. 13B) Schematic of sgRNA design for each candidate peak with CRISPRa. Three different sgRNAs are designed. (FIG. 13C) Representative dot plot showing dCas9-Vp64 (GFP) and gRNA containing vector (RFP). (FIG. 13D) Validation CRISPRa and CRISPRi experiment for #2 peak independent from FIG. 5B (n=4). (FIG. 13E) Lentiviral shRNA-based gene knockdown for IRF4 and BATF in human primary Tregs. Human primary Tregs are isolated by FACS and stimulated with anti-CD3/CD28 antibodies. Lenti particles were transduced at day 1 and GFP+ cells were sorted by FACS at day 4-5. Expressions of each gene assessed by qPCR are shown. P*<0.05, P**<0.01, P****<0.0001; Statistical significance computed by unpaired t test.

FIG. 14 shows dysfunctional Foxo1 and Foxo3 KO Treg signatures in MS and SLE mTregs. Dot plots showing gene set enrichment analysis (GSEA) for Foxo signaling on Tregs in MS (left) and SLE (right) Tregs. Gene sets generated by Foxo1/3 KO Tregs and Foxo1 constitutive active (CA) Tregs are used. NES; Normalized enrichment score.

FIG. 15 shows aetiology of MS cannot be explained by just genetic risk variants.

FIG. 16 shows a majority of causal variants for MS map to immune-cell specific cis-regulatory element.

FIG. 17 shows cell type and context specific genetic effects.

FIG. 18 shows multiple sclerosis is shown as a T cell mediated autoimmune disease.

FIG. 19 shows no difference of gene expression signature in circulating CD4+ T cells in MS.

FIG. 20 shows human CD4+ T cells are highly heterogeneous.

FIG. 21A shows a workflow for bulk RNA-seq and bulk ATAC-seq.

FIG. 21B shows that PRDM1 is significantly upregulated in both MS Treg and Tconv.

FIG. 21C shows upregulation of PRDM1 in MS mTreg by bulk population.

FIG. 22 shows upregulation of PRDM1 in MS mTreg at single-cell resolution.

FIG. 23 shows CITE-seq (Cellular Indexing of Transcriptomes and Epitopes by Sequencing). Figure discloses SEQ ID NOS 166-168, 167, 169, 167-168, 167, and 170, respectively, in order of appearance.

FIG. 24A shows single cell RNA-seq analysis on CD4+ T cells: GEX vs CITE-seq.

FIG. 24B shows single cell RNA-seq analysis on CD4+ T cells: GEX vs CITE-seq.

FIG. 25A shows single cell RNA-seq analysis on CD4+ T cells: CITE-seq.

FIG. 25B shows CITE-seq analysis on memory CD4+ T cells: T helper subtypes.

FIG. 25C shows differential expression analysis on memory Tregs: MS vs Healthy controls (HC).

FIG. 26 shows upregulation of PRDM1 in Treg among patients with autoimmune diseases (Ota et al., Dynamic landscape of immune cell-specific gene regulation in immune-mediated diseases. Cell. 2021 May 27;184(11):3006-3021.e17).

FIG. 27 shows function of PRDM1 (Blimp-1) in Treg.

FIG. 28 shows HiDRA (High-resolution Dissection of Regulatory Activity) peak in PRDM1 locus.

FIGS. 29A-29B show two major PRDM1 isoforms in humans.

FIG. 30 shows PRDM1 short isoform is upregulated in MS-mTregs.

FIG. 31 shows evolution of PRDM1 short isoform.

FIG. 32 shows overexpression of short PRDM1 induces SGK1.

FIGS. 33A-33B show SGK1 represses Treg function.

FIGS. 34A-34B show overexpression of short PRDM1 induces Treg dysfunction.

FIG. 35 shows epigenetic profiles (bulk ATAC-seq). Figure discloses SEQ ID NO: 171.

FIG. 36 shows no significant change of chromatin accessibility in MS Tregs.

FIG. 37 shows TF motif analysis and footprint analysis using ATAC-seq data.

FIG. 38 shows AP-1 footprints are more enriched in MS Treg regulatory elements.

FIG. 39 shows MS susceptible CD28 locus can be linked with AP-1 priming in MS Treg.

FIG. 40 shows the exploration of factors that regulate short PRDM1 expression in mTreg.

FIG. 41 shows PRDM1 enhancer perturbation using CRISPRa.

FIG. 42 shows short PRDM1 enhancer element harbors AP-1 binding motif. Figure discloses SEQ ID NO: 165.

FIG. 43 shows a summary of factors leading to Treg dysfunction.

### DETAILED DESCRIPTION

Disruption of peripheral CD4⁺ T cell homeostasis is a central component driving pathogenesis of autoimmune disease where autoreactive T cells lose tolerance to self-antigen by both intrinsic and extrinsic mechanisms. Treg-mediated surveillance is a central gatekeeper for controlling activation of autoreactive CD4⁺ T cells and dysfunctional Tregs are a hallmark of MS and other autoimmune diseases (10, 11). In addition, recent analysis of genome-wide association studies emphasizes the substantial contribution of CD4⁺ T cells, including Tregs, as potentially causal mediators of autoimmune disease (6). Although several phenotypic changes have been identified in dysfunctional Tregs, the underlying molecular mechanisms leading to breakdown of Treg suppressive function in patients with autoimmune diseases are unknown. Here, by using MS as a model for studying the molecular mechanisms of human Treg dysfunction, transcriptional and epigenetic alteration in human Tregs were examined, identifying a previously unknown role of an alternative short PRDM1 isoform in dysfunctional Treg that disrupted the long PRDM1-mediated Treg maintenance. SGK1 was identified as a target of short PRDM1, which has been reported to confer the pathogenic function of Treg in both human and mouse (17, 18, 62). Moreover, both PRDM1 and SGK1 were upregulated in Tregs from different autoimmune diseases such as SLE and ANCA-associated vasculitis (1), suggesting that the PRDM1 SGK1 axis could serve as a common feature of Treg dysfunction in the context of human autoimmunity. Finally, exploration of epigenetic changes in MS Teg revealed an active enhancer element that induces short PRDM1 transcription, which was validated by CRISPRa experiments. AP-1 family and IRF TFs directly bound to this enhancer region, implicating the role of these TFs in contributing to dysfunctional Tregs in autoimmune disease. Thus, this study provides a novel mechanistic insight of dysfunctional Tregs in the context of MS and potential therapeutic targets to reverse Treg dysfunction in autoimmune diseases.

Previous studies exploring transcriptional alterations that occur in CD4+ T cells in patients with MS as compared to control subjects did not identify significant differences (7, 8). It was critical to examine CD4+ T cell subpopulations. CD4+ T cells were further segregated into four subpopulations and performed transcriptional characterization using bulk RNA-seq with deeper sequencing. This allowed the detection of significant differences in gene expression between patients with MS and control subjects at both the gene and transcript level. Bulk RNA-seq based findings in the discovery cohort were further confirmed by two different means; (1) utilization of a validation cohort with the same method as the discovery cohort, and (2) utilization of CITE-seq to determine the subpopulations and assess the difference within the third cohort, leading to highly reproducible findings. One caveat is that an attempt to quantitate PRDM1 isoform expression at single cell resolution in the 10× genomics dataset was unsuccessful. Full length mRNA-capturing scRNA-seq has the potential to differentiate between PRDM1 isoforms; thus, this technique could be applied to elucidate further insight at single-cell resolution (88). This approach will also enable characterization of further differences in T cell transcriptomics between MS and healthy subjects.

A transcriptomic analysis of PRDM1 isoforms identified expression patterns between PRDM1-S and PRDM1-L that are highly cell type specific. Memory T cells and NK cells expressed higher levels of PRDM1-S as compared to PRDM1-L, while naïve T cells, monocytes/DCs, and B cells preferentially express PRDM1-L. Of note, PRDM1 expression across all B cell linage (naïve, unswitched memory, switched memory, double negative B cells, and plasmablasts) is strictly limited to PRDM1-L over PRDM1-S(ImmuNexUT dataset). It was also observed that the balance between PRDM1-S and -L is dynamic and changes after T cell activation in vitro. These findings demonstrate a tight linkage between PRDM1 isoforms and immune pathways related to AP-1/IRFs in autoimmune diseases and highlight a fundamental role in primates for PRDM1 isoform switching in regulating immune responses.

Epigenetic alterations in MS Tregs were assessed by using bulk ATAC-seq. Conventional characterization with differential analysis of chromatin accessibility did not reveal significant alterations of chromatin accessibility between patients with MS and healthy donors at a genome-wide level. ATAC-seq data were used to elucidate TF footprint enrichment and it was found that AP-1 and IRF family TFs are significantly enriched in MS mTreg compared to that of healthy controls. This suggests that AP-1 and IRFs contribute to shaping the MS transcriptional signature, which agrees with previous studies highlighting the important role of AP-1 in establishing epigenetic state and linking genetic susceptibility to T cell activation (75). Of note, the eQTL analysis identified a cell type specific and MS dependent eQTL effect with GWAS associated SNP nearby CD28 locus in mTreg. Given that CD28 signaling is central in the activation of AP-1 function in T cells, these findings of AP-1 enrichment in MS mTregs suggest that the genomic susceptibility of MS could be mediated by high activity of the AP-1 family in mTregs, resulting in increased PRDM1-S. Further studies focused on the functional properties of the MS-associated SNP at the CD28 locus are warranted.

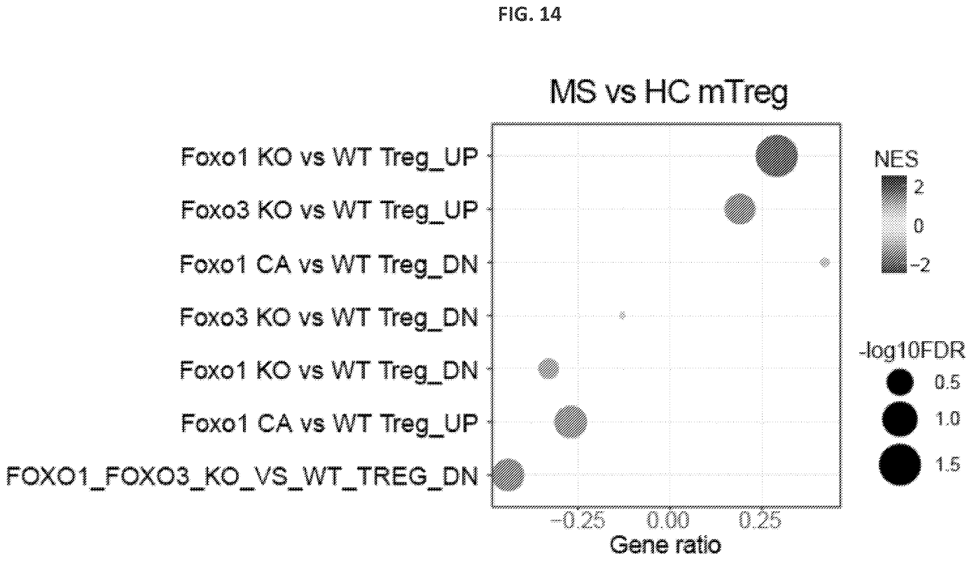

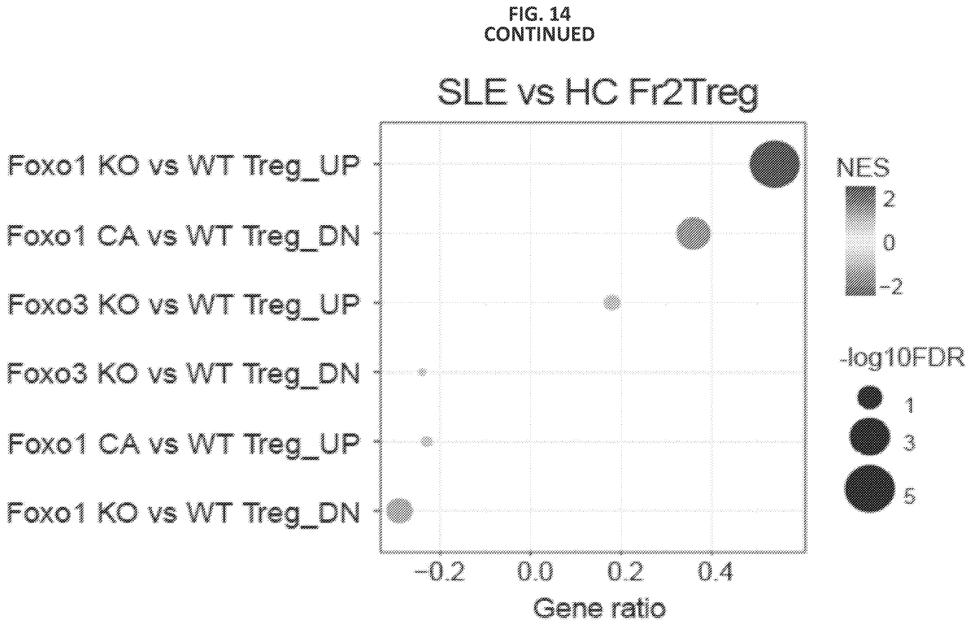

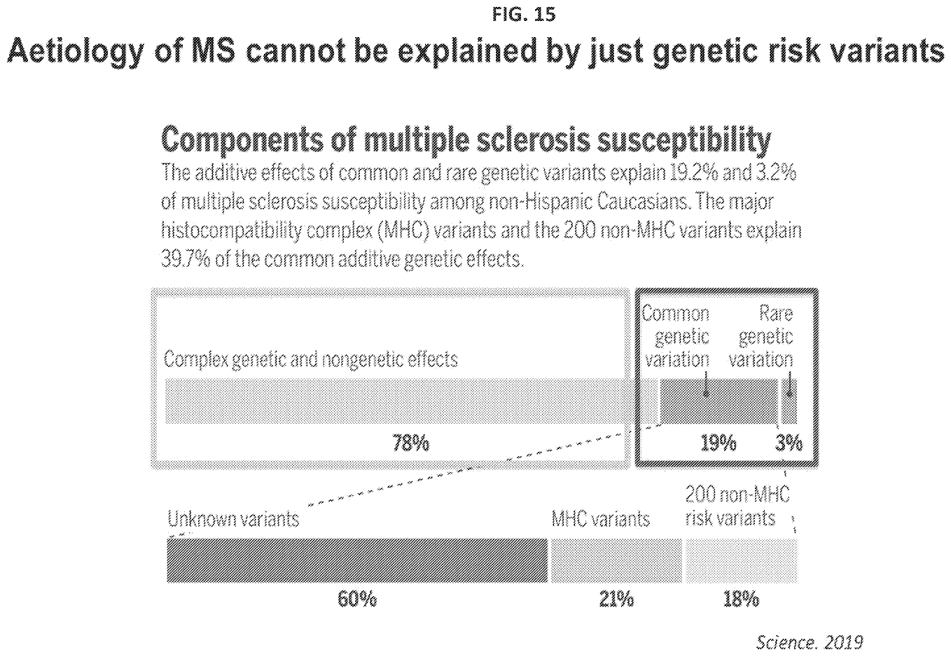

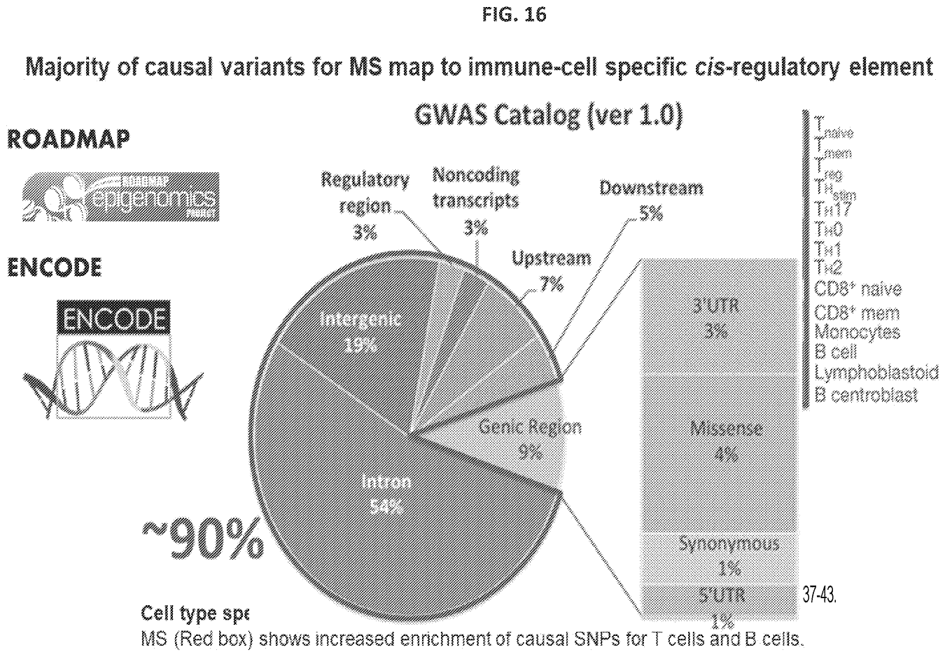

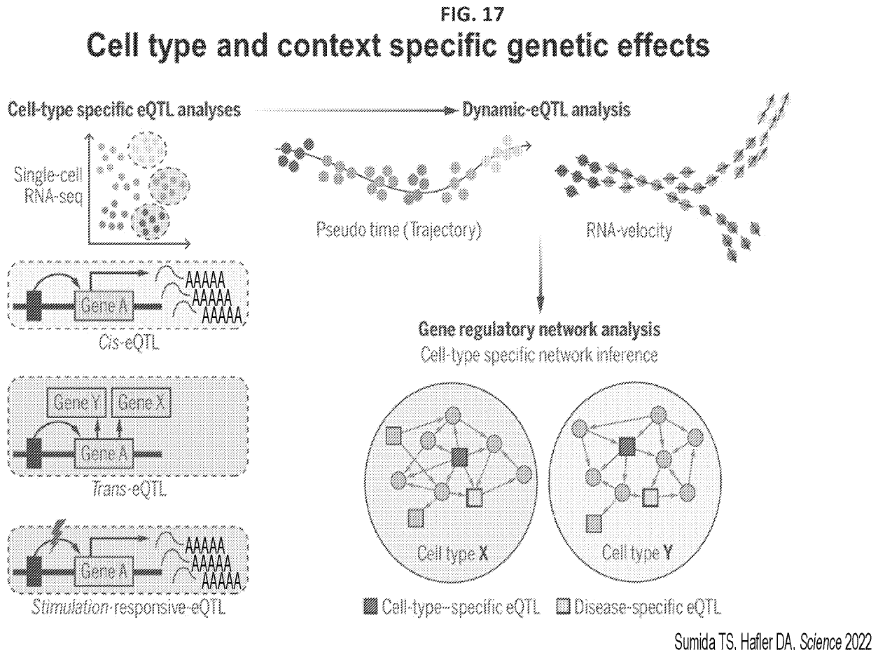

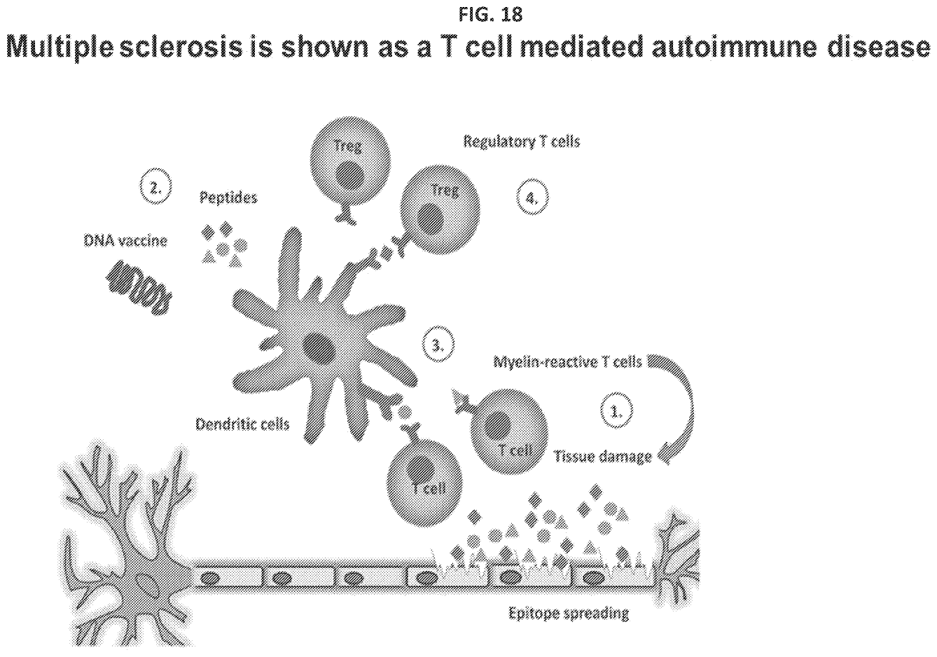

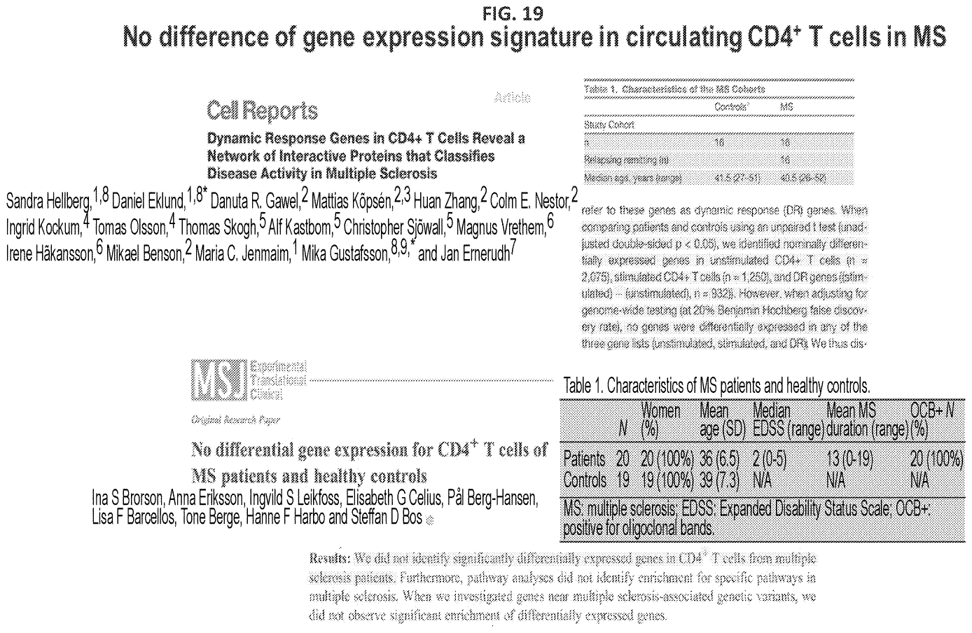

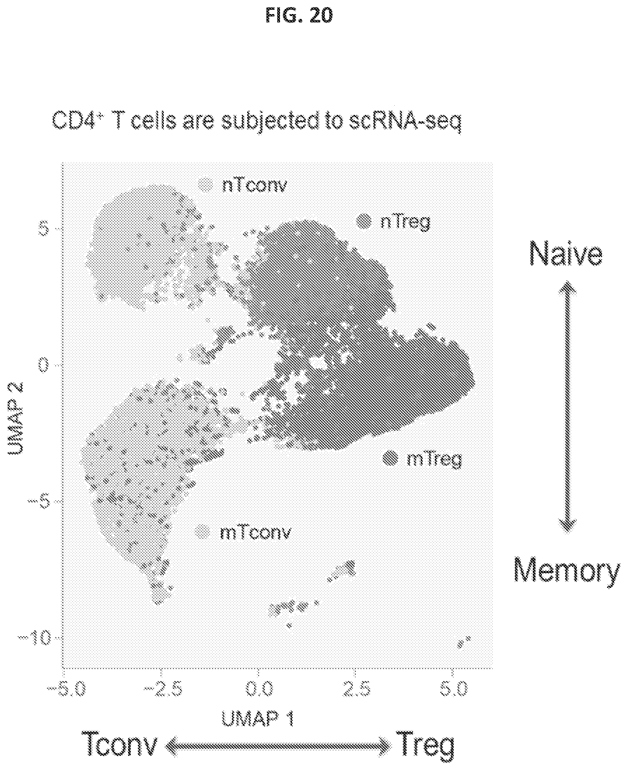

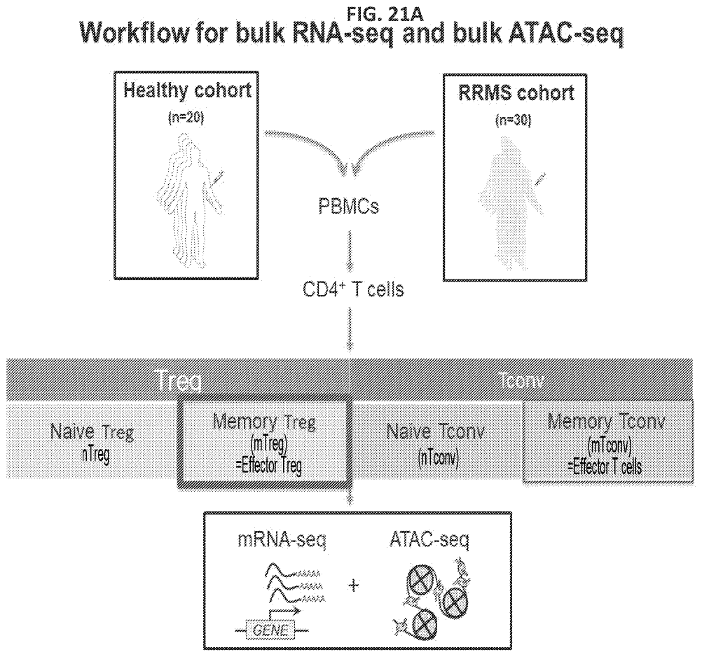

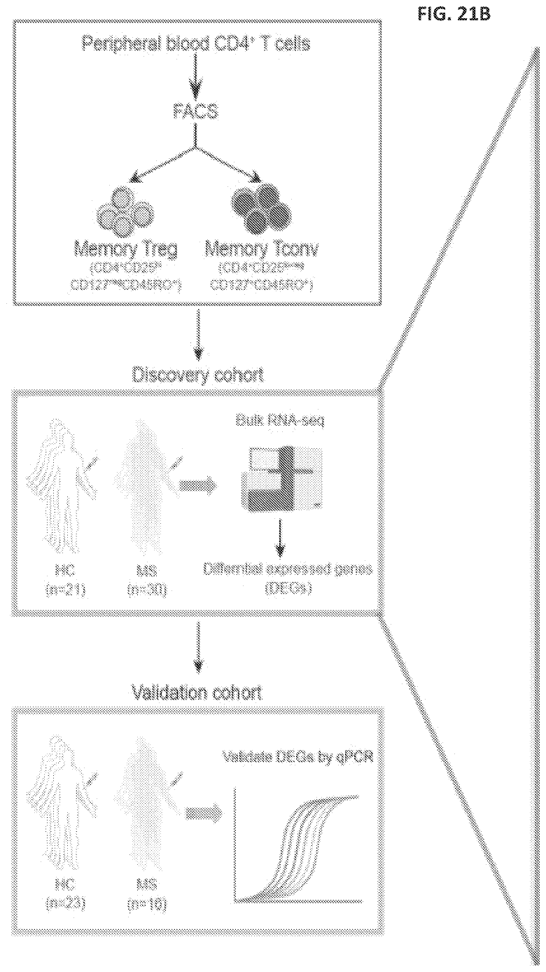

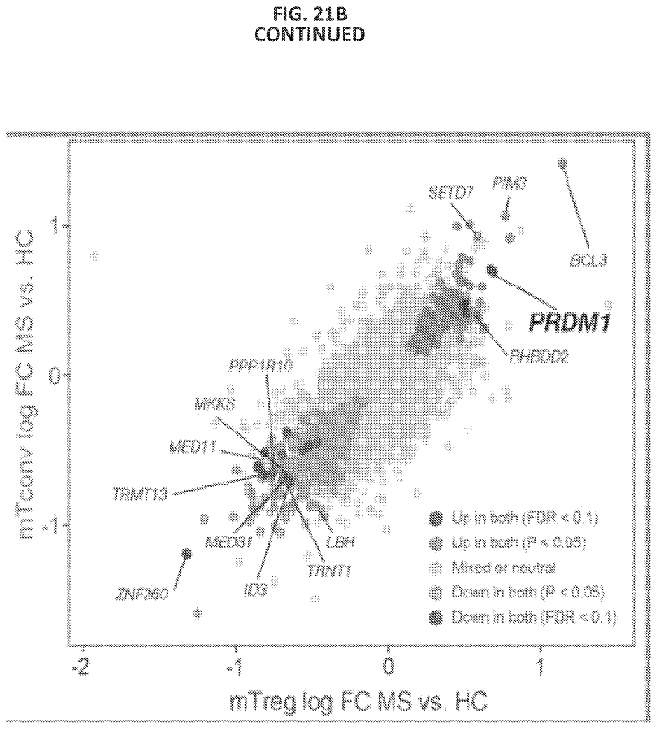

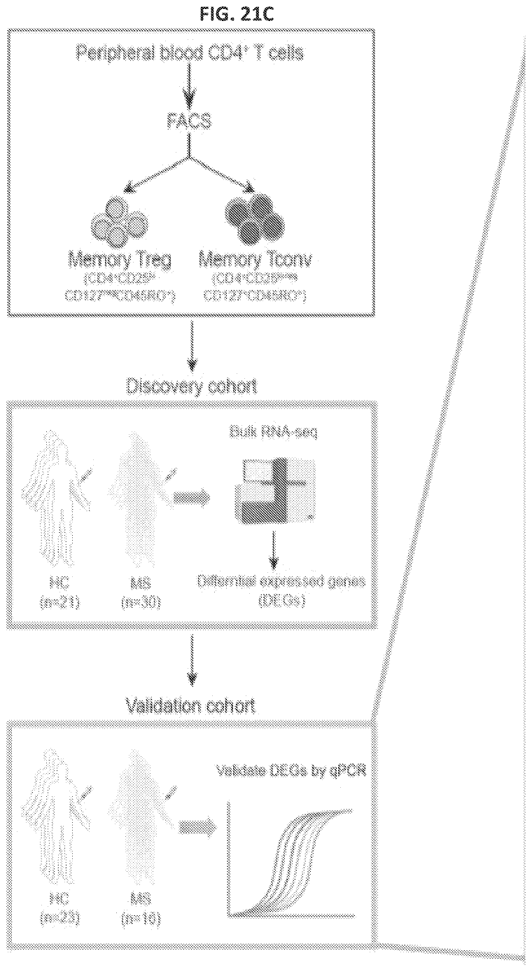

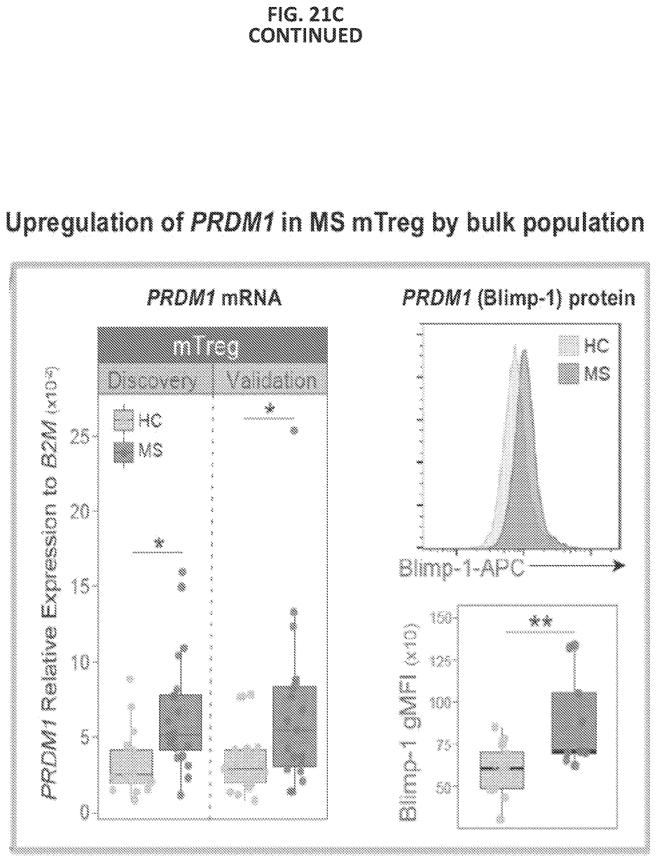

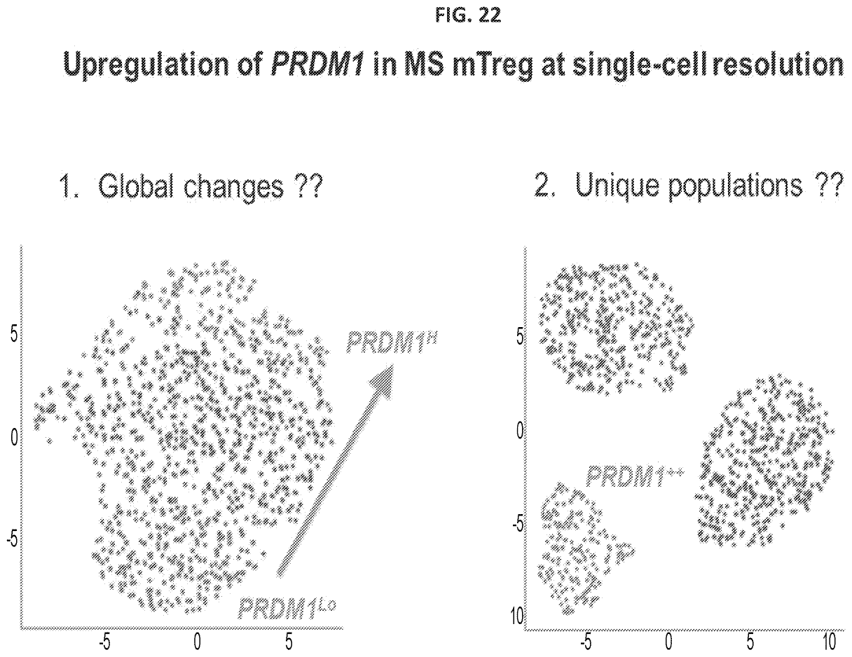

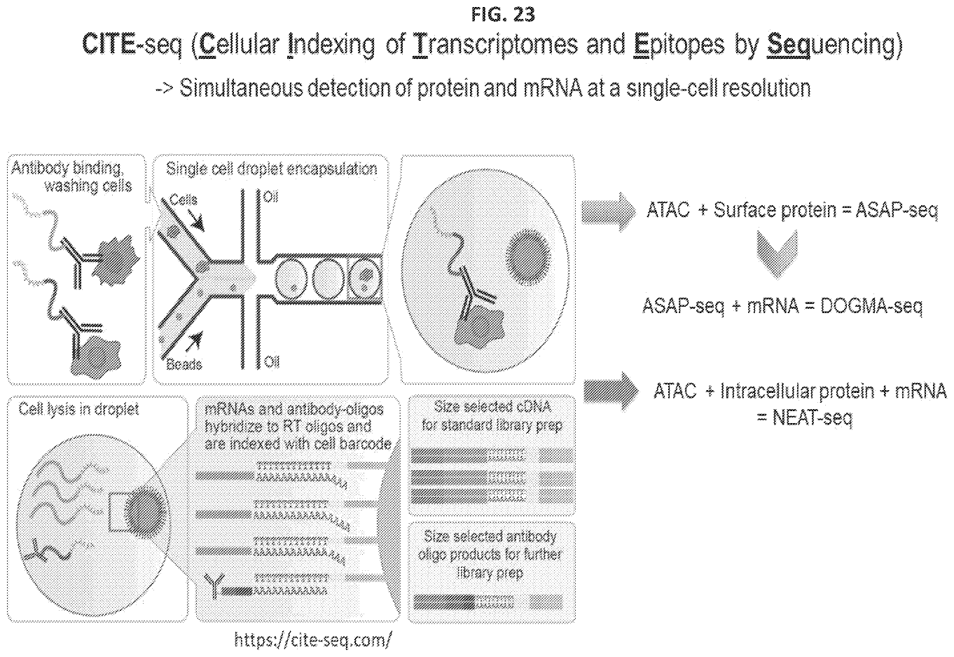

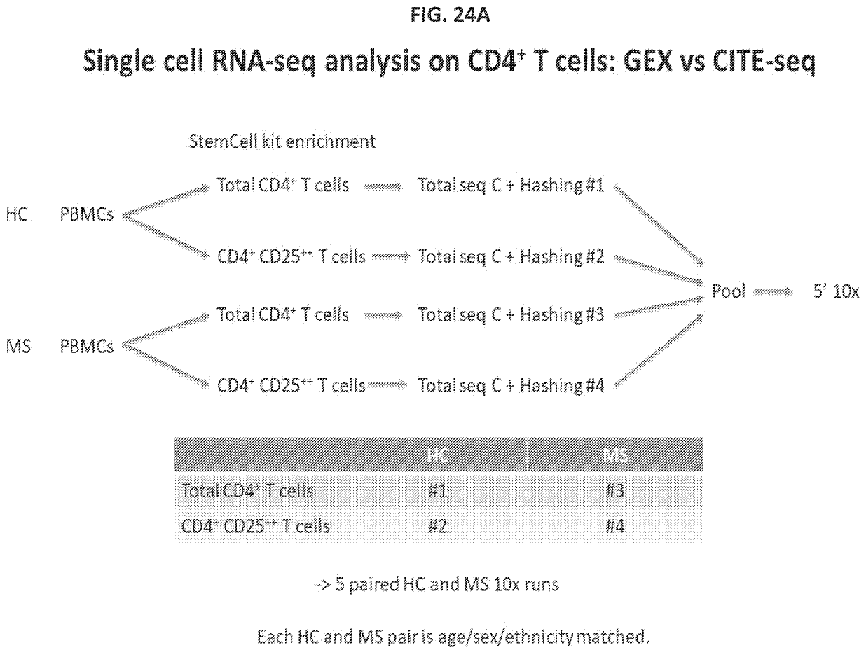

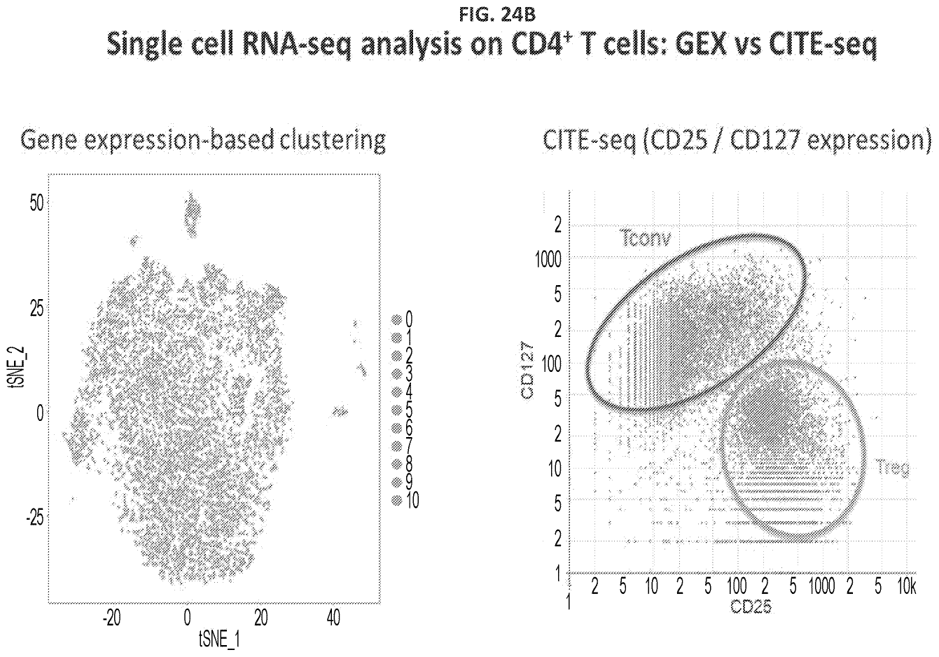

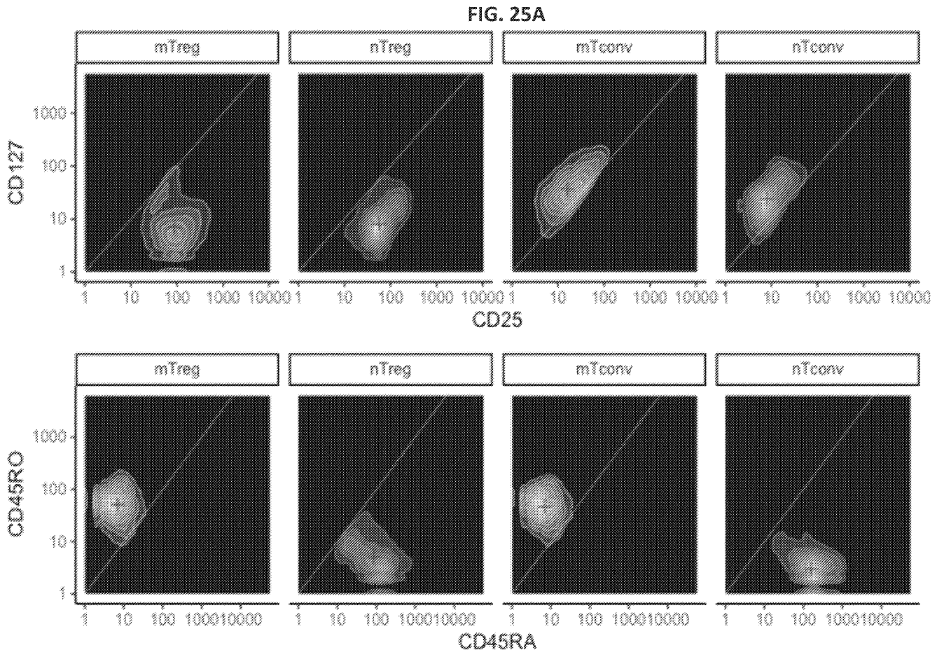

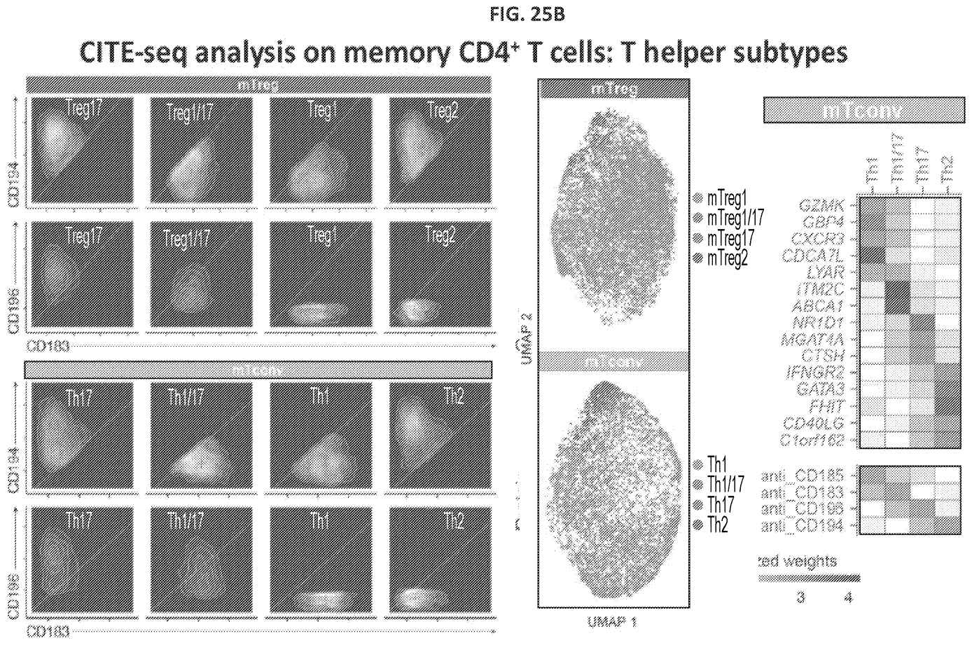

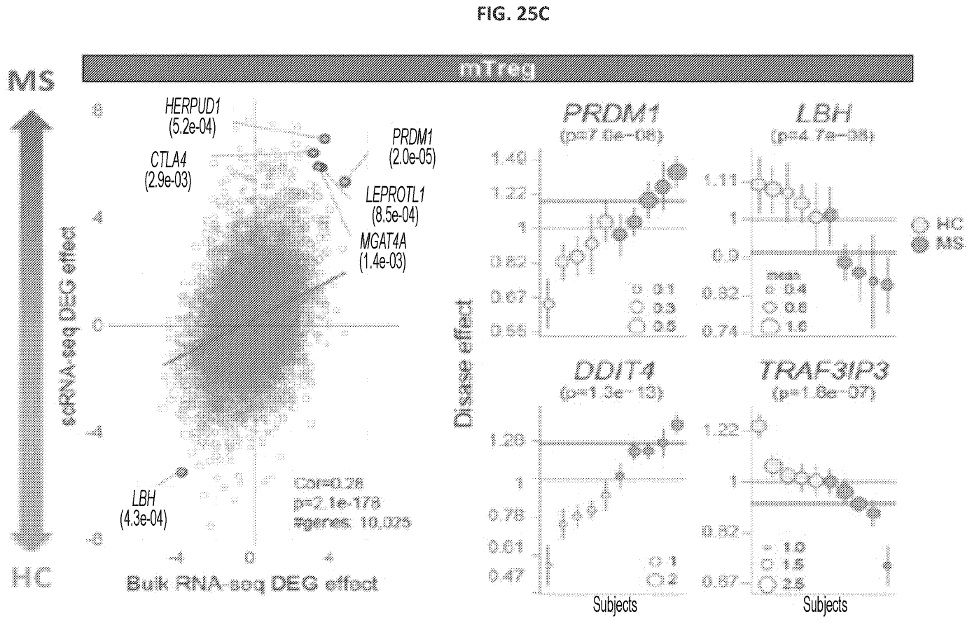

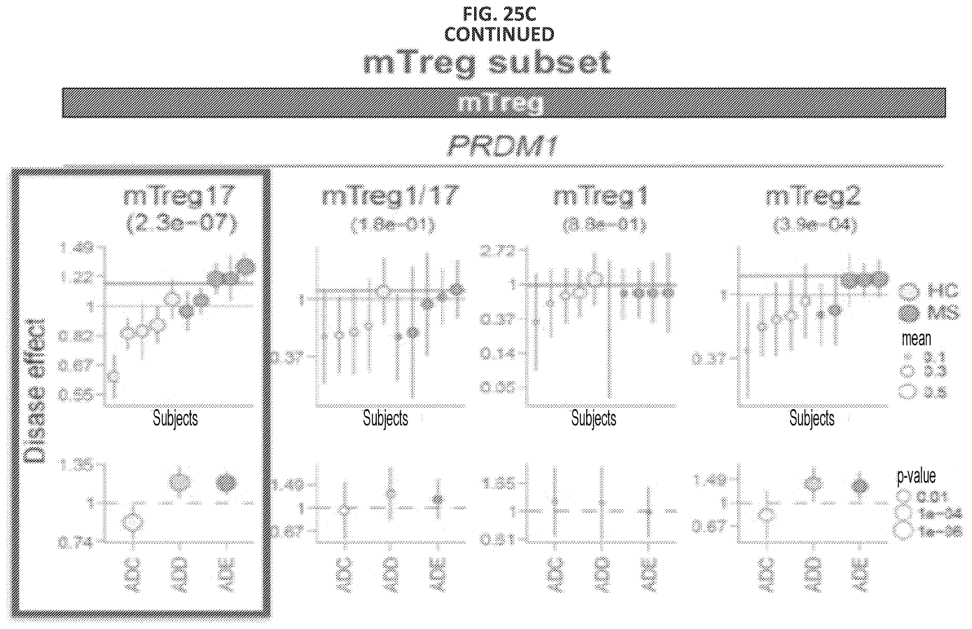

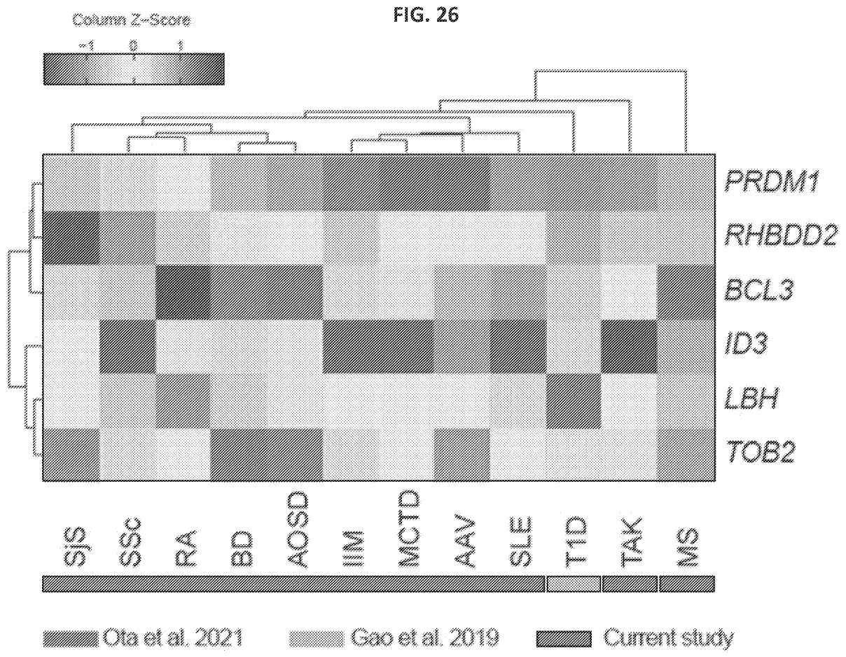

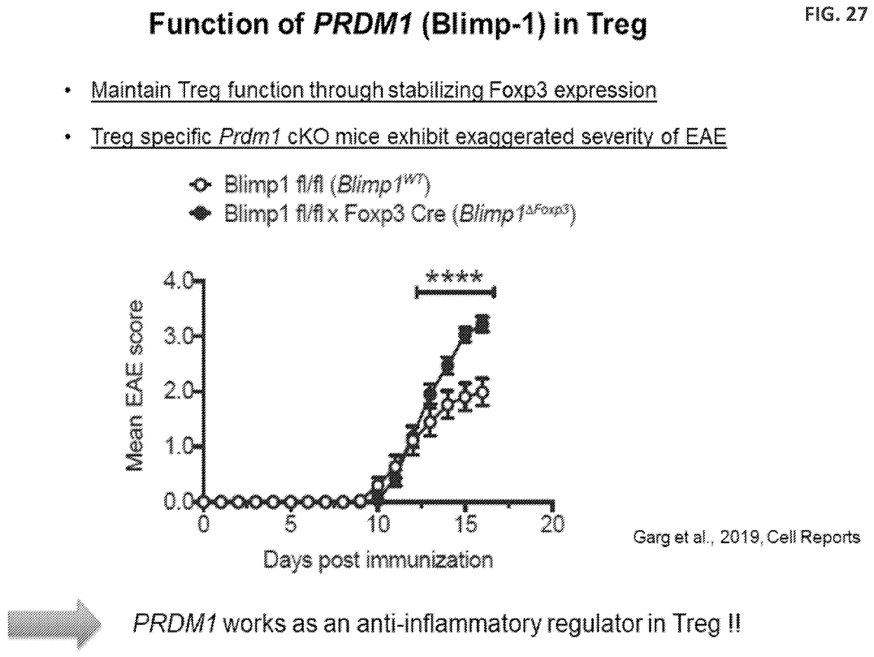

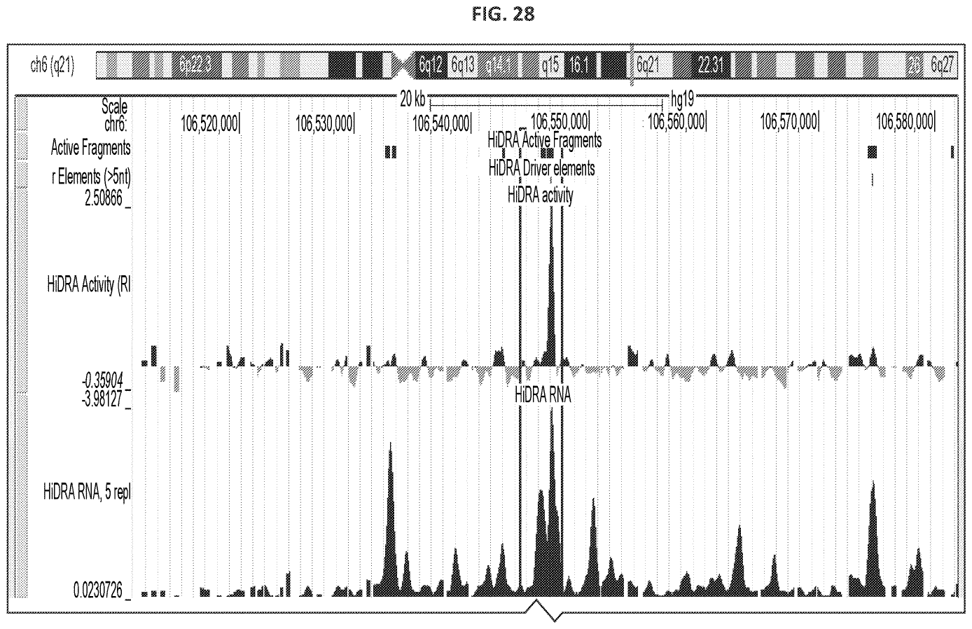

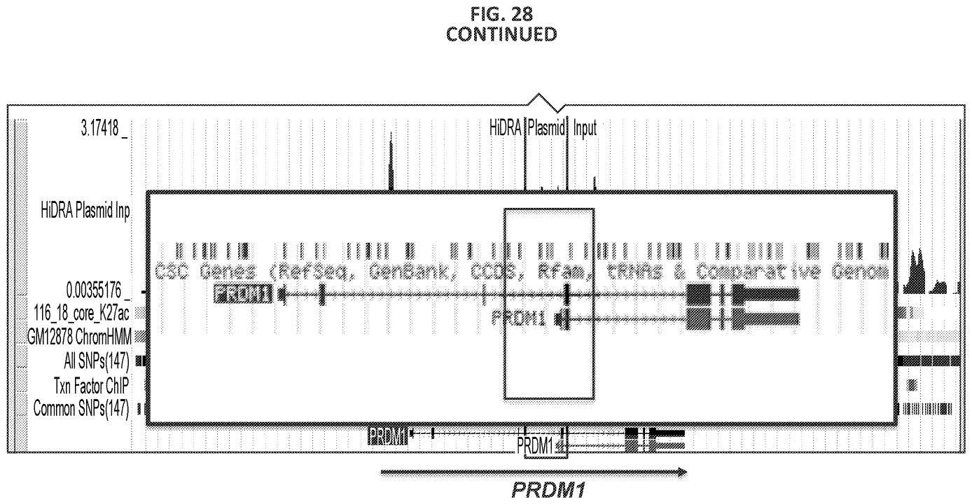

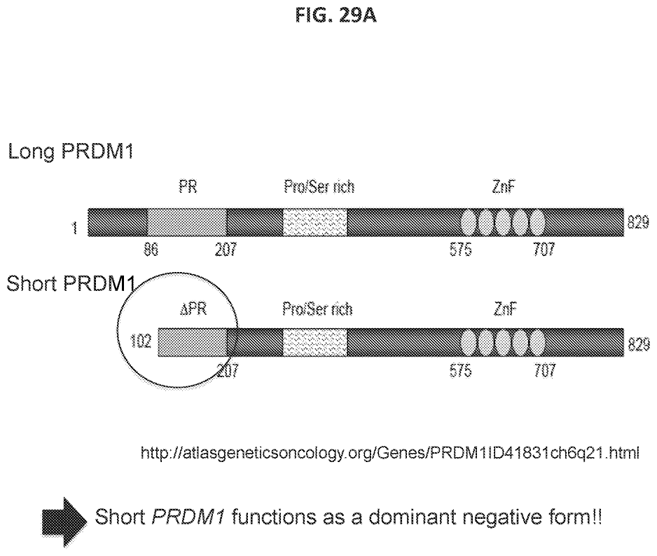

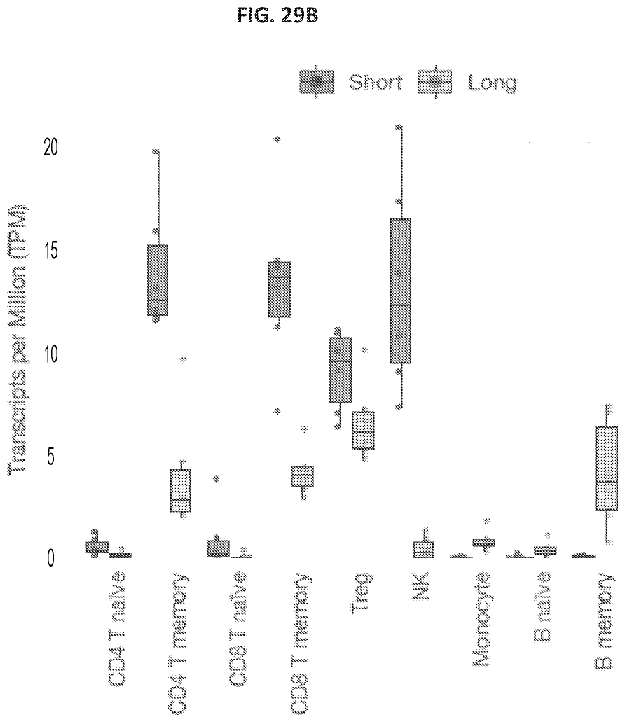

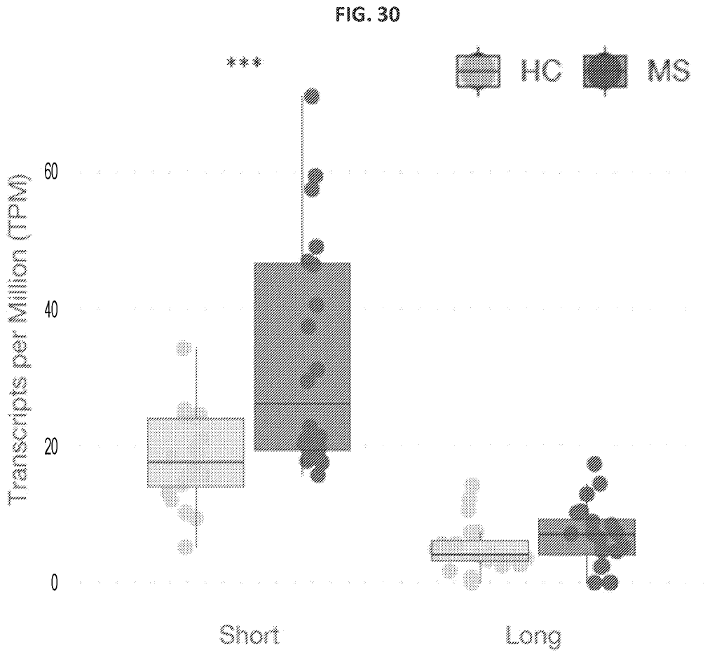

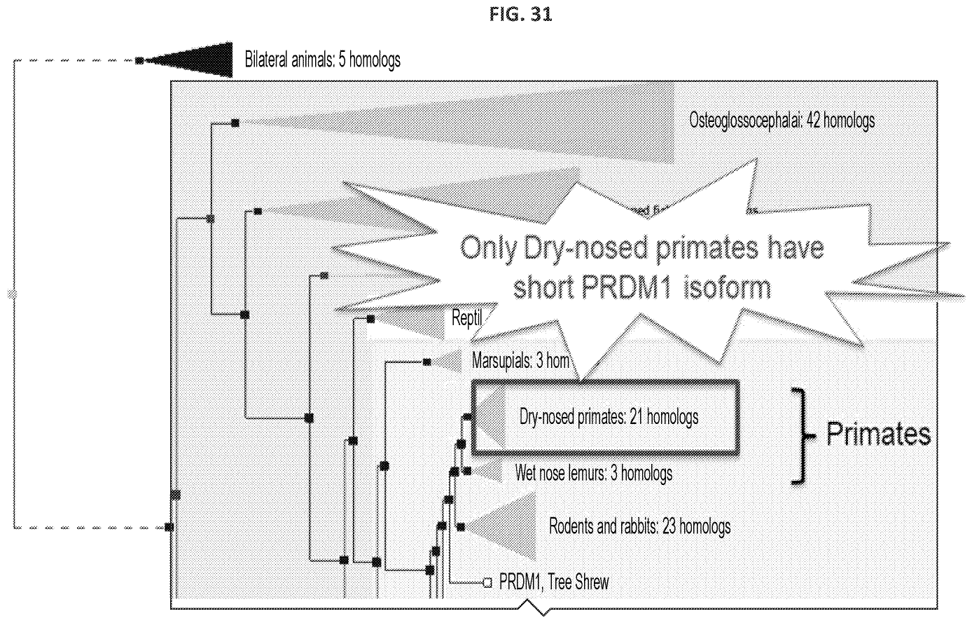

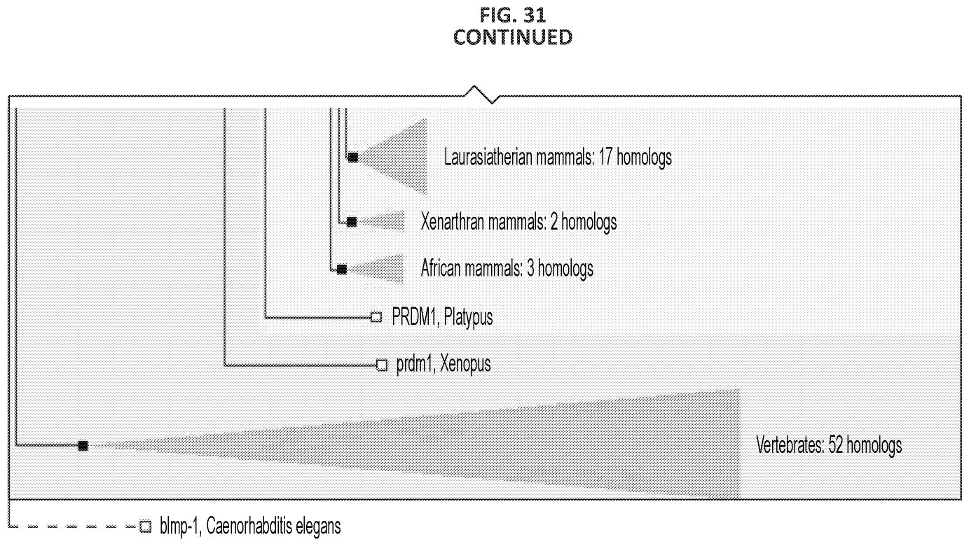

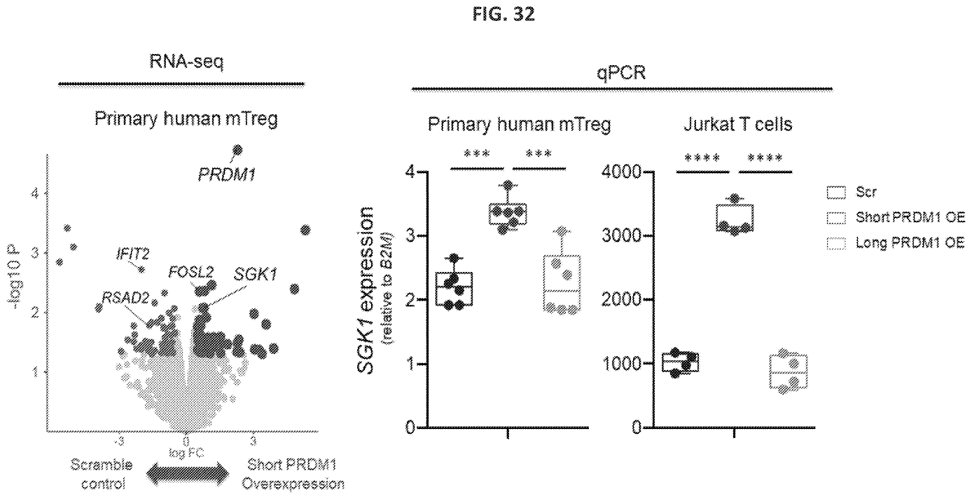

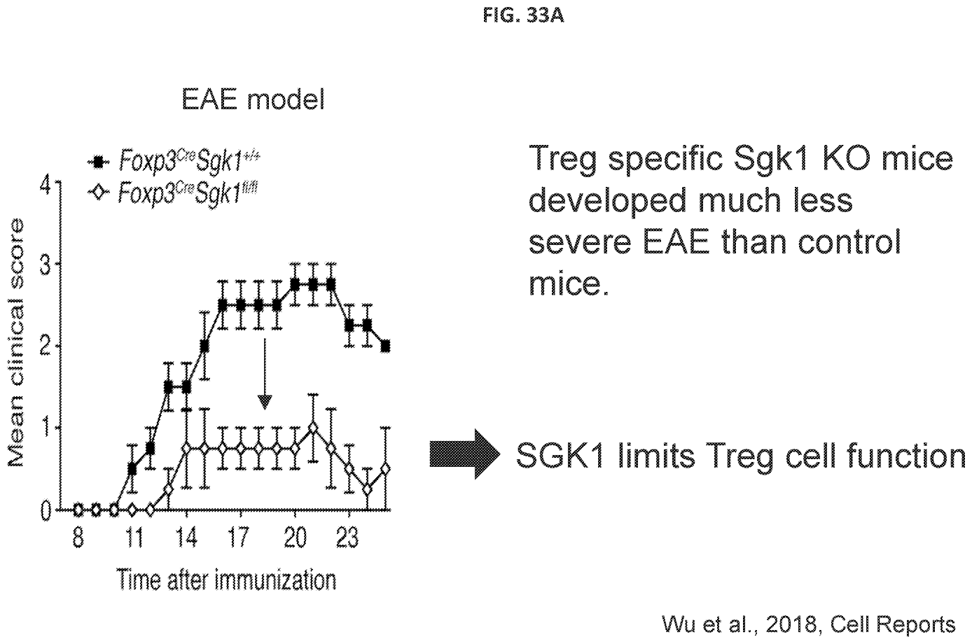

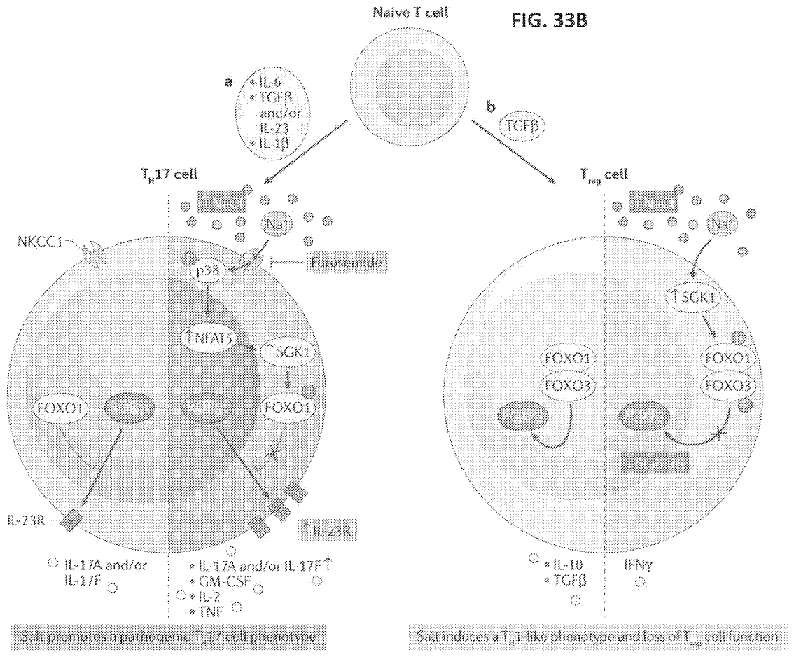

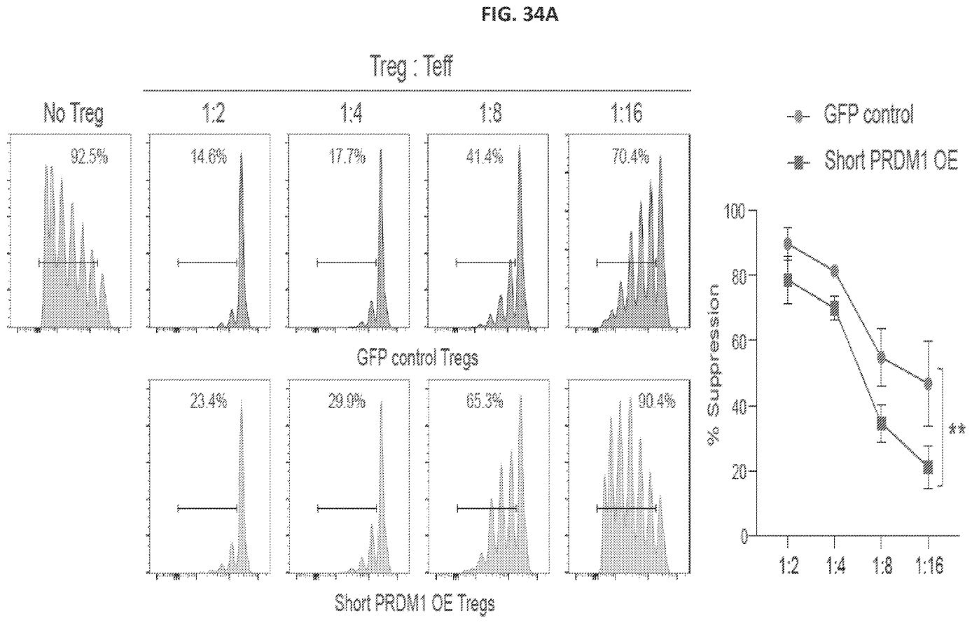

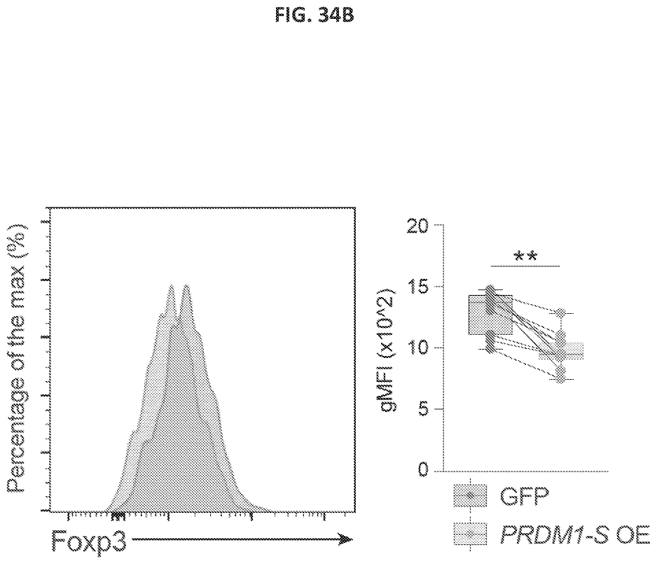

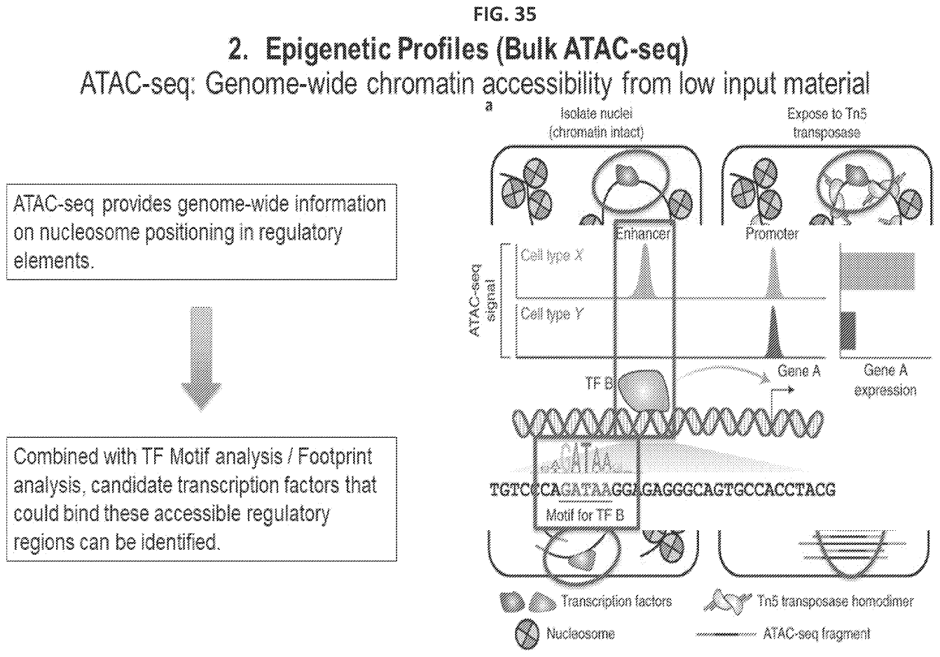

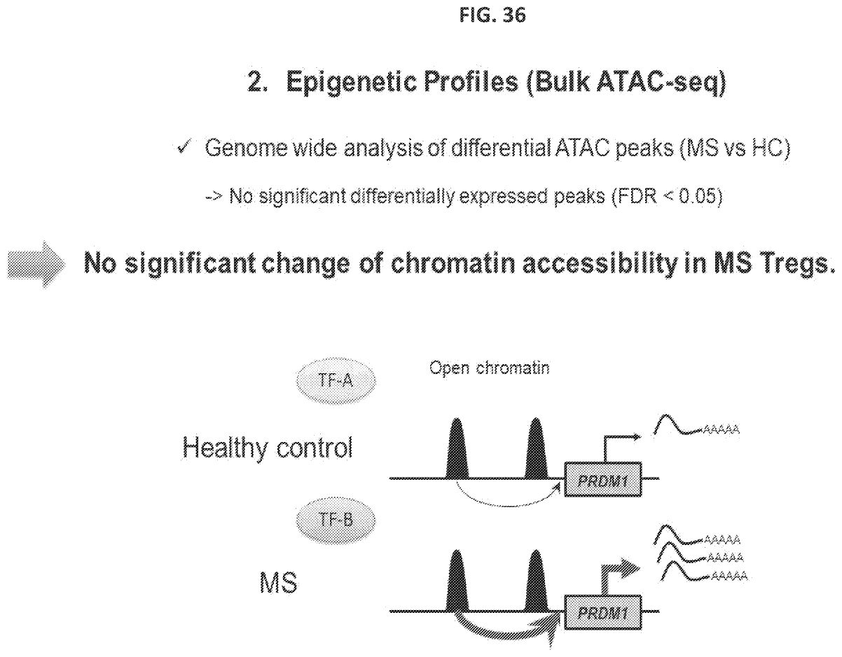

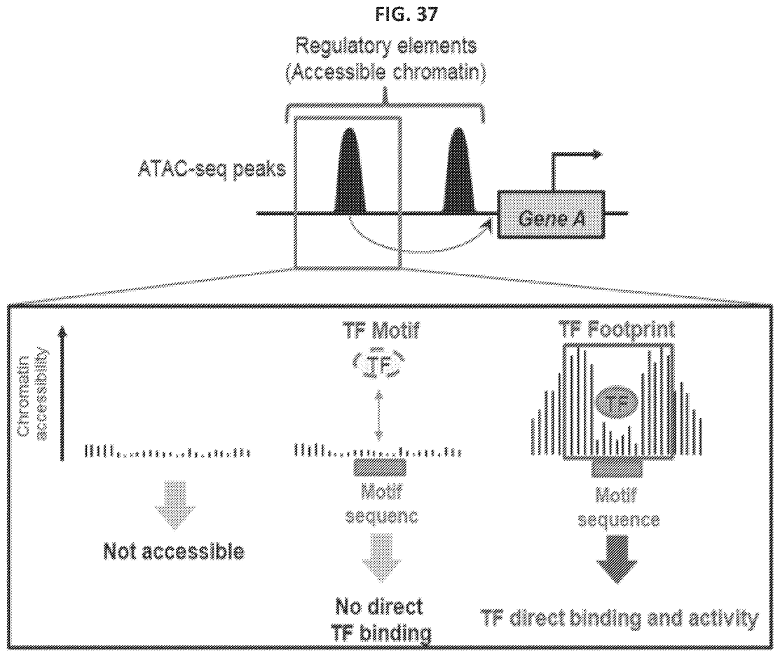

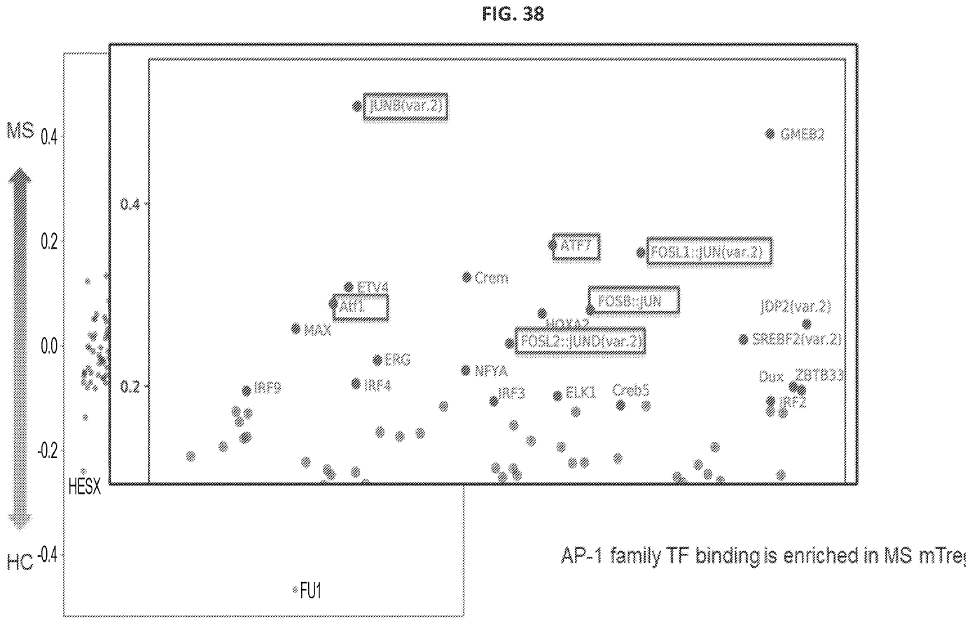

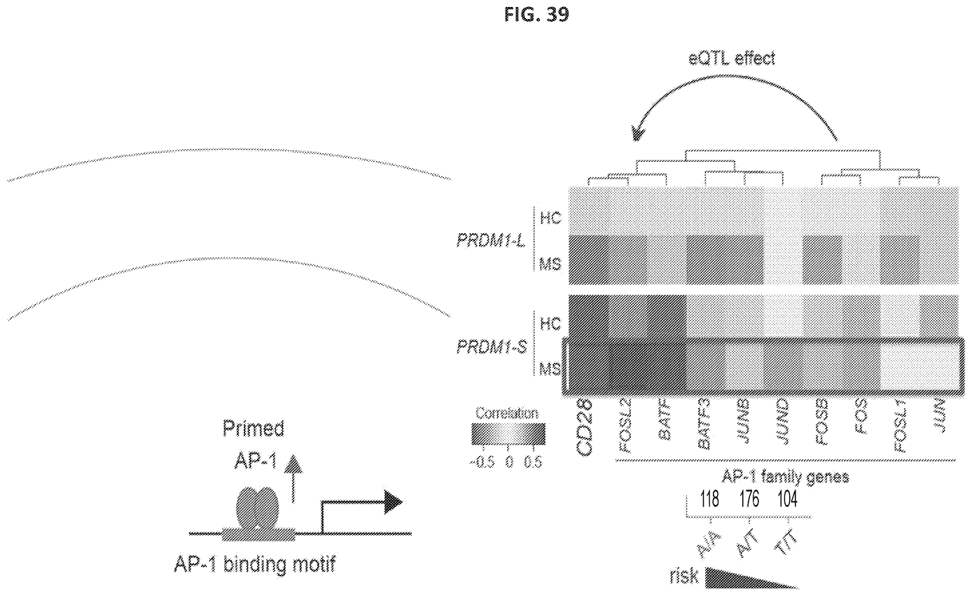

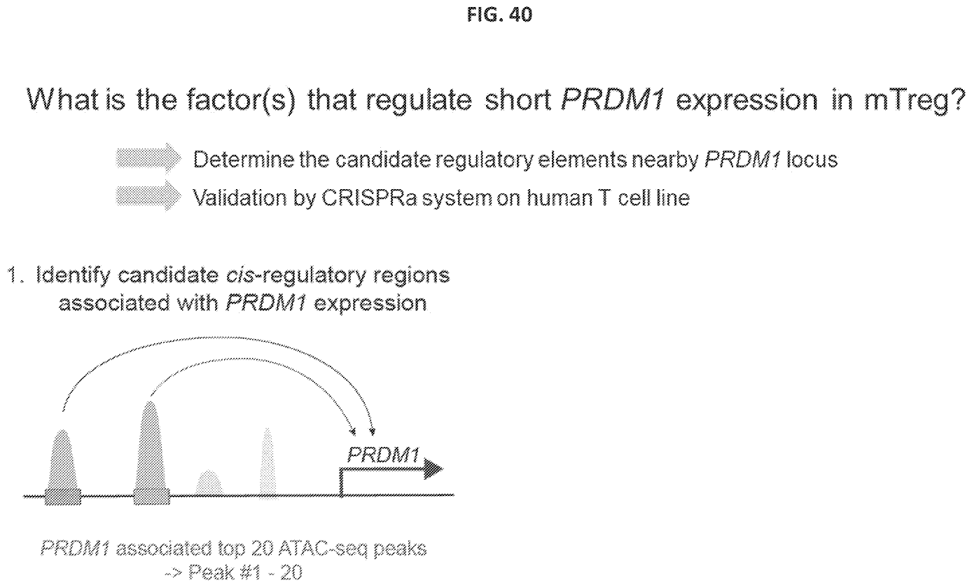

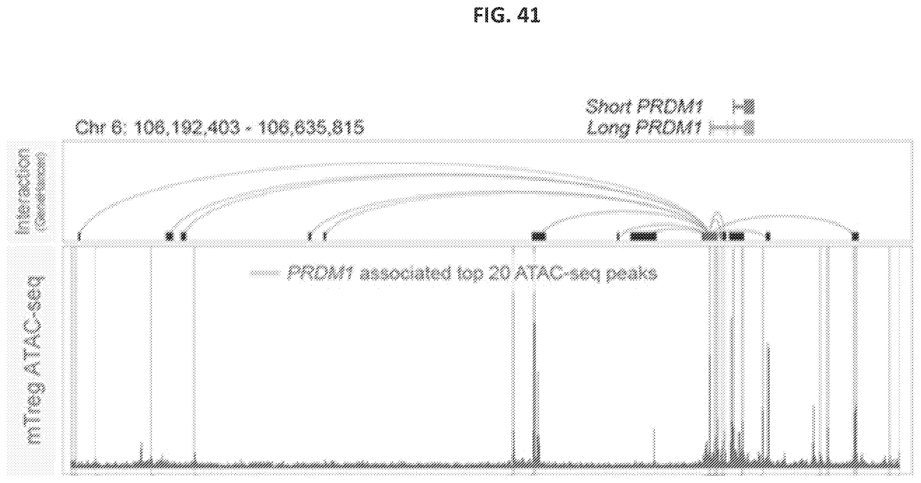

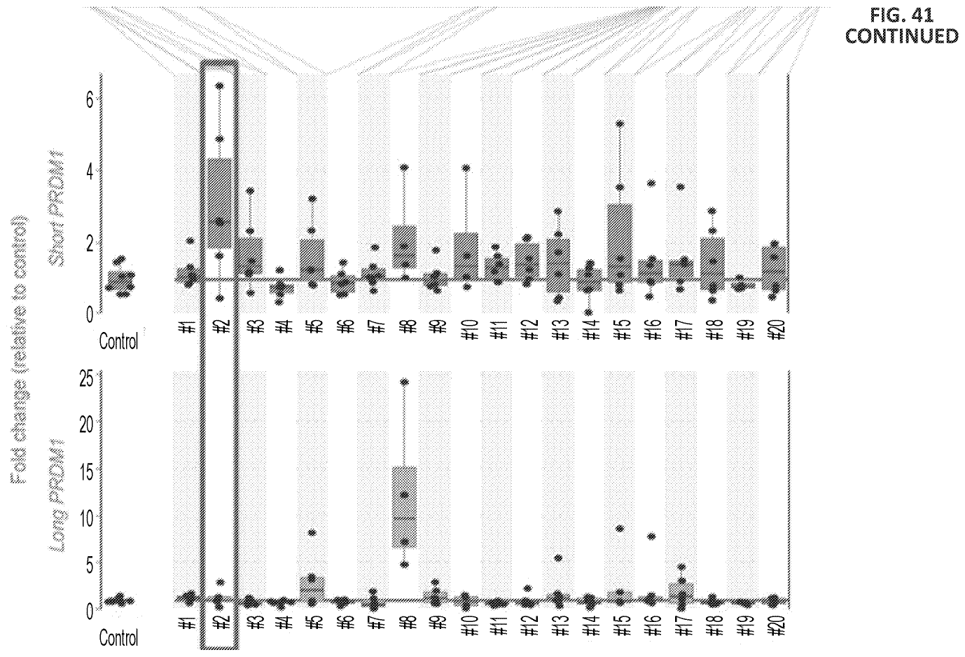

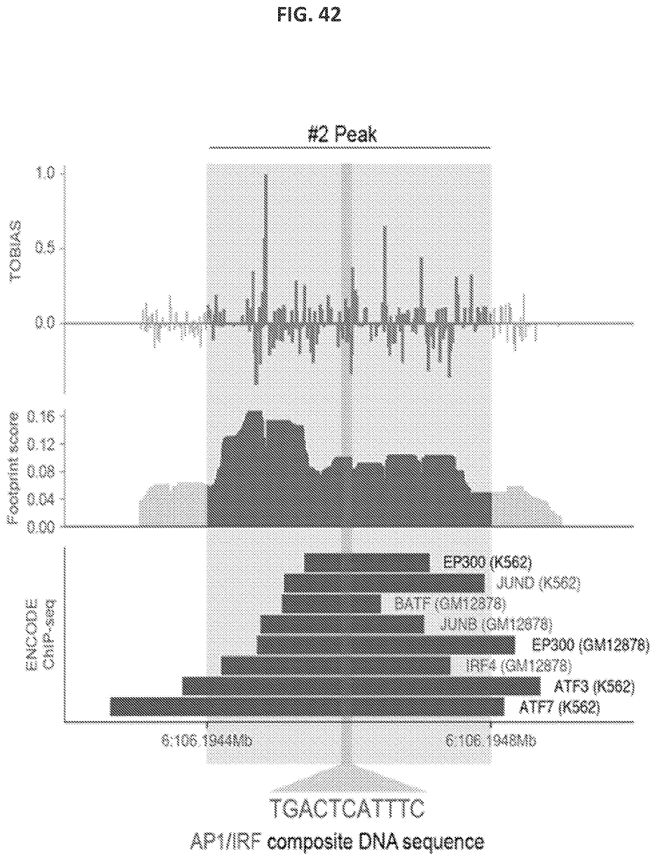

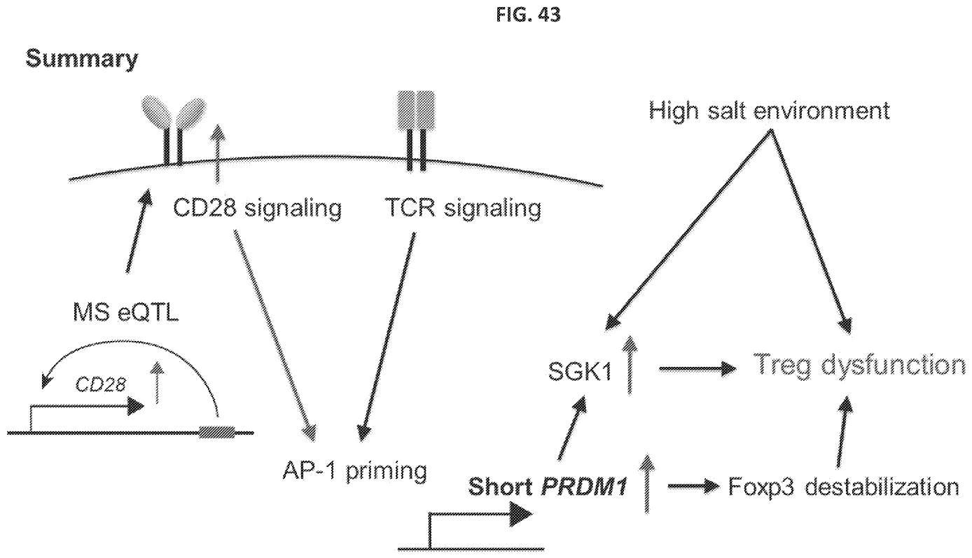

A fundamental question relates to the elucidation of molecular interactions between environment triggers and gene transcription driven by allelic variation associated with disease risk that lead to autoimmune disease. The data reported herein suggest a model where both environmental and genetic mechanisms may lead to dysfunctional Tregs. That is, a genetic variant associated with risk of MS in the intronic CD28 gene region was identified that leads to increased CD28 expression and thus higher AP-1 activity driving PRDM1-S expression that induces SGK1. Previous reports have shown that high Na⁺ induces SGK-1 leading to dysfunctional Tregs and other recent studies have shown higher Na⁺ tissue levels in a subset of patients with MS (89). Of note, gene set enrichment analysis (GSEA) demonstrated a dysfunctional Foxo1 and Foxo3 KO Treg signature that was enriched in MS and SLE mTreg (FIG. 14), consistent with the previous studies showing impaired function of Foxo1 in dysfunctional Tregs in MS (18, 110, 111). Thus, it is hypothesized that molecular interactions leading to dysfunctional Tregs in autoimmunity can be driven in part through SGK-1 mediated both by genetic variation in CD28 and high sodium concentration as an environmental factor. However, it should be pointed out that this could be just one of many hundreds of gene-environmental interactions that drive autoimmune disease.

In summary, the data reported herein uncover fundamental molecular mechanisms by which Treg dysfunction is triggered in patients with MS and potentially other autoimmune diseases. Identification of the primate-specific alternative short PRDM1 isoform induction in MS Tregs highlights the importance of studying human tissues in addition to mouse models in obtaining insight into disease pathogenesis. Enhancement of the PRDM1 SGK1 axis in mTreg was observed in the other autoimmune diseases, suggesting shared mechanisms among dysfunctional Tregs in the context of autoimmunity. Furthermore, these findings link epigenetic priming to pathogenic short PRDM1 expression with contribution of AP-1 and IRF regulatory elements in MS Tregs that is potentially associated with genetic susceptibility of MS via the CD28/AP-1 pathway (6, 61, 75). Finally, the rich data reported herein of both transcriptome and epigenome profiles on human memory CD4⁺ T cells and Tregs will be a useful tool to explore further insights into pathogenic mechanisms of dysfunctional T cells in autoimmune diseases.

### Definitions

Unless specifically indicated otherwise, all technical and scientific terms used herein have the same meaning as commonly understood by those of ordinary skill in the art to which this application belongs. In addition, any method or material similar or equivalent to a method or material described herein can be used in the practice of the present application. For purposes of the present application, the following terms are defined.

The term “PRDM1” as used herein refers to a gene PRDM1 (PR/SET domain 1), which encodes the protein Blimp1 (B lymphocyte-induced maturation protein-1). The human PRDM1 gene is described by Ensembl identifier ENSG00000057657 (see also, e.g., useast.ensembl.org/Homo_sapiens/Gene/Summary?db=core;g=ENSG=:0000057657: 105993463-106109939). Ten different isoforms of human PRDM1 have been identified. “PRDM1-S,” “short PRDM1,” “short PRDM1 isoform,” or “PRDM1 short isoform,” and the like, as used herein refer to a short PRDM11 isoform as described according to Ensernbl identifier FNSTOOO(0369089 3 (PRDM1-201) and ENST00000450060.5 (PRDM1-205). “PRDM1-L,” “long PRDM1,” “long PRDM1 isoform,” or “PRDM1 long isoform,” and the like, as used herein refer to along PRDM1 isoform as described according to Ensembl identifier ENSTC00000369096.9 (PRDM1-203), ENST00000369091 6 (PRDM1-202), ENST00000424894.1 (PRDM1-204) and ENST00000648754.1 (PRDM1-208).

The terms “patient”, “individual”, “subject”, “mammal”, and “animal” are used interchangeably herein and refer to mammals, including, without limitation, human, veterinary animals (e.g., cats, dogs, cows, horses, sheep, pigs, etc.) and experimental animal models (e.g., mouse, rabbit, rat). Animals include all vertebrates, e.g., mammals and non-mammals, such as mice, sheep, dogs, cows, avian species, ducks, geese, pigs, chickens, amphibians, and reptiles. In a preferred embodiment, the subject is a dry-nosed mammal. In a preferred embodiment, the subject is a human. In some embodiments, a subject is in need of prevention or treatment for an autoimmune disease, an acute viral infection, a chronic viral infection, a cardiovascular disease, a neurodegenerative disease, a metabolic syndrome, or a cancer, or a related disorder or condition.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like.

The term “pharmaceutically acceptable”, as used herein, refers to molecular entities and other ingredients of such compostions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” or “a pharmaceutically acceptable carrier” as used herein, refers to any clinically useful solvents, diluents, adjuvants, excipients, recipients, vehicles and the like for use in preparing admixtures of a pharmaceutical composition.

The term “antibody” refers to all isotypes of immunoglobulins (e.g., IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies. Immunoglobulin molecules can be of any class (e.g., IgG1, IgG2, IgG3, IgG4, IgM1, IgM2, IgAQ1 and IgA2) or subclass.

The term “antigen-binding fragment” refers to any proteinaceous structure that may exhibit binding affinity for a particular antigen. Antigen-binding fragments include those produced by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more complementarity determining regions (CDRs) of an antibody known to bind a particular antigen. Examples of suitable antigen-binding fragments include, but not limited to, single-chain molecules such as Fab, F(ab′)2, Fc, Fabc, Fv molecules, scFv, and disulfide-linked Fvs (sdFv), intrabodies, diabodies, minibodies, linear antibodies, single domain antibodies such as sdAb (either VL or VH), camelid nanobodies (VHH domains), multi-specific antibodies formed from antibody fragments, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782 (which is incorporated herein by reference in its entirety), bivalent fragments comprising two Fab fragments linked by a disulfide bridge at the hinge region, a Fd fragment consisting essentially of the VH and CH1 domains, a dAb fragment, or an isolated CDR, and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. The phrase “an antibody or antigen-binding fragment thereof” may be used to denote that a given antigen-binding fragment incorporates one or more amino acid segments of the antibody referred to in the phrase.

As used herein, the singular forms “a”, “an” and “the” include plural references unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value, Where particular values are described in the application and claims, unless otherwise stated, the term “about” is implicit and in this context means within an acceptable error range for the particular value.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

In accordance with the present invention, there may be employed conventional molecular-biology, microbiology, and recombinant DNA techniques within the skill of the art. See, e.g., Sambrook, Fritsch and Maniatis, Molecular Cloning: A Laboratory Manual, 2nd ed., Cold Spring Harbor Laboratory Press, Cold Spring Harbor, N.Y., 1989 (herein “Sambrook et al., 1989”); DNA Cloning: A Practical Approach, Volumes I and II (Glover ed. 1985): Oligonucleotide Synthesis (Gait ed. 1984); Nucleic Acid Hybridization (Haines and Higgins eds. 1985); Transcription And Translation (Hames and Higgins eds. 1984); Animal Cell Culture (Freshney ed. 1986), Immobilized Cells And Enzymes (IRL Press, 1986); B. Perbal, A Practical Guide To Molecular Cloning (1984); Ausubel et al. eds., Current Protocols in Molecular Biology, John Wley and Sons, Inc. 1994; among others.

### Methods of Restoration of Treg Function

In one aspect, provided herein are methods of restoring, improving, or enhancing a regulatory T cell (Treg) function. In some embodiments, the Treg function is a Treg suppressive function.

In some embodiments, the Treg is a dysfunctional Treg. Treg function in human can be assessed by in vitro Treg suppression assay, for example, as described in the Examples section herein. In some embodiments, dysfunctional Tregs are characterized by higher Interferon gamma (IFNg) and/or Programmed cell death protein 1 (PD1) expression as compared to a control Treg.

In some embodiments, the method of described herein comprises contacting the Treg with an effective amount of an agent capable of reducing the expression and/or function of a short isoform of PRDM1 (PRDM1-S).

In some embodiments, the agent capable of reducing the expression and/or function of PRDM1-S inhibits the expression and/or function of the protein (Blimp1-S) encoded by PRDM1-S gene.

In some embodiments, the agent capable of reducing the expression and/or function of PRDM1-S inhibits the expression of the mRNA transcribed from PRDM1-S promoter.

In some embodiments, the agent capable of reducing the expression and/or function of PRDM1-S blocks one or more of a regulatory element(s) that controls the transcription of PRDM1-S mRNA. The regulatory element may be a promoter, an enhancer, or an insulator.

In one embodiment, the PRDM1-S promoter is located at approximately hg19 chr6:106,545,545-106,546,737. In one embodiment, the PRDM1-S promoter comprises or consists of the following sequence

(SEQ ID NO: 172)
GTGTGAGTGACTTCATGGCACCGACATTGCTGTTTTTAAATGAGGATACA
GTAAATTGCAGTCCGAGGAAGGCTAACTGGAATCAACATACCCGTAGCTT
TAGAAAGCAGTTTCCGCACCAGCGAAGAGTACAAGAGCGATGGAACCCCA
TGTTCCTGGAAGTTTGCACATCAGAGTAAACAAACTTGAAAACCCCTCTT
GATAGCAGAATTCACCCAGCCTTGTTCCATTTTCTCTTAACAAAACACAC
CGCAAAAGCTCTCACAAGCTGCTTTGATGAAGCCACATGTATTTCCCCCT
TCACAATTTACAGGAAGTTACTCTTAAAAGAAAGTGATTCTGGTGTTTAC
CGCCTGTGTTAAAGGGACAGAGTTCCTTTTTATTTCTGATAACGTTTGAG
CGAAATACAGAAACTATCTGTAGACTAGCATAGTCGGTACGTGAGTAAGG
AAAAGCAATAACCTGCTGTCCGGTGAGCACAAAATTCCTGCTACGAACAG
TGCCTTACTGCTGCTTGGAGACTGCAAGTCGCAGATCACACTAGGTATTG
ACTGATTGTATAAGGAAATTTCTTAAAGTCTAAAGTAAAGGTGGTACCTC
CTAAAAAGAGGGGAAGAGAGAAAACTTTGTGTGGAAGGATAAGGAGTGTG
TTTATAGTTTCAGTAAGAGTGTACGTTTTAATTTTTCTTCTTCCTCTGCC
TCTTTGCCAAGTAGCCTGAGTGCATCTGTTATCCAGAAGTAGTATTACTC
TAGGACAAACTTCAAATTCTTCATTCTGCGTTGCCTTTAAGGAACAACAT
ACTTTCTTCCTGTTCTTTTTCCAAAAACACACGCCTATGGCTCTGTGTGT
GGTGTTTTAGCCAGCCTCCTCCCAGATAAGGGGTTCCCTTCCCTCCTTTG
CATTGAAAGGAAAGTGCAAGTCTGGACATGTTTATCAAGAGGAAAAGTGA
CTTCTCAGTAATAGACTGTCAAATTCGGGCTGCTGCCCGAGTGTTCGCTT
TGTTATGGCAGGTGAAGTTCACCTTTGCCCCACCCAGTGTTTCCACAAAA
AGGCAAGGTTCCAAGTATTCATATGAACAAGTGTTACTTTAGGACTTGGA
GGGTTGGGGGTGGAGGATGTTTGCATAGTTGAAGCCTTGGGGGGGGGTGT
AGGAAACGGCGAGTACAGAGGCCATAGAAAAAGCTAAGACTCA

In one embodiment, the PRDM1-S enhancer is located at approximately hg19 chr6:106,194,443-106,194,840. This region corresponds to #2 peak referenced in the Examples section herein. In one embodiment, the PRDM1-S enhancer comprises or consists of the following sequence

(SEQ ID NO: 174)
CACAAGCCACATGAGACTTGTTCTTCTTTTCTTGAATAAAAGTTTTATCA
AAGTTATGCAAATTAGTATAGACCCACTGTTAGGGGCTGGGAGGAGAAGC
AGTCAATGTAACATGAAATAAAATAGACTAAGATCAAAAACAAACTATTC
TGTTTAGCTGACTCATTTCAAAATGAAATCAGTAATTTTGTAACAAGGGG
TGGGGGATCAGCAGATGTTTTCAAAGGATACGAAATTTCAGTTAGATAAG
AGATGTAAGTTCAAGAGATCTATTGTAGAACAAGGTGTTTATATAGTTAA
TAACAGTGTATTTCTGTAAATTGCTAGGAGAGTAGATTTTAAGTGTTCTC
ACCACAAAAAAAGAGACATGAGATAATGCATATTGCTAATTAGCTCAA

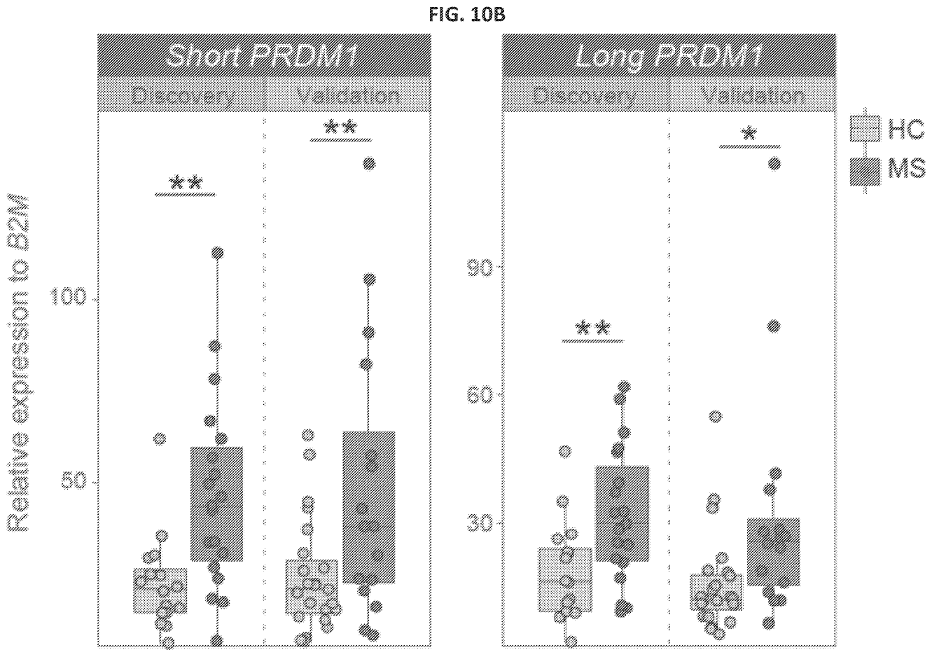

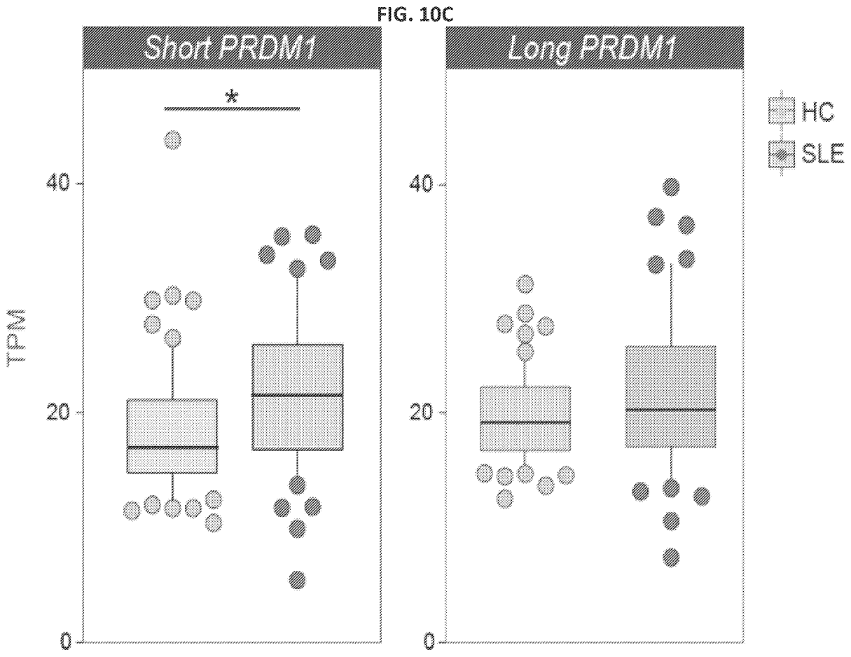

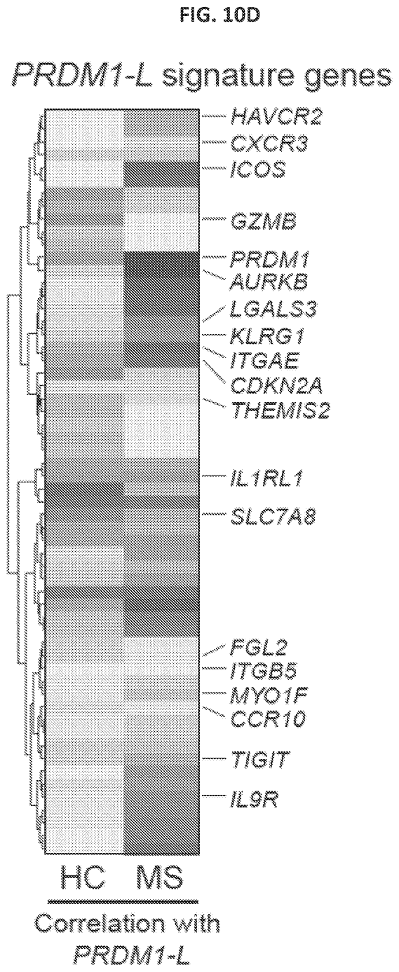

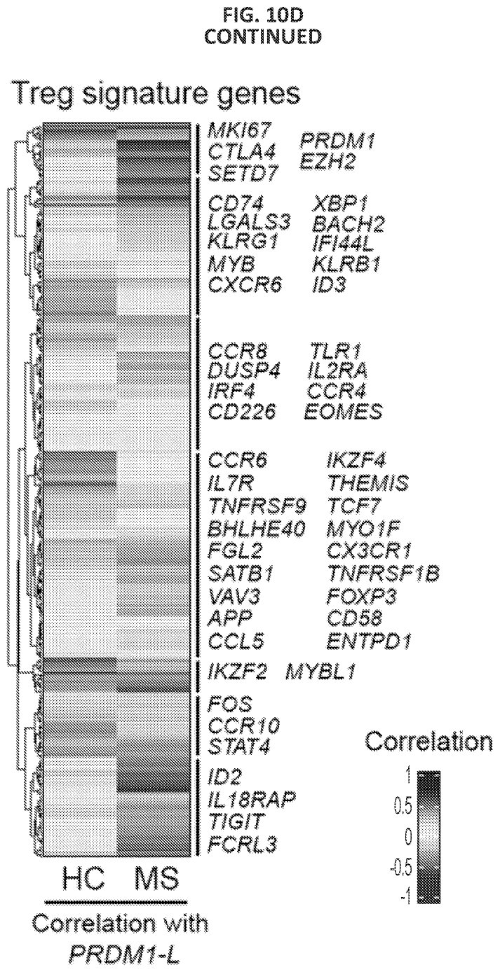

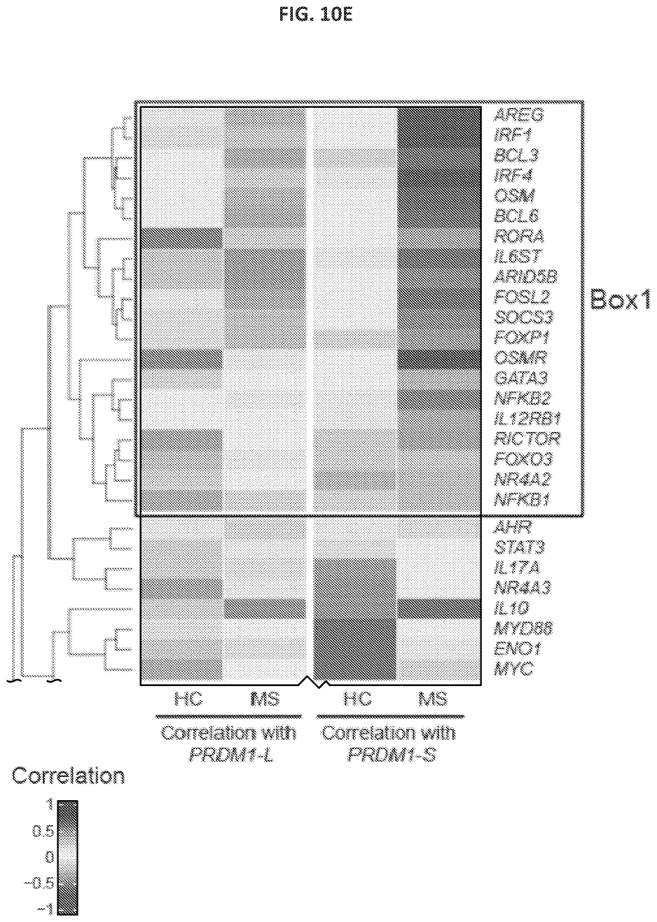

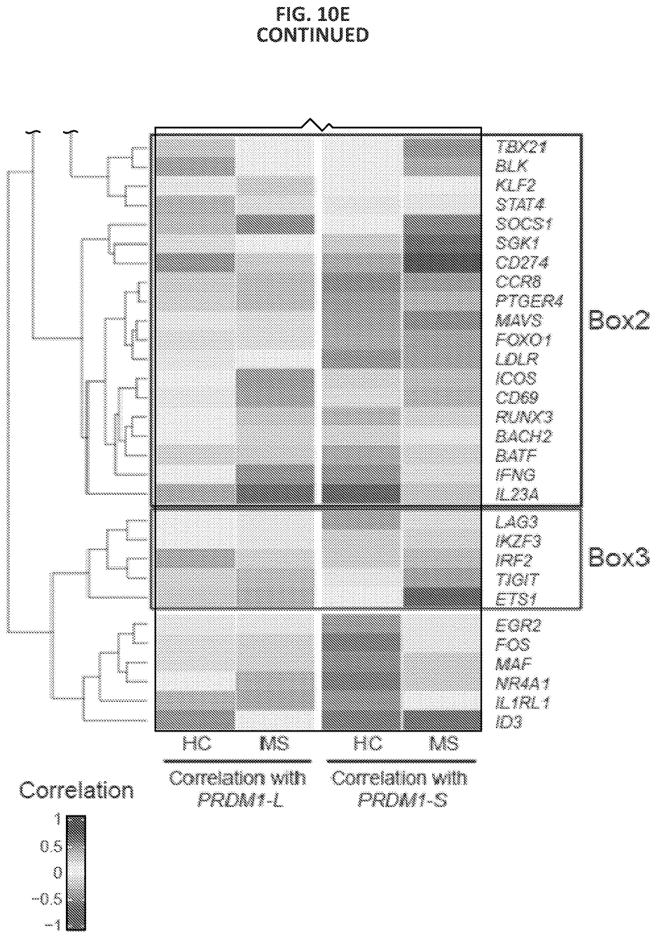

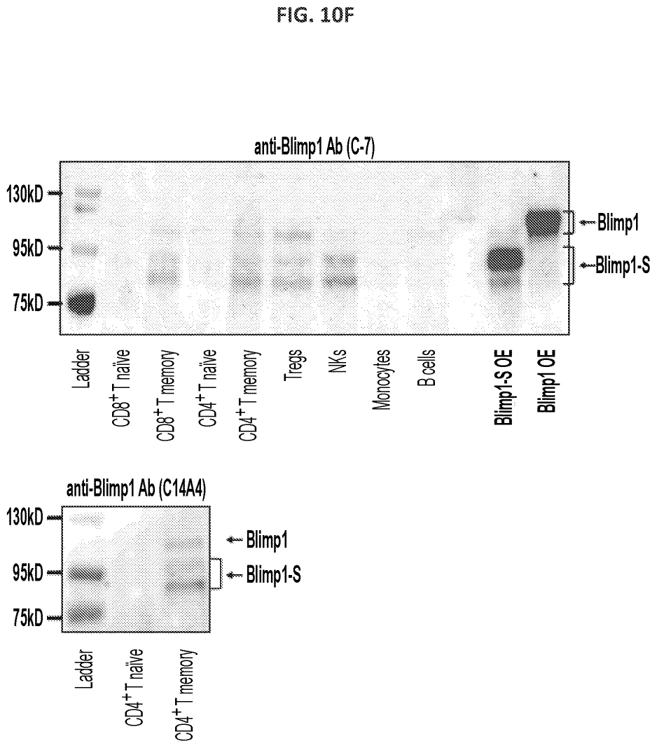

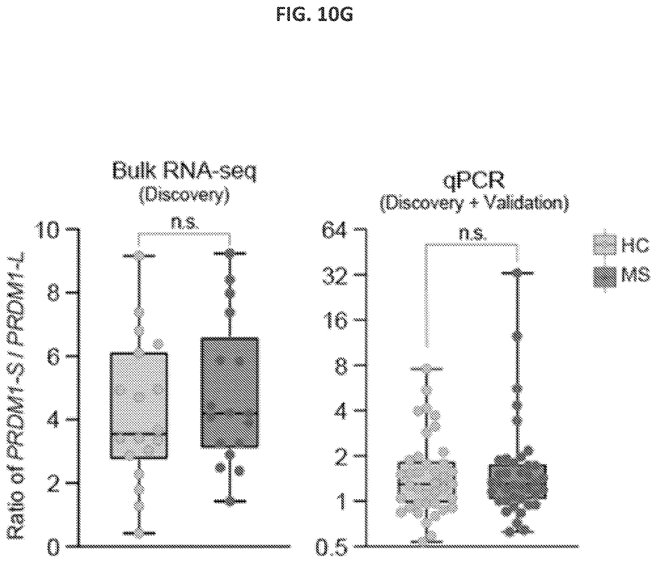

In some embodiments, the agent capable of reducing the expression and/or function of PRDM1-S is a siRNA, a shRNA, a miRNA, or an antisense oligonucleotide. In some embodiments, the siRNA, a shRNA, a miRNA, or an antisense oligonucleotide targets a unique exonic region in PRDM1-S(see, e.g., FIG. 10).

In some embodiments, the agent capable of reducing the expression and/or function of PRDM1-S may be a small molecule, an antibody or antigen-binding fragment thereof, or an aptamer.

In some embodiments, the agent capable of reducing the expression and/or function of PRDM1-S is a gene regulation system such as a CRISPR interference (CRISPRi) or CRISPRoff molecule.

In some embodiments, the agent reduces the expression and/or function of a short isoform of PRDM1 (PRDM1-S) by at least or about 10%, 20%, 30%, 40%, 50%, 60%, 70%, 80%, 90%, or 100% in the Treg, as compared to the level of expression and/or function of the short isoform of PRDM1 (PRDM1-S) in the absence of the agent. In some embodiments, the agent restores the level of expression and/or function of a short isoform of PRDM1 (PRDM1-S) in the Treg to the level of expression and/or function of the short isoform of PRDM1 (PRDM1-S) in a functional Treg.

In some embodiments, the method described herein comprises contacting the Treg with an effective amount of an agent capable of increasing the expression and/or function of a long isoform of PRDM1 (PRDM1-L).

In some embodiments, the agent capable of increasing the expression and/or function of PRDM1-L activates one or more regulatory element(s) that controls the transcription of PRDM1-L mRNA. The regulatory element may be a promoter, an enhancer, or an insulator.

In one embodiment, the PRDM1-L promoter is located at approximately hg19 chr6:106,533,676-106,534,195. In one embodiment, the PRDM1-L promoter comprises or consists of the following sequence:

(SEQ ID NO: 173)
AGTTCTGCCAAGTTTGAACGTTAGCAGGAGAAACGGAATGTTACAACTTT
TGGGGCGGGGGGCGGGGAAACGTGCGTTACATACACAACAGCTTGAGGAC
CAGACAGCTCCACTGTATTACACTAGCTGCAAAAACAATTTAACTTGCTC
TTTTGAAGTAAGATTTGTGTCTTTTGTACCTGGGGATTTGAGCTGAGAAA
TCAGAAACTGTGTAGGTAAATTTTAAGTTTCCTTAATTTAAGGAACGTGC
GCCCCCTAATTCTGCCGCGCCAGGAAGGAGGGCGATCTGGAGTGTTTAGA
ATACAATAGAGCCCAAGTAAGCGTTGAGGTTAAGTGCCTTCAAAGGGAAG
TAAGAAGATTCCAAGTCAATGTTGAAATACACATGCGAAGAGAGGAAGCT
CTCGGCGGCTGTGCTAGCAATCTGGGGGAAAGCCCTGGGCTCGGCCAGGT
GGTGTTGGCCACGTTGGCCACGCCCCCACTTCGCGCAGCCGAGTGGCTAA
GGAAATCTTAAGCAGGGAGG

In some embodiments, the agent capable of increasing the expression and/or function of PRDM1-L is a gene regulation system such as a CRISPR activation (CRISPRa) molecule.

In some embodiments, the agent increases the expression and/or function of a long isoform of PRDM1 (PRDM1-L) by at least or about 10%, 20%, 30%, 40%, 50%, 60%, 70%, 80%, 90%, or 100% in the Treg, as compared to the level of expression and/or function of the long isoform of PRDM1 (PRDM1-L) in the absence of the agent. In some embodiments, the agent restores the level of expression and/or function of a long isoform of PRDM1 (PRDM1-L) in the Treg to the level of expression and/or function of the long isoform of PRDM1 (PRDM1-L) in a functional Treg.

In some embodiments, the method described herein comprises contacting the Treg with an effective amount of an agent capable of reducing the expression and/or function of a short isoform of PRDM1 (PRDM1-S) and/or an effective amount of an agent capable of increasing the expression and/or function of a long isoform of PRDM1 (PRDM1-L).

In some embodiments, the agent(s) restore the ratio of the short isoform of PRDM1 (PRDM1-S) to the long isoform of PRDM1 (PRDM1-L) in the Treg to the ratio of the short isoform of PRDM1 (PRDM1-S) to the long isoform of PRDM1 (PRDM1-L) in a functional Treg.

In various embodiments, contacting of the Treg with an agent described herein can occur in vivo or ex vivo.

In some embodiments, the Treg is in a subject and the agent is administered to the subject.

### Methods of Treatment

In one aspect, provided herein are methods of treating a disease or disorder in a subject in need thereof by modulating the expression and/or function of short and/or long isoforms of PRDM1.

In some embodiments, the method of treating a disease or disorder described herein comprises administering to the subject an effective amount of an agent capable of reducing the expression and/or function of a short isoform of PRDM1 (PRDM1-S).

In some embodiments, the agent inhibits the expression and/or function of the protein (Blimp1-S) encoded by PRDM1-S gene.

In some embodiments, the agent capable of reducing the expression and/or function of PRDM1-S inhibits the expression and/or function of the protein (Blimp1-S) encoded by PRDM1-S gene.

In some embodiments, the agent capable of reducing the expression and/or function of PRDM1-S inhibits the expression of the mRNA transcribed from PRDM1-S promoter.

In some embodiments, the agent capable of reducing the expression and/or function of PRDM1-S blocks one or more of a regulatory element(s) that controls the transcription of PRDM1-S mRNA. The regulatory element may be a promoter, an enhancer, or an insulator.

In one embodiment, the PRDM1-S promoter is located at approximately hg19 chr6:106,545,545-106,546,737. In one embodiment, the PRDM1-S promoter comprises or consists of the sequence SEQ ID NO: 172.

In one embodiment, the PRDM1-S enhancer is located at approximately hg19 chr6:106,194,443-106,194,840. In one embodiment, the PRDM1-S enhancer comprises or consists of the sequence SEQ ID NO: 174.

In some embodiments, the agent capable of reducing the expression and/or function of PRDM1-S is a siRNA, a shRNA, a miRNA, or an antisense oligonucleotide.

In some embodiments, the agent capable of reducing the expression and/or function of PRDM1-S may be a small molecule, an antibody or antigen-binding fragment thereof, or an aptamer.

In some embodiments, the agent capable of reducing the expression and/or function of PRDM1-S is a gene regulation system such as a CRISPR interference (CRISPRi) or CRISPRoff molecule.

In some embodiments, the method of treating a disease or disorder described herein comprises administering to the subject an effective amount of an agent capable of increasing the expression and/or function of a long isoform of PRDM1 (PRDM1-L).

In some embodiments, the agent capable of increasing the expression and/or function of PRDM1-L activates one or more regulatory element(s) that controls the transcription of PRDM1-L mRNA. The regulatory element may be a promoter, an enhancer, or an insulator.

In one embodiment, the PRDM1-L promoter is located at approximately hg19 chr6:106,533,676-106,534,195. In one embodiment, the PRDM1-L promoter comprises or consists of the sequence SEQ ID NO: 173.

In some embodiments, the agent capable of increasing the expression and/or function of PRDM1-L is a gene regulation system such as a CRISPR activation (CRISPRa) molecule.

In various embodiments, the disease or disorder that is treatable with the methods or compositions described herein is an autoimmune disease, an acute viral infection, a chronic viral infection, a cardiovascular disease, a neurodegenerative disease, a metabolic syndrome, or a cancer.

In some embodiments, the disease is an autoimmune disease. Non-limiting list of autoimmune diseases that can is treatable with the methods or compositions described herein include Multiple Sclerosis, Lupus, Alopecia Areata, Ankylosing Spondylitis, Antiphospholipid Syndrome, Autoimmune Addison's Disease, Autoimmune Hemolytic Anemia, Autoimmune Hepatitis, Celiac Sprue-Dermatitis, Chronic Fatigue Immune Dysfunction Syndrome, Chronic Inflammatory Demyelinating Polyneuropathy, Churg-Strauss Syndrome, Cicatricial Pemphigoid, CREST Syndrome, Cold Agglutinin Disease, Crohn's Disease, Essential Mixed Cryoglobulinemia, Fibromyalgia-Fibromyositis, Graves' Disease, Rheumatoid Arthritis, Sarcoidosis, Scleroderma, Sjögren's Syndrome, Guillain-Barre, Hashimoto's Thyroiditis, Hypothyroidism, Idiopathic Pulmonary Fibrosis, Idiopathic Thrombocytopenia Purpura, IgA Nephropathy, Juvenile Arthritis, Lichen Planus, Meniere's Disease, Behcet's Disease, Bullous Pemphigoid, Cardiomyopathy, Mixed Connective Tissue Disease, Primary Biliary Cirrhosis, Psoriasis, Raynaud's Phenomenon, Reiter's Syndrome, Myasthenia Gravis, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammaglobulinemia, Rheumatic Fever, Stiff-Man Syndrome, Takayasu Arteritis, type I diabetes, Temporal Arteritis/Giant Cell Arteritis, Ulcerative Colitis, Uveitis, Vasculitis, Vitiligo, and Wegener's Granulomatosis.

In some embodiments, the autoimmune disease is multiple sclerosis.

In various embodiments of the methods described herein, the subject is a dry-nosed mammal. In some embodiments, the dry-nosed mammal is a dry-nosed primate. Dry-nosed primates, also known as haplorhines, generally include tarsiers, monkeys, apes, and humans.

In some embodiments, the subject is a human.

The dose of the described agents administered to a subject (such as a human) may vary with the particular composition, the method of administration, and the particular kind and stage of disease or disorder (such as an autoimmune disease) being treated. The amount should be sufficient to produce a desirable response, such as a therapeutic response against the disease or disorder (such as an autoimmune disease). In some embodiments, the amount of the composition (e.g., the described agent) is a therapeutically effective amount.

In some embodiments, the amount of the composition is an amount sufficient to produce a reduction in the expression and/or function of a short isoform of PRDM1 (PRDM1-S) by at least or about 10%, 20%, 30%, 40%, 50%, 60%, 70%, 80%, 90%, or 100% post administration of the composition, as compared to the level of expression and/or function of the short isoform of PRDM1 (PRDM1-S) in the absence of the agent. In some embodiments, the amount of the composition is an amount sufficient to restore the level of expression and/or function of a short isoform of PRDM1 (PRDM1-S) in one or more Tregs in the subject post administration of the composition to the level of expression and/or function of the short isoform of PRDM1 (PRDM1-S) in a functional Treg.

In some embodiments, the amount of the composition is an amount sufficient to produce an increase in the expression and/or function of a long isoform of PRDM1 (PRDM1-L) by at least or about 10%, 20%, 30%, 40%, 50%, 60%, 70%, 80%, 90%, or 100% post administration of the composition, as compared to the level of expression and/or function of the long isoform of PRDM1 (PRDM1-L) in the absence of the agent. In some embodiments, the amount of the composition is an amount sufficient to restore the level of expression and/or function of a long isoform of PRDM1 (PRDM1-L) in one or more Tregs in the subject post administration of the composition to the level of expression and/or function of the long isoform of PRDM1 (PRDM1-L) in a functional Treg.

In some embodiments, the amount of the composition is an amount sufficient to restore the ratio of the short isoform of PRDM1 (PRDM1-S) to the long isoform of PRDM1 (PRDM1-L) in one or more Tregs in the subject post administration of the composition to the ratio of the short isoform of PRDM1 (PRDM1-S) to the long isoform of PRDM1 (PRDM1-L) in a functional Treg.

Assays to measure the above changes in PRDM1-S and PRDM1-L expression and/or function include, but are not limited to, quantitative polymerase chain reaction (qPCR), microarray, RNA sequencing (RNA-Seq), single-cell RNA-Seq (scRNA-Seq), enzyme-linked immunoassay (ELISA), mass spectrometry, and Western blot.

In some embodiments, the amount of the composition is an amount sufficient to restore a Treg function. In some embodiments, the amount of the composition is an amount sufficient to restore Treg function by at least or about 5%, 10%, 15%, 20%, 25%, 30%, 35%, 40%, 45%, 50%, 55%, 60%, 65%, 70%, 75%, 80%, 85%, 90%, 95%, or 99% post administration of the composition. In some embodiments, the amount of the composition is an amount sufficient to restore Treg function in at least or about 5%, 10%, 15%, 20%, 25%, 30%, 35%, 40%, 45%, 50%, 55%, 60%, 65%, 70%, 75%, 80%, 85%, 90%, 95%, or 99% of the Treg cells in the subject post administration of the composition.

Treg function in human can be assessed by in vitro Treg suppression assay, for example, as described in the Examples section herein.

Agents that Modulate Expression and/or Function of PRDM1 Isoforms

The agents described herein include agents that are capable of reducing the expression and/or function of a short isoform of PRDM1 (PRDM1-S) and agents that are capable of increasing the expression and/or function of a long isoform of PRDM1 (PRDM1-L).

In some embodiments, the agent capable of reducing the expression and/or function of PRDM1-S is a siRNA, a shRNA, a miRNA, or an antisense oligonucleotide. The siRNA, shRNA, miRNA, an antisense oligonucleotide described herein can include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence (e.g., PRDM1-S mRNA sequence).

In some embodiments, the interfering nucleic acid molecule is an siRNA, also known as short interfering RNA or silencing RNA. In some embodiments, the siRNA molecules are 10, 11, 12, 13, 14, 15, 16, 17, 18, 19, 20, 21, 22, 23, 24, 25, 26, 27, 28, 29, 30, 35, 40, 45, 50, 55, 60, 65, 70, or more base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, or 21 to 24 base pairs in length.

In some embodiments, the siRNA is chemically modified. The siRNA may be chemically modified within the ribose sugar moiety, the nucleobase, and/or the nucleic acid backbone.

The ribose sugar modifications may include 2′-ribose substitutions (e.g., 2′-O-methyl, 2′-deoxy, 2′-fluoro, 2′-O-methoxyethyl, 2′-O-aminopropyl, 2′-O-dimethylaminoethyl, 2′-O-dimethylaminopropyl, 2′-O-dimethylaminoethyloxyethyl, and/or 2′-O-N-methylacetamido), creation of bridged nucleic acids (e.g., locked nucleic acid (LNA), 2′,4′-constrained 2′-0,4′-C-ethylene bridged nucleic acid and/or 2′-O-ethyl bridged nucleic acid), and/or creation of a phosphorodiamidate morpholino oligonucleotide (i.e., the five-membered ribose sugar is replaced by a six-membered morpholine ring). In some embodiments, each nucleotide of the siRNA molecule can a modified nucleotide (e.g., a 2′-modified nucleotide).

Nucleobases can be conventional bases (A, G, C, T, U), analogs thereof (e.g., modified uridines such as pseudouridine, 5-methoxyuridine, or N1-methylpseudouridine, or others); inosine; derivatives of purines or pyrimidines (e.g., N4-methyl deoxyguanosine, deaza-or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 6-O-methylguanine, 4-thio-pyrimidines, 4-amino-pyrimidines, 4-dimethylhydrazine-pyrimidines, and 4-O-alkyl-pyrimidines. Nucleic acids can also include one or more “abasic” residues where the backbone includes no nitrogenous base for position(s) of the polymer.

In some embodiments, the nucleobase modifications include a pyrimidine methylation, such as a 5-methyluridine, or a 5-methylcytidine, an abasic nucleotide, or an inverted abasic residues.

An siRNA molecule described herein may comprise one or more 2′-4′ bicyclic nucleosides in which the ribose ring may comprise a bridge moiety, e.g., connecting two atoms in the ring (e.g., connecting the 2′-0 atom to the 4′-C atom via an ethylene (ENA) bridge, a methylene (LNA) bridge, or a (S)-constrained ethyl (cEt) bridge).

In addition, the siRNA molecule may be modified or include nucleoside surrogates. Single-stranded regions of an siRNA molecule may be modified or include nucleoside surrogates, e.g., the unpaired region or regions of a hairpin structure, or a region which links two complementary regions, can have modifications or nucleoside surrogates. Modifications may also include those that stabilize one or more 3′- or 5-terminus of an siRNA molecule, e.g., against exonucleases, or favor the antisense siRNA agent to enter into RNA-induced silencing complex (RISC). Modifications can include C3 (or C6, C7, C12)amino linkers, thiol linkers, carboxyl linkers, non-nucleotidic spacers (C3, C6, C9, C12, abasic, tri ethylene glycol, hexaethylene glycol), special biotin or fluorescein reagents that come as phosphoramidites and that have another DMT-protected hydroxyl group, allowing multiple couplings during RNA synthesis.

The siRNA molecule may comprise a mix of nucleosides of different kinds. For example, the siRNA molecule may comprise a mix of deoxyribonucleosides or ribonucleosides and 2′-O-methyl modified nucleosides. A siRNA described herein may comprise a mix of 2′-fluoro modified nucleosides and 2′-O-methyl modified nucleosides. An siRNA described herein may comprise a mix of 2′-4′ bicyclic nucleosides and 2′-fluoro, 2′-O-methoxyethyl, or 2′-O-methyl modified nucleosides. An siRNA described herein may comprise a mix of non-bicyclic 2′-modified nucleosides (e.g., 2′-fluoro, 2′-O-methoxyethyl, or 2′-O-methyl) and 2′-4′ bicyclic nucleosides (e.g., ENA, cEt, LNA). An siRNA described herein may comprise a mix of 2′-deoxyribonucleosides or ribonucleosides and 2′-fluoro modified nucleosides.

The backbone modifications may include the incorporation of phosphorothioate linkages, in which one of the non-bridging oxygen atoms is replaced with sulfur and/or a peptide nucleic acid (PNA), in which a pseudo peptide polymer backbone substitutes the standard phosphodiester backbone of the RNA, morpholino backbones (see U.S. Pat. No. 5,034,506); amide backbones (see De Mesmaeker et al. Ace. Chem. Res. 1995, 28:366-374); or MMI or methylene(methylimino) backbones.

The siRNA molecule may comprise a phosphorothioate or other modified internucleotide linkage. The modified internucleotide linkages may comprise phosphorus-containing linkages. Examples of phosphorus-containing linkages include, but are not limited to, aminoalkylphosphotriesters, phosphotriesters, chiral phosphorothioates, phosphorothioates, phosphorodithioates, methyl and other alkyl phosphonates comprising 3′ alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

The siRNA molecule may comprise a phosphorothioate internucleoside linkage(s) between two or more nucleotides. The siRNA molecule may comprise a phosphorothioate internucleoside linkage(s) between all nucleotides. The siRNA molecule may comprise modified intemucleotide linkages at the first, second, and/or third internucleoside linkage at the 5′ or 3′ end of the siRNA molecule.

In some embodiments, the agent capable of reducing the expression and/or function of PRDM1-S may be a small molecule, an antibody or antigen-binding fragment thereof, or an aptamer.

In some embodiments, the antibody or antigen-binding fragment described herein is a human antibody, a monoclonal antibody, a humanized antibody, a single-chain Fv (scFv), a Fab, a Fab′, a F(ab′)2, an Fv fragment, a disulfide stabilized Fv fragment (dsFv), a (dsFv)₂, a V_{H}H, a Fv-Fc fusion, a scFv-Fc fusion, a scFv-Fv fusion, a diabody, a tribody, or a tetrabody.

The antibodies or antigen-binding fragments described herein may of any one of various antibody isotypes, such as IgM, IgD, IgG, IgA and IgE. In some embodiments, the antibody isotype is IgG1, IgG2, IgG3, or IgG4 isotype. In some embodiments, the antibody isotype is IgAQ1 or IgA2. Antibody or antigen-binding fragment thereof specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

In some embodiments, the methods described herein employ an agent that is a gene regulation system. A gene regulation system described herein can comprise a DNA binding protein such as an engineered (e.g., programmable or targetable) DNA nuclease which is preferably catalytically deactivated, coupled with a transcriptional effector (e.g., repressor, activator) to modulate target gene expression. Any suitable DNA nuclease can be used including, but not limited to, deactivated CRISPR-associated protein (Cas) nucleases, deactivated zinc finger nucleases (ZFNs), deactivated transcription activator-like effector nucleases (TALENs), deactivated meganucleases, other deactivated endo- or exo-nucleases, variants thereof, fragments thereof, and combinations thereof.

In some embodiments, the agent capable of reducing the expression and/or function of PRDM1-Sis a CRISPR interference (CRISPRi) molecule. In one embodiment, the CRISPRi molecule comprises a variant of CRISPR (such as a catalytically dead Cas9) coupled with a transcriptional repressor to inhibit target gene expression (e.g., PRDM1-S). The CRISPRi molecule can be directed by its guide RNA to the target genome locus (e.g., promoter of PRDM1-S) along with the effector arm, and represses the downstream gene expression (e.g., PRDM1-S). Another option to suppress the expression and/or function of PRDM1-S is CRISPRoff, which can be a programmable epigenetic regulation system comprising a dead Cas9 fusion protein that establishes DNA methylation and repressive histone modifications (Nunez et al., Cell. 2021 Apr. 29; 184(9):2503-2519.e17, which is incorporated herein by reference in its entirety).

In some embodiments, the agent capable of increasing the expression and/or function of PRDM1-L is a CRISPR activation (CRISPRa) molecule. In one embodiment, the CRISPRa molecule comprises a variant of CRISPR (such as a catalytically dead Cas9) coupled with a transcriptional activator to activate target gene expression (e.g., PRDM1-L). The CRISPRa molecule can be directed by its guide RNA to the target genome locus (e.g., promoter of PRDM1-L) along with the effector arm, and activate the downstream gene expression (e.g., PRDM1-L).

### Formulation, Dosing, and Routes of Administration

Any of the agents described herein can be present in a composition such as a formulation that includes other agents, excipients, or stabilizers.

In some embodiments, the composition further comprises a target agent or a carrier that promotes the delivery of the agent to a Treg. Exemplary carriers include liposomes, micelles, nanodisperse albumin and its modifications, polymer nanoparticles, dendrimers, inorganic nanoparticles of different compositions.

In some embodiments, the composition is suitable for administration to a human. In some embodiments, the composition is suitable for administration to a mammal such as, in the veterinary context, domestic pets and agricultural animals.

In some embodiments, the composition is administered to a subject (e.g., a human subject), after the onset of the target disease or disorder described herein (e.g., autoimmune disease). In some embodiments, the composition is administered 1 day, 2 days, 3 days, 4 days, 5 days, 6 days, or 7 days or more days after the onset of the target disease or disorder. In some embodiments, the composition is administered 1 week, 2 weeks, 3 weeks, 4 weeks, 5 weeks, 6 weeks, 7 weeks, 8 weeks, 9 weeks, 10 weeks, 20 week, 30 weeks, 40 weeks, 50 weeks, or more after the onset of the target disease or disorder. In some embodiments, the composition is administered 1 week, 2 years, 3 years, 4 years, 5 years, 6 years, 7 years, 8 years, 9 years, 10 years, 20 week, 30 years, 40 years, 50 years, or more after the onset of the target disease or disorder.

In some embodiments, the composition is administered to a subject (e.g., a human subject), prior to the onset of the target disease or disorder described herein (e.g., autoimmune disease). In some embodiments, the composition is administered 1 day, 2 days, 3 days, 4 days, 5 days, 6 days, or 7 days or more prior to the onset of the target disease or disorder. In some embodiments, the composition is administered 1 week, 2 weeks, 3 weeks, 4 weeks, 5 weeks, 6 weeks, 7 weeks, 8 weeks, 9 weeks, 10 weeks, 20 week, 30 weeks, 40 weeks, 50 weeks, or more prior to the onset of the target disease or disorder. In some embodiments, the composition is administered 1 week, 2 years, 3 years, 4 years, 5 years, 6 years, 7 years, 8 years, 9 years, 10 years, 20 week, 30 years, 40 years, 50 years, or more prior to the onset of the target disease or disorder.

There are a wide variety of suitable formulations of the composition comprising a described agent disclosed herein. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. In some embodiments, the composition comprising the described agent with a carrier as discussed herein is present in a dry formulation (such as lyophilized composition). The formulations can additionally include lubricating agents, wetting agents, emulsifying and suspending agents, preserving agents, sweetening agents or flavoring agents.

In some embodiments, the compositions are formulated to be administered by any route which results in a therapeutically effective outcome. These include but are not limited to administered intravenously, intraarterially, intraperitoneally, intravesicularly, subcutaneously, intrathecally, intrapulmonarily, intramuscularly, intratracheally, intraocularly, transdermally, orally, or by inhalation.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

In certain embodiments, the compositions of the disclosure are formulated using one or more pharmaceutically acceptable excipients or carriers. In certain embodiments, the pharmaceutical compositions of the disclosure comprise a therapeutically effective amount of an agent of the disclosure and a pharmaceutically acceptable carrier.

Pharmaceutically acceptable carriers can include a physiologically acceptable compound that acts to, e.g., stabilize, or increase or decrease the absorption or clearance rate of a pharmaceutical composition. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of glycopeptides, or excipients or other stabilizers and/or buffers. Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. Detergents can also be used to stabilize or to increase or decrease the absorption of the pharmaceutical composition, including liposomal carriers. Pharmaceutically acceptable carriers and formulations are known to the skilled artisan and are described in detail in the scientific and patent literature, see e.g., the latest edition of Remington's Pharmaceutical Science, Mack Publishing Company, Easton, Pa. (“Remington's”). One skilled in the art would appreciate that the choice of a pharmaceutically acceptable carrier including a physiologically acceptable compound depends, for example, on the route of administration of the composition, and on its particular physio-chemical characteristics.

Compositions may be administered by any suitable means, for example, orally, such as in the form of pills, tablets, capsules, granules or powders; sublingually; buccally; parenterally, such as by subcutaneous, intravenous, intramuscular, intraperitoneal or intrastemal injection or using infusion techniques (e.g., as sterile injectable aqueous or non-aqueous solutions or suspensions); nasally, such as by inhalation spray, aerosol, mist, or nebulizer; topically, such as in the form of a cream, ointment, salve, powder, or gel; transdermally, such as in the form of a patch; transmucosally; or rectally, such as in the form of suppositories. The present compositions may also be administered in a form suitable for immediate release or extended release. Immediate release or extended release may be achieved by the use of suitable pharmaceutical compositions, or, particularly in the case of extended release, by the use of devices such as subcutaneous implants or osmotic pumps.

In various embodiments, the pharmaceutical composition is formulated for oral administration. Suitable forms for oral administration include, but are not limited to, tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinyl acetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, using hydroxymethylcellulose or gelatin-microcapsules or poly(methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadeca ethyleneoxy cetanol), or condensation products of ethylene oxide with partial esters derived from fatty acids and a hexitol (e.g., polyoxy ethylene sorbitol rnonooleate), or condensation products of ethylene oxide with partial esters derived from fatty acids and hexitol anhydrides (e.g., polyethylene sorbitanmonooleate). The aqueous suspensions may also contain one or more preservatives.

Other suitable formulations for oral use include oily suspensions. Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soybean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions of the disclosure can be produced in useful dosage units for administration by various routes including, among others, topical, oral, subcutaneous, intravenous, and intranasal administration.

The pharmaceutical compositions of the disclosure can also include other biologically active substances in combination with the compounds of the disclosure. Such additional biologically active substances can be also formulated as separate compositions and can be administered simultaneously or sequentially with the compounds of the disclosure. Non-limiting examples of useful biologically active substances include statins, niacin, bile-acid resins, fibric acid derivatives, cholesterol absorption inhibitors, and other lipid-lowering drugs.

The optimal therapeutically effective amount of a compound or composition of this disclosure may be determined experimentally, taking into consideration the exact mode of administration, the form in which the drug is administered, the indication toward which the administration is directed, the subject involved (e.g., body weight, health, age, sex, etc.), and the preference and experience of the physician or veterinarian in charge.

Following methodologies which are well-established in the art, effective doses and toxicity of the compounds and compositions of the present disclosure, which performed well in in vitro tests, can be determined in studies using small animal models (e.g., mice, rats) in which they have been found to be therapeutically effective and in which these drugs can be administered by the same route proposed for the human trials.

For any pharmaceutical composition used in the methods of the disclosure, dose-response curves derived from animal systems can be used to determine testing doses for administration to humans. In safety determinations for each composition, the dose and frequency of administration should meet or exceed those anticipated for use in any clinical trial.

As disclosed herein, the dose of the compounds or compositions of the present disclosure is determined to ensure that the dose administered continuously or intermittently will not exceed an amount determined after consideration of the results in test animals and the individual conditions of a patient. A specific dose naturally varies (and is ultimately decided according to the judgment of the practitioner and each patient's circumstances) depending on the dosage procedure, the conditions of a patient or a subject animal such as age, body weight, sex, sensitivity, feed, dosage period, drugs used in combination, seriousness of the disease, etc.

Toxicity and therapeutic efficacy of the compositions of the disclosure can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀.

The compounds the disclosure can be formulated for parenteral, oral, topical, transdermal, transmucosal, intranasal, buccal administration, or by any other standard route of administration. Parenteral administration includes, among others, intravenous (i.v.), subcutaneous (s.c.), intraperitoneal (i.p.), intramuscular (i.m.), subdermal (s.d.), intradermal (i.d.), intra-articular, intra-synovial, intra-arteriole, intraventricular, intrathecal, intrasternal, intrahepatic, intralesional, or intracranial administration, by direct injection, via, for example, bolus injection, continuous infusion, or gene gun. A preferred route of administration according to the present disclosure will depend primarily on the indication being treated and includes, among others, topical, oral, subcutaneous, intravenous, and intranasal administration.

Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use. Suitable formulations for parenteral administration may contain substances which increase viscosity, for example, sodium carboxymethyl cellulose, sorbitol, and/or dextran. Optionally, the formulation may also contain stabilizers. Additionally, the compounds of the present disclosure may also be administered encapsulated in liposomes. The compounds, depending upon their solubilities, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

In specific embodiments, the compounds and/or compositions of the present disclosure are formulated for oral administration. For oral administration, the formulations of the disclosure can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. The compositions of the disclosure can be also introduced in microspheres or microcapsules, e.g., fabricated from poly glycolic acid/lactic acid (PGLA) (see, U.S. Pat. Nos. 5,814,344; 5,100,669 and 4,849,222; PCT Publication Nos. WO 95/11010 and WO 93/07861). Liquid preparations for oral administration can take the form of, for example, solutions, syrups, emulsions or suspensions, or they can be presented as a dry product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For administration by inhalation, the therapeutics according to the present disclosure can be conveniently delivered in the form of an aerosol spray presentation from pressurized packs or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In addition to the formulations described previously, the compositions can also be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In some embodiments, the methods of the disclosure further comprise administering at least one additional therapeutic agent useful for treating or preventing a target disease or disorder described herein (e.g., autoimmune disease). This additional therapeutic agent may comprise therapeutic agents identified herein or another therapeutic agent, e.g., commercially available therapeutic agent, known to treat, prevent or reduce the symptoms of the target disease or disorder (e.g., autoimmune disease). For example, the additional therapeutic agent may be an agent capable of restoring a Treg function. The agents of the present disclosure and the additional therapeutic agent(s) may be present in the same composition or different compositions.

Non-limiting examples of additional therapeutic agents contemplated for use in accordance with the disclosure include conventional disease modifying drugs, chemotherapies, radiotherapies, surgery, or bone marrow transplantation.

A synergistic effect of the combination therapy may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

Methods of Screening for Compounds that Restore Treg Function

In one aspect, provided herein is a method of screening for a candidate compound that restores a regulatory T cell (Treg) function (e.g., Treg suppressive function), comprising: (a) determining the expression or function of a short isoform of PRDM1 (PRDM1-S) and/or a long form of PRDM1 (PRDM1-L) in the Treg before and after contacting said Treg with a test compound; and (b) selecting a compound that i) reduces the expression and/or function of PRDM1-S in comparison with the expression and/or function of PRDM1-S determined in the absence of the test compound; and/or ii) increases the expression or function of long form of PRDM1 (PRDM1-L) in comparison with the expression and/or function of PRDM1-L determined in the absence of the test compound.

In some embodiments, there is provided a method of screening for a candidate compound that is useful for treating a target disease or disorder described herein, comprising: a) providing a plurality of candidate compounds; and b) identifying the candidate compound that restores a regulatory T cell (Treg) function (e.g., Treg suppressive function), thereby obtaining an agent that is useful for treating the target disease or disorder. In some embodiments, the disease or disorder is an autoimmune disease, an acute viral infection, a chronic viral infection, a cardiovascular disease, a neurodegenerative disease, a metabolic syndrome, or a cancer.

The techniques for determining the expression or function of PRDM1-S or PRDM1-L include, but are not limited to, quantitative polymerase chain reaction (qPCR), microarray, RNA sequencing (RNA-Seq), single-cell RNA-Seq (scRNA-Seq), enzyme-linked immunoassay (ELISA), mass spectrometry, and Western blot.

In some embodiments, the candidate compound is a siRNA, a shRNA, a miRNA, an antisense oligonucleotide, a small molecule, an antibody or antigen-binding fragment thereof, an aptamer, or a gene regulation molecule (such as a CRISPR interference (CRISPRi) or CRISPR activation (CRISPRa) molecule).

The candidate or test compounds or agents of or employed by the present application can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al. (1997) Anticancer Drug Des. 12: 145, incorporated by reference in its entirety for all purposes).

Examples of methods for the synthesis of molecular libraries can be found in the art, for example in: DeWitt et al. (1993) Proc. Natl. Acad. Sci. U.S.A. 90: 6909; Erb et al. (1994) Proc. Natl. Acad. Sci. USA 91: 11422; Zuckermann et al. (1994). J. Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and in Gallop et al. (1994) J. Med. Chem. 37: 1233, each of which are incorporated by reference in their entirety for all purposes. Libraries of compounds may be presented in solution (e.g., Houghten (1992) Biotechniques 13: 412), or on beads (Lam (1991) Nature 354: 82), chips (Fodor (1993) Nature 364: 555), bacteria (Ladner, U.S. Pat. No. 5,223,409), spores (Ladner'409), plasmids (Cull et al. (1992) Proc Natl Acad Sci USA 89: 1865) or on phage (Scott and Smith (1990) Science 249: 386); (Devlin (1990) Science 249: 404); (Cwirla et al. (1990) Proc. Natl. Acad. Sci. 87: 6378); (Felici (1991) J. Mol. Biol. 222: 301); (Ladner, supra), each of which are incorporated by reference in their entirety for all purposes.

Also provided are agents identified by any of the methods described herein. Accordingly, it is within the scope of the application to further use an agent identified as described herein in an appropriate animal model. For example, an agent identified as described herein (e.g., an agent capable of restoring Treg function) can be used in an animal model to determine the efficacy, toxicity, or side effects of treatment with such an agent. Alternatively, an agent identified as described herein can be used in an animal model to determine the mechanism of action of such an agent. Furthermore, this application pertains to uses of novel agents identified by the above-described screening assays for treatments as described herein.

### EXAMPLES

The following examples are provided to further describe some of the embodiments disclosed herein. The examples are intended to illustrate, not to limit, the disclosed embodiments.

### Example 1. Deep Transcriptomic Analysis of Memory Treg and Tconv Highlight PRDM1 Upregulation in MS

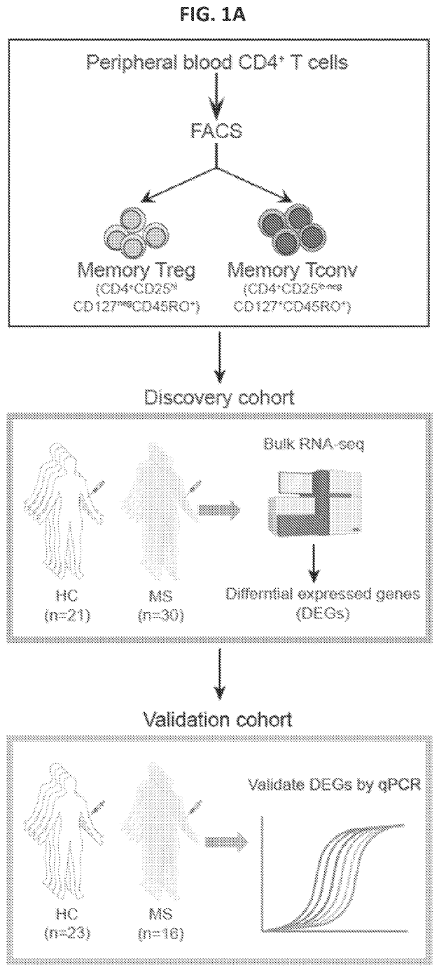

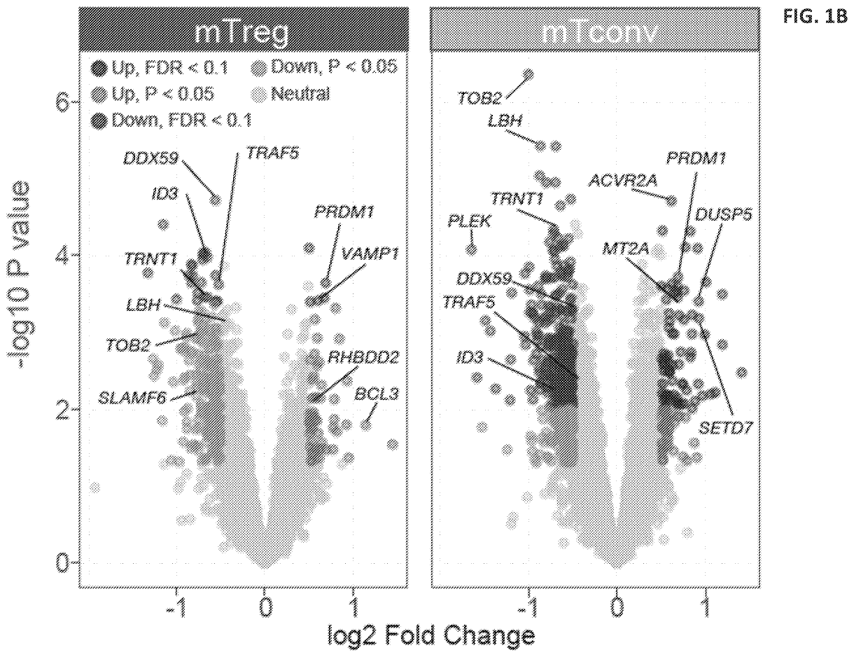

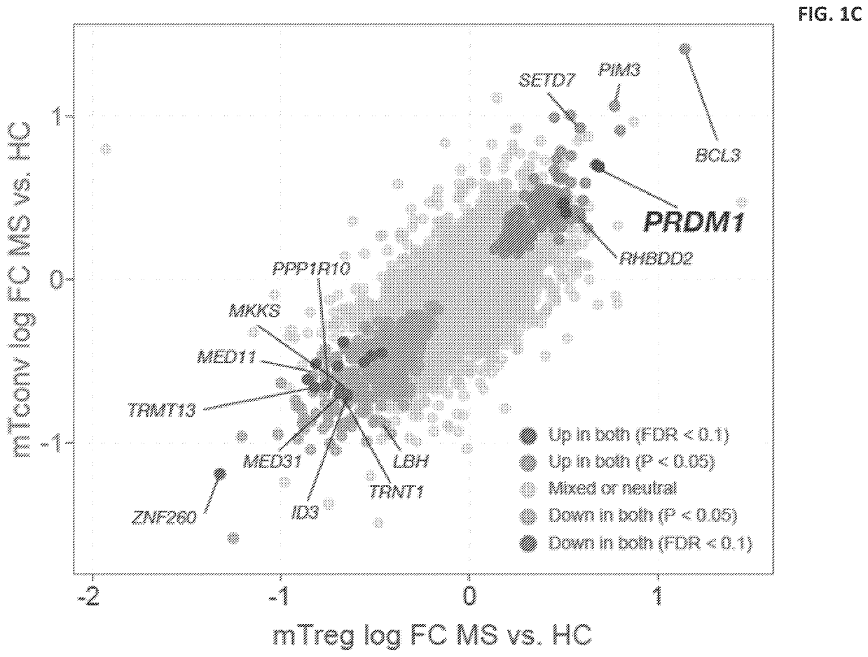

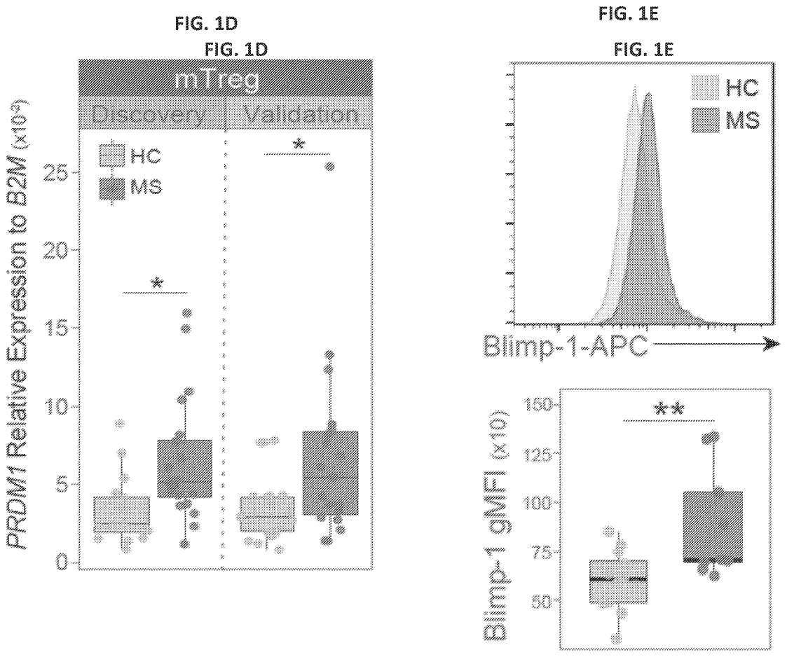

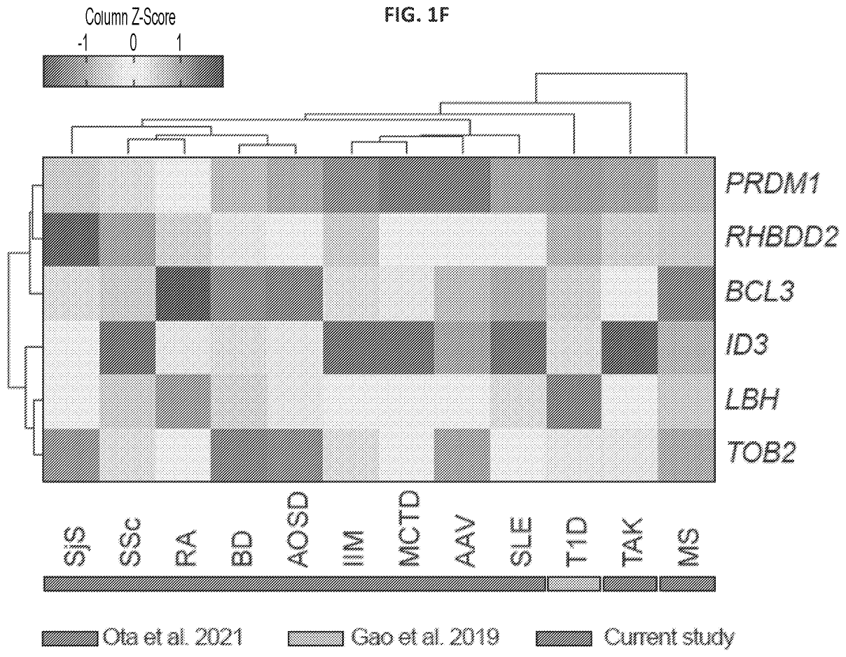

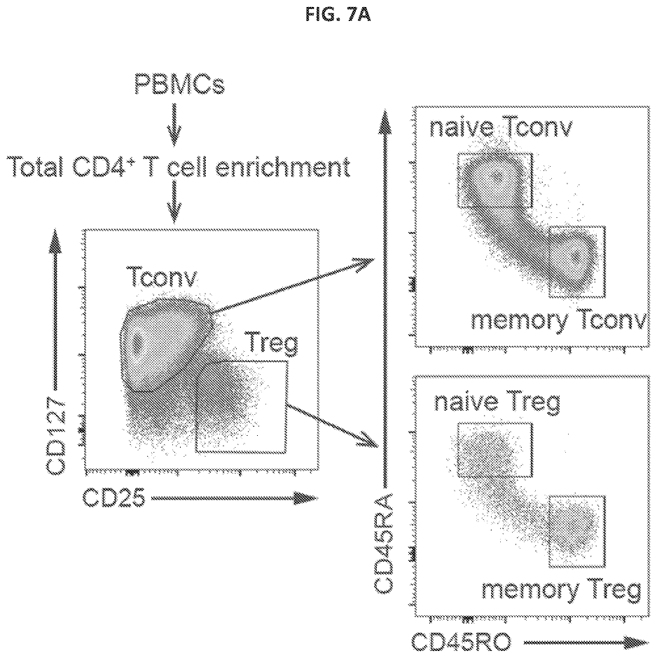

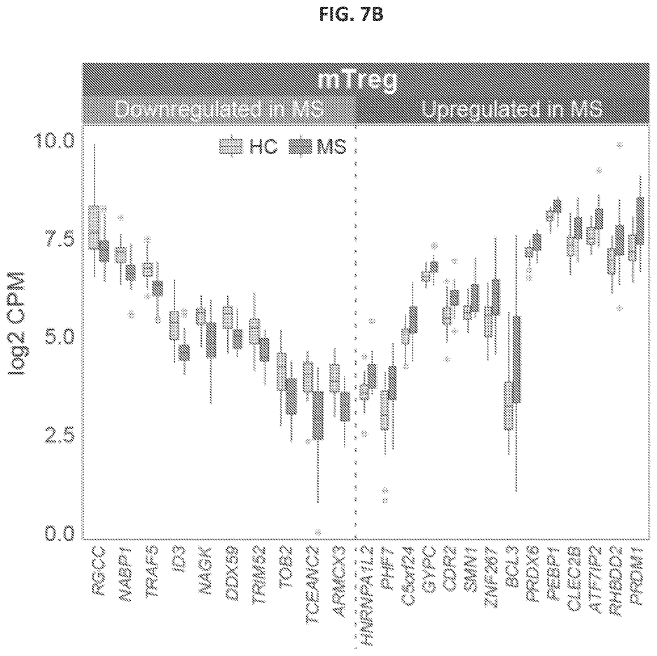

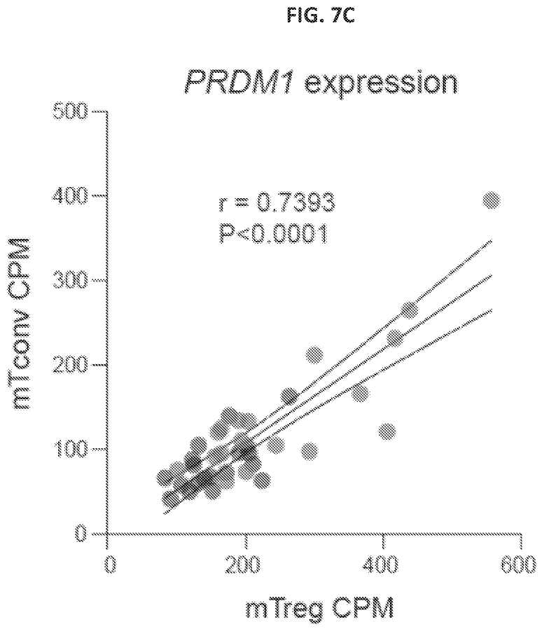

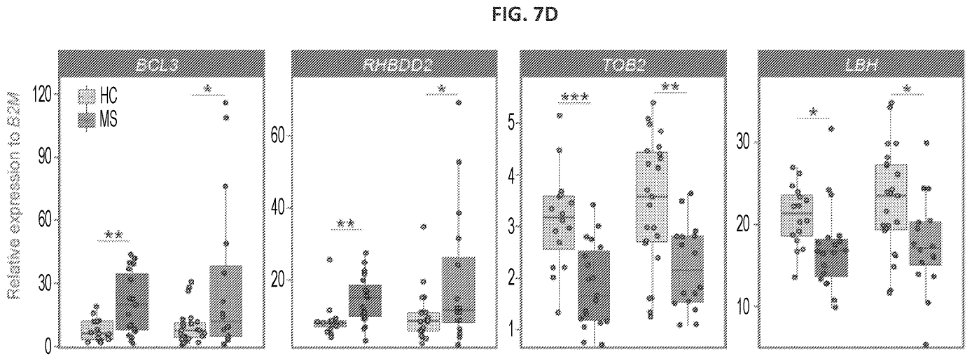

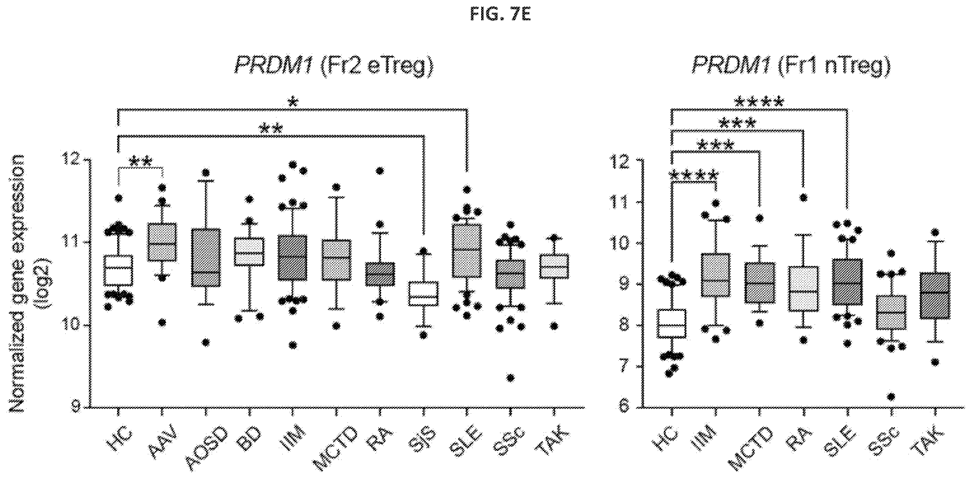

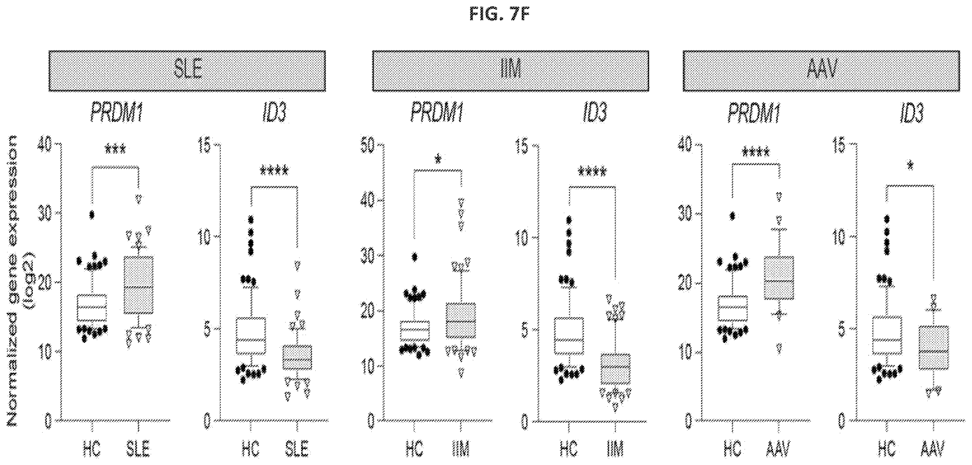

An effort to identify CD4+ T cell transcriptional differences between patients with MS and healthy controls was carried out. As previous studies had not identified significant differences in bulk transcriptional profiles of whole CD4+ T cells between MS subjects and healthy controls (7, 8), CD4+ T cells were divided into four major subpopulations based on two categories; Tconv vs. Treg, and naïve vs. memory (FIG. 7A), where each population is hypothesized to be involved in MS pathogenesis. For example, mTconv contains pathogenic CD4+ T cells in patients with MS, such as myelin-reactive T cells, which display the signatures of Th1 and/or Th17 cells (23-26). Moreover, mTregs with reduced suppressive function are implicated in MS pathophysiology (27, 28). To provide a comprehensive transcriptomic catalogue of memory CD4+ T cell subpopulations in patients with relapsing-remitting MS (RRMS), performed RNA-seq was performed on ex vivo mTreg and mTconv isolated from the peripheral blood of untreated RRMS patients free of steroid treatment and healthy control subjects as a discovery cohort (FIG. 1A). The control subjects were matched by age, sex, and ethnicity (clinical characteristics are described in Table 1A). Among a total of 90 RNA-seq samples (48 mTconv and 42 mTreg samples) in the discovery cohort (HC; n=21, MS; n=30), 84 samples (HC; n=20, MS; n=26) passed quality control (Method Details). 21 and 243 differentially expressed genes (DEGs, defined as |log2FC|>0.6, FDR<0.1) were identified between MS and healthy subjects for mTreg and mTconv, respectively (FIG. 1B). Several DEGs were up- or down-regulated in the same direction in both mTreg and mTconv: PRDM1, BCL3, and PIM3 were upregulated genes, and ID3, TOB2, and LBH were downregulated genes in MS (FIG. 1C, 7B). PRDM1 was identified as one of the top genes significantly upregulated in MS in both mTreg and mTconv (FIG. 7C). Reduced expression of ID3 in both cell types in MS reflects the negative regulation of ID3 by PRDM1 and ID3 helps to maintain Foxp3 expression in Treg (29) (FIG. 1D).

TABLE 1A
Patient Information (Healthy Controls)

| | | | Age (at | | | |
|---|---|---|---|---|---|---|
| | | | time of | Cell | | Passed |
| HC# | Gender | Ethnicity | collection) | types | Analysis | QC |
| hc#2 | M | Caucasian/Non- | 36 | mTeff | Bulk RNA-seq | Yes |
| | | Hispanic | | | | |
| hc#3 | M | Asian/Non-Hispanic | 37 | mTeff | Bulk RNA-seq | Yes |
| hc#4 | F | Caucasian/Non- | 23 | mTeff | Bulk RNA-seq | Yes |
| | | Hispanic | | | | |
| hc#5 | F | Caucasian/Non- | 31 | mTeff | Bulk RNA-seq | Yes |
| | | Hispanic | | | | |
| hc#6 | M | Caucasian/Non- | 55 | mTeff | Bulk RNA-seq | Yes |
| | | Hispanic | | | | |
| hc#7 | M | Asian/Non-Hispanic | 56 | mTeff | Bulk RNA-seq | Yes |
| hc#8 | M | Asian/Non-Hispanic | 35 | mTeff | Bulk RNA-seq | Yes |
| hc#9 | M | Caucasian/Non- | 41 | mTeff | Bulk RNA-seq | No |
| | | Hispanic | | | | |
| hc#10 | F | Caucasian/Non- | 37 | mTeff | Bulk RNA-seq | Yes |
| | | Hispanic | | | | |
| hc#11 | F | Asian/Non-Hispanic | 29 | mTeff | Bulk RNA-seq | Yes |
| hc#12 | F | Caucasian/Non- | 24 | mTeff | Bulk RNA-seq | Yes |
| | | Hispanic | | | | |
| hc#13 | F | Asian/Non-Hispanic | 33 | mTeff | Bulk RNA-seq | Yes |
| hc#14 | F | Hispanic | 23 | mTeff | Bulk RNA-seq | Yes |
| hc#15 | F | Caucasian/Non- | 23 | mTeff | Bulk RNA-seq | Yes |
| | | Hispanic | | | | |
| hc#16 | M | Caucasian/Non- | 43 | mTeff | Bulk RNA-seq | Yes |
| | | Hispanic | | | | |
| hc#17 | F | Caucasian/Non- | 46 | mTeff | Bulk RNA-seq | Yes |
| | | Hispanic | | | | |
| hc#18 | M | Caucasian/Non- | 45 | mTeff | Bulk RNA-seq | Yes |
| | | Hispanic | | | | |
| hc#19 | F | Caucasian/Non- | 26 | mTeff | Bulk RNA-seq | Yes |
| | | Hispanic | | | | |
| hc#20 | F | Caucasian/Non- | 39 | mTeff | Bulk RNA-seq | Yes |
| | | Hispanic | | | | |
| hc#21 | F | Caucasian/Non- | 37 | mTeff | Bulk RNA-seq | Yes |
| | | Hispanic | | | | |
| hc#22 | F | Caucasian/Non- | 62 | mTeff | Bulk RNA-seq | Yes |
| | | Hispanic | | | | |
| hc#1 | M | Asian/Non-Hispanic | 36 | mTreg | Bulk RNA-seq | Yes |
| hc#2 | M | Caucasian/Non- | 36 | mTreg | Bulk RNA-seq | Yes |
| | | Hispanic | | | | |
| hc#3 | M | Asian/Non-Hispanic | 37 | mTreg | Bulk RNA-seq | Yes |
| hc#4 | F | Caucasian/Non- | 23 | mTreg | Bulk RNA-seq | Yes |
| | | Hispanic | | | | |
| hc#5 | F | Caucasian/Non- | 31 | mTreg | Bulk RNA-seq | Yes |
| | | Hispanic | | | | |
| hc#6 | M | Caucasian/Non- | 55 | mTreg | Bulk RNA-seq | Yes |
| | | Hispanic | | | | |
| hc#7 | M | Asian/Non-Hispanic | 56 | mTreg | Bulk RNA-seq | Yes |
| hc#10 | F | Caucasian/Non- | 37 | mTreg | Bulk RNA-seq | Yes |
| | | Hispanic | | | | |
| hc#11 | F | Asian/Non-Hispanic | 29 | mTreg | Bulk RNA-seq | Yes |
| hc#12 | F | Caucasian/Non- | 24 | mTreg | Bulk RNA-seq | Yes |
| | | Hispanic | | | | |
| hc#13 | F | Asian/Non-Hispanic | 33 | mTreg | Bulk RNA-seq | Yes |
| hc#14 | F | Hispanic | 23 | mTreg | Bulk RNA-seq | Yes |
| hc#15 | F | Caucasian/Non- | 23 | mTreg | Bulk RNA-seq | Yes |
| | | Hispanic | | | | |
| hc#16 | M | Caucasian/Non- | 43 | mTreg | Bulk RNA-seq | Yes |
| | | Hispanic | | | | |
| hc#17 | F | Caucasian/Non- | 46 | mTreg | Bulk RNA-seq | Yes |
| | | Hispanic | | | | |
| hc#18 | M | Caucasian/Non- | 45 | mTreg | Bulk RNA-seq | Yes |
| | | Hispanic | | | | |
| hc#19 | F | Caucasian/Non- | 26 | mTreg | Bulk RNA-seq | Yes |
| | | Hispanic | | | | |
| hc#20 | F | Caucasian/Non- | 39 | mTreg | Bulk RNA-seq | Yes |
| | | Hispanic | | | | |
| hc#21 | F | Caucasian/Non- | 37 | mTreg | Bulk RNA-seq | Yes |
| | | Hispanic | | | | |
| hc#22 | F | Caucasian/Non- | 62 | mTreg | Bulk RNA-seq | Yes |
| | | Hispanic | | | | |
| hc#2 | F | Caucasian/Non- | 48 | CD4+T | multi-omics | Yes |
| | | Hispanic | | cells | single cell | |
| | | | | | analysis | |
| hc#24 | F | Caucasian/Non- | 23 | CD4+ T | multi-omics | Yes |
| | | Hispanic | | cells | single cell | |
| | | | | | analysis | |
| hc#25 | F | Caucasian/Non- | 33 | CD4+ T | multi-omics | Yes |
| | | Hispanic | | cells | single cell | |
| | | | | | analysis | |
| hc#26 | F | Caucasian/Non- | 25 | CD4+ T | multi-omics | Yes |
| | | Hispanic | | cells | single cell | |
| | | | | | analysis | |
| hc#27 | M | Caucasian/Non- | 39 | CD4+ T | multi-omics | Yes |
| | | Hispanic | | cells | single cell | |
| | | | | | analysis | |

TABLE 1B
Patient Information (MS Patients)

| | | | Age (at | | | | | | Reason |
|---|---|---|---|---|---|---|---|---|---|
| | | | time of | Clinical_{—} | | Cell_{—} | analysis | | for |
| Pt# | Gender | Ethnicity | collection) | Type | EDSS | Type | type | Include | exclusion |
| pt#01 | M | African | 32 | RRMS | na | mTreg | Bulk RNA-seq | Yes | |
| | | american/Non- | | | | | | | |
| | | Hispanic | | | | | | | |
| pt#02 | F | Hispanic | 37 | RRMS | 3.5 | mTreg | Bulk RNA-seq | Yes | |
| pt#02 | F | Hispanic | 37 | RRMS | 3.5 | mTeff | Bulk RNA-seq | Yes | |
| pt#03 | M | Caucasian/Non- | 31 | RRMS | 6 | mTreg | Bulk RNA-seq | Yes | |
| | | Hispanic | | | | | | | |
| pt#03 | M | Caucasian/Non- | 31 | RRMS | 6 | mTeff | Bulk RNA-seq | Yes | |
| | | Hispanic | | | | | | | |
| pt#04 | M | Caucasian/Non- | 42 | RRMS | 1.5 | mTreg | Bulk RNA-seq | Yes | |
| | | Hispanic | | | | | | | |
| pt#04 | M | Caucasian/Non- | 42 | RRMS | 1.5 | mTeff | Bulk RNA-seq | Yes | |
| | | Hispanic | | | | | | | |
| pt#05 | F | Caucasian/Non- | 48 | SPMS | 6.5 | mTreg | Bulk RNA-seq | No | Diagnosed |
| | | Hispanic | | | | | | | was |
| | | | | | | | | | changed |
| | | | | | | | | | as SPMS |
| | | | | | | | | | later |
| pt#05 | F | Caucasian/Non- | 48 | SPMS | 6.5 | mTeff | Bulk RNA-seq | No | Diagnosed |
| | | Hispanic | | | | | | | was |
| | | | | | | | | | changed |
| | | | | | | | | | as SPMS |
| | | | | | | | | | later |
| pt#06 | F | Caucasian/Non- | 34 | RRMS | 4 | mTreg | Bulk RNA-seq | Yes | |
| | | Hispanic | | | | | | | |
| pt#06 | F | Caucasian/Non- | 34 | RRMS | 4 | mTeff | Bulk RNA-seq | Yes | |
| | | Hispanic | | | | | | | |
| pt#07 | F | Caucasian/Non- | 29 | RRMS | 2 | mTreg | Bulk RNA-seq | Yes | |
| | | Hispanic | | | | | | | |
| pt#07 | F | Caucasian/Non- | 29 | RRMS | 2 | mTeff | Bulk RNA-seq | Yes | |
| | | Hispanic | | | | | | | |
| pt#08 | M | African | 57 | RRMS | 4.5 | mTreg | Bulk RNA-seq | No | Sample |
| | | american/Non- | | | | | | | collection |
| | | Hispanic | | | | | | | was done |
| | | | | | | | | | within |
| | | | | | | | | | 6months |
| | | | | | | | | | after |
| pt#08 | M | African | 57 | RRMS | 4.5 | mTeff | Bulk RNA-seq | No | Rituximab |
| | | american/Non- | | | | | | | injection |
| | | Hispanic | | | | | | | Sample |
| | | | | | | | | | collection |
| | | | | | | | | | was done |
| | | | | | | | | | within |
| | | | | | | | | | 6months |
| | | | | | | | | | after |
| | | | | | | | | | Rituximab |
| | | | | | | | | | injection |
| pt#09 | M | Caucasian/Non- | 39 | RRMS | 0 | mTreg | Bulk RNA-seq | Yes | |
| | | Hispanic | | | | | | | |
| pt#09 | M | Caucasian/Non- | 39 | RRMS | 0 | mTeff | Bulk RNA-seq | Yes | |
| | | Hispanic | | | | | | | |
| pt#10 | F | African | 44 | RRMS | 1 | mTreg | Bulk RNA-seq | Yes | |
| | | american/Non- | | | | | | | |
| | | Hispanic | | | | | | | |
| pt#10 | F | African | 44 | RRMS | 1 | mTeff | Bulk RNA-seq | Yes | |
| | | american/Non- | | | | | | | |
| | | Hispanic | | | | | | | |
| pt#11 | F | Caucasian/Non- | 47 | RRMS | 2.5 | mTreg | Bulk RNA-seq | Yes | |
| | | Hispanic | | | | | | | |
| pt#11 | F | Caucasian/Non- | 47 | RRMS | 2.5 | mTeff | Bulk RNA-seq | Yes | |
| | | Hispanic | | | | | | | |
| pt#12 | F | Caucasian/Non- | 32 | RRMS | 1.5 | mTreg | Bulk RNA-seq | Yes | |
| | | Hispanic | | | | | | | |
| pt#12 | F | Caucasian/Non- | 32 | RRMS | 1.5 | mTeff | Bulk RNA-seq | Yes | |
| | | Hispanic | | | | | | | |
| pt#13 | M | Caucasian/Non- | 41 | RRMS | 1.5 | mTreg | Bulk RNA-seq | Yes | |
| | | Hispanic | | | | | | | |
| pt#13 | M | Caucasian/Non- | 41 | RRMS | 1.5 | mTeff | Bulk RNA-seq | Yes | |
| | | Hispanic | | | | | | | |
| pt#14 | F | Caucasian/Non- | 41 | RRMS | 3 | mTreg | Bulk RNA-seq | Yes | |
| | | Hispanic | | | | | | | |
| pt#14 | F | Caucasian/Non- | 41 | RRMS | 3 | mTeff | Bulk RNA-seq | Yes | |
| | | Hispanic | | | | | | | |
| pt#15 | F | Caucasian/Non- | 53 | SPMS | 4.5 | mTreg | Bulk RNA-seq | No | Diagnosed |
| | | Hispanic | | | | | | | was |
| | | | | | | | | | changed |
| | | | | | | | | | as SPMS |
| | | | | | | | | | later |
| pt#15 | F | Caucasian/Non- | 53 | SPMS | 4.5 | mTeff | Bulk RNA-seq | No | Diagnosed |
| | | Hispanic | | | | | | | was |
| | | | | | | | | | changed |
| | | | | | | | | | as SPMS |
| | | | | | | | | | later |
| pt#16 | F | Caucasian/Non- | 32 | RRMS | na | mTreg | Bulk RNA-seq | Yes | |
| | | Hispanic | | | | | | | |
| pt#16 | F | Caucasian/Non- | 32 | RRMS | na | mTeff | Bulk RNA-seq | Yes | |
| | | Hispanic | | | | | | | |
| pt#17 | F | Caucasian/Non- | 29 | RRMS | 1 | mTreg | Bulk RNA-seq | Yes | |
| | | Hispanic | | | | | | | |
| pt#17 | F | Caucasian/Non- | 29 | RRMS | 1 | mTeff | Bulk RNA-seq | Yes | |
| | | Hispanic | | | | | | | |
| pt#18 | F | Caucasian/Non- | 51 | RRMS | 3.5 | mTreg | Bulk RNA-seq | Yes | |
| | | Hispanic | | | | | | | |
| pt#18 | F | Caucasian/Non- | 51 | RRMS | 3.5 | mTeff | Bulk RNA-seq | Yes | |
| | | Hispanic | | | | | | | |
| pt#19 | F | Caucasian/Non- | 43 | RRMS | 2.5 | mTreg | Bulk RNA-seq | Yes | |
| | | Hispanic | | | | | | | |
| pt#19 | F | Caucasian/Non- | 43 | RRMS | 2.5 | mTeff | Bulk RNA-seq | Yes | |
| | | Hispanic | | | | | | | |
| pt#20 | F | Caucasian/Non- | 24 | RRMS | 1 | mTreg | Bulk RNA-seq | Yes | |
| | | Hispanic | | | | | | | |
| pt#20 | F | Caucasian/Non- | 24 | RRMS | 1 | mTeff | Bulk RNA-seq | Yes | |
| | | Hispanic | | | | | | | |
| pt#21 | F | Caucasian/Non- | 59 | SPMS | 6 | mTreg | Bulk RNA-seq | No | Diagnosed |
| | | Hispanic | | | | | | | was |
| | | | | | | | | | changed |
| | | | | | | | | | as SPMS |
| | | | | | | | | | later |
| pt#21 | F | Caucasian/Non- | 59 | SPMS | 6 | mTeff | Bulk RNA-seq | No | Diagnosed |
| | | Hispanic | | | | | | | was |
| | | | | | | | | | changed |
| | | | | | | | | | as SPMS |
| | | | | | | | | | later |
| pt#22 | M | Caucasian/Non- | 29 | RRMS | 1 | mTreg | Bulk RNA-seq | Yes | |
| | | Hispanic | | | | | | | |
| pt#22 | M | Caucasian/Non- | 29 | RRMS | 1 | mTeff | Bulk RNA-seq | Yes | |
| | | Hispanic | | | | | | | |
| pt#23 | F | Caucasian/Non- | 35 | RRMS | 1.5 | mTreg | Bulk RNA-seq | Yes | |
| | | Hispanic | | | | | | | |
| pt#23 | F | Caucasian/Non- | 35 | RRMS | 1.5 | mTeff | Bulk RNA-seq | Yes | |
| | | Hispanic | | | | | | | |
| pt#24 | F | Caucasian/Non- | 31 | RRMS | 2 | mTreg | Bulk RNA-seq | Yes | |
| | | Hispanic | | | | | | | |
| pt#24 | F | Caucasian/Non- | 31 | RRMS | 2 | mTeff | Bulk RNA-seq | Yes | |
| | | Hispanic | | | | | | | |
| pt#25 | M | Hispanic | 22 | RRMS | na | mTreg | Bulk RNA-seq | Yes | |
| pt#25 | M | Hispanic | 22 | RRMS | na | mTeff | Bulk RNA-seq | Yes | |
| pt#26 | F | Caucasian/Non- | 31 | RRMS | na | mTreg | Bulk RNA-seq | Yes | |
| | | Hispanic | | | | | | | |
| pt#26 | F | Caucasian/Non- | 31 | RRMS | na | mTeff | Bulk RNA-seq | Yes | |
| | | Hispanic | | | | | | | |
| pt#27 | M | Caucasian/Non- | 35 | RRMS | 5.5 | mTreg | Bulk RNA-seq | Yes | |
| | | Hispanic | | | | | | | |
| pt#27 | M | Caucasian/Non- | 35 | RRMS | 5.5 | mTeff | Bulk RNA-seq | Yes | |
| | | Hispanic | | | | | | | |
| pt#28 | F | Caucasian/Non- | 30 | RRMS | 1.5 | mTreg | Bulk RNA-seq | Yes | |
| | | Hispanic | | | | | | | |
| pt#28 | F | Caucasian/Non- | 30 | RRMS | 1.5 | mTeff | Bulk RNA-seq | Yes | |
| | | Hispanic | | | | | | | |
| pt#29 | F | Caucasian/Non- | 34 | RRMS | 1.5 | mTreg | Bulk RNA-seq | Yes | |
| | | Hispanic | | | | | | | |
| pt#29 | F | Caucasian/Non- | 34 | RRMS | 1.5 | mTeff | Bulk RNA-seq | Yes | |
| | | Hispanic | | | | | | | |
| pt#30 | F | Caucasian/Non- | 44 | RRMS | 2.5 | mTreg | Bulk RNA-seq | Yes | |
| | | Hispanic | | | | | | | |
| pt#30 | F | Caucasian/Non- | 44 | RRMS | 2.5 | mTeff | Bulk RNA-seq | Yes | |
| | | Hispanic | | | | | | | |
| pt#31 | F | Caucasian/Non- | 45 | RRMS | na | CD4+ | multi-omics | Yes | |
| | | Hispanic | | | | T cells | single cell | | |
| | | | | | | | analysis | | |
| pt#32 | F | Caucasian/Non- | 25 | RRMS | na | CD4+ | multi-omics | Yes | |
| | | Hispanic | | | | T cells | single cell | | |
| | | | | | | | analysis | | |
| pt#33 | F | Caucasian/Non- | 32 | RRMS | na | CD4+ | multi-omics | Yes | |
| | | Hispanic | | | | T cells | single cell | | |
| | | | | | | | analysis | | |
| pt#34 | F | Caucasian/Non- | 29 | RRMS | na | CD4+ | multi-omics | Yes | |
| | | Hispanic | | | | T cells | single cell | | |
| | | | | | | | analysis | | |
| pt#35 | M | Caucasian/Non- | 43 | RRMS | na | CD4+ | multi-omics | Yes | |
| | | Hispanic | | | | T cells | single cell | | |
| | | | | | | | analysis | | |

Several of the top DEGs (PRDM1, BCL3, RHBDD2, TOB2, LBH) in mTreg were validated by using qPCR with an independent cohort of patients with MS (n=16) and healthy controls (n=23) (FIGS. 1A and E, FIG. 7D, patient characteristics are described in Tables 1A and 1). The up regulation of PRDM1 in MS mTreg and mTconv at the protein level was confirmed by intracellular staining of Blimp1 using flow cytometry (FIG. 1E). It was next questioned whether this Treg transcriptional signature observed in MS is shared across different autoimmune diseases using ImmuNexUT data (1) where transcriptomic profiles of multiple peripheral immune cells, including mTreg and mTconv, were explored at population scale across multiple autoimmune diseases. Notably, the transcriptional signature that was observed in MS Treg was also observed in Treg among most of the autoimmune diseases analyzed (total 12 diseases: 10 diseases from ImmuNexUT, T1D from (3), and MS from this study) (FIG. 1F, 7E). Inverse regulation of PRDM1 and ID3 observed in MS was highlighted in Tregs from patients with systemic lupus erythematosus (SLE), idiopathic inflammatory myopathy (IIM), and ANCA-associated vasculitis (AAV) (FIG. 7F). The bulk RNA-seq transcriptional profiling of memory Tconv and Treg highlighted PRDM1 as a key regulatory factor in dysfunctional Tregs in autoimmune diseases.

### Example 2. Single-Cell Dual Omics Analysis Reveals Elevated PRDM1 in Th17-Like Treg in MS

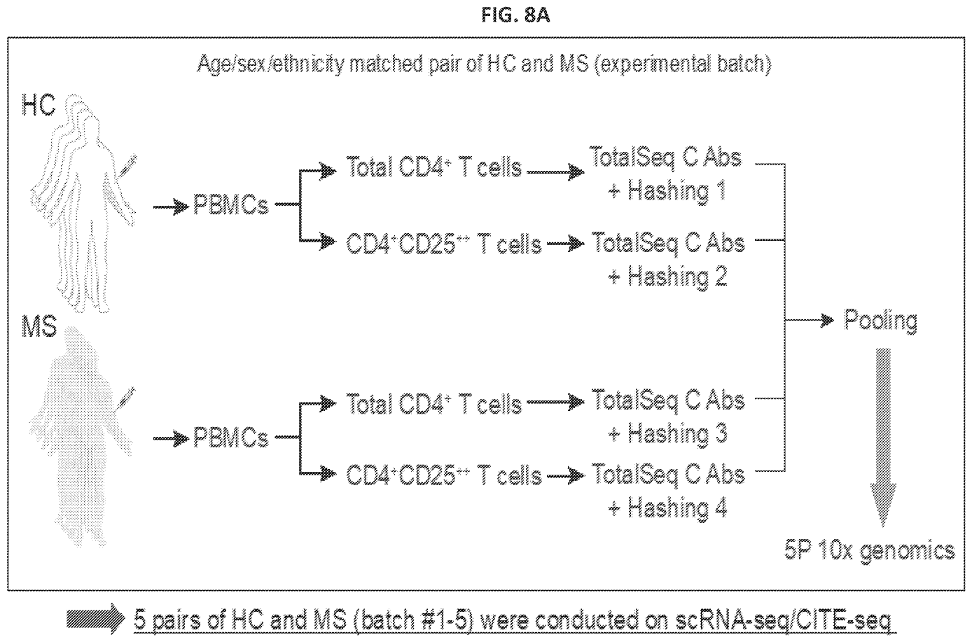

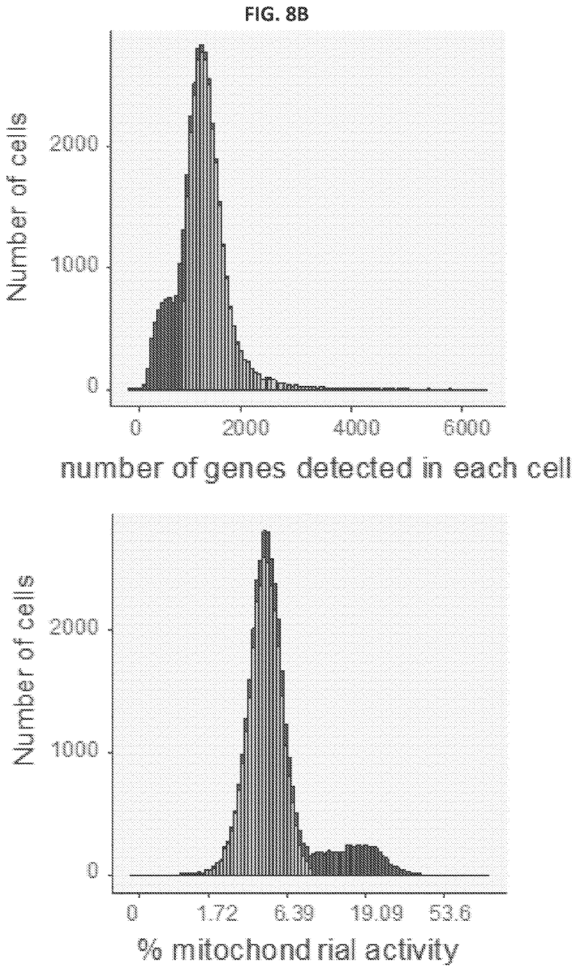

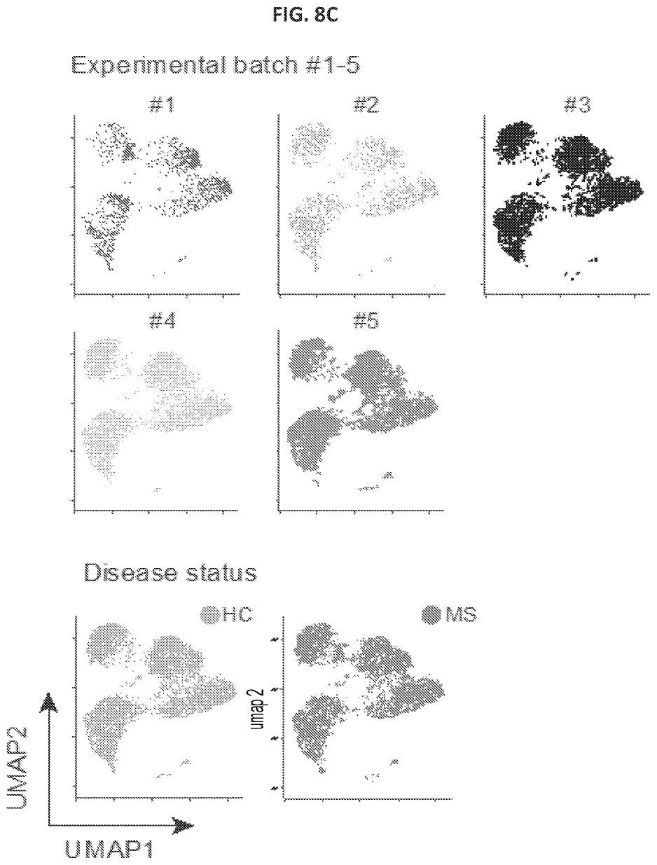

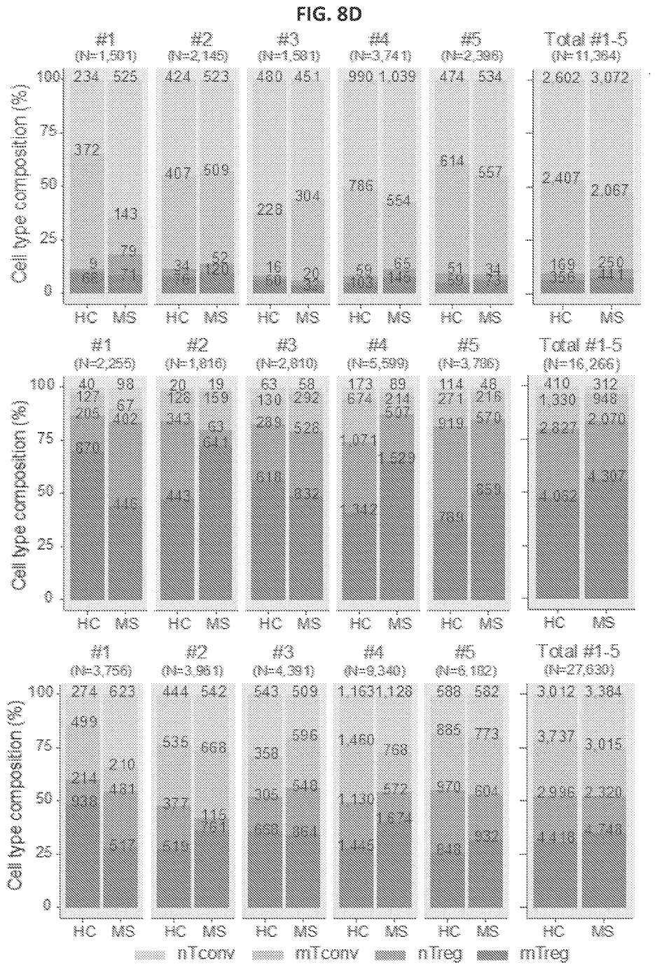

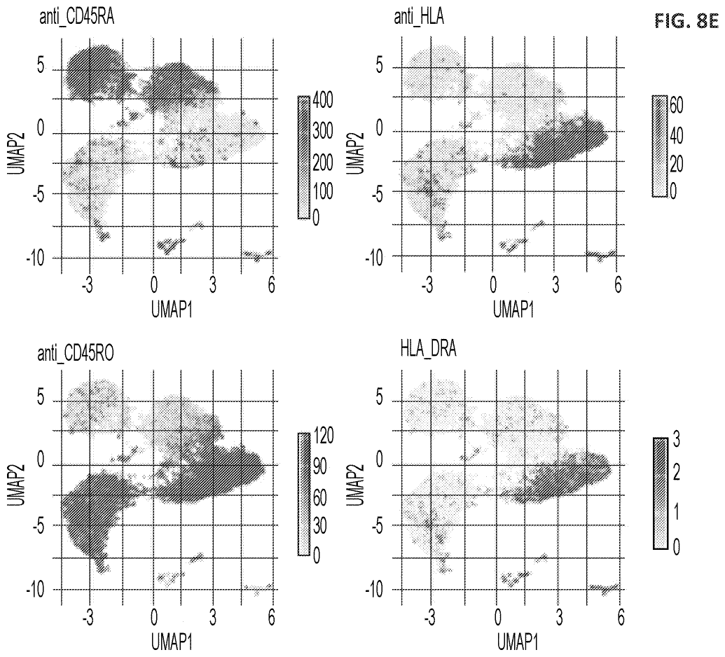

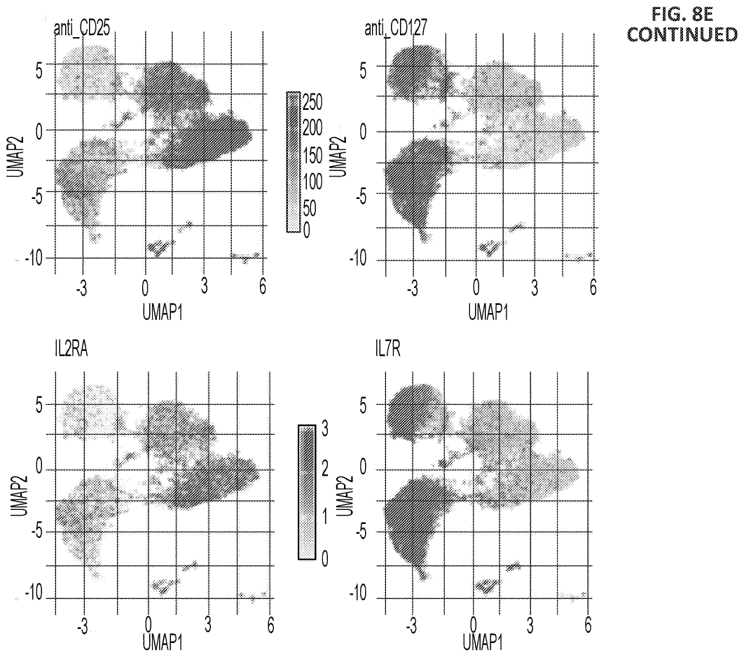

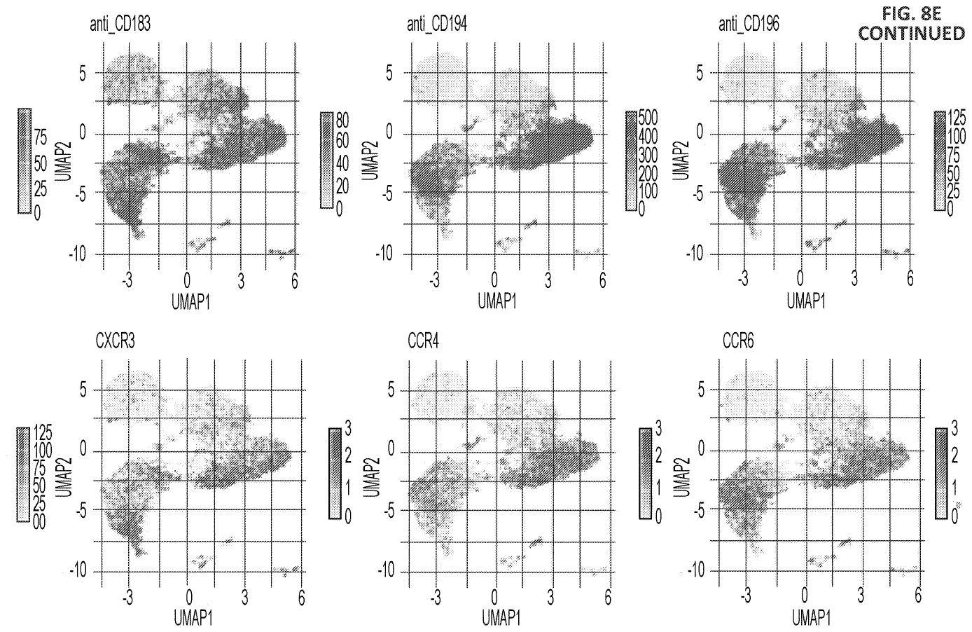

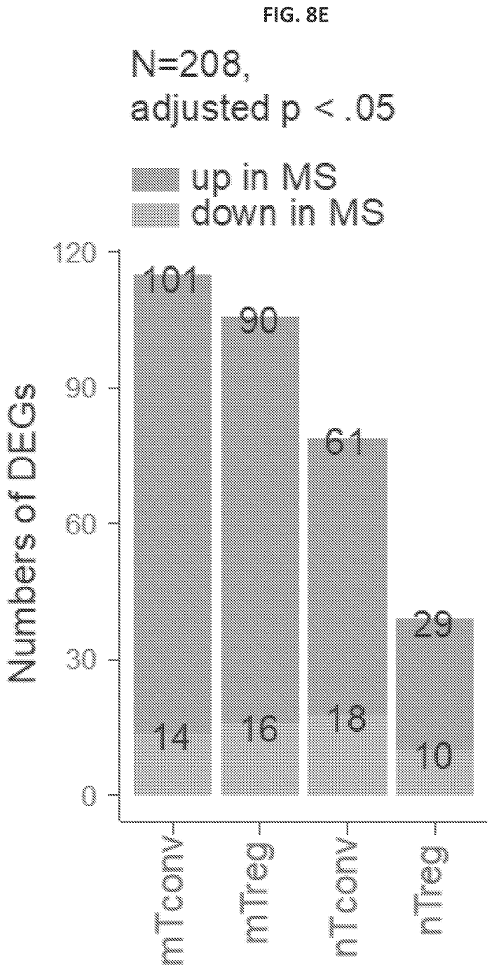

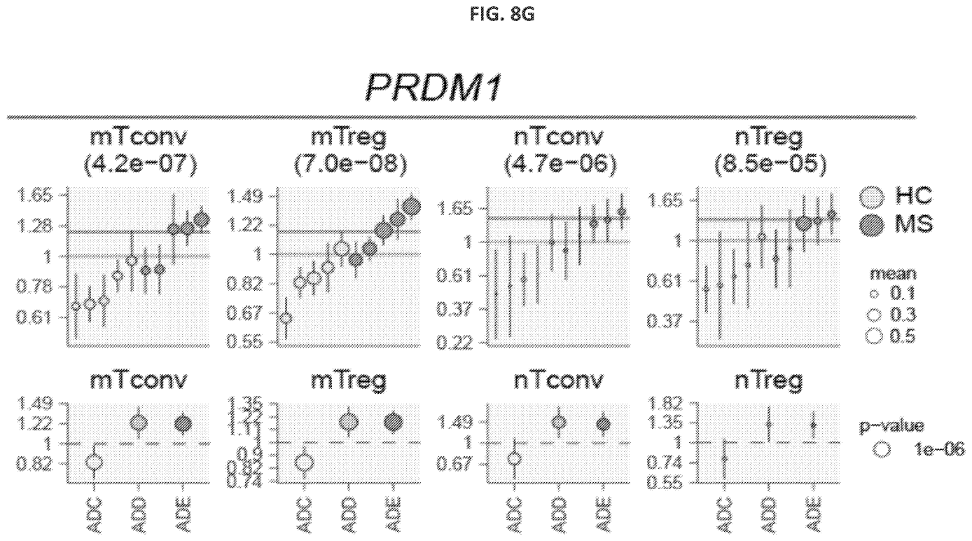

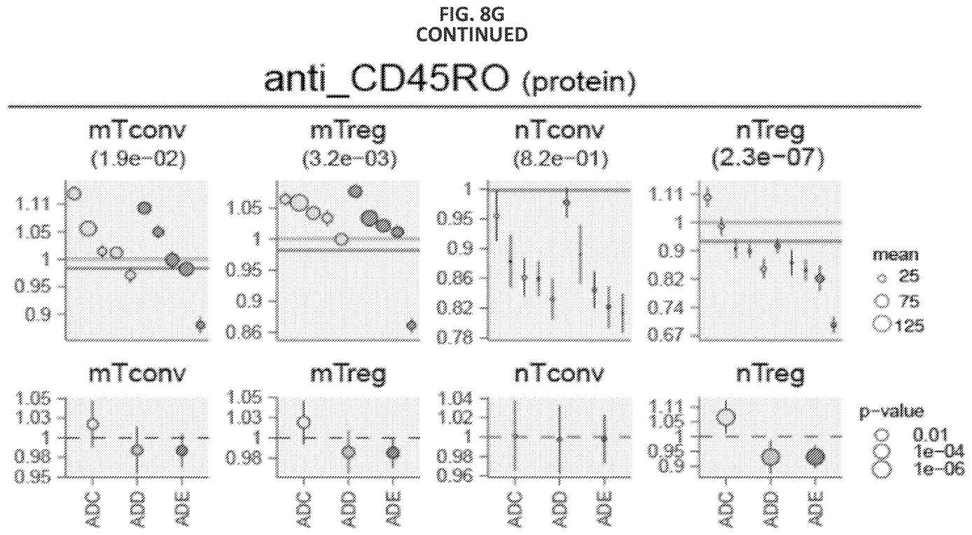

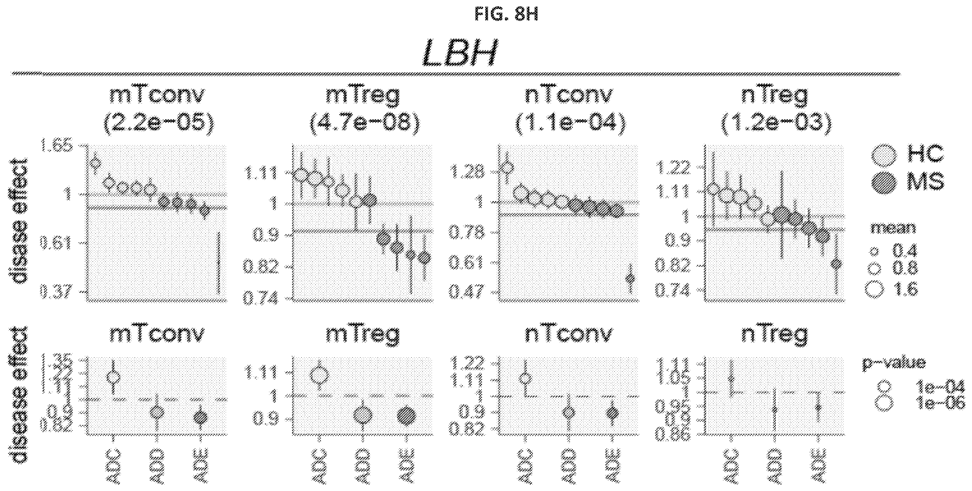

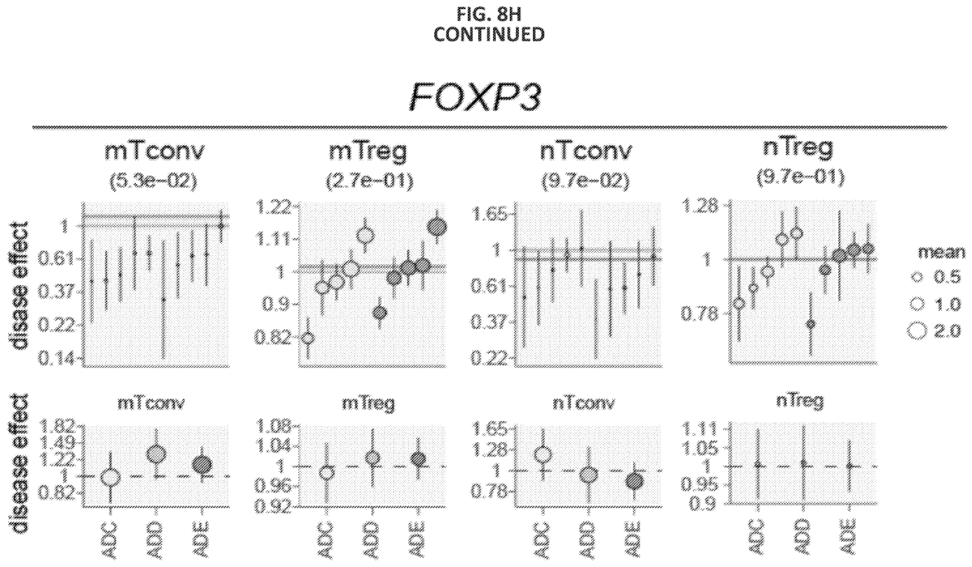

To gain a deeper understanding of cellular heterogeneity and novel cell types concerning the disease mechanisms, single-cell RNA-sequencing (scRNA-seq) was designed and performed to profile CD4+ T cells. To overcome the sparsity of scRNA-seq data (30, 31), Cellular Indexing of Transcriptomes and Epitopes by sequencing (CITE-seq) was used (32, 33), and 44 surface protein markers (Tables 2A and 2B) and mRNA expression were profiled simultaneously in total CD4+ T cells enriched with Treg cells. Additionally, to avoid experimental batch effects between controls and MS, hashing technology was adopted to pool cells into a single run of the 10× Genomics platform. Across five experimental batches, a comparable number of Treg and Tconv cells were obtained (FIG. 8A). Demographic backgrounds (age, sex, and ethnicity) are controlled in each batch of 10× Genomics processing between controls and MS, and a total of five paired MS and control samples were included (Tables 1A and 1B).

TABLE 2A
Totalseq™M-C Profiling

| | | | | | SEQ | |
|---|---|---|---|---|---|---|
| | | | | | ID | |
| 5' prime | | Target | Clone | | NO: | Sequence |
| TotalSeq™-C | C0154 | CD27 | O323 | Human | 1 | GCACTCCTGCATGTA |
| TotalSeq™-C | C0034 | CD3 | UCHT1 | Human | 2 | CTCATTGTAACTCCT |
| TotalSeq™-C | C0087 | CD45RO | UCHL1 | Human | 3 | CTCCGAATCATGTTG |
| TotalSeq™-C | C0389 | CD38 | HIT2 | Human | 4 | TGTACCCGCTTGTGA |
| TotalSeq™-C | C0368 | CD226 | 11A8 | Human | 5 | TCTCAGTGTTTGTGG |
| | | (DNAM-1) | | | | |
| TotalSeq™-C | C0390 | CD127(IL-7a) | A019D5 | Human | 6 | GTGTGTTGTCCTATG |
| TotalSeq™-C | C0148 | CD197(CCR7) | G043H7 | Human | 7 | AGTTCAGTCAACCGA |
| TotalSeq™-C | C0085 | CD25 | BC96 | Human | 8 | TTTGTCCTGTACGCC |
| TotalSeq™-C | C0171 | CD278(ICOS) | C398.4A | Human | 9 | CGCGCACCCATTAAA |
| TotalSeq™-C | C0088 | CD279(PD-1) | EH12.2H7 | Human | 10 | ACAGCGCCGTATTTA |
| TotalSeq™-C | C0072 | CD4 | RPA-T4 | Human | 11 | TGTTCCCGCTCAACT |
| TotalSeq™-C | C0063 | CD45RA | HI100 | Human | 12 | TCAATCCTTCCGCTT |
| TotalSeq™-C | C0080 | CD8a | RPA-T8 | Human | 13 | GCTGCGCTTTCCATT |
| TotalSeq™-C | C0089 | TIGIT(VSTM3) | A15153G | Human | 14 | TTGCTTACCGCCAGA |
| TotalSeq™-C | C0169 | anti-human | F38-2E2 | Human | 15 | TGTCCTACCCAACTT |
| | | CD366 (Tim-3) | | | | |
| TotalSeq™-C | C0140 | anti-human | G025H7 | Human | 16 | GCGATGGTAGATTAT |
| | | CD183 | | | | |
| | | (CXCR3) | | | | |
| TotalSeq™-C | C0159 | anti-human | L243 | Human | 17 | AATAGCGAGCAAGTA |
| | | HLA-DR | | | | |
| TotalSeq™-C | C0071 | CD194 (CCR4) | L291H4 | Human | 18 | AGCTTACCTGCACGA |
| TotalSeq™-C | C0143 | CD196 (CCR6) | G034E3 | Human | 19 | GATCCCTTTGTCACT |
| TotalSeq™-C | C0144 | CD185 | J252D4 | Human | 20 | AATTCAACCGTCGCC |
| | | (CXCR5) | | | | |
| TotalSeq™-C | C0174 | CD58 (LFA-3) | TS2/9 | Human | 21 | GTTCCTATGGACGAC |
| TotalSeq™-C | C0149 | CD161 | HP-3G10 | Human | 22 | GTACGCAGTCCTTCT |
| TotalSeq™-C | C0251 | anti-human | | Human | 23 | GTCAACTCTTTAGCG |
| | | Hashtag 1 | | | | |
| TotalSeq™-C | C0252 | anti-human | | Human | 24 | TGATGGCCTATTGGG |
| | | Hashtag 2 | | | | |
| TotalSeq™-C | C0253 | anti-human | | Human | 25 | TTCCGCCTCTCTTTG |
| | | Hashtag 3 | | | | |
| TotalSeq™-C | C0253 | anti-human | | Human | 26 | AGTAAGTTCAGCGTA |
| | | Hashtag 4 | | | | |
| TotalSeq™-C | C0050 | CD19 | HIB19 | Human | 27 | CTGGGCAATTACTCG |
| TotalSeq™-C | C0100 | CD20 | 2H7 | Human | 28 | TTCTGGGTCCCTAGA |
| TotalSeq™-C | C0180 | CD24 | ML5 | Human | 29 | AGATTCCTTCGTGTT |
| TotalSeq™-C | C0136 | IgM | MHM88 | Human | 30 | TAGCGAGCCCGTATA |
| TotalSeq™-C | C0375 | IgG Fc | M1310G5 | Human | 31 | CTGGAGCGATTAGAA |
| TotalSeq™-C | C0355 | CD137 (4-1BB) | 4B4-1 | Human | 32 | CAGTAAGTTCGGGAC |
| TotalSeq™-C | C0051 | CD14 | 63D3 | Human | 33 | CAATCAGACCTATGA |
| TotalSeq™-C | C0083 | CD16 | 3G8 | Human | 34 | AAGTTCACTCTTTGC |
| TotalSeq™-C | C0064 | anti-human | 6H6 | Human | 35 | CTTCACTCTGTCAGG |
| | | CD123 | | | | |
| TotalSeq™-C | C0160 | anti-human | L161 | Human | 36 | GAGCTACTTCACTCG |
| | | CD1c | | | | |
| TotalSeq™-C | C0224 | TCR α/β | IP26 | Human | 37 | CGTAACGTAGAGCGA |
| TotalSeq™-C | C0366 | CD184 | 12G5 | Human | 38 | TCAGGTCCTTTCAAC |
| | | (CXCR4) | | | | |
| TotalSeq™-C | C0141 | CD195 (CCR5) | J418F1 | Human | 39 | CCAAAGTAAGAGCCA |
| TotalSeq™-C | C0582 | TCR Vδ2 | B6 | Human | 40 | TCAGTCAGATGGTAT |

TABLE 2B
Hashing Abs For Each Batch

| Hashing | Subject | Cell type |
|---|---|---|
| #1 | HA5875 | CD25+ CD4T cells |
| #2 | HA5875 | Total CD4T cells |
| #3 | MS1140 | Total CD4T cells |
| #4 | MS1140 | CD25+ CD4T cells |
| #1 | HA5954 | Total CD4T cells |
| #2 | HA5954 | CD25+ CD4T cells |
| #3 | MS1152 | Total CD4T cells |
| #4 | MS1152 | CD25+ CD4T cells |
| #1 | HA6032 | Total CD4T cells |
| #2 | HA6032 | CD25+ CD4T cells |
| #3 | MS1163 | Total CD4T cells |
| #4 | MS1163 | CD25+ CD4T cells |
| #1 | HA6078 | Total CD4T cells |
| #2 | HA6078 | CD25+ CD4T cells |
| #3 | MS1164 | Total CD4T cells |
| #4 | MS1164 | CD25+ CD4T cells |
| #1 | HA6079 | Total CD4T cells |
| #2 | HA6079 | CD25+ CD4T cells |
| #3 | MS1165 | Total CD4T cells |
| #4 | MS1165 | CD25+ CD4T cells |

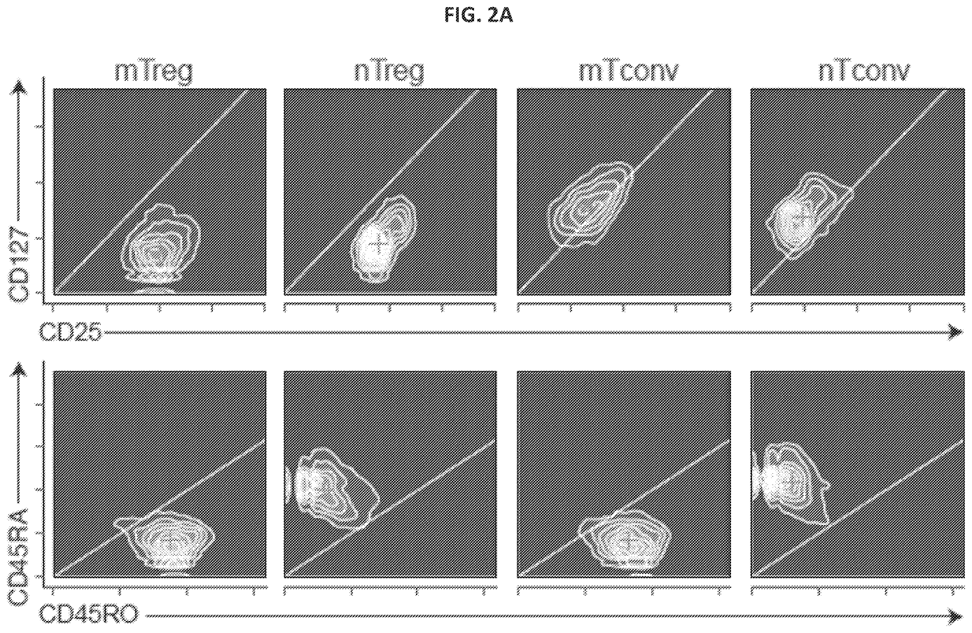

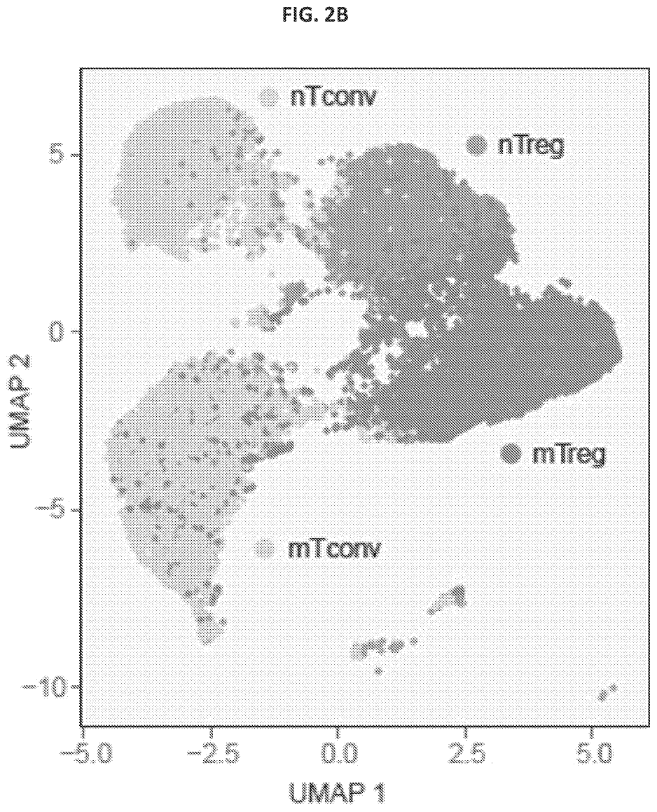

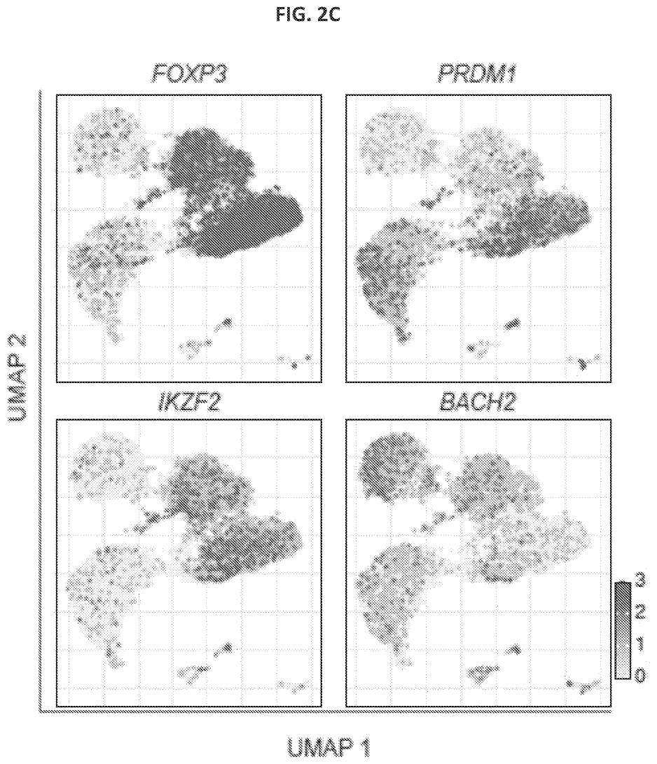

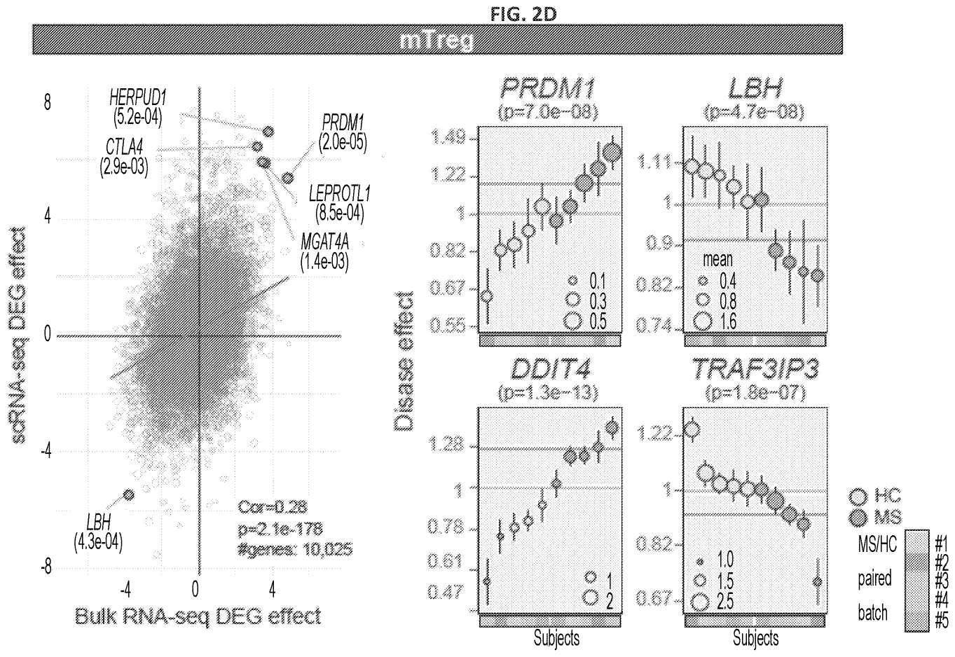

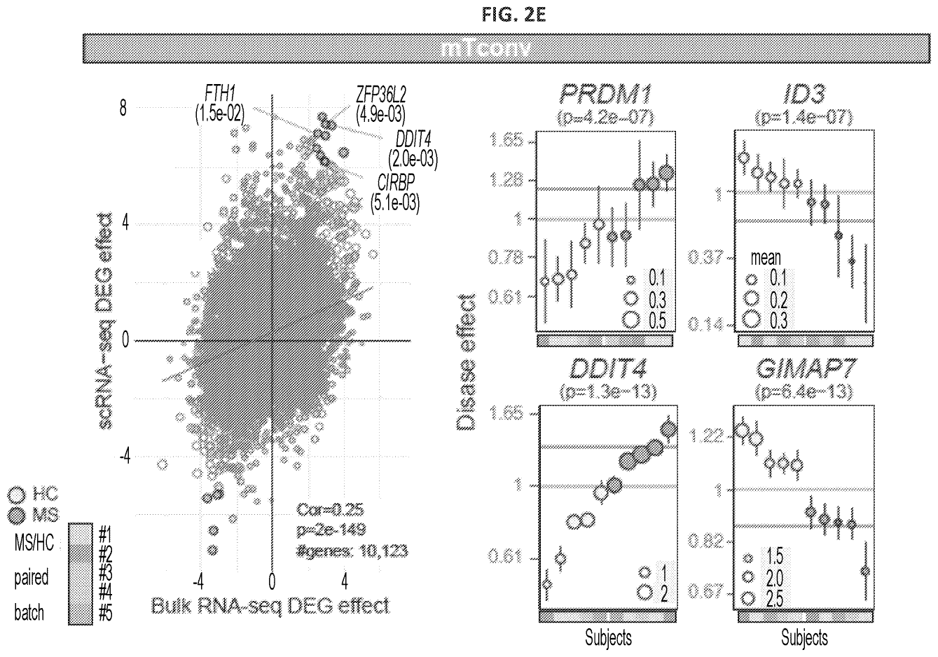

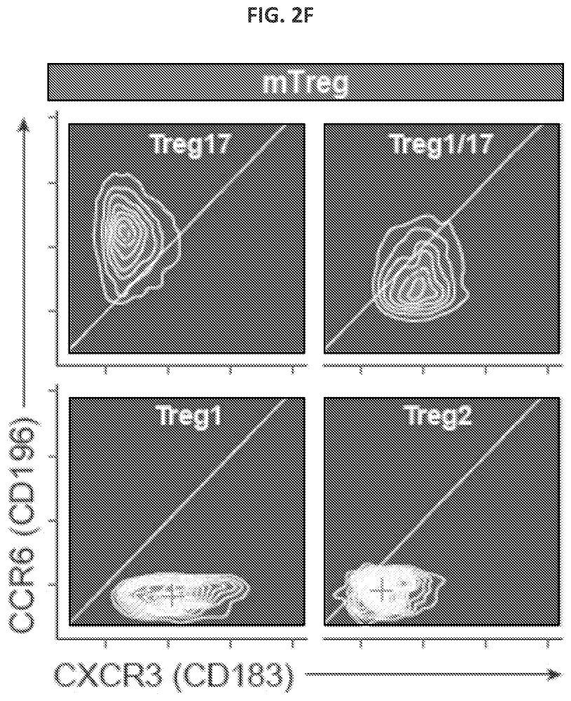

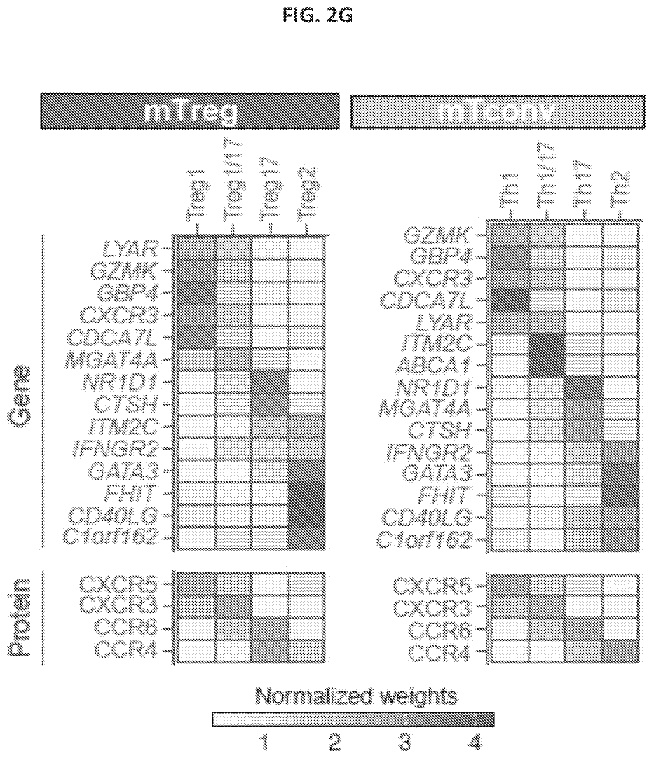

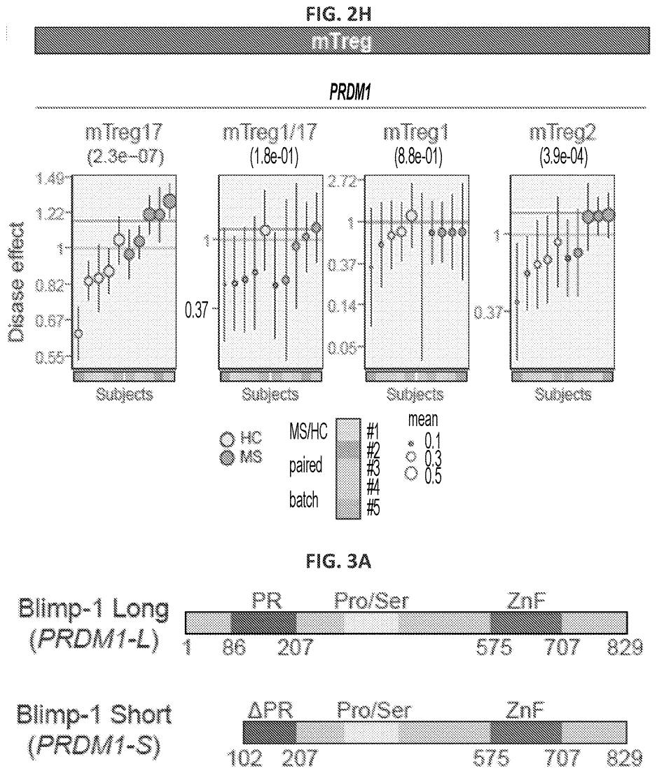

Cell identities based on surface marker proteins were established, eliciting prior knowledge of cell-type-specific signatures. A data matrix of 25,267 features (proteins and genes) and 36,983 cells was built, combining five batches of CITE-seq profiles. A basic quality control procedure was conducted to remove low-quality cells (FIG. 8B) followed by batch normalization to control batch-specific bias (Methods). Cell type identities were annoted in semi-supervised training guided by combinations of surface markers (FIG. 2A), clearly demonstrating distinctive cell populations in both the protein and transcriptomic space as can be seen in the gene-expression-based clustering patterns in the UMAP visualization (FIG. 2B). Cell type annotation results were not affected by batch labels or disease groups (FIG. 8C). The experimental design also consistently enriched rare Treg populations and provided sufficient cells to study the variation within Tregs (FIG. 8D). The markers for Treg vs Tconv (FOXP3, IKZF2, IL2RA CD25, and IL7R/CD127), and memory vs naïve (CD45RA, CD45RO) clearly distinguished four CD4+ T cell subpopulations (FIG. 2C, FIG. 8E).

For differentially expressed gene (34) analysis between MS and control subjects at the pseudo-bulk level (35), subject-level gene expression profiles were quantified after adjusting contributions of putative confounding factors, which stem from unmeasured technical covariates but may create spurious associations with disease status and cell types (see, e.g., Methods described herein for details)(2). Single-cell-based analysis identified 90 up-regulated genes, and 16 down-regulated genes in MS mTreg (FIG. 8F). The DEG effect size calculated in the scRNA-seq data (y-axis) was compared with the bulk DEG results (x-axis) (FIG. 2D-E), which confirmed that a substantial number of DEGs found in the bulk RNA-seq analysis are replicated in the matched subpopulations (mTreg and mTconv) with a statistically significant correlation in both mTreg and mTconv (mTreg; r=0.28, p=2.1⁻¹⁷⁸, mTconv; r=0.25, p=2⁻¹⁴⁹). For example, upregulation of PRDM1 and downregulation of LBH were demonstrated in MS mTreg (FIG. 2D). PRDM1 was also upregulated in mTconv, which further validated the importance of PRDM1 in the MS T cell signature (FIGS. 2D and E, FIG. 8G). DDIT4, which suppresses mTOR function, was upregulated in mTreg as well as mTconv and downregulation of TRAF3IP3 in MS mTreg, supporting the dysfunctional Treg property and skewed Th17 signature in MS (36-39). Of note, expression of CD45RO and FOXP3 was not significantly altered between MS and control subjects (FIG. 8G), indicating the differential analysis based on dual omics single-cell analysis was not biased by skewed memory differentiation or significant loss of FOXP3 gene expression in MS mTreg.

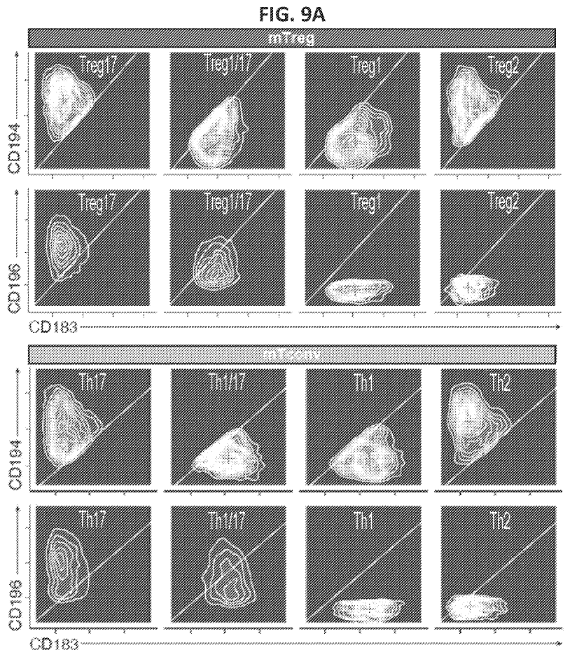

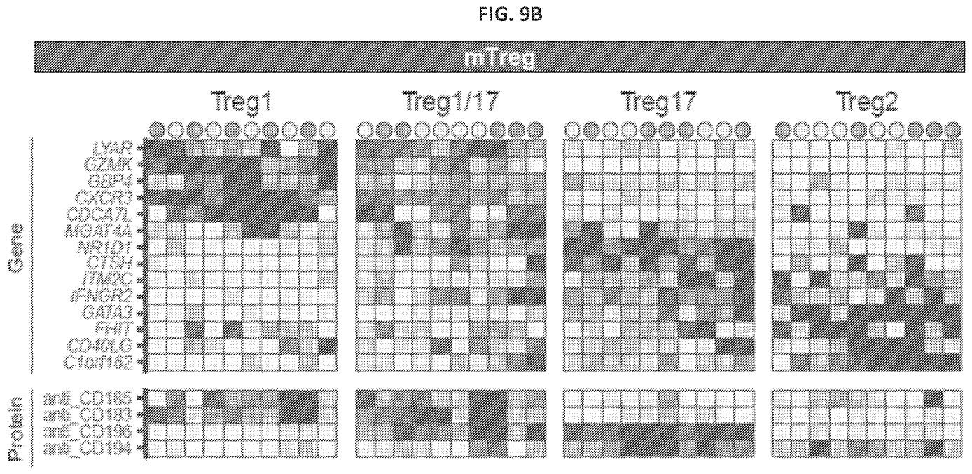

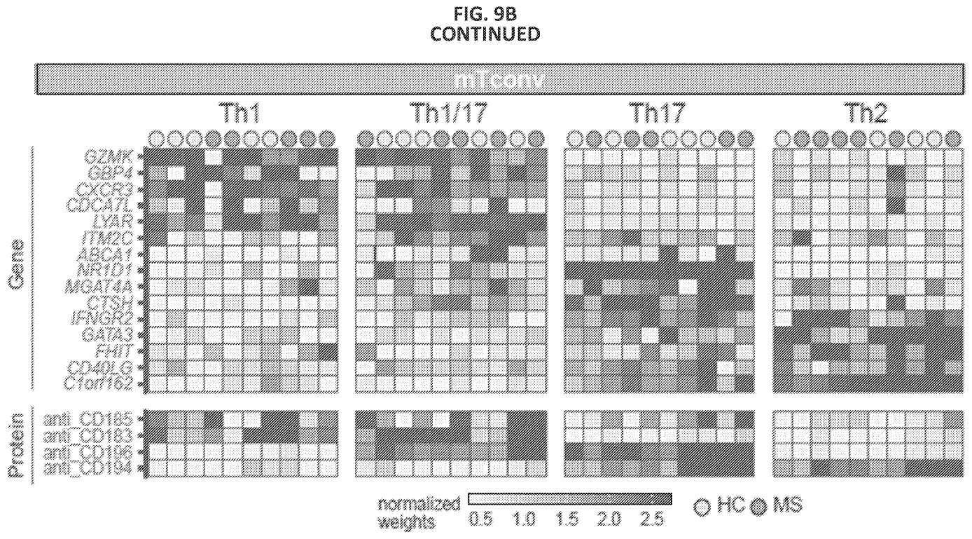

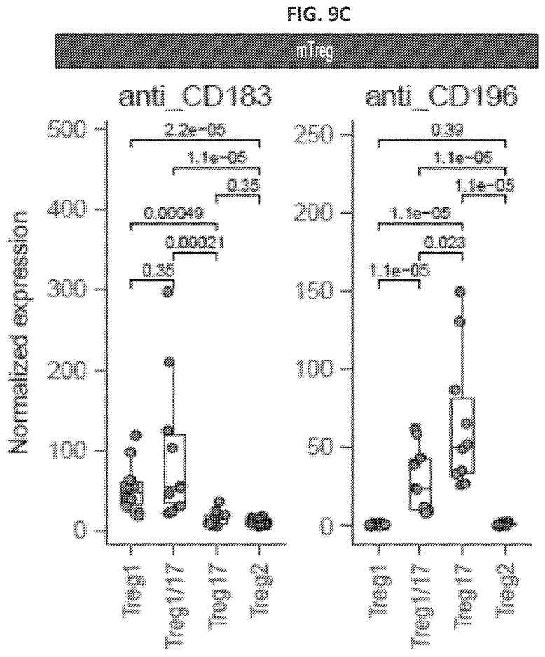

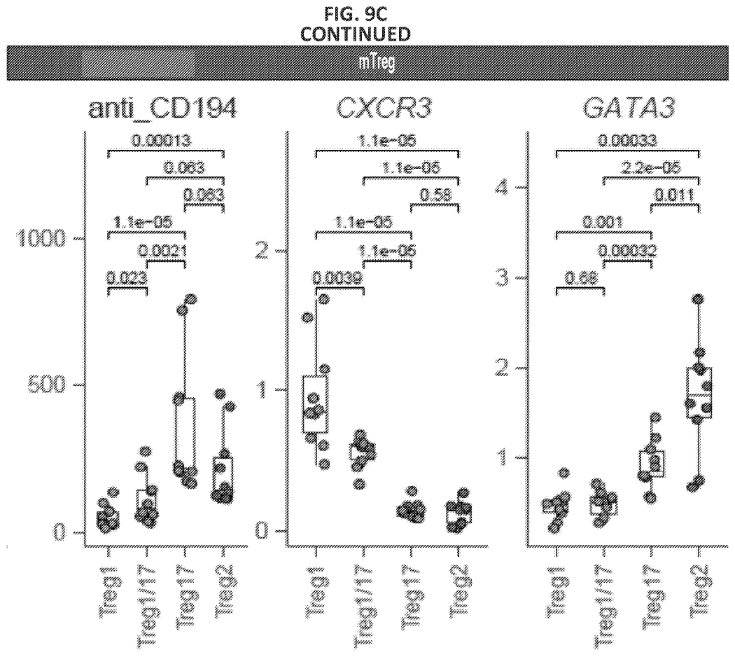

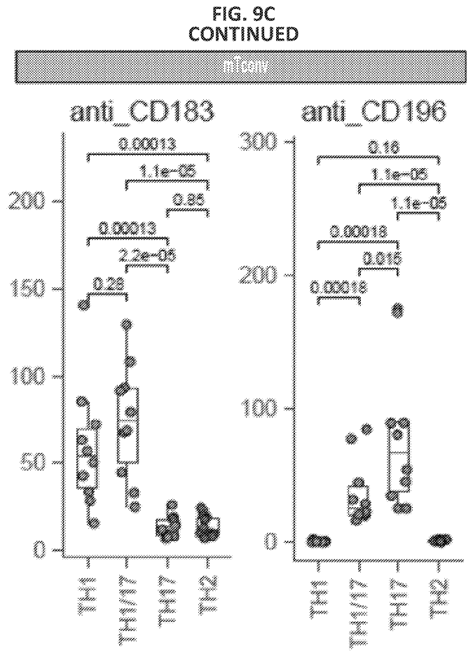

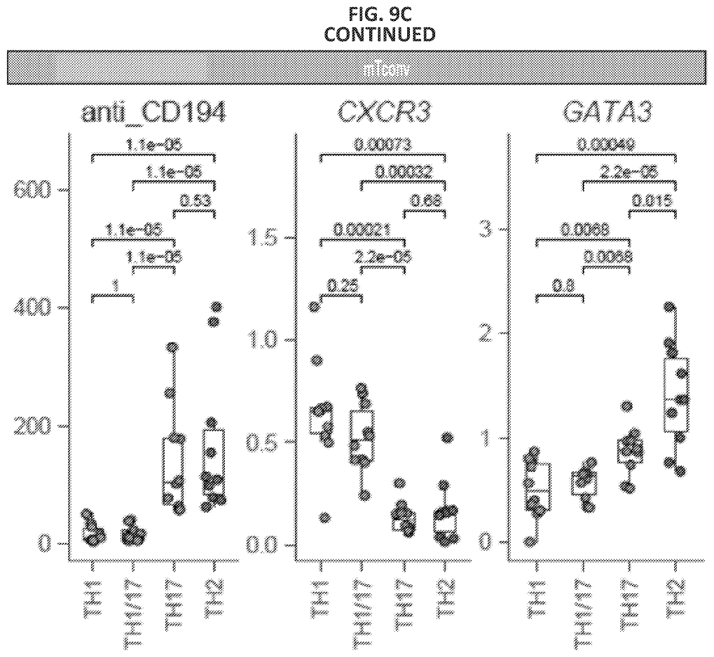

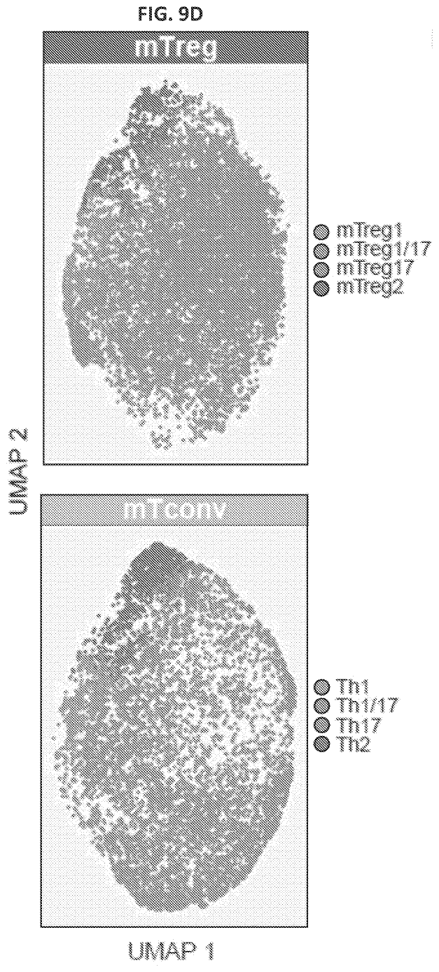

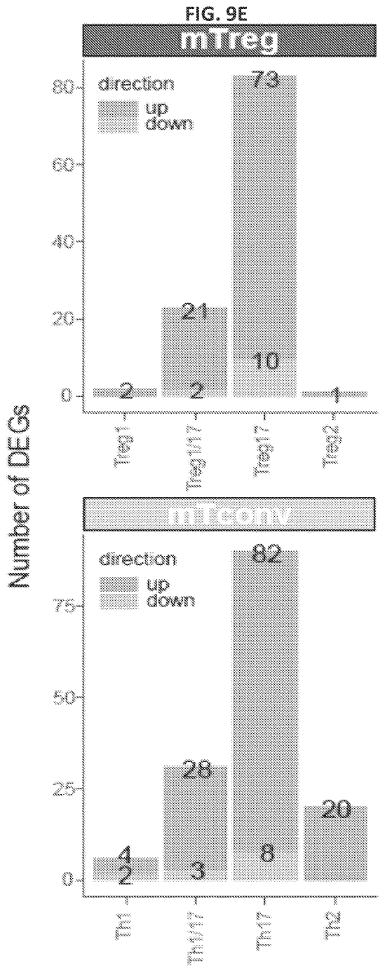

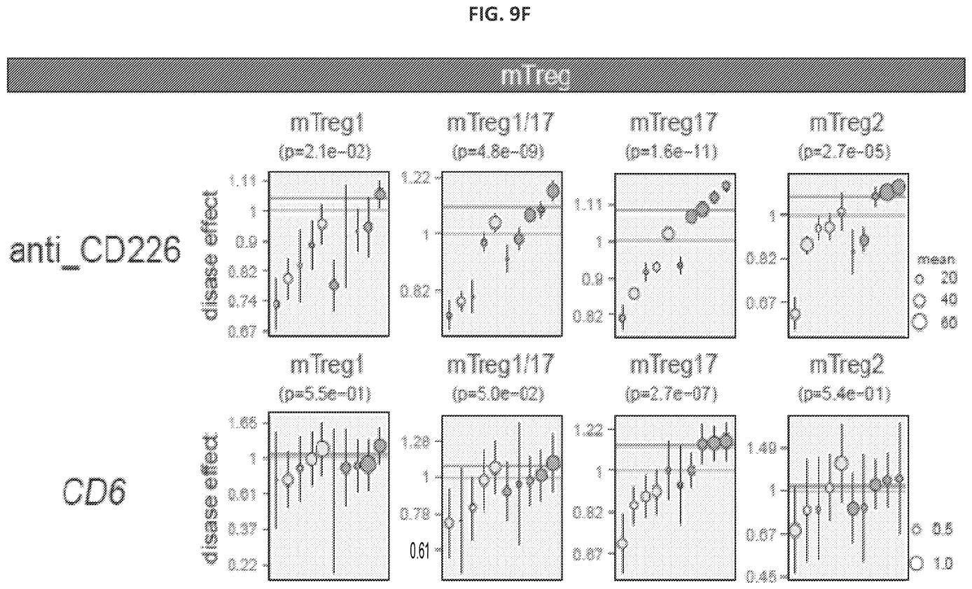

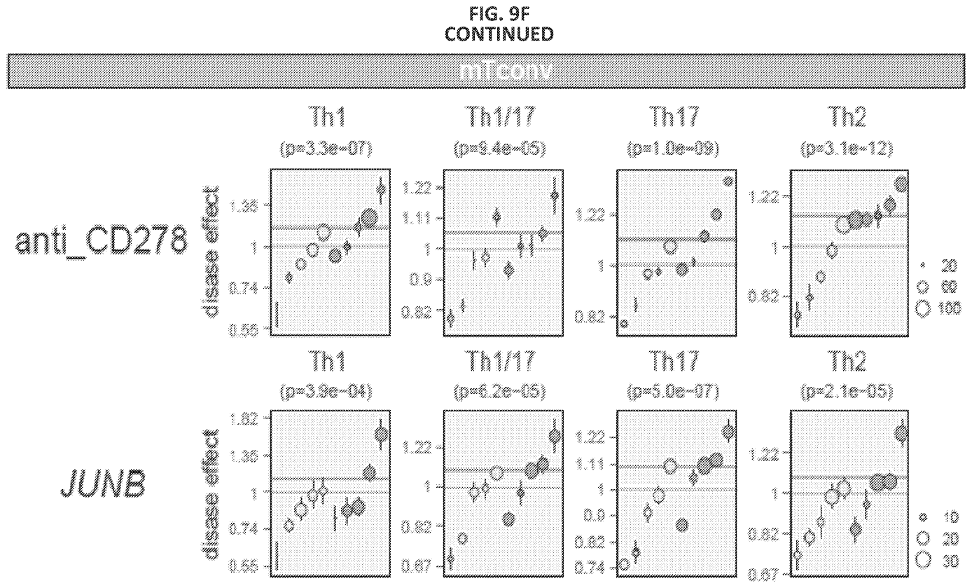

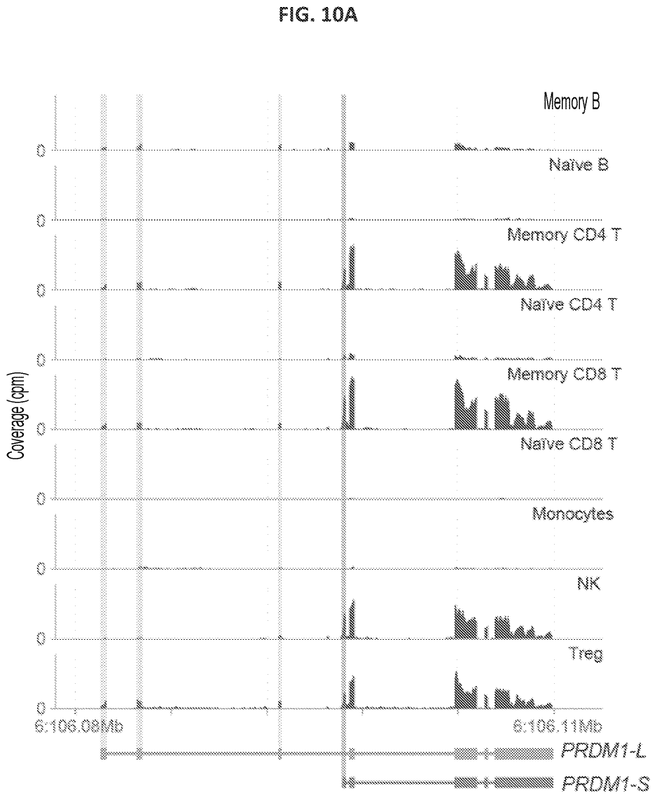

To elucidate the plasticity of the memory CD4+ T cell population, the T helper cell subtypes (Th1, Th2, Th17, Th1/17) in mTreg and mTconv were further determined by using key marker expression with CITE-seq (FIG. 2F, G, FIG. 9A-E, see also, e.g., methods described herein). The gene expression changes between MS and controls at each mTreg subtype were analyzed and it was found that PRDM1 was significantly upregulated in Th17-like mTreg (mTreg17) (FIG. 2H). Upregulation of CD226 and CD6 in mTreg17 and CD278 (ICOS) and JUNB in Th17 indicates the skewed pathogenic Th17-like signature in MS memory T cells (FIG. 9F) (40-43). Given the causative role of Th17 in MS pathophysiology, the results at single-cell resolution further dissected the features of MS mTreg and highlighted the potent role of PRDM1 in skewing mTreg and mTconv toward the pathogenic Th17-like signature in the context of MS.

### Example 3. Elevated Alternative Short PRDM1 Isoform in MS mTreg

An unbiased transcriptomic profiling of MS mTreg using bulk and single-cell RNA-seq identified PRDM1 as a top candidate transcription factor accounting for dysfunctional Treg properties in MS. PRDM1 encodes the Blimp1 protein which functions as a zinc finger transcriptional repressor initially identified as a protein that binds to the promoter of IFNB1 and suppresses its expression (44). The development and function of a variety of immune cells are also under the control of Blimp1. CD4+ T cell-specific PRDM1 deletion exaggerates the proinflammatory reaction in multiple murine models of autoimmune diseases, including EAE (45, 46); in contrast, previous studies demonstrated Blimp1 as an essential factor driving inflammatory programs in Th17 differentiation (47). This contradictory evidence suggests the context-dependent roles of PRDM1 in CD4+ T cells in mice. Recently, the role of PRDM1 in Treg cells was studied by using Treg-specific PRDM1 knockout mice in the context of autoimmunity. Loss of their suppressive capacity in PRDM1-deficient Treg indicates that PRDM1 plays a critical role in maintaining Treg homeostasis in tissue and positively regulates its suppressive function (48-50). Thus, the result showing upregulation of PRDM1 in MS mTreg fundamentally contradicted observations in mice.

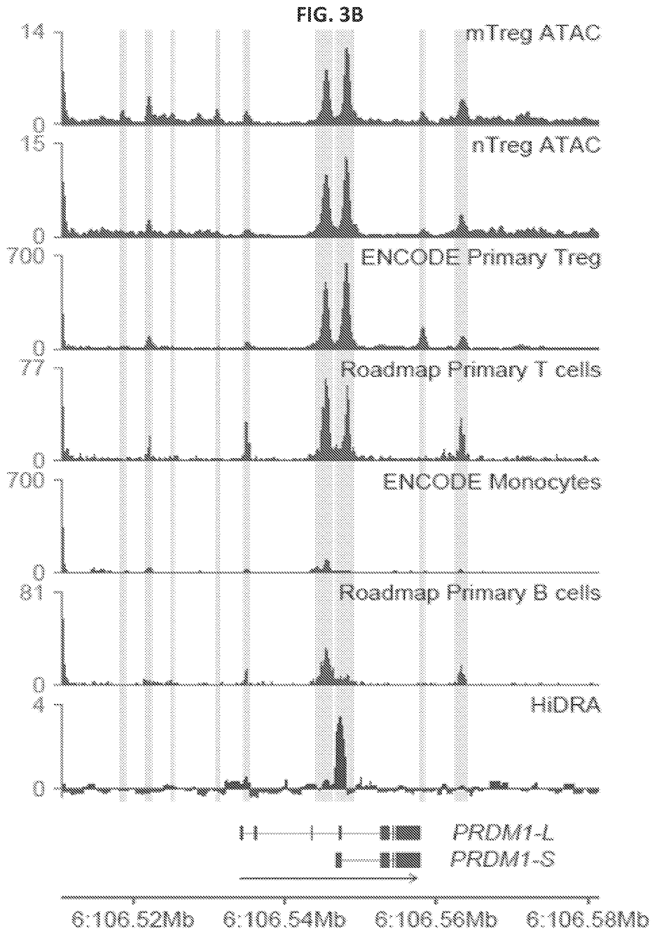

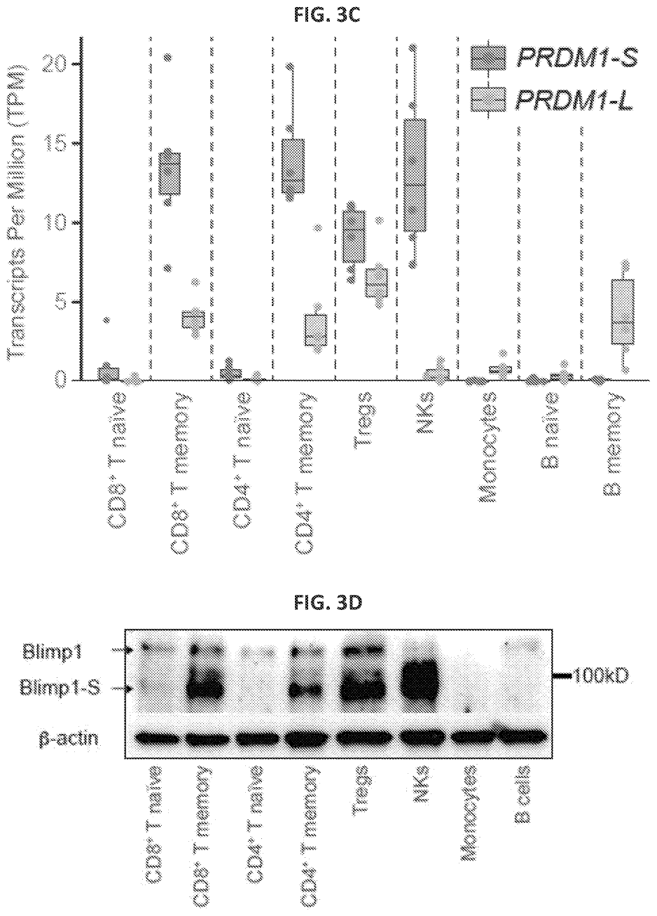

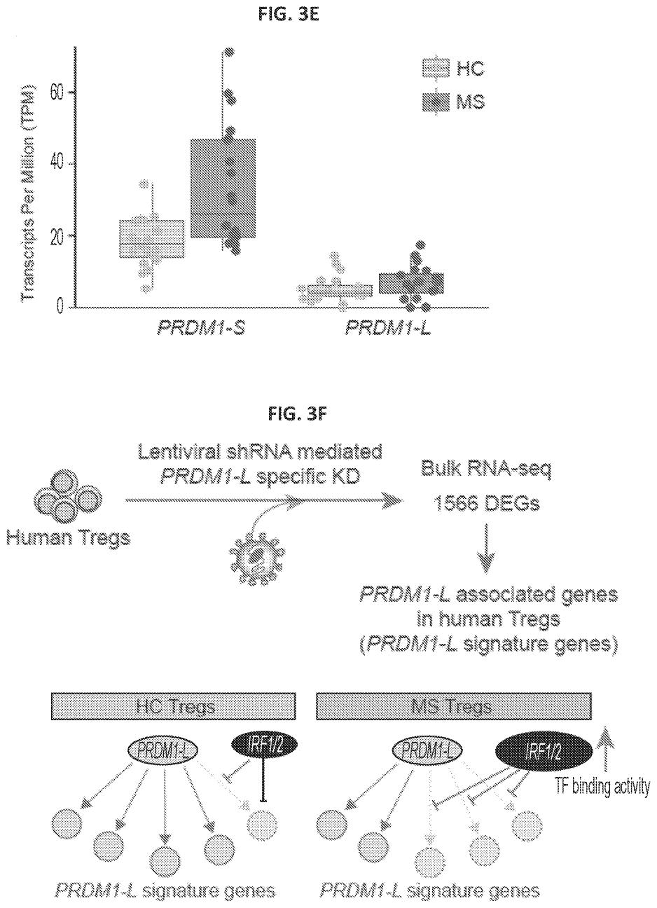

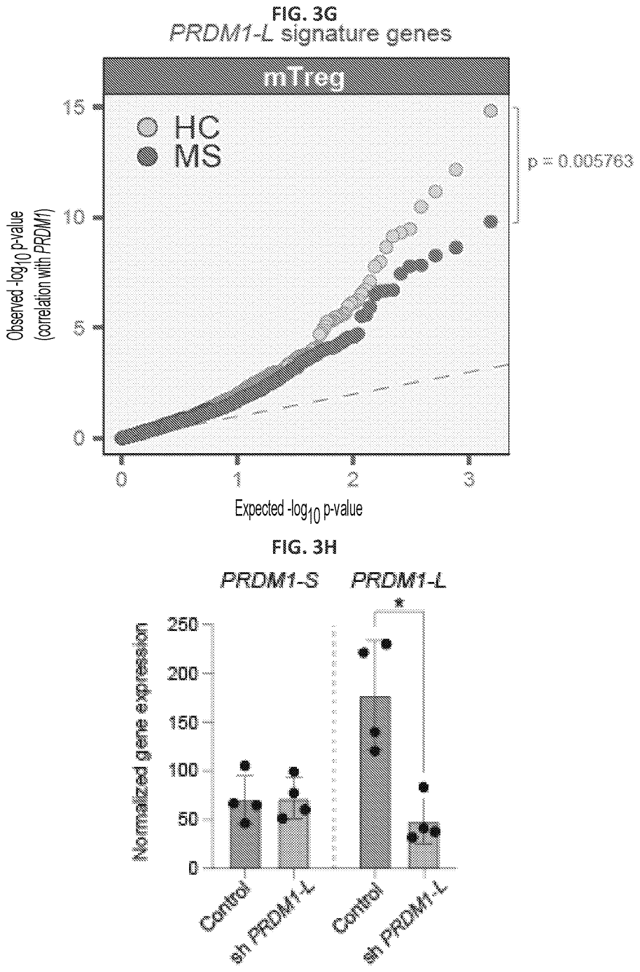

In humans, it has been appreciated that PRDM1 has two major isoforms: the original full-length isoform and another short isoform generated by alternative promoter usage (51). The short PRDM1 isoform (PRDM1-S; encoding a short form of Blimp1, Blimp1-S) arose during dry-nosed primate evolution, and thus is not coded in the mouse genome. Blimp1-S lacks the N-terminal region of Blimp1, which results in missing a part of the PR domain that is important in mediating interaction with co-factors of Blimp1 (FIG. 3A). Thus, Blimp1-S is implicated as a dominant negative form against the full-length Blimp1 (51, 52). Chromatin accessibility analysis at the PRDM1 locus showed that the PRDM1-S promoter region is significantly accessible in human Treg and a previous HiDRA analysis (53) revealed stronger activity at the promoter of PRDM1-S than that of full-length PRDM1 isoform (PRDM1-L), which is consistent with DNase I hypersensitive site (DHS) data of human primary total Treg (54) and T cells (55) (FIG. 3B). In addition, bulk RNA-seq with nine different peripheral blood human immune cells confirmed that both short and long PRDM1 isoforms were expressed in a cell-type-dependent manner. More specifically, monocytes and B cells mainly express PRDM1-L. In contrast, NK cells dominantly express PRDM1-S, and CD4+ T cells (including Treg) and CD8+ T cells express more PRDM1-S than PRDM1-L, especially in the memory population, suggesting cell type-specific roles for each isoform (FIG. 3C, 10A). This cell-type specific expression patterns of PRDM1-S and -L were further validated at protein level by western blot (FIGS. 3D, 10F). There are three bands detected between 80-110 kD consistent with previous studies (109), though the band sizes are slightly higher in the present study. To confirm the band size of Blimp1-S and Blimp1 protein, the open reading frame of Blimp1-S or Blimp1 were overexpressed in 293T cells by lentiviral transduction. The result shows that the lower two bands are derived from Blimp1-S while the higher molecular weight band is from Blimp1 (FIG. 10F). It was found that PRDM1-S is significantly increased in MS mTregs compared to healthy controls, though there is a moderate difference for PRDM1-L expression between MS and control subjects (FIG. 3E). This alteration was further validated by qPCR in both discovery and validation cohorts (FIG. 10B). Notably, this upregulation of PRDM1-S expression was also observed in SLE Tregs from ImmuNexUT data(1) (FIG. 10C). These data suggest that PRDM1-S is the key regulator of Tregs in autoimmune diseases.

These lines of data prompted the hypothesis that the aberrant induction of PRDM1-S may confer dysfunctional properties to autoimmune, MS Tregs by disrupting the homeostatic function of PRDM1-L (Blimp1) in Treg (FIG. 3F). Given that Blimp1-S can serve as a dominant negative isoform against conventional Blimp1, it was first examined whether the ratio of PRDM1-S and PRDM1-L is altered between MS vs HC. However, there was no significant difference observed in this ratio with either bulk RNA-seq or qPCR data, suggesting that the balance between PRDM1-S and PRDM1-L was not significantly disrupted in MS Tregs (FIG. 10G). Although PRDM1-S level was significantly upregulated in MS Tregs, PRDM1-L levels were not changed or slightly increased in MS Treg (FIGS. 3E, 10B), thus PRDM1-S mediated effects in MS Tregs could be independent from its dominant negative function against PRDM1-L.

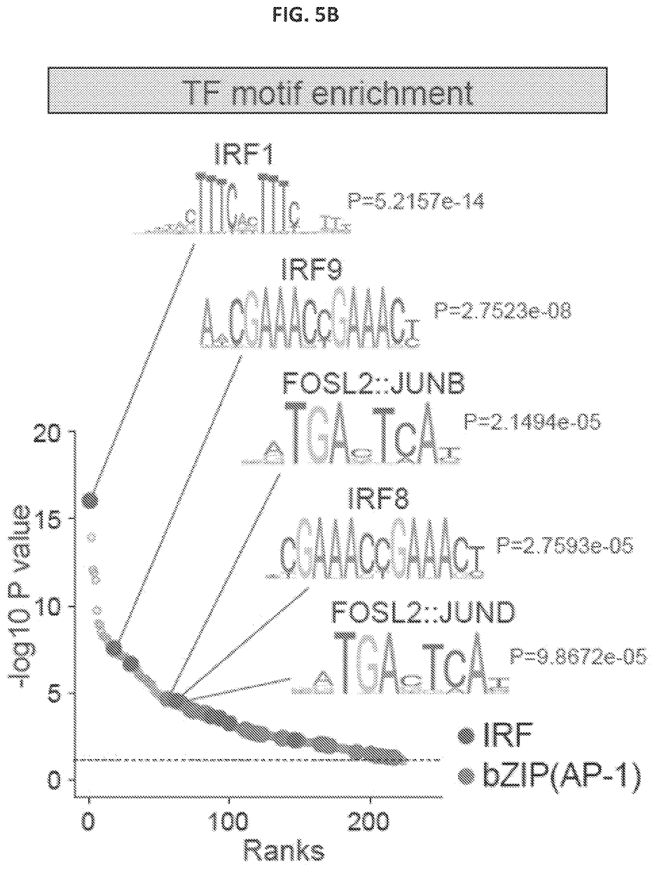

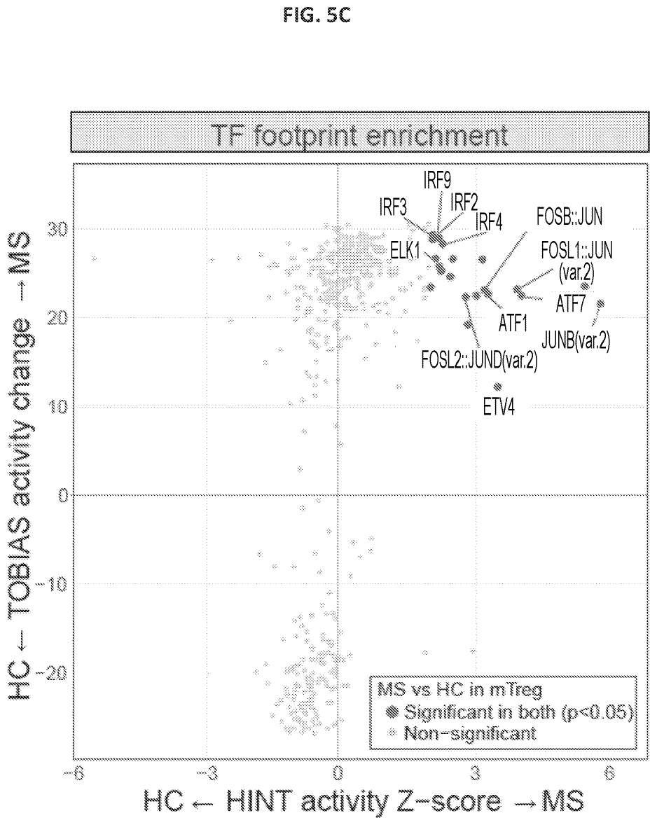

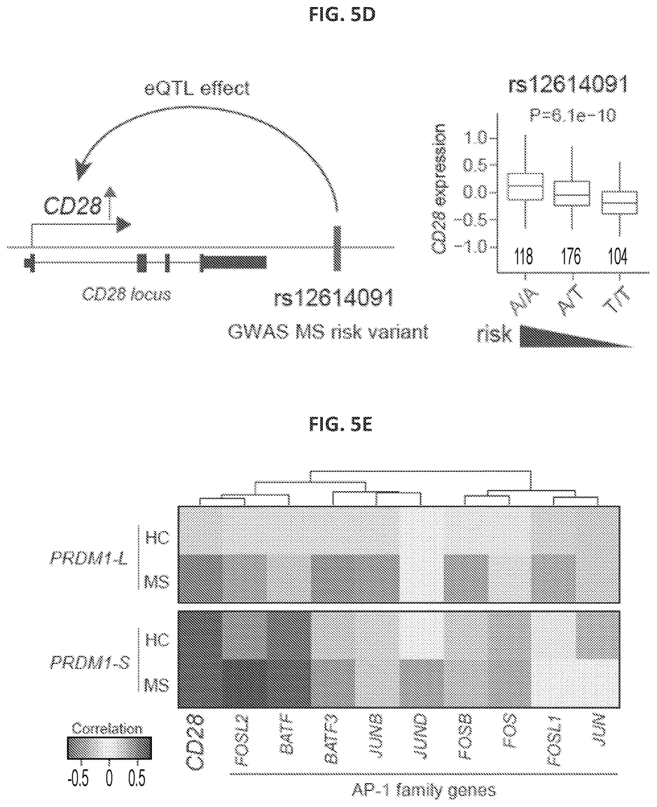

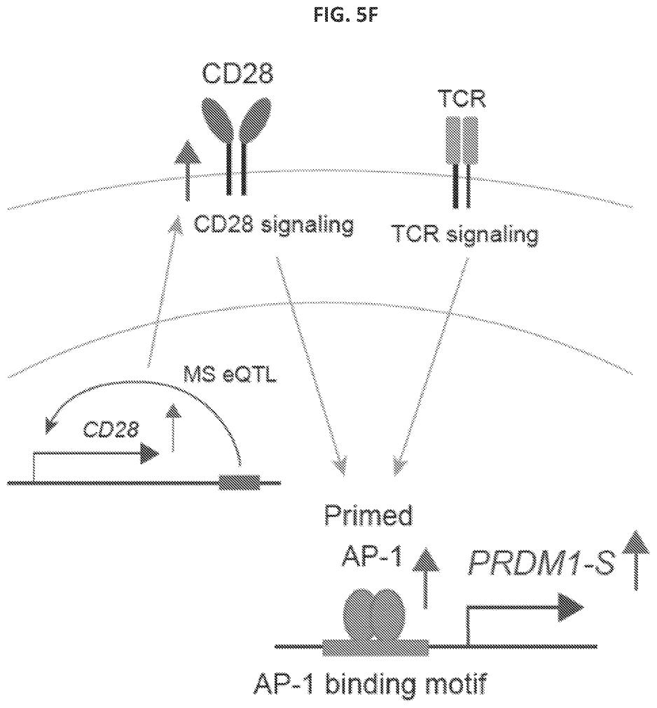

IRF family TFs share a common DNA binding sequence (IRF binding element). Of interest, IRF-1 and IRF-2, but not IRF-4 nor IRF-8, are known to compete with evolutionally conserved PRDM1-L (79, 80). IRF-1 plays a critical role in Treg differentiation and maintenance (59, 81) and it was of interest that IRF1 was more co-expressed with PRDM1-S as compared to PRDM1-L in MS mTreg (FIG. 10E). In addition, the enrichment of IRF-1 TF motif and footprint in MS mTreg (FIGS. 5B, 5C) could reflect the disrupted PRDM1-L-mediated gene regulation in MS mTreg. These results suggest that PRDM1-L mediated gene regulation could be disrupted in the context of MS (FIG. 3F). To test this hypothesis, the set of genes that are specifically regulated by PRDM1-L were defined by performing PRDM1-L-specific gene knockdown in human primary Tregs. A total of 1566 DEGs (defined as |log2FC| >1, FDR<0.05; 753 upregulated and 813 downregulated genes) were identified and defined as “PRDM1-L signature genes” (FIGS. 3F, 3H). Next, single-cell level correlation analysis was performed with the scRNA-seq data using a scalable negative binomial mixed model, NEBULA(56). Strong concordance between the bulk and scRNA-seq DEG analysis suggests that the increased abundance of PRDM1 in scRNA-seq of the MS samples largely attributes to PRDM1-S expression. It was reasoned that if PRDM1-S interferes with PRDM1-L mediated gene regulation, the correlation between PRDM1 and PRDM1-L signature genes will be disturbed due to higher abundance of PRDM1-S in MS Tregs (FIG. 3F). The correlations between PRDM1 and PRDM1-L signature genes were examined at the single-cell level, and it was found that PRDM1-L signature genes are less correlated with PRDM1 expression in MS mTregs compared to HC mTregs (FIG. 3G). The isoform level transcriptional co-regulation was then explored by using the bulk RNA-seq data. Approximately half of positively correlated PRDM1-L signature genes in Tregs from healthy controls lost their positive correlation in MS Tregs (FIG. 10D). Given that PRDM1-L plays a crucial role in maintaining Treg function (48-50), the correlation between PRDM1-L and Treg signature genes was assessed next (57, 58). As observed with PRDM1-L signature genes, half of positively correlated Treg signature genes in healthy controls lost their positive correlation in MS Tregs (FIG. 10D).

The patterns of co-expression for the curated immune-related genes were further determined (FIG. 10E); genes associated with effector and tissue resident Treg signature (i.e. BATF, CCR8, CD69, ILIRL1, AREG) were strongly co-expressed with PRDM1-S, particularly in MS Tregs, which reflects the skewing feature of MS mTreg towards effector/tissue resident properties. DEGs in MS mTreg, such as BCL3, were positively correlated with both PRDM1 isoforms in MS but negatively correlated in healthy controls. Of note, these positive correlations in MS are stronger for PRDM1-S compared to PRDM1-L. This pattern (positive correlation for MS but negative for control Tregs and higher correlation with PRDM1-S than PRDM1-L) was observed for the following genes; IRF1, which acts as a negative regulator for Foxp3 expression (59); BATF and FOSL2, which belong to AP-1 family, with crucial functions for Treg differentiation and maintenance (60, 61); SGK1, which is known to disrupt Treg homeostasis (17, 18) and play pathogenic roles in EAE (62). It was also noted that suppressive molecules enhancing Treg function (i.e. IKZF4, TIGIT, LAG3, ID3) are negatively correlated with PRDM1-S in MS but not in healthy controls, supporting the association between PRDM1-S and dysfunctional Treg properties in MS. Taken together, it was determined that PRDM1-S accounts for upregulation of PRDM1 in MS mTreg and its aberrant induction decouples the gene regulation by PRDM1-L in MS, which ultimately disturbs a homeostatic Treg gene program leading to Treg dysfunction in patients with MS.

### Example 4. Short PRDM1 Induces SGK1 and Treg Dysfunction

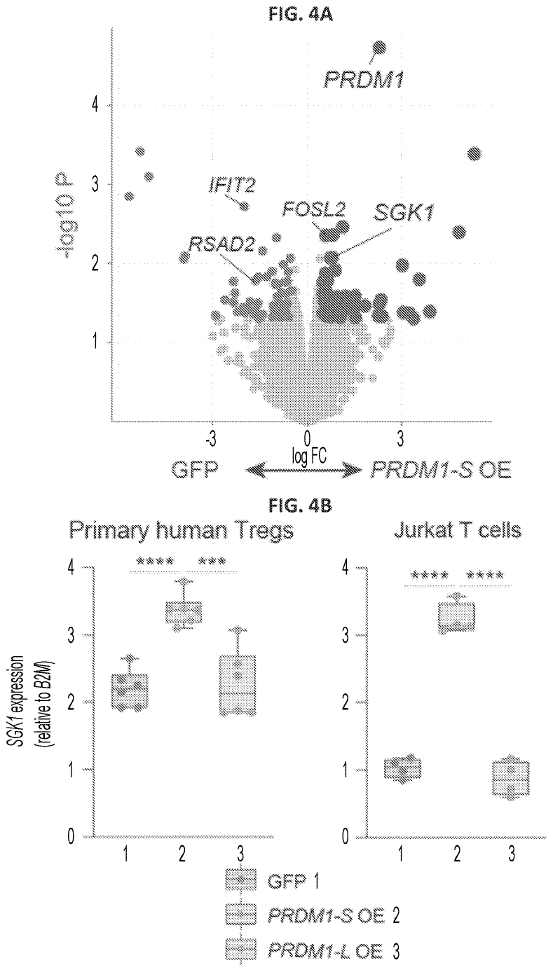

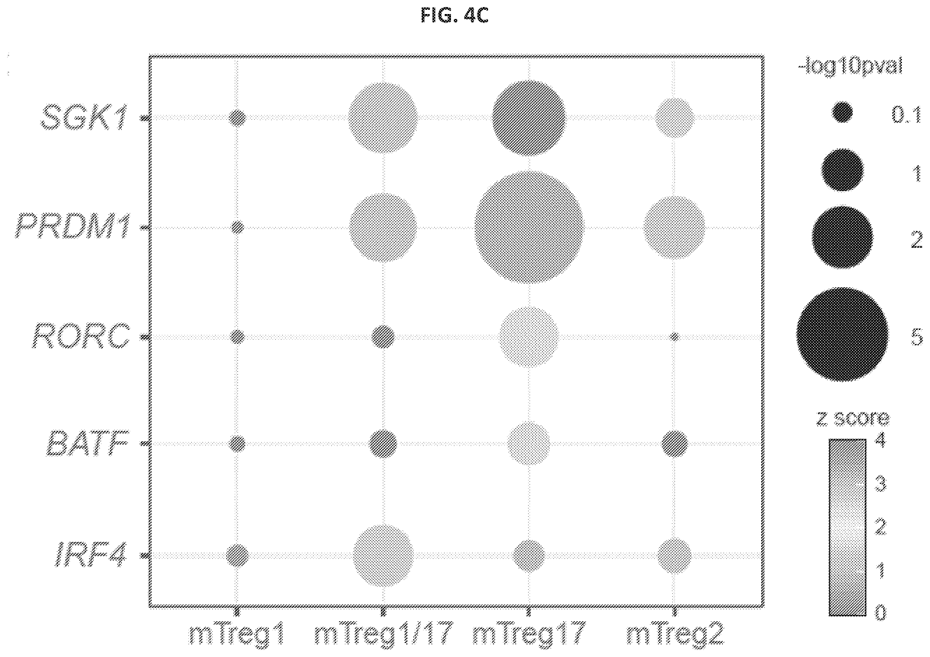

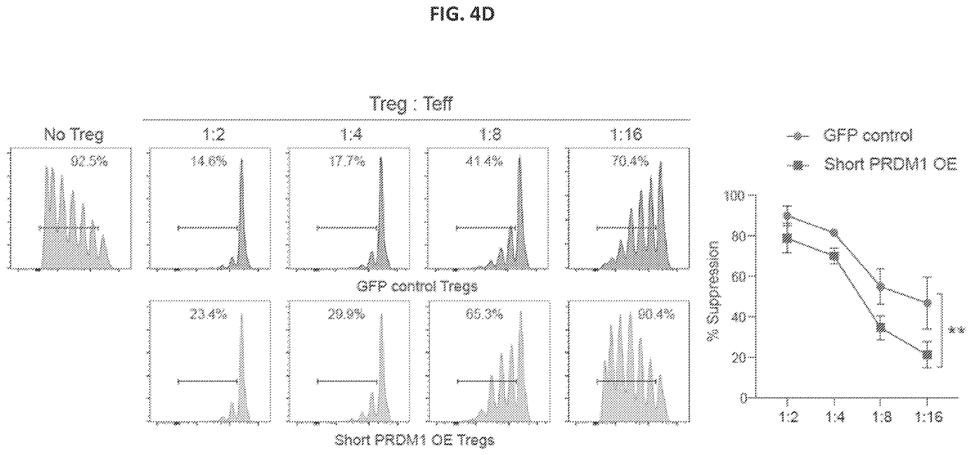

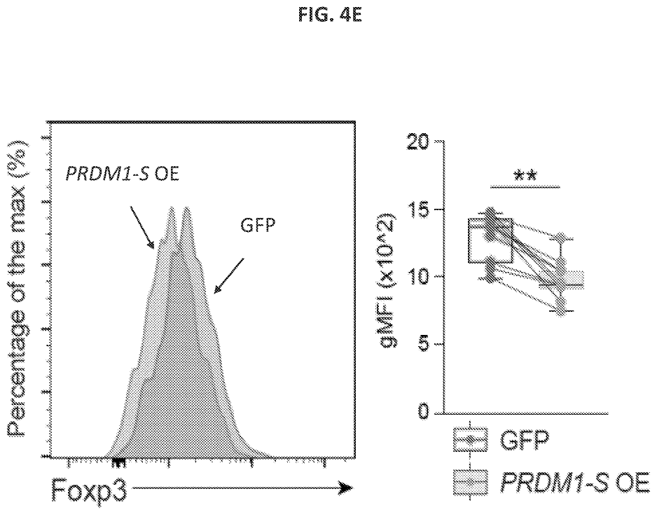

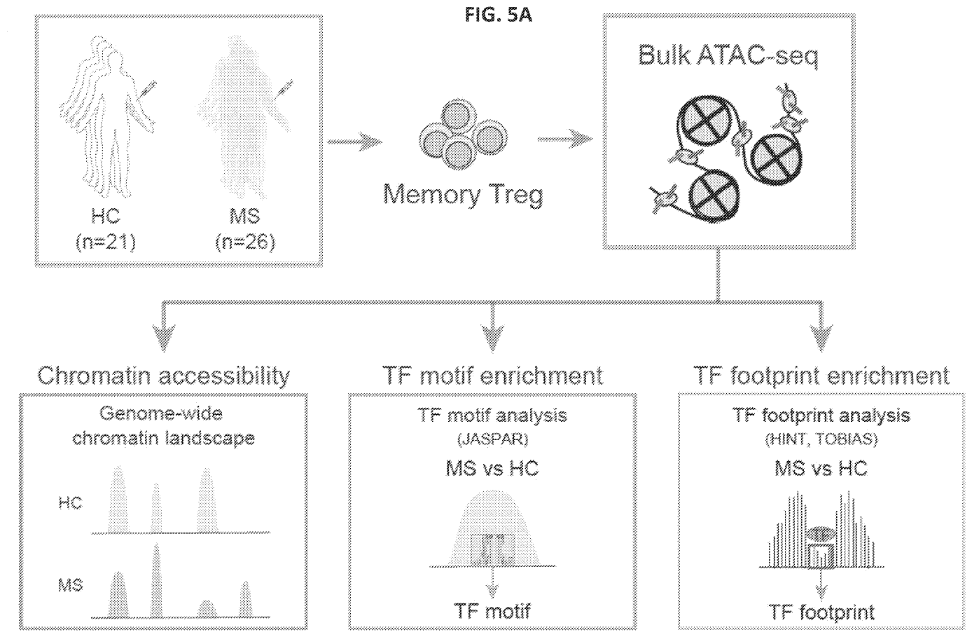

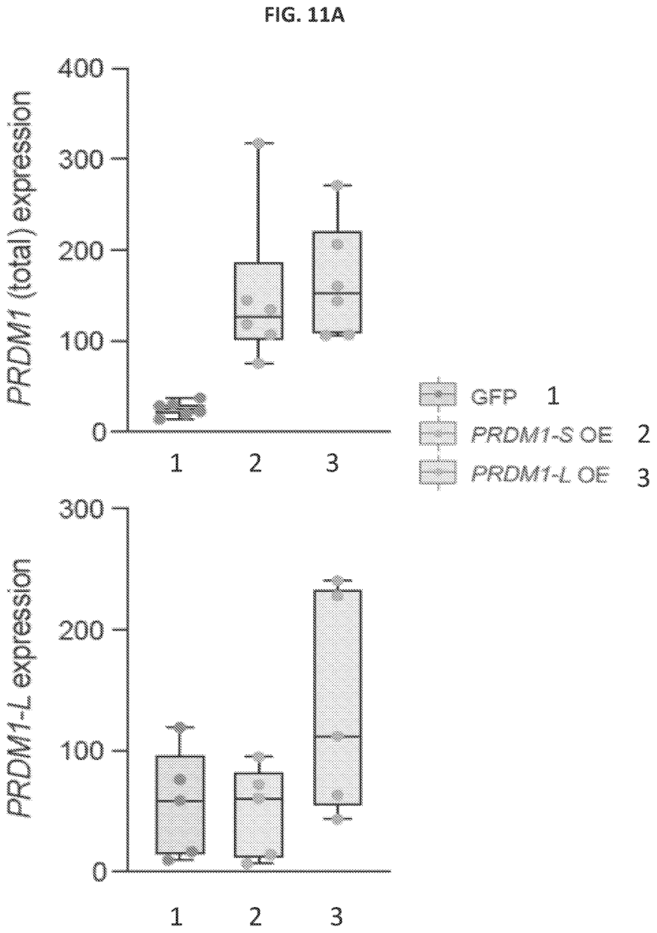

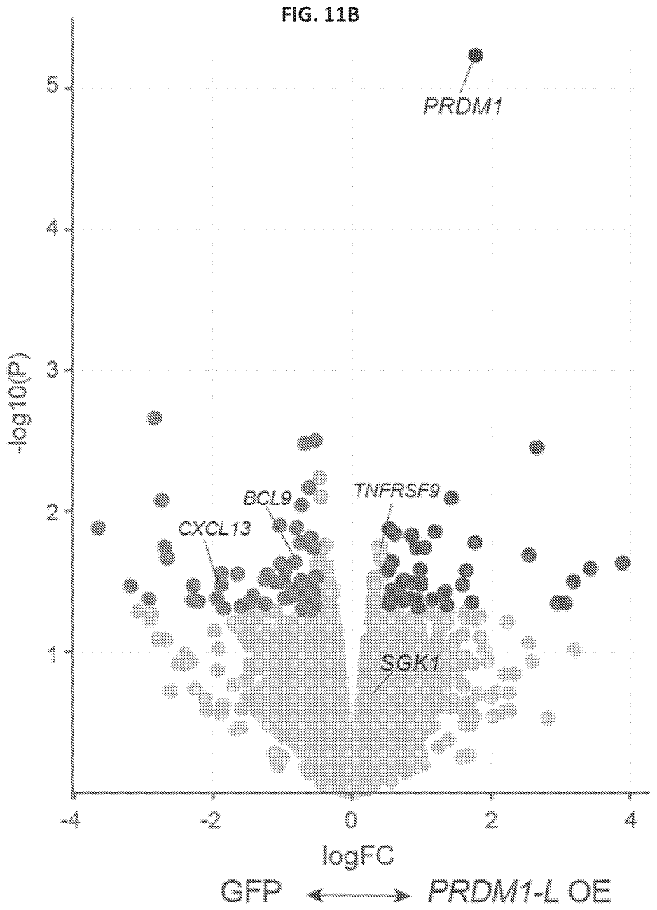

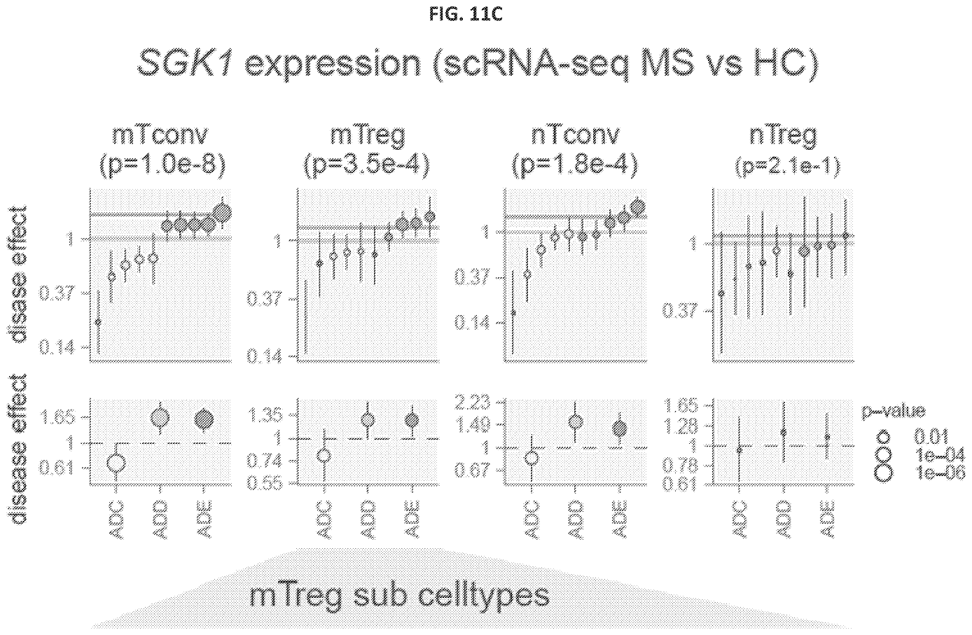

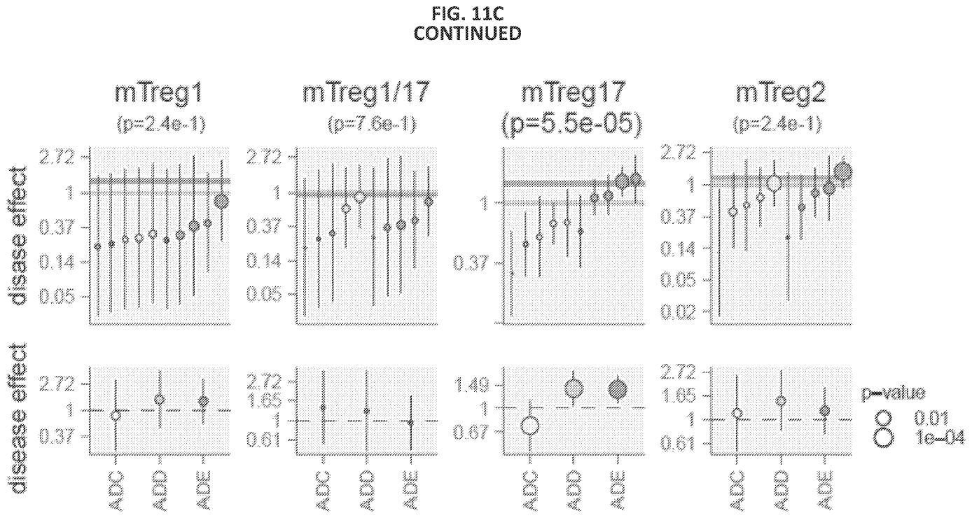

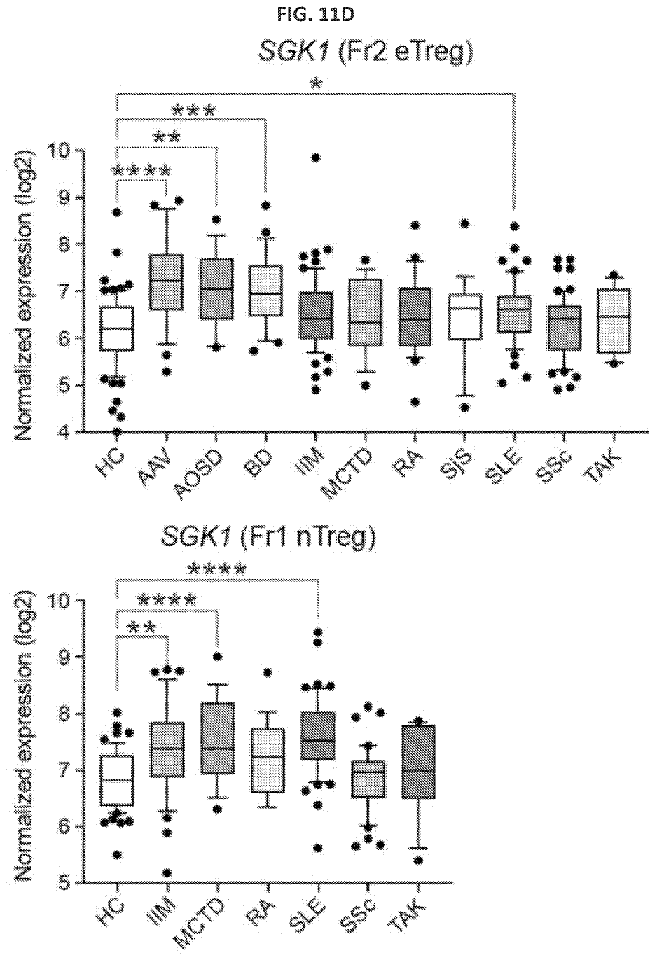

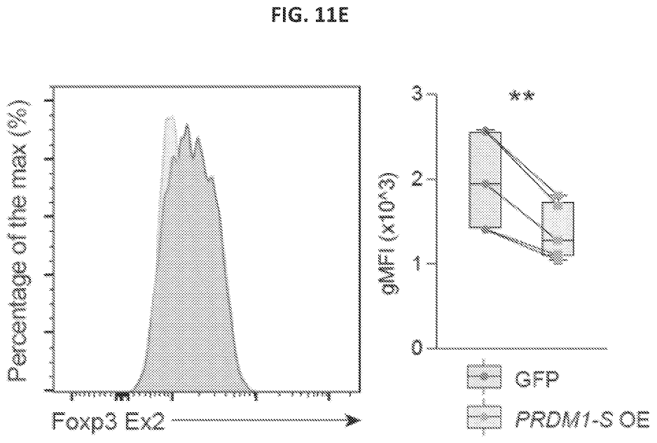

To further understand the molecular mechanisms underlining MS Treg dysfunction, PRDM1-S or PRDM1-L were transduced into primary human Tregs by using a lentivirus-based overexpression system. Isolated human primary Tregs were infected with lentivirus encoding PRDM1-S or PRDM1-L with GFP reporter and GFP positive transduced cells were sorted by FACS after four days of culture. Overexpression for each transcript was confirmed by qPCR (FIG. 11A). Bulk RNA-seq was performed on sorted GFP positive cells and highlighted 100 genes exhibiting nominal evidence of differential expression (|log 2FC|>0.6, P value<0.05) between PRDM1-S overexpression and GFP control (FIG. 4A). It was found that SGK1 was one of the major upregulated genes induced by PRDM1-S but not by PRDM1-L overexpression (FIG. 4A, 11B). This observation was validated by qPCR in human primary Treg cells and Jurkat T cells (FIG. 4B). Moreover, SGK1 was upregulated in the scRNA-seq analyses, particularly in the Th17-like Treg subset together with PRDM1, consistent with the role of SGK1 skewing pathogenic Th17 like signature in Tregs (62) (FIGS. 4C, 11C). This PRDM1-SGK1 axis was a common feature among other autoimmune diseases in a published dataset (1), where both PRDM1 and SGK1 were significantly increased in Tregs from patients with SLE and ANCA-associated vasculitis (FIGS. 7E, 11D). Finally, the impact of PRDM1-S on Treg function was examined by in vitro Treg suppression assay and it was found that Tregs with PRDM1-S overexpression (OE) exhibited lesser suppressive function than control GFP OE Treg, strongly indicating that aberrant PRDM1-S expression causes Treg dysfunction (FIG. 4D). PRDM1-S OE decreased the level of both full-length Foxp3 and the exon 2-containing suppressive Foxp3 isoform, further confirming the impaired suppressive function on Tregs with PRDM1-S OE (FIG. 4E, 11E). These data support the unique role of PRDM1-S as a positive regulator for SGK1 in human Treg cells, especially in the subset of potentially pathogenic Th17-like Treg, where SGK1 plays a proinflammatory role under high sodium conditions and is responsible for pathogenic features in a murine MS model via suppressing Foxp3 expression (17, 18). Thus, these data indicate that the PRDM1-S SGK1 axis underlies Treg dysfunction in MS.

### Example 5. Comprehensive Analysis for Chromatin Accessibility Reveals AP-1 and IRF Enrichment in MS mTreg

To further elucidate the regulatory mechanisms underlying the dysfunctional properties of MS mTregs, Assay for Transposase Accessible Chromatin sequencing (ATAC-seq) was performed to identify chromatin-accessible signatures and epigenetic regulatory elements. First, the change of mTreg chromatin accessibility between MS and healthy controls was determined. Surprisingly, there was no significant difference in genome-wide chromatin accessibility between MS and healthy controls in mTreg (FDR<0.05), suggesting that global chromatin accessibility is not per se the major factor regulating gene expression in MS mTreg. It was hypothesized that differential binding of regulatory TFs may account for the changes in mTreg gene expression between MS and control subjects. To identify TFs that potentially drive the observed gene expression signature in MS mTreg, the enrichment of TF motifs and TF footprints was analyzed within accessible regions between MS and healthy controls in mTreg (FIG. 5A). An enrichment of AP-1 and TRFs TF motifs that are important for CD4+ T cell activation and differentiation in MS mTreg was observed (FIG. 5B).

To gain deeper insight into captured accessible regulatory elements, a differential footprint analysis was performed on ATAC-seq peaks by using HINT (Hmm-based IdeNtification of Transcription factor footprints) (63) and TOBIAS (64). Consistent with the motif analysis, footprint analysis demonstrated the enrichment of AP-1 family TFs and TRFs in mTregs from MS compared to control subjects (65-68) (FIG. 5C). AP-1 transcriptional activity is negatively regulated by direct interaction with Foxp3 (69) and AP-1 is postulated to serve as a pioneer factor at Treg-specific regulatory elements where Foxp3 subsequently replaces it to establish Treg-specific enhancer architecture and DNA methylation (70, 71). The observation of AP-1 enrichment in dysfunctional Tregs in MS could thus stem from the lower or impaired activity of Foxp3 in MS mTreg and reflect a more effector-like mTreg function in patients with MS (72, 73).

Fundamental pathways that activate AP-1 are the combination of T cell receptor and CD28 co-stimulatory signaling (74). Recent findings highlighted that AP-1 binding events associated with CD28 co-stimulation signals are highly enriched in MS risk loci (75). Moreover, the CD28 locus harbors multiple eQTLs and variants associated with multiple autoimmune diseases, including MS (22, 76). Given the tightly linked CD28 signaling and AP-1 activation, it was reasoned that a MS GWAS risk variant near CD28 (rs12614091) (77) can confer AP-1 enrichment observed in MS mTreg. By mining of existing datasets ImmuNexUT(1) and DICE(78), it was found rs12614091 exerted a cell-type specific eQTL effect primarily on Tregs (FIG. 5D, 12A), which is consistent with a previous report (76). Indeed, a co-expression analysis with PRDM1 isoforms in HC and MS mTregs showed an enriched positive correlation between PRDM1-S and CD28/AP-1 family genes especially in MS mTreg (FIG. 5E). These findings and previous evidence imply a causal link between genetic susceptibility at CD28 locus and AP-1 enrichment, which potentially induce PRDM1-S in MS Tregs (FIG. 5F).

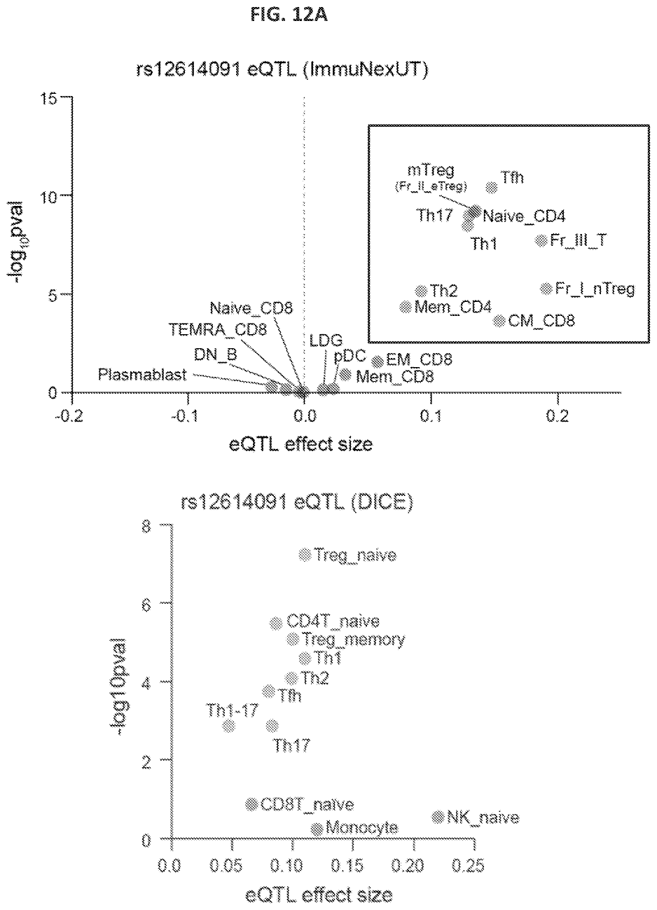

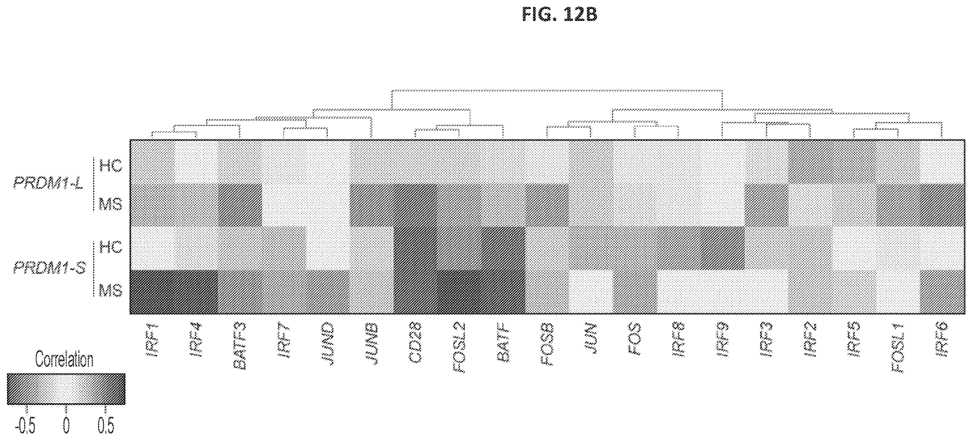

IRF family TFs share a common DNA binding sequence (IRF binding element) and compete with conventional PRDM1-L (79, 80). Therefore, the enrichment of IRF TF motifs and footprints in MS mTreg supports disrupted PRDM1-L-mediated gene regulation in MS mTreg. Among IRF family TFs, IRF-1 and IRF-4 are known to function as critical regulators for Treg differentiation and maintenance (59, 81, 82). Thus, it was of interest that IRF1 and IRF4 were positively co-expressed with PRDM1-S as well as AP-1 family TFs genes in MS mTreg (FIG. 12B) suggesting their role in disrupting PRDM1-L mediated gene regulation in the context of MS. Taken together, these ATAC-seq results revealed that while genome-wide chromatin accessibility was not significantly altered, differential binding of TFs (AP-1 and TRFs) to regulatory elements could serve as key upstream regulatory factors and drive dysfunctional Treg gene programs in patients with MS.

### Example 6. Identification of Active Enhancer for Short PRDM1 in Human T Cells

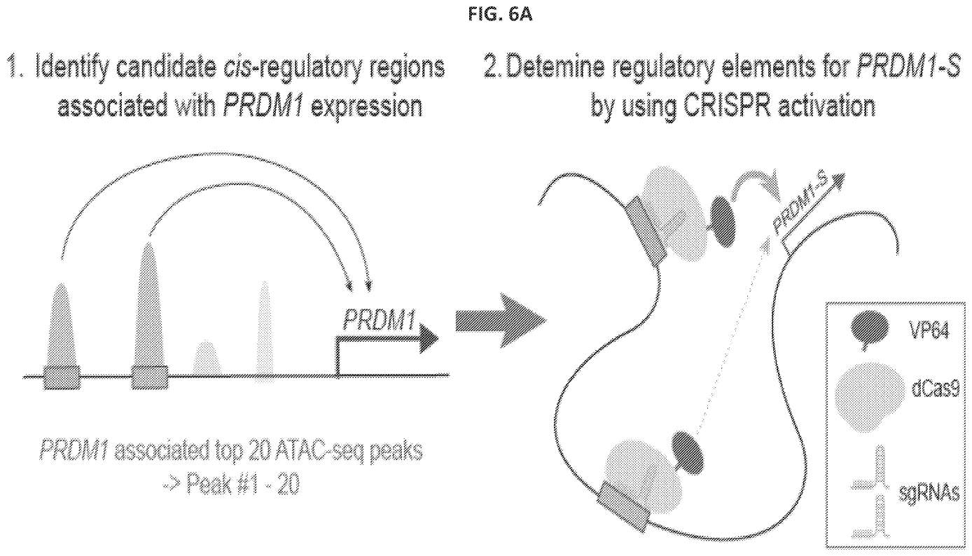

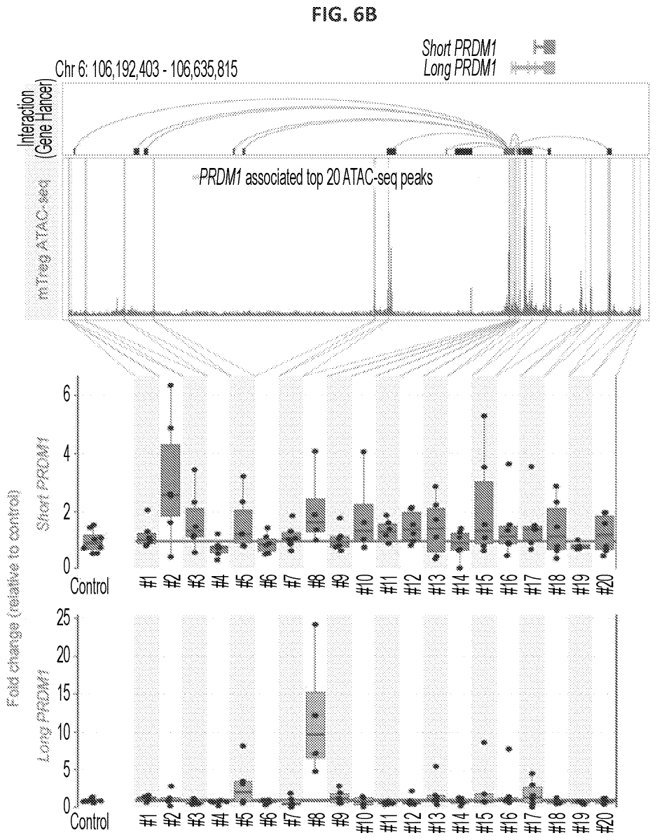

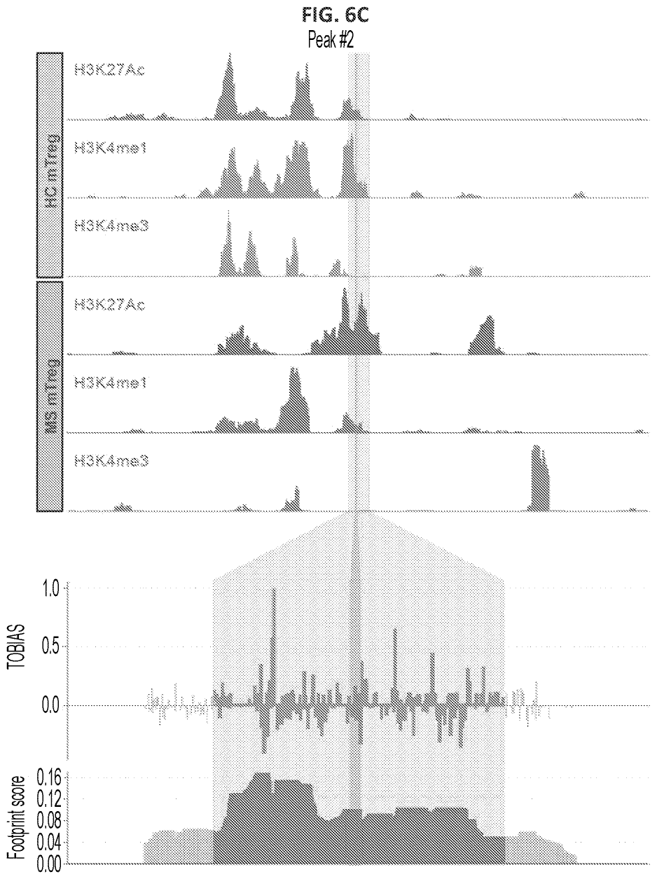

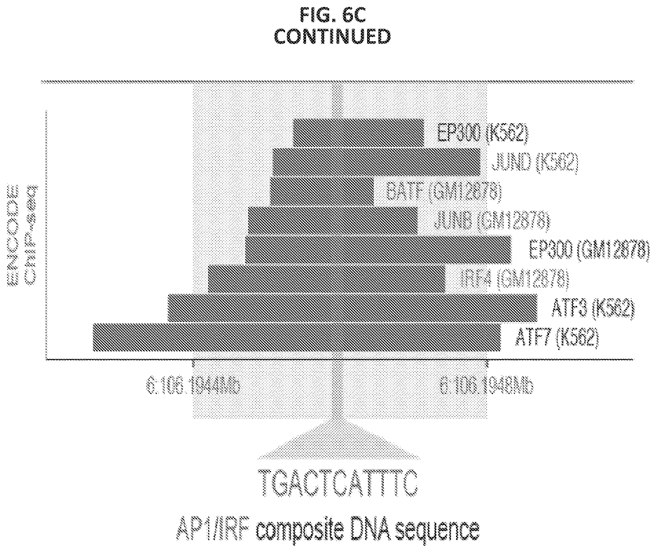

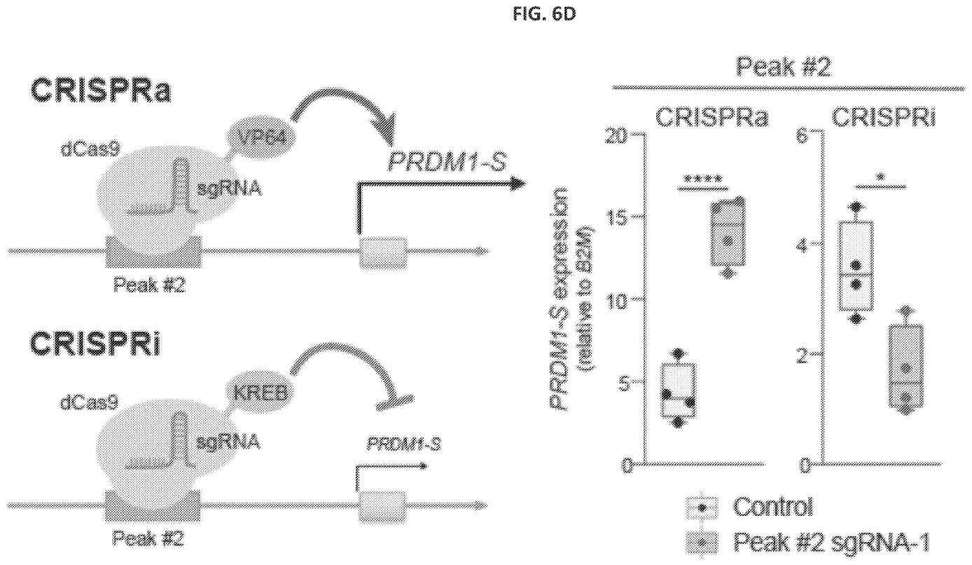

An effort was carried out to determine the regulatory mechanisms that induce upregulation of PRDM1-S as opposed to PRDM1-L in human mTreg. It was reasoned that the enriched binding of AP-1 and IRFs exert its regulatory function through binding to cis-regulatory elements for PRDM1, especially PRDM1-S. To identify the cis-regulatory elements for PRDM1-S, the accessible chromatin regions surrounding the PRDM1 locus (+/−0.5 MB window around the transcriptional start site of PRDM1) were prioritized and the association between PRDM1 expression and chromatin accessibility in the human primary T cell ATAC-seq and RNA-seq data were examined. Twenty significant accessible regions (p<0.001) were identified as potential regulatory elements associated with PRDM1 expression (FIG. 6A). The majority of these peaks overlapped with H3K27ac ChIP-seq signals for primary human Tregs (55), nominating them as potential enhancers. To functionally validate these accessible chromatin regions, the CRISPR activation (CRISPRa) system was adopted in Jurkat T cells that stably expresses catalytically inactive Cas9 fused to the transcriptional activator VP64 (dCas9-VP64) (83) (FIG. 6A).

TABLE 3
PRDM1 Signature Genes

| gene | coexp_con_short | coexp_MS_short | coexp_con_long | coexp_MS_long |
|---|---|---|---|---|
| ID2 | −0.494736842 | −0.269349845 | −0.056140351 | −0.199174407 |
| APP | −0.352631579 | −0.108359133 | 0.314035088 | −0.277605779 |
| KLRG1 | 0.349122807 | 0.093911249 | −0.189473684 | 0.327141383 |
| CCR6 | 0.392982456 | 0.471620227 | 0.529824561 | 0.19504644 |
| CTLA4 | 0.314035088 | 0.69246646 | 0.301754386 | 0.768833849 |
| RNASE4 | −0.242105263 | −0.401444788 | −0.10877193 | −0.205366357 |
| ITGAE | −0.215789474 | −0.098039216 | −0.280701754 | 0.436532508 |
| GZMB | −0.373684211 | −0.011351909 | 0.329824561 | 0.050567595 |
| FRMD4B | −0.136842105 | 0.046439628 | 0.184210526 | 0.364293086 |
| IL1RL1 | −0.454385965 | 0.081527348 | −0.324561404 | −0.335397317 |
| OSBPL3 | −0.026315789 | 0.139318885 | 0.18245614 | −0.168214654 |
| MATN2 | −0.41754386 | 0.180598555 | 0.585964912 | −0.048503612 |
| RGS16 | −0.41754386 | 0.209494324 | −0.045614035 | 0.005159959 |
| NFIL3 | 0.1 | 0.488132095 | 0.066666667 | 0.547987616 |
| PRR13 | 0.071929825 | −0.28998968 | −0.39122807 | 0.091847265 |
| TIGIT | 0.131578947 | −0.380804954 | −0.171929825 | −0.294117647 |
| SLC40A1 | 0.1 | −0.296181631 | −0.036842105 | −0.362229102 |
| FGL2 | −0.236842105 | −0.230134159 | 0.161403509 | −0.10629515 |
| LGALS3 | 0.349122807 | −0.207430341 | −0.135087719 | 0.358101135 |
| CCR10 | 0.342105263 | 0.038183695 | −0.143859649 | −0.114551084 |
| MYO1F | −0.036842105 | 0.178534572 | 0.080701754 | −0.223942208 |
| PRDM1 | 0.921052632 | 0.849329205 | 0.305263158 | 0.585139319 |
| IL17RB | −0.214035088 | −0.570691434 | −0.236842105 | −0.397316821 |
| LGALS1 | −0.154385965 | −0.50877193 | −0.257894737 | −0.011351909 |
| PTGS1 | −0.19122807 | −0.475748194 | 0.271929825 | −0.261093911 |
| LRP1 | −0.024561404 | −0.457172343 | 0.287719298 | −0.574819401 |
| IL18R1 | 0.089473684 | −0.448916409 | 0.157894737 | −0.172342621 |
| HEBP1 | 0.147368421 | −0.424148607 | −0.528070175 | −0.269349845 |
| NAV2 | −0.452631579 | −0.376676987 | 0.205263158 | −0.463364293 |
| THEMIS2 | 0.305263158 | −0.323013416 | −0.250877193 | 0.126934985 |
| AXL | −0.431578947 | −0.308565531 | −0.033333333 | −0.444788442 |
| TTC39C | −0.138596491 | −0.296181631 | −0.245614035 | −0.044375645 |
| GFRA2 | −0.492982456 | −0.285861713 | 0.089473684 | −0.389060888 |
| ITGB5 | −0.294736842 | −0.271413829 | 0.068421053 | −0.062951496 |
| VIM | 0.515789474 | −0.234262126 | −0.164912281 | 0.029927761 |
| SLC7A8 | 0.078947368 | −0.223942208 | 0.357894737 | −0.281733746 |
| PLTP | 0.203508772 | −0.192982456 | 0.298245614 | −0.310629515 |
| FCGRT | 0.333333333 | −0.186790506 | −0.277192982 | 0.149638803 |
| ARL5A | −0.263157895 | −0.186790506 | 0.045614035 | 0.232198142 |
| TGFBI | −0.263157895 | −0.182662539 | −0.1 | −0.15995872 |
| NEK2 | −0.121052632 | −0.178534572 | 0.145614035 | 0.347781218 |
| CDC42EP2 | −0.352631579 | −0.151702786 | −0.135087719 | −0.052631579 |
| TJP2 | 0.238596491 | −0.149638803 | 0.071929825 | −0.265221878 |
| AMZ1 | −0.498245614 | −0.128998968 | 0.154385965 | −0.267285862 |
| AURKA | −0.184210526 | −0.122807018 | −0.192982456 | 0.06501548 |
| RGL1 | −0.089473684 | −0.114551084 | −0.301754386 | −0.287925697 |
| SLC39A4 | −0.042105263 | −0.102167183 | −0.350877193 | 0.108359133 |
| ACOT11 | −0.501754386 | −0.098039216 | 0.335087719 | −0.298245614 |
| RRM2 | 0.356140351 | −0.087719298 | 0.171929825 | 0.312693498 |
| ADAM8 | −0.373684211 | −0.081527348 | 0.212280702 | −0.469556244 |
| EGLN3 | −0.10877193 | −0.075335397 | −0.003508772 | −0.019607843 |
| NR1H3 | 0.187719298 | −0.073271414 | −0.080701754 | −0.166150671 |
| CDKN2A | −0.08245614 | −0.062951496 | −0.315789474 | 0.42002064 |
| FUT7 | 0.159649123 | −0.060887513 | −0.028070175 | −0.496388029 |
| LTBR | −0.457894737 | −0.007223942 | 0.407017544 | −0.500515996 |
| GCNT1 | 0.315789474 | −0.005159959 | 0.136842105 | −0.073271414 |
| CASS4 | 0.18245614 | 0.003095975 | 0.229824561 | −0.502579979 |
| IL9R | 0.171929825 | 0.007223942 | −0.1 | −0.389060888 |
| COPZ2 | −0.050877193 | 0.013415893 | −0.154385965 | 0.432404541 |
| CXCR3 | 0.3 | 0.02373581 | −0.014035088 | 0.120743034 |
| PTGIS | −0.398245614 | 0.02373581 | 0.110526316 | −0.339525284 |
| MATK | −0.073684211 | 0.027863777 | −0.49122807 | −0.477812178 |
| LGMN | 0.336842105 | 0.027863777 | −0.105263158 | 0.455108359 |
| TMBIM1 | 0.428070175 | 0.031991744 | −0.052631579 | −0.054695562 |
| CDC25B | 0.173684211 | 0.042311662 | 0.075438596 | −0.164086687 |
| PLEKHG3 | 0.168421053 | 0.046439628 | −0.131578947 | −0.353973168 |
| CCL4 | −0.138596491 | 0.050567595 | 0.449122807 | 0.292053664 |
| NID2 | −0.378947368 | 0.07120743 | −0.036842105 | −0.353973168 |
| FRMD5 | −0.445614035 | 0.077399381 | 0.242105263 | 0.013415893 |
| CRIP2 | 0.240350877 | 0.079463364 | −0.331578947 | 0.116615067 |
| CFP | 0.149122807 | 0.095975232 | 0.150877193 | 0.176470588 |
| VSTM4 | −0.543859649 | 0.098039216 | 0.196491228 | −0.335397317 |
| AURKB | 0.124561404 | 0.100103199 | 0.135087719 | 0.576883385 |
| BSPRY | 0.057894737 | 0.1124871 | 0.247368421 | 0.15995872 |
| RAP1GAP2 | −0.324561404 | 0.143446852 | 0.068421053 | −0.215686275 |
| EHD4 | 0.268421053 | 0.155830753 | 0.373684211 | 0.164086687 |
| ASB2 | 0.077192982 | 0.176470588 | −0.18245614 | −0.219814241 |
| TBXAS1 | 0.133333333 | 0.184726522 | −0.029824561 | 0.265221878 |
| MXD3 | −0.249122807 | 0.19504644 | 0.038596491 | 0.547987616 |
| FKBP5 | 0.605263158 | 0.199174407 | 0.068421053 | −0.114551084 |
| FAM83G | −0.266666667 | 0.20123839 | 0.075438596 | −0.302373581 |
| RACGAP1 | 0.528070175 | 0.203302374 | −0.257894737 | 0.042311662 |
| HAVCR2 | 0.21754386 | 0.215686275 | 0.054385965 | 0.254901961 |
| CTSA | 0.631578947 | 0.246646027 | −0.021052632 | 0.362229102 |
| CD68 | 0.063157895 | 0.246646027 | 0.315789474 | 0.170278638 |
| INCENP | 0.1 | 0.265221878 | 0.054385965 | 0.618163055 |
| CSF1R | −0.122807018 | 0.285861713 | −0.154385965 | 0.131062951 |
| RXRA | 0.405263158 | 0.287925697 | −0.092982456 | −0.102167183 |
| MRC1 | 0.285964912 | 0.327141383 | −0.203508772 | 0.003095975 |
| KIF22 | 0.612280702 | 0.341589267 | −0.264912281 | 0.353973168 |
| ICOS | −0.166666667 | 0.341589267 | 0.059649123 | 0.459236326 |
| PRC1 | −0.338596491 | 0.351909185 | 0.456140351 | 0.630546956 |
| GRN | 0.4 | 0.362229102 | −0.373684211 | 0.141382869 |
| GNA15 | −0.207017544 | 0.366357069 | 0.19122807 | 0.06501548 |
| PDCD1 | 0.398245614 | 0.37874097 | 0.050877193 | 0.162022704 |
| METRNL | −0.187719298 | 0.401444788 | −0.043859649 | 0.42002064 |
| PLK1 | 0.370175439 | 0.411764706 | −0.110526316 | 0.496388029 |
| HFE | −0.526315789 | 0.424148607 | 0.336842105 | −0.042311662 |
| TRPM2 | 0.005263158 | 0.477812178 | −0.147368421 | 0.228070175 |
| PAQR4 | 0.473684211 | 0.611971104 | 0.285964912 | 0.001031992 |
| LSR | 0.328070175 | 0.620227038 | −0.335087719 | 0.217750258 |
| CCNF | −0.066666667 | 0.657378741 | 0.698245614 | 0.279669763 |
| MAFG | 0.435087719 | 0.682146543 | −0.231578947 | 0.199174407 |
| CD300A | 0.259649123 | −0.302373581 | −0.185964912 | −0.430340557 |
| TIMD4 | −0.061403509 | −0.277605779 | −0.150877193 | −0.145510836 |
| AIF1 | −0.036842105 | −0.102167183 | −0.00877193 | −0.446852425 |
| PTPRM | 0.377192982 | 0.162022704 | 0.087719298 | −0.42002064 |
| GLRX | −0.214035088 | −0.351909185 | −0.298245614 | 0.192982456 |
| PIGZ | −0.389473684 | −0.339525284 | −0.063157895 | −0.06501548 |
| SLC16A9 | −0.012280702 | −0.287925697 | 0.075438596 | −0.422084623 |
| HSBP1L1 | −0.192982456 | −0.203302374 | 0.140350877 | −0.075335397 |
| MMP25 | −0.131578947 | −0.155830753 | 0.094736842 | −0.521155831 |
| SYNGR3 | −0.257894737 | −0.143446852 | 0.085964912 | 0.116615067 |
| JAG1 | −0.247368421 | −0.143446852 | 0.312280702 | −0.374613003 |
| NACC2 | 0.036842105 | −0.135190918 | −0.085964912 | −0.223942208 |
| COL1A1 | −0.133333333 | −0.098039216 | 0.28245614 | −0.329205366 |
| RAPH1 | −0.540350877 | 0.01754386 | 0.215789474 | 0.019607843 |
| FBXO2 | 0.189473684 | 0.10629515 | −0.121052632 | −0.246646027 |
| SLC16A10 | −0.059649123 | 0.108359133 | 0.489473684 | −0.327141383 |
| NEBL | 0.277192982 | 0.15376677 | 0.066666667 | 0.135190918 |
| CDC42BPB | 0.275438596 | 0.207430341 | 0.580701754 | −0.343653251 |
| MYADM | −0.254385965 | 0.236326109 | 0.19122807 | −0.126934985 |
| SNTA1 | 0.131578947 | 0.24251806 | −0.021052632 | 0.141382869 |
| ADAM12 | −0.173684211 | 0.244582043 | 0.345614035 | 0.091847265 |
| SRGN | −0.133333333 | 0.296181631 | 0.061403509 | 0.762641899 |
| ERMN | −0.159649123 | 0.459236326 | 0.173684211 | 0.091847265 |
| F11R | 0.142105263 | 0.481940144 | 0.114035088 | 0.20123839 |
| TBC1D2 | −0.163157895 | 0.484004128 | 0.257894737 | 0.178534572 |
| SCML4 | −0.10877193 | −0.120743034 | 0.068421053 | −0.176470588 |
| BACH2 | −0.170175439 | 0.168214654 | 0.022807018 | 0.217750258 |
| TCF7 | 0.154385965 | 0.192982456 | 0.121052632 | −0.192982456 |
| SATB1 | 0.075438596 | 0.277605779 | 0.131578947 | −0.122807018 |
| ATP1B1 | −0.161403509 | 0.463364293 | 0.122807018 | 0.304437564 |
| AMIGO2 | −0.328070175 | −0.409700722 | −0.105263158 | −0.323013416 |
| LTA | −0.289473684 | −0.186790506 | −0.161403509 | −0.172342621 |
| IGFBP4 | −0.203508772 | −0.166150671 | −0.347368421 | −0.025799794 |
| ADAMTS6 | −0.596491228 | −0.147574819 | −0.10877193 | −0.256965944 |
| ENO3 | 0.019298246 | −0.104231166 | −0.215789474 | −0.081527348 |
| SLC22A5 | 0.080701754 | −0.069143447 | 0.10877193 | −0.574819401 |
| UGCG | −0.324561404 | 0.034055728 | 0.456140351 | 0.091847265 |
| IGF2R | 0.266666667 | 0.234262126 | 0.480701754 | 0.403508772 |
| ST8SIA1 | −0.08245614 | 0.267285862 | 0.292982456 | 0.172342621 |
| ID3 | −0.580701754 | −0.628482972 | −0.480701754 | 0.001031992 |
| ST8SIA6 | 0.243859649 | −0.434468524 | 0.045614035 | −0.217750258 |
| SELP | −0.236842105 | −0.294117647 | 0.149122807 | −0.599587203 |
| CYTH3 | 0.042105263 | 0.215686275 | 0.231578947 | −0.02373581 |
| UPP1 | −0.371929825 | −0.448916409 | −0.428070175 | −0.415892673 |
| RAB3IP | 0.068421053 | −0.405572755 | −0.171929825 | 0.007223942 |
| BCL2 | −0.010526316 | −0.277605779 | 0.205263158 | −0.199174407 |
| DAPK1 | −0.575438596 | −0.259029928 | −0.084210526 | −0.438596491 |
| AHCYL2 | −0.114035088 | −0.230134159 | 0.166666667 | −0.442724458 |
| CARD6 | −0.257894737 | −0.205366357 | 0.019298246 | −0.302373581 |
| SLC17A9 | −0.356140351 | −0.203302374 | 0.166666667 | −0.358101135 |
| WNT10A | −0.059649123 | −0.197110423 | −0.054385965 | −0.108359133 |
| ADCY6 | −0.538596491 | −0.164086687 | −0.149122807 | −0.300309598 |
| SOX4 | −0.507017544 | −0.128998968 | 0.19122807 | −0.223942208 |
| SELL | 0.050877193 | −0.042311662 | −0.189473684 | 0.162022704 |
| SH3BP5 | 0.370175439 | 0.01754386 | 0.073684211 | 0.046439628 |
| TNFSF8 | 0.443859649 | 0.038183695 | −0.052631579 | −0.143446852 |
| NOD1 | −0.380701754 | 0.046439628 | 0.056140351 | −0.42621259 |
| CCR7 | 0.221052632 | 0.135190918 | −0.154385965 | 0.137254902 |
| AFF3 | −0.285964912 | 0.155830753 | −0.152631579 | −0.15995872 |
| PARP8 | 0.08245614 | 0.190918473 | 0.184210526 | 0.170278638 |
| EVL | −0.064912281 | 0.362229102 | −0.064912281 | −0.07120743 |
| PIK3IP1 | 0.610526316 | 0.368421053 | 0.142105263 | 0.512899897 |
| LRRC32 | 0.033333333 | 0.37874097 | 0.103508772 | 0.254901961 |
| NEDD4L | 0.366666667 | 0.463364293 | 0.222807018 | 0.110423117 |
| PDLIM1 | −0.285964912 | −0.345717234 | −0.126315789 | −0.37254902 |
| SPTB | −0.540350877 | −0.10629515 | 0.052631579 | −0.223942208 |
| IQCK | −0.271929825 | −0.003095975 | −0.168421053 | 0.060887513 |
| ZBTB18 | −0.222807018 | 0.221878225 | 0.315789474 | 0.075335397 |

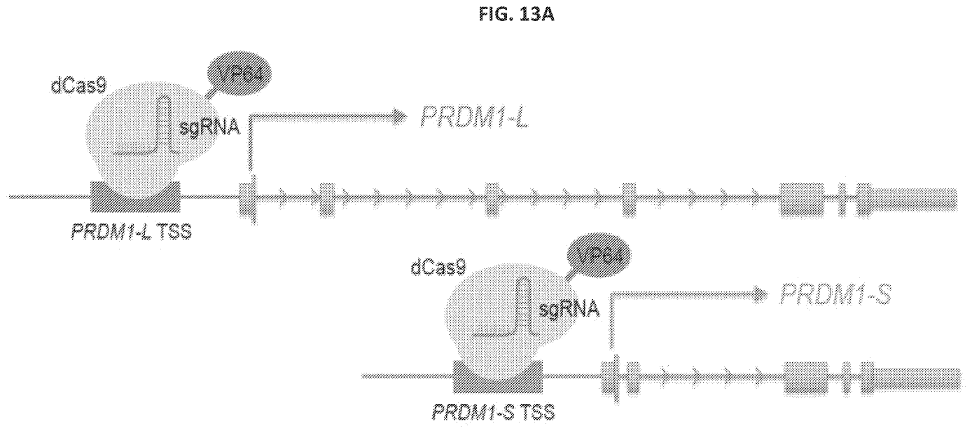

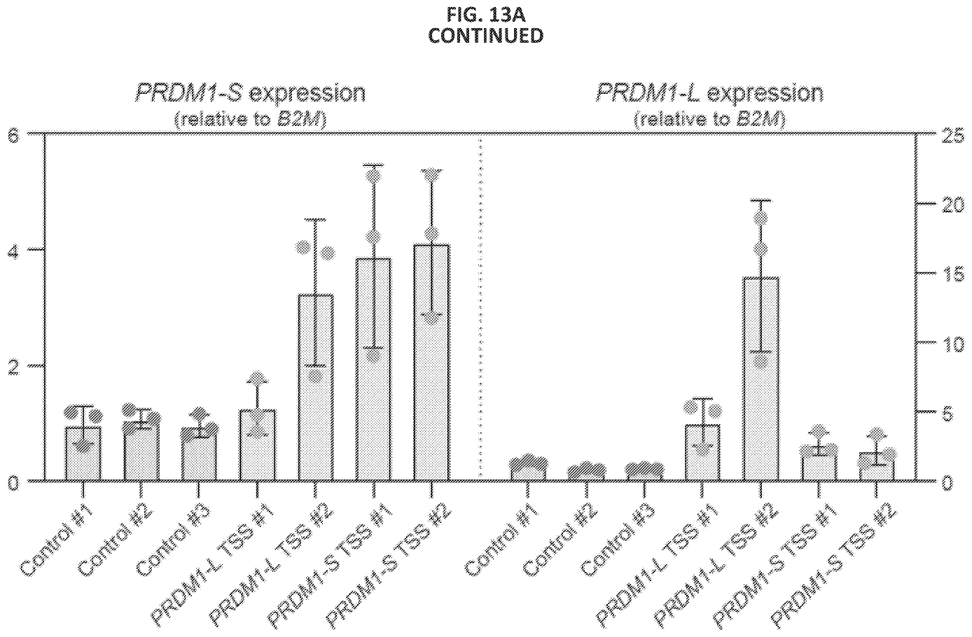

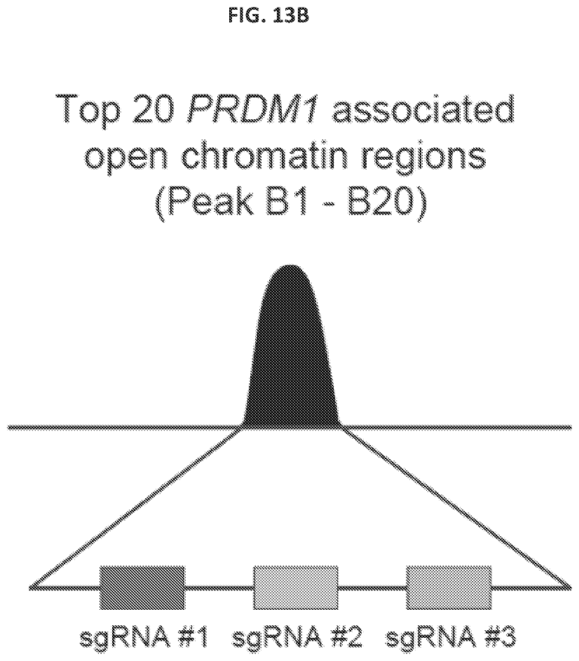

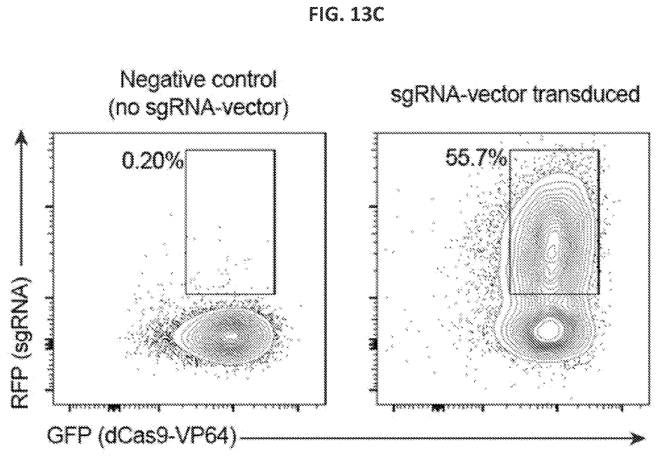

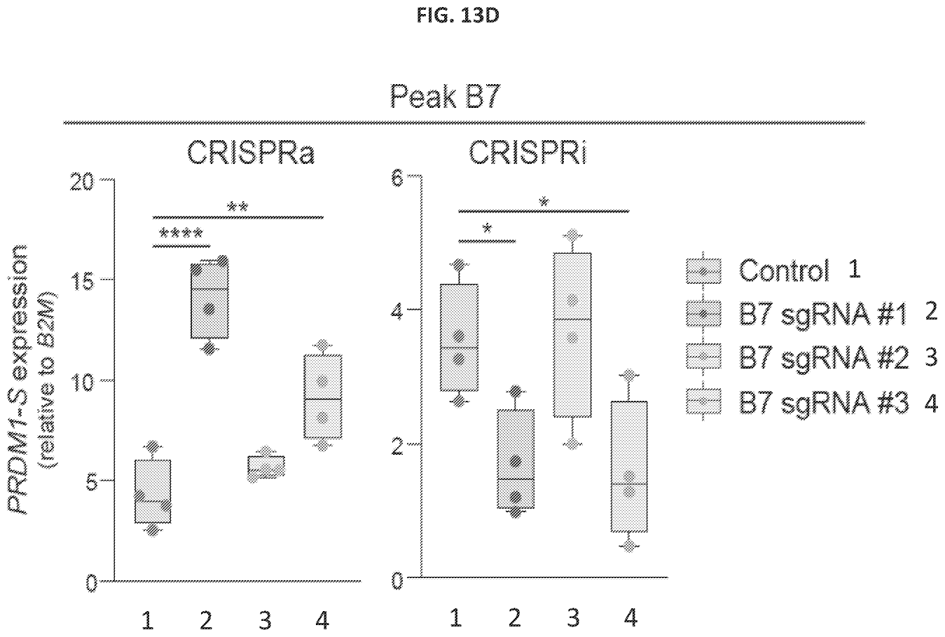

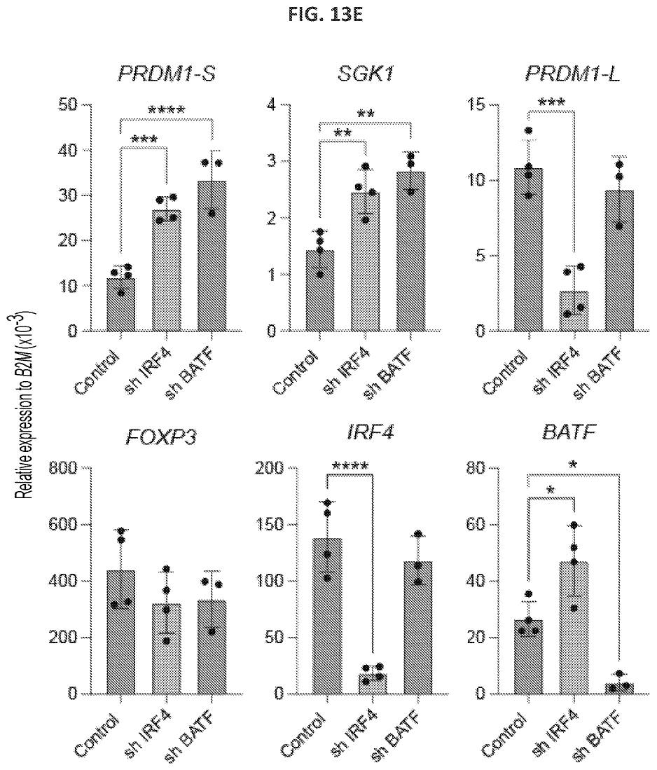

First, it was confirmed that PRDM1-S and PRDM1-L are independently regulated through different promoter activity by the CRISPRa method with sgRNAs targeting each promoter region, though there may be interactions between the transcription start site (TSS) of PRDM1-L and PRDM1-S(FIG. 13A). Next, three sgRNAs for each accessible region were designed and sgRNA expressing lentiviral particles were generated for twenty candidate regulatory elements (coded as #1 to #20) (Table 4, FIG. 13B). dCas9-VP64 expressing Jurkat T cells were infected by lentivirus encoding each sgRNA, then the double positive cells for GFP (dCas9-VP64) and RFP (sgRNA) were sorted by FACS (FIG. 13C). sgRNAs targeting the #2 peak region (−339,554 bp upstream of the TSS) mediated a unique induction of PRDM1-S but not PRDM1-L compared to control sgRNAs (FIG. 6B). sgRNAs targeting the #8 peak, which is located within the PRDM1-L TSS region, dominantly induced PRDM1-L but had a minor impact on PRDM1-S induction. The enhancer function of the #2 peak was further validated by both CRISPRa and CRISPRi (FIG. 13D). Moreover, the #2 peak region is reported as one of the “double elite” regulatory elements for PRDM1 in the GeneHancer dataset (84), which reflects a higher likelihood of prediction accuracy for both enhancer and target gene (FIG. 6B; top).

TABLE 4
PRDM1 Associated Accessible Elements

| Peak | | ave_peak | | | | Annotation | Annotation | Annotation |
|---|---|---|---|---|---|---|---|---|
| ID | p value | height | Chr | Start | end | 1 | 2 | 3 |
| B1 | 2.89E− | 3.08 | chr6 | 106610966 | 106612875 | 9_EnhA1 | 3_TssFlnkU | 9_EnhA1 |
| | 18 | | | | | | | |
| B2 | 2.02E− | −0.04 | chr6 | 106540956 | 106541106 | 9_EnhA1 | NA | NA |
| | 07 | | | | | | | |
| B3 | 2.10E− | 1.57 | chr6 | 106562437 | 106562784 | 3_TssFlnkU | NA | NA |
| | 07 | | | | | | | |
| B4 | 2.85E− | 1.46 | chr6 | 106542204 | 106542468 | 9_EnhA1 | NA | NA |
| | 07 | | | | | | | |
| B5 | 4.01E− | 1.71 | chr6 | 106597171 | 106598016 | 11_EnhWk | 18_Quies | NA |
| | 07 | | | | | | | |
| B6 | 7.70E− | 1.86 | chr6 | 106428847 | 106429894 | 2_TssFlnk | 4_TssFlnkD | NA |
| | 07 | | | | | | | |
| B7 | 1.12E− | −0.21 | chr6 | 106194443 | 106194840 | 18_Quies | NA | NA |
| | 06 | | | | | | | |
| B8 | 1.74E− | 2.95 | chr6 | 106533719 | 106534479 | 9_EnhA1 | 3_TssFlnkU | 1_TssA |
| | 06 | | | | | | | |
| B9 | 3.93E− | 3.55 | chr6 | 106439749 | 106440818 | 3_TssFlnkU | 1_TssA | 3_TssFlnkU |
| | 06 | | | | | | | |
| B10 | 5.54E− | −0.01 | chr6 | 106538993 | 106539162 | 1_TssA | 3_TssFlnkU | NA |
| | 06 | | | | | | | |
| B11 | 8.18E− | 0.19 | chr6 | 106536808 | 106537010 | 2_TssFlnk | 1_TssA | NA |
| | 06 | | | | | | | |
| B12 | 2.05E− | 0.83 | chr | 106537901 | 106538145 | 3_TssFlnkU | NA | NA |
| | 05 | | | | | | | |
| B13 | 9.01E− | 2.32 | chr6 | 106551516 | 106552068 | 8_EnhG2 | NA | NA |
| | 05 | | | | | | | |
| B14 | 1.83E− | −0.23 | chr6 | 106593125 | 106593276 | 11_EnhWk | NA | NA |
| | 04 | | | | | | | |
| B15 | 2.22E− | −0.42 | chr6 | 106205453 | 106205669 | 18_Quies | NA | NA |
| | 04 | | | | | | | |
| B16 | 2.51E− | 0.52 | chr6 | 106635455 | 106635815 | 9_EnhA1 | 11_EnhWk | NA |
| | 04 | | | | | | | |
| B17 | 2.76E− | 0.99 | chr6 | 106258201 | 106258803 | 11_EnhWk | NA | NA |
| | 04 | | | | | | | |
| B18 | 3.12E− | 0.14 | chr6 | 106235137 | 106235560 | 11_EnhWk | NA | NA |
| | 04 | | | | | | | |
| B19 | 4.47E− | −0.02 | chr6 | 106192403 | 106193016 | 11_EnhWk | NA | NA |
| | 04 | | | | | | | |
| B20 | 5.94E− | −0.08 | chr6 | 106630411 | 106630671 | 11_EnhWk | NA | NA |
| | 04 | | | | | | | |

To further clarify the function of this region as a cis-regulatory element, an effort to decode the histone modification was carried out. The main obstacle in the investigation of chromatin state by using conventional ChIP-seq technique is the limited number of ex vivo primary Tregs available for determining multiple histone marks from the same sample. Recent development of multiplexed, indexed T7 ChIP-seq (Mint-ChIP) (85) allows for identification of both active and repressive epigenetic marks by using histone modification-specific antibodies with limited cell numbers. Mint-ChIP was performed on human primary Treg in collaboration with the ENCODE project. Histone modifications (H3K27ac, H3K4me1, and H3K4me3)distinguishing active enhancers were assessed on ex vivo human primary Treg from MS and control subjects. The #2 peak region was marked by H3K4me1 and H3K27ac but without H3K4me3, suggesting the #2 peak region functions as an active enhancer (FIG. 6C; top). Importantly, this #2 peak region overlaps with AP-1 family and IRF4 ChIP-seq peaks and contains AP-1/IRF composite motif (86), suggesting this enhancer element induces PRDM1-S via AP-1 and IRF binding that is enriched in MS mTreg (FIG. 6C; bottom). Given that BATF and IRF4 are known to bind cooperatively on AP-1/IRF composite motif to facilitate Th17 differentiation (65, 87), the role of IRF4 and BATF on PRDM1-S expression in Tregs was examined by performing IRF4 and BATF knockdown experiments. Surprisingly, PRDM1-S was upregulated by knocking down IRF4 or BATF while in contrast, PRDM1-L was downregulated by IRF4 KD (FIG. 13E). These data indicate that IRF4 differentially regulates PRDM1-S and PRDM1-L in human primary Tregs. Of note, IRF4 or BATF KD did not affect FOXP3 expression, suggesting that the loss of IRF4 and BATF in human Tregs can induce PRDM1-S and SGK1 expression without significant reduction of FOXP3 expression, further indicating that the dysfunctional PRDM1-S/SGK1 axis observed in MS Tregs is counter regulated by the core effector Treg regulator IRF4 and BATF. These data also suggest that the cis-regulatory element (#2 peak region identified as upstream of PRDM1-S, FIG. 6) serves as a negative regulatory element for PRDM1-S expression via IRF4/BATF binding. Indeed, CRISPRi targeting on this #2 peak region suppress PRDM1-S expression (FIG. 6D). Together with the co-expression analysis for PRDM-1S which demonstrated both BATF and IRF4 as positively correlated genes among AP-1/IRF family TFs in MS mTreg (FIG. 12B), these data highlight IRF4/BATF as potential upstream TFs driving PRDM1-S in MS mTreg. Taken together, these data indicate that a newly identified cis-regulatory element that contains AP-1/IRF composite motif accounts for aberrant PRDM1-S induction in MS mTreg. Integration of comprehensive transcriptomic and epigenetic profiling on mTreg revealed an aberrant gene regulatory circuit. Rewiring of this circuit could be a therapeutic strategy for Treg targeted approach in autoimmune diseases including MS.

Below are the methods used in the Examples described above.

### Study Subjects

Peripheral blood was drawn from people with MS and healthy controls who were recruited as part of an Institutional Review Board (IRB)-approved study at Yale University. Human T cell isolation and culture

Peripheral blood mononuclear cells (PBMCs) were prepared from whole blood by Ficoll gradient centrifugation (Lymphoprep, STEMCELL Technologies) and used directly for total CD4+ T cell enrichment by negative magnetic selection using Easysep magnetic separation kits (STEMCELL Technologies). Cell suspension was stained with anti-CD4 (RPA-T4), anti-CD25 (clone 2A3), anti-CD45RO (UCHL1), anti-CD45RA (HI100) and anti-CD127 (hIL-7R-M21, all from BD Biosciences) for 30 minutes at 4° C. Naïve Tconv (CD4+/CD25neg/CD127+/CD45ROneg/CD45RA+), Naïve Treg (CD4+/CD25hi/CD127neg/CD45ROneg/CD45RA+), Memory Tconv (CD4+/CD127+/CD45RO+/CD45RAneg), and Memory Treg (CD4+/CD25hi/CD127neg/CD45RO+/CD45RAneg) were sorted on a FACSAria (BD Biosciences). Sorted cells were plated in 96-well round-bottom plates (Corning) and cultured in RPMI 1640 medium supplemented with 5% Human serum, 2 nM L-glutamine, 5 mM HEPES, and 100 U/ml penicillin, 100 g/ml streptomycin, 0.5 mM sodium pyruvate, 0.05 mM nonessential amino acids, and 5% human AB serum (Gemini Bio-Products). Cells were seeded (30,000-50,000/well) into wells pre-coated with anti-human CD3 (2 μg/ml, clone UCHT1, BD Biosciences) along with soluble anti-human CD28 (1 g/ml, clone 28.2, BD Biosciences) in the presence or absence of human IL-2 (50 U/ml).

### Lentiviral Transduction

### Lentiviral Production

Lentiviral plasmids encoding shRNA for gene knockdown for PRDM1-L or open reading frame (89) of overexpression for PRDM1-S and PRDM1-L were obtained from Sigma-Aldrich (MISSION shRNA) and Horizon Discovery Biosciences (Precision LentiORF), respectively. dCas9-VP64-2A-GFP (Addgene 61422) and pHR-SFFV-dCas9-BFP-KRAB (addgene 46911) were used for generating Jurkat T cell lines for CRISPRa and CRISPRi, respectively. EF1a-RFP-H1-gRNA vector (CASLV502PA-R from System bioscience) was modified to introduce BsaI cut site and single sgRNAs were cloned into it by using Golden Gate Assembly kit (BsmBI-v2, New England Biolabs #E1602). All single sgRNAs used in this study are listed in Table 5. Each plasmid was transformed into One Shot Stb13 chemically competent cells (Invitrogen) and purified by ZymoPURE plasmid Maxiprep kit (Zymo research). Lentiviral pseudoparticles were obtained after plasmid transfection of 293T cells using TurboFectin 8.0 Transfection Reagent (Origene). The medium was replaced after 6-12 h with fresh media with 1X Viral boost (Alstem). The lentivirus containing media was harvested 72 h after transfection and concentrated 80 times using Lenti Concentrator (Origene). LV particles were then resuspended in RPMI 1640 media without serum and stored at −80° C. before use. Virus titer was determined by using Jurkat T cells and Lenti-X GoStix Plus (Takara Clontech).

TABLE 5
sgRNA Oligonucleotide Sequences

| | SEQ | |
|---|---|---|
| | ID | |
| Name | NO: | OLIGO SEQUENCE |
| B1-1_Top | 41 | ACCGCAGTCATATGTGCTACCCCA |
| B1-1 Bottom | 42 | AAACTGGGGTAGCACATATGACTG |
| B1-2_Top | 43 | ACCGGGTCACATGAAATCCAGGGG |
| B1-2 Bottom | 44 | AAACCCCCTGGATTTCATGTGACC |
| B1-3_Top | 45 | ACCGGCAGACGAATCAGACTGGGT |
| B1-3 Bottom | 46 | AAACACCCAGTCTGATTCGTCTGC |
| B2-1_Top | 47 | ACCGTGAATTTGTAAGGTTAGAGA |
| B2-1 Bottom | 48 | AAACTCTCTAACCTTACAAATTCA |
| B2-2_Top | 49 | ACCGGAGATGGCAAGAGCTACTTC |
| B2-2 Bottom | 50 | AAACGAAGTAGCTCTTGCCATCTC |
| B2-3_Top | 51 | ACCGGTGCTGCTTTATAGCTTACT |
| B2-3 Bottom | 52 | AAACAGTAAGCTATAAAGCAGCAC |
| B3-1_Top | 53 | ACCGCACAAGCTCGTGGAAGACAG |
| B3-1 Bottom | 54 | AAACCTGTCTTCCACGAGCTTGTG |
| B3-2_Top | 55 | ACCGCCTTGAACACAAAAAACCTG |
| B3-2 Bottom | 56 | AAACCAGGTTTTTTGTGTTCAAGG |
| B3-3_Top | 57 | ACCGAAGCAGGTGATATCCTCCAG |
| B3-3 Bottom | 58 | AAACCTGGAGGATATCACCTGCTT |
| B4-1_Top | 59 | ACCGTTGTAAAGGACTACTCACAG |
| B4-1 Bottom | 60 | AAACCTGTGAGTAGTCCTTTACAA |
| B4-2_Top | 61 | ACCGTGTGTGATGCATCCAGTCTG |
| B4-2 Bottom | 62 | AAACCAGACTGGATGCATCACACA |
| B4-3_Top | 63 | ACCGCAGATCTTAGAACCTACAGC |
| B4-3 Bottom | 64 | AAACGCTGTAGGTTCTAAGATCTG |
| B5-1_Top | 65 | ACCGTCACATCAGACCACATCCAG |
| B5-1 Bottom | 66 | AAACCTGGATGTGGTCTGATGTGA |
| B5-2 Top | 67 | ACCGGGAGCAGAGAACTTGTGTTG |
| B5-2 Bottom | 68 | AAACCAACACAAGTTCTCTGCTCC |
| B5-3_Top | 69 | ACCGTGGTGGGATTCCTGCTCACA |
| B5-3 Bottom | 70 | AAACTGTGAGCAGGAATCCCACCA |
| B6-1_Top | 71 | ACCGTGAGTGGATGTATTCCCAGA |
| B6-1 Bottom | 72 | AAACTCTGGGAATACATCCACTCA |
| B6-2_Top | 73 | ACCGTATTCCCAGACGGATGCGGG |
| B6-2 Bottom | 74 | AAACCCCGCATCCGTCTGGGAATA |
| B6-3_Top | 75 | ACCGTACCCGAGAGTACCCTCTGG |
| B6-3 Bottom | 76 | AAACCCAGAGGGTACTCTCGGGTA |
| B7-1_Top | 77 | ACCGCATGAGACTTGTTCTTCTTT |
| B7-1 Bottom | 78 | AAACAAAGAAGAACAAGTCTCATG |
| B7-2_Top | 79 | ACCGACCCACTGTTAGGGGCTGGG |
| B7-2 Bottom | 80 | AAACCCCAGCCCCTAACAGTGGGT |
| B7-3_Top | 81 | ACCGAAGAGATCTATTGTAGAACA |
| B7-3 Bottom | 82 | AAACTGTTCTACAATAGATCTCTT |
| B8-1_Top | 83 | ACCGTAGAGCCCAAGTAAGCGTTG |
| B8-1 Bottom | 84 | AAACCAACGCTTACTTGGGCTCTA |
| B8-2_Top | 85 | ACCGCGAAGAGAGGAAGCTCTCGG |
| B8-2 Bottom | 86 | AAACCCGAGAGCTTCCTCTCTTCG |
| B8-3_Top | 87 | ACCGAAAACGTGTGGGTACGACCT |
| B8-3 Bottom | 88 | AAACAGGTCGTACCCACACGTTTT |
| B9-1_Top | 89 | ACCGGCGATAATGACTGATAAGGG |
| B9-1 Bottom | 90 | AAACCCCTTATCAGTCATTATCGC |
| B9-2_Top | 91 | ACCGCGATAATGACTGATAAGGGC |
| B9-2 Bottom | 92 | AAACGCCCTTATCAGTCATTATCG |
| B9-3_Top | 93 | ACCGTCAGTGTTGCCCTTGCAATG |
| B9-3 Bottom | 94 | AAACCATTGCAAGGGCAACACTGA |
| B10-1_Top | 95 | ACCGCCCTTATCAGTCATTATCGC |
| B10-1 Bottom | 96 | AAACGCGATAATGACTGATAAGGG |
| B10-2_Top | 97 | ACCGTCGGTTGAGACTGCTTGCAC |
| B10-2 Bottom | 98 | AAACGTGCAAGCAGTCTCAACCGA |
| B10-3_Top | 99 | ACCGCCCTGTATCGTGGCTGATAA |
| B10-3 Bottom | 100 | AAACTTATCAGCCACGATACAGGG |
| B11-1_Top | 101 | ACCGGATGGTATAAGCCGAGAAAG |
| B11-1 Bottom | 102 | AAACCTTTCTCGGCTTATACCATC |
| B11-2_Top | 103 | ACCGCTGTTTATCTGCAGCAGCTC |
| B11-2 Bottom | 104 | AAACGAGCTGCTGCAGATAAACAG |
| B11-3_Top | 105 | ACCGCTGAAGTGGGTACACTCTTT |
| B11-3 Bottom | 106 | AAACAAAGAGTGTACCCACTTCAG |
| B12-1_Top | 107 | ACCGATATAGGTAAAGCCAGACCG |
| B12-1 Bottom | 108 | AAACCGGTCTGGCTTTACCTATAT |
| B12-2_Top | 109 | ACCGGCTTCTCGATTATGCTACAC |
| B12-2 Bottom | 110 | AAACGTGTAGCATAATCGAGAAGC |
| B12-3_Top | 111 | ACCGCTCTTGGTGTCACTGTGGTT |
| B12-3 Bottom | 112 | AAACAACCACAGTGACACCAAGAG |
| B13-1_Top | 113 | ACCGGCATCATTTATTGTGTAGCG |
| B13-1 Bottom | 114 | AAACCGCTACACAATAAATGATGC |
| B13-2_Top | 115 | ACCGCATTGCTGCTGTTCCTAAAT |
| B13-2 Bottom | 116 | AAACATTTAGGAACAGCAGCAATG |
| B13-3_Top | 117 | ACCGTGGTGAAGTGCTTAGGCTGT |
| B13-3_Bottom | 118 | AAACACAGCCTAAGCACTTCACCA |
| B14-1_Top | 119 | ACCGGTATCTTTGCACATCCTGCC |
| B14-1 Bottom | 120 | AAACGGCAGGATGTGCAAAGATAC |
| B14-2_Top | 121 | ACCGGCAGTCACCTCATATGTCAG |
| B14-2 Bottom | 122 | AAACCTGACATATGAGGTGACTGC |
| B14-3_Top | 123 | ACCGCATATGTCAGAGGGCACTGT |
| B14-3 Bottom | 124 | AAACACAGTGCCCTCTGACATATG |
| B15-1_Top | 123 | ACCGCATATGTCAGAGGGCACTGT |
| B15-1 Bottom | 124 | AAACACAGTGCCCTCTGACATATG |
| B15-2_Top | 125 | ACCGGGAAATGCCATGAACTCTAG |
| B15-2 Bottom | 126 | AAACCTAGAGTTCATGGCATTTCC |
| B15-3_Top | 127 | ACCGAGTCATCACTGCGTGAATGC |
| B15-3 Bottom | 128 | AAACGCATTCACGCAGTGATGACT |
| B16-1_Top | 129 | ACCGCTCAGCTTCCTGGTGACAGA |
| B16-1 Bottom | 130 | AAACTCTGTCACCAGGAAGCTGAG |
| B16-2_Top | 131 | ACCGACGTTTTCCTTTCTGCTCTG |
| B16-2 Bottom | 132 | AAACCAGAGCAGAAAGGAAAACGT |
| B16-3_Top | 133 | ACCGTCCCAGGTGACAGTCAACAG |
| B16-3_Bottom | 134 | AAACCTGTTGACTGTCACCTGGGA |
| B17-1_Top | 135 | ACCGCAAACACCAGATGTTCAGAG |
| B17-1 Bottom | 136 | AAACCTCTGAACATCTGGTGTTTG |
| B17-2_Top | 137 | ACCGATTACATTCTGAGGACTCAG |
| B17-2 Bottom | 138 | AAACCTGAGTCCTCAGAATGTAAT |
| B17-3_Top | 139 | ACCGAGAGTCAAGAAACCTGACAG |
| B17-3 Bottom | 140 | AAACCTGTCAGGTTTCTTGACTCT |
| B18-1_Top | 141 | ACCGCCATTACTAGGTTCTCCCTG |
| B18-1 Bottom | 142 | AAACCAGGGAGAACCTAGTAATGG |
| B18-2_Top | 143 | ACCGCTGATTTCAGTAGTGAAACT |
| B18-2_Bottom | 144 | AAACAGTTTCACTACTGAAATCAG |
| B18-3_Top | 145 | ACCGCTTGTCCTTCACGAAGCCGG |
| B18-3 Bottom | 146 | AAACCCGGCTTCGTGAAGGACAAG |
| B19-1_Top | 147 | ACCGTGAATCGGCTGGTAGTTGGT |
| B19-1 Bottom | 148 | AAACACCAACTACCAGCCGATTCA |
| B19-2_Top | 149 | ACCGAATAGCATTCAGGATGTACG |
| B19-2 Bottom | 150 | AAACCGTACATCCTGAATGCTATT |
| B19-3_Top | 151 | ACCGAAAGAGCTAAGAAACCTCTG |
| B19-3 Bottom | 152 | AAACCAGAGGTTTCTTAGCTCTTT |
| B20-1_Top | 153 | ACCGGAAACACAAGATATGTGCTG |
| B20-1 Bottom | 154 | AAACCAGCACATATCTTGTGTTTC |
| B20-2_Top | 155 | ACCGCGCAAGGATCTGGTCCACAG |
| B20-2 Bottom | 156 | AAACCTGTGGACCAGATCCTTGCG |
| B20-3_Top | 157 | ACCGCAGTGGTTCCTACCGAAAGT |
| B20-3 Bottom | 158 | AAACACTTTCGGTAGGAACCACTG |
| short pro-1_Top | 159 | ACCGAATCACCTGACTTACAATGG |
| short pro-1_ | 160 | AAACCCATTGTAAGTCAGGTGATT |
| Bottom | | |
| short pro-2_Top | 161 | ACCGGTAGCAAAAGTAGTACTCTG |
| short pro-2 | 162 | AAACCAGAGTACTACTTTTGCTAC |
| Bottom | | |
| long pro-1_Top | 163 | ACCGCACTCGGCTGCGCGAAGTGG |
| long pro-1_ | 164 | AAACCCACTTCGCGCAGCCGAGTG |
| Bottom | | |
| long pro-2_Top | 85 | ACCGCGAAGAGAGGAAGCTCTCGG |
| long pro-2_ | 86 | AAACCCGAGAGCTTCCTCTCTTCG |
| Bottom | | |

### Lentiviral Transduction

FACS-sorted Tregs were plated at 50,000 cells/well in round bottom 96 well plates pre-coated with anti-human CD3 (2 mg/ml, clone UCHT1, BD Biosciences) and soluble anti-human CD28 (1 mg/ml, clone 28.2, BD Biosciences), in the presence of human IL-2 (50 U/ml). After 24 h, cells were transferred into Retronectin coated 96 well plates and 25-50 ml of lenti particles were added to each well, then spinfected with high-speed centrifugation (1000 g) for 1.5 hour at 32° C. Immediately after centrifugation, cells are placed back to the culture. On day 5, cells are harvested and GFP positive cells are sorted by FACSAria or analyzed by Fortessa.

Jurkat T cells were plated at 50,000 cells/well in round bottom 96 well plates and 25 μl of lenti particles were added to each well, and spinfected as well as above. On day 3-5, cells were scaled up to 12 well plates and followed by the second scale-up at day 7-9 into 6 well plates. Cells were stimulated with PMA and Ionomycin (50 ng/ml and 250 ng/ml respectively) for four hours and GFP⁺/RFP⁺ double positive cells were sorted directly into RNA lysis buffer by FACS Aria.

### Suppression Assay

CD4+CD25+CD127neg Treg cells and CD4+CD127+ T effector cells were sorted on a FACS Aria (BD Biosciences). Treg cells were transduced with lentiviral particles containing PRDM1-S ORF or GFP control. GFP positive cells were sorted by FACS at day 5, and T effector cells were labeled with cell trace violet dye and then co-cultured with Treg cells (1×10⁴) at different ratio with human Treg inspector beads at 2:1 bead-to-cell ratio. The proliferation of Teff cells was determined at day 4 on a BD Fortessa instrument (BD Bioscience).

### Flow Cytometry Analysis

Cells were stained with LIVE/DEAD Fixable Near-IR Dead Cell Stain kit (Invitrogen) and surface antibodies for 30 min at 4° C. For intracellular cytokine staining, cells were fixed with BD Cytofix™ Fixation Buffer (BD Biosciences) for 10 min at RT, then washed with PBS. Intracellular staining was performed in Foxp3 permeabilization buffer (Thermo Fisher) for 30 min at 4° C. The following antibodies were used: anti-Foxp3 (clone 150D, Biolegend, and clone PCH101, Thermo Fisher), anti-Blimp1 (clone 3H2-EB, Thermo Fisher). All antibody information is listed in Table 6. Cells were acquired on a BD Fortessa flow cytometer and data were analyzed with FlowJo software v10 (Threestar).

TABLE 6
Antibodies for Flow Cytometry Analysis

| Antigen | Clone | Fluorophore | Supplier | Dilution | Product# |
|---|---|---|---|---|---|
| CD4 | (RPA-T4) | FITC | (BD Pharmingen) | 1:100 | 561005 |
| CD25 | (M-A251) | V450 | (BD Pharmingen) | 1:100 | 560356 |
| CD45RO | (UCHL1) | PE-Cy7 | (BD Pharmingen) | 1:100 | 560608 |
| CD45RA | (HI100) | APC-H7 | (BD Pharmingen) | 1:100 | 560674 |
| CD127 | (hIL-7R-M21) | Alexa647 | (BD Pharmingen) | 1.25 | 558598 |
| Blimp1 | (3H2-E8) | DyLight 650 | (Thermo Fisher) | 1:100 | MA5-16114 |
| Foxp3 | (150D) | Alexa647 | (BioLegend) | 1:100 | 320012 |
| Foxp3 | (PCH101) | PE Cy7 | (Thermo Fisher) | 1:100 | 25-4776-42 |

### Immunoblotting

Cells were lysed with RIPA buffer containing protease inhibitor and phosphatase inhibitor. Extracted protein was quantified with a BCA kit (Thermo Scientific). 20 μg of protein extract was loaded in each lane, followed by separation by 10% SDS-PAGE and transfer to a nitrocellulose membrane. Primary antibodies were detected by the secondary antibody horseradish peroxidase-conjugated anti-rabbit (Cell Signaling Technology), and images were obtained with a ChemiDoc Imaging system (Bio-Rad).

### Bulk RNA-Seq and ATAC-Seq Library Preparation and Sequencing

Bulk RNA-seq: FACS sorted cells (5,000 cells) were subjected to cDNA synthesis using SMART-Seq v4 Ultra Low Input RNA Kit for sequencing (Takara/Clontech). Barcoded libraries were generated by the Nextera XT DNA Library Preparation kit (Illumina) and sequenced with a 2×100 bp paired-end protocol on the HiSeq 4000 Sequencing System (Illumina).

Bulk ATAC-sea: FAST-ATAC was adopted for FACS sorted CD4⁺ T cells (5,000 cells) (Corces et al., 2016). Cells were pelleted by centrifugation at 500 g for 7 min at 4° C., then resuspended with 50 μl of transposase mixture (25 μL of 2× TD buffer (Illumina), 2.5 μL of TDE1 (Illumina), 0.5 μL of 1% digitonin (Thermo Fisher), 22 μL of nuclease-free water). Transposition reactions were incubated at 37° C. for 30 minutes in a thermal shaker with agitation at 300 RPM. Transposed DNA was purified using a MinElute Reaction Cleanup kit (QIAgen) and purified DNA was eluted in 20 μL elution buffer. Transposed fragments were amplified and purified as described previously (90) with modified primers (91). Libraries were quantified using qPCR (KAPA Library Quantification Kit) prior to sequencing. All Fast-ATAC libraries were sequenced using a 2×100 bp paired-end protocol on the HiSeq 4000 Sequencing System (Illumina).

### Mint-ChIP Library Preparation and Sequencing

800,000-1,000,000 cryopreserved sorted human primary Tregs were used for Mint-ChIP profiling. Cells were thawed and immediately pelleted by centrifugation at 400 g for 7 min at 4° C., then resuspended with 100 μL of ice-cold PBS. Approximately 100,000 cells per antibody are lysed in detergent and chromatin is digested with micrococcal nuclease. Double stranded adapters (that contain both a promoter for transcription by T7 RNA polymerase and a demultiplexing adapter) are ligated to the chromatin. Chromatin is mixed overnight with antibodies recognizing histone modifications, and immune complexes are captured using protein A/protein G magnetic bead mixtures. In addition, some chromatin is set aside overnight to enable preparation of an antibody free, input control library. Immobilized immune complexes are washed, and immunoprecipitated DNA is eluted using proteinase K. Recovered DNA is purified with SPRI beads and subject to T7 RNA synthesis, thus creating an RNA copy of the immunoprecipitated DNA. RNA is copied back to cDNA using a random primer containing a 5′ extension, enabling subsequent PCR amplification with Illumina indexed sequencing primers. PCR products are purified and mixed together to enable multiplex Illumina sequencing. DNA is sequenced using a paired end protocol; in Mint-ChIP3, the first 8 bases of Illumina Read2 serve as an inline barcode enabling demultiplexing of the chromatin using the ligated barcoded adapter. The detailed protocol and primer/adaptor sequences are described in dx.doi.org/10.17504/protocols.io.wbefaje.

### Mint-ChIP Data Processing

The Mint-ChTP FASTQ files of each sample were processed using the ENCODE3 ChIP-seq pipeline provided by Anshul Kundaje (https://github.com/ENCODE-DCC/chip-seq-pipeline2) with the following parameters “chip.pipeline_type=histone, chip.aligner=bowtie2, chip.true_rep_only=true, chip.paired_end=true, chip.ctl_paired_end=true, chip.always_use_pooled_ctl=false” specified in its json file. The default value was used for all the other parameters. Briefly, the pipeline first mapped the reads to the hg19 human reference genome using bowtie2. The aligned reads were filtered and duplicated reads were removed. The peak calling was then performed using MACS2 with a control sample for each individual. Sample quality was assessed with a cross-correlation plot.

### Bulk RNA-Seq Analysis

After sequencing, adapter sequences and poor-quality bases (quality score<3) were trimmed with Trimmomatic. Remaining bases were trimmed if their average quality score in a 4 bp sliding window fell below 5. FastQC (https://www.bioinformatics.babraham.ac.uk/projects/fastqc/) was used to obtain quality control metrics before and after trimming. Remaining reads were aligned to the GRCh38 human genome assembly with STAR 2.5.2 (92). Picard (https://github.com/broadinstitute/picard) was used to remove optical duplicates and to compile alignment summary statistics and RNA-seq summary statistics. After alignment, reads were quantitated to gene level with RSEM (93) using the GENCODE annotation (94).

An initial quality control assessment was conducted on the entire dataset, including memory and naïve Tconv and Treg cells obtained from MS patients and healthy controls. A subset of these were stimulated with IL-2 as described above, with the remainder collected in the ex vivo state. Principal component analysis was used to identify potential sample swaps. Genes that were quantitated>1 count per million (cpm) in >15 samples were considered, normalizing expression values by the trimmed mean of M-values as implemented in edgeR (95). Limma (96) was used with the voom transformation (97) to identify differentially expressed genes within mTconv and mTreg cell populations separately. RUV-seq (98) was used to account for batch effect and other sources of systematic variation; RUV parameters along with a sex covariate were included in the final model.

Co-Expression Analysis in Bulk RNA-Seq and scRNA-Seq

The co-expression between two genes was computed as the Spearman correlation coefficient of normalized gene expression. The normalized gene expression was calculated by dividing the raw count by the library size of the sample and its scaling factor obtained from the TMM normalization.

The co-expression analysis of PRDM1 in the scRNA-seq data were conducted for genes that had the average count per cell>0.005 in the mTreg and mTconv. The co-expression was measured by the log(fold-change) between PRDM1 and the gene of interest in a negative binomial mixed model with the subjects as random effects implemented in NEBULA (56). Normalized expression of PRDM1 (the raw count divided by the library size of the cell) was used as the explanatory variable and included in the model the proportion of reads from ribosomal protein genes and mitochondrial genes as covariates. To assess the differential co-expression, the model was fitted for 4896 and 4676 cells from the five MS patients and five healthy controls separately.

### Bulk ATAC-Seq Analysis

The official pipeline of the Encyclopedia of DNA Elements (54) consortium (github.com/kundajelab/atac_dnase_pipelines) {kundajelab/atac_dnase_pipelines: 0.3.0} was adopted to preprocess the ATAC-seq raw data. The preprocessing started with the paired-end ATAC-seq fastq files of each subject. More specifically, reads were trimmed for adapters using cutadapt (99) and mapped to the human reference genome (hg19) using bowtie2 (100). The output raw bam files were filtered, deduped, and converted to single-ended Tn5-shifted tagalign files, which were then used as the input for peak calling. The deduped bam files were used in the downstream motif and footprint analyses described in a later section.

Sample-specific narrow peaks (FDR<0.01) were called using MACS2 (101) from the tagalign file of each of the samples, separately, with the command “macs2 callpeak -f BED -g hs -q 0.01--nomodel --shift -75--extsize 150-B --SPMR --keep-dup all --call-summits”. The fraction of reads in called peak regions (FRiP score) was then calculated for each sample. Group-specific narrow peaks (FDR<0.01) were then called by providing MACS2 with the tagalign files of all high-quality samples (FRiP>0.1) within each of the eight groups (healthy/MS mTconv, mTreg, naïve Treg (nTreg), and naïve Tconv (nTconv)) using the command “macs2 callpeak -t<tagalign files of the high-quality samples within the group>-f BED -g hs -q 0.01--nomodel --shift -75--extsize 150-B --SPMR --keep-dup all --call-summits”. To obtain a unified set of peak regions across all groups, the group-specific peak regions that were overlapping or had maximum distance<100 bp using BEDTools (102) were merged with the command “bedtools merge -1-d 100”.

Given the unified set of peak regions, the number of reads overlapping a peak was called for each subject using BEDTools. This raw count matrix was used for the downstream analyses. In the analysis of differential accessibility, peaks with low counts were filtered out and the counts of each subject were normalized using TMM (95). Then the voom function in the limma package (96) was used to perform the differential analysis between the cases and controls within each cell type with FRiP, sex and ethnicity as covariates.

### Correlation Analysis Between RNA-Seq and ATAC-Seq Data

The analysis of the correlation between the accessibility of the adjacent open chromatin regions and the PRDM1 expression was performed using a linear regression model in which the normalized PRDM1 expression (raw count divided by the total library size and the scaling factor) was the dependent variable and the adjusted ATAC-seq peak height was the explanatory variable. The adjusted ATAC-seq peak height was the residual obtained by fitting a weighted linear model using limma-voom for the peak with FRiP as the covariate. 106 samples were included from all the cell populations and both groups and these samples had both RNA-seq and ATAC-seq measurements. 72 ATAC-seq peaks were interrogated for which PRDM1 was annotated as the nearest gene by Homer. These peaks span from ˜350k bp upstream to -100k bp downstream of the transcription start site of the long isoform of PRDM1.

### ATAC-Seq Footprint Analysis

To conduct differential footprint analysis between the disease groups and cell types, group-level bam files were generated by merging the deduped bam files within each of the eight groups. A total of 113 high-quality ATAC-seq samples with FRiP>0.1 were included (MS group: 26 mTregs, 28 mTconvs, 5 nTregs, 1 nTconv; control group: 21 mTregs, 21 mTconvs, 8 nTregs, 3 nTconvs). The differential footprint analysis was performed using HINT v0.12.3 (103) and TOBIAS v0.10.1 (64). In the analysis using HINT, footprints were first called using the command “rgt-hint footprinting --atac-seq --paired-end” with the group-specific bam files and peaks as the input files. Predicted binding sites were then identified using the command “rgt-motifanalysis matching” for the 579 JASPAR (2018) core motifs for vertebrates. Finally, footprints showing differential binding activity between cell types or disease groups were identified using the command “rgt-hint differential” based on the results from the previous two steps. The same bam files and input files were used in the analysis adopting TOBIAS, in which “TOBIAS ATACorrect” was first run to obtain bias-corrected signals and then ran “TOBIAS FootprintScores” to obtain footprint scores. Finally, the differential footprint analysis was performed with the command “TOBIAS BINDetect”.

### Single-Cell RNA-Seq Using 10× Genomics Platform

CD4+ T cells and CD25^{hi} CD4⁺ T cells were negatively isolated from PBMCs separately by using Easysep human CD4⁺ T cell isolation kits and EasySep Human CD4⁺CD127^{low}CD25⁺ Regulatory T Cell Isolation Kit (STEMCELL Technologies), respectively. To avoid batch effects between healthy and MS samples, and to increase the numbers of Tregs to analyze, hashing technology (Biolegend) was used to pool samples in a single run of the 10× Genomics platform. Each MS sample was processed with a paired healthy control subject matched for age, sex, and ethnicity. A total of five healthy and MS sample pairs were analyzed. 100,000 cells for each cell type were subjected to Total-seq C and Hashtag antibody staining. 2 μg per 1 million cells for Total-seq C and 1 μg per 1 million cells for Hashing antibodies were used for the staining. Cells were washed three times with PBS containing 2% FBS and four hashed samples (total CD4⁺ T cells and CD25^{hi}CD4⁺ T cells from each of HC and MS) were pooled into one sample. The cellular concentration was adjusted to 1,000/μL and loaded into the 10× Genomics instrument aiming to recover 10,000 cells for library preparation and sequencing. Generated libraries were then sequenced on the NovaSeq (Illumina) with a target of 50,000 reads/cell (2×150 paired-end reads).

### Dual Omics Single-Cell Analysis

### Cell Type Assignment by Protein Surface Markers

A latent indicator variable zᵢₖ is defined to mark the assignment of a cell j to a cell type k and estimate the posterior probability of zⱼₖ=1 by the stochastic expectation maximization (EM) algorithm. A normalized vector is assumed for each cell xⱼ follows von Mises-Fisher (vMF) distribution (104) with cell type k-specific mean vector μₖ and shared concentration parameter κ: P(xⱼ;zⱼₖ=1) ˜exp (κ θₖ^{T}xⱼ). The existing EM algorithm was modified (105) and the sparsity of the mean vector p was enforced based on the prior knowledge of the cell-type-specific activity of marker proteins/genes/features. Simply, μ_{gk} is allowed to take non-zero values if and only if a feature g is a known marker for the cell type k.

To improve the quality of inference, “negative” marker protein labels were also taken advantage of and an “adversarial” model was built for each cell type and contrast with the likelihood of the corresponding “positive” model. nTconv: CD3+, CD4+, CD8−, CD25−/CD127+, CD45RA+/CD45RO−; mTconv: CD3+, CD4+, CD8−, CD25−/CD127+, CD45RA−/CD45RO+; nTreg: CD3+, CD4+, CD8−, CD25+/CD127−, CD45RA+/CD45RO−; mTreg: CD3+, CD4+, CD8−, CD25+/CD127−, CD45RA−/CD45RO+.

To further dissect the cell types within mTreg cells, single-cell CITE-seq vectors were sorted based on the following definitions: Treg1: CD183+/CD194−/ CD196−; Treg1/17: CD183+/CD194−/ CD196+; Treg17: CD183−/ CD194+/CD196+; Treg2: CD183−/ CD194+/CD196−.

Batch Correction and Visualization of scRNA-Seq Data

The top 100 principal components of the log-transformed scRNA-seq data matrix were used to characterize intercellular similarity and clustering patterns across ˜45k cells and ˜15k genes. Discrepancies across five different batches were adjusted using the batch-balancing k-nearest neighborhood method (106) followed by adjustment of principal component, subtracting out the mean difference between batches (107).

### Single-Cell Differential Expression Analysis

Cell-type-specific gene expression profiles between the MS and HC subjects were compared by estimating unbiased subject-level pseudo-bulk profiles for each gene using CoCoA-diff (2). CocoA-diff can improve the statistical power in case-control scRNA-seq study while adjusting for unwanted confounding effects existing across individuals. Latent factors were estimated, which may confound gene and cell-type-specific expressions with the disease labels. A controlled baseline was established for each cell derived from the MS subjects by imputing counterfactual gene expression values based on the 100 cells found in the HC cells in the top 10 PC space (BBKNN-based weighted average). Likewise, counterfactual values for the HC cells were imputed using the MS cells. More precisely, for each gene g and cell j, it was observed Y_{gi}⁽¹⁹⁾ and counterfactual (imputed) Y_{gi}^{(HC)} if a cell j were derived from the MS; it was observed Y_{gi}^{(HC)} and counterfactual Y_{gi}^{(MS} if a cell j were from the HC. Both factual (observed) and counterfactual cell profiles within each subject and cell type were aggregated to estimate causal effects by comparing the average disease effect (ADE), average disease effect in the disease subject (108), and average disease effect in the control subject (ADC). Denoting the subject-level aggregate profiles λ_{gi}⁽¹⁹⁾ and λ_{gi}^{(HC)} (for gene g and subject i), ADE of a gene g is defined as: ADE(g)=Σ_{i=1..10} log [λ_{gi}⁽¹⁹⁾/λ_{gi}^{(HC)}]/10, ADC(g(=Σ_{i in 5 HC subjects} log [λ_{gi}⁽¹⁹⁾/λ_{gi}^{(HC)}]/5, ADD(g)=Σ_{i in 5 MS subjects} log [λ_{gi}⁽¹⁹⁾/λ_{gi}^{(HC)}]/5 Bayesian inference methods were implemented in a C++ program that calculates gene-level statistics, including posterior mean and standard error, efficiently handling ten thousand genes and hundred thousand cells (2).

### REFERENCES

1. M. Ota et al., Dynamic landscape of immune cell-specific gene regulation in immune-mediated diseases. Cell 184, 3006-3021 e3017 (2021).
2. Y. P. Park, M. Kellis, CoCoA-diff: counterfactual inference for single-cell gene expression analysis. Genome Biol 22, 228 (2021).
3. P. Gao et al., Risk variants disrupting enhancers of TH1 and TREG cells in type 1 diabetes. Proc Natl Acad Sci USA 116, 7581-7590 (2019).
4. M. T. Maurano et al., Systematic localization of common disease-associated variation in regulatory DNA. Science 337, 1190-1195 (2012).
5. H. Yoshida et al., The cis-Regulatory Atlas of the Mouse Immune System. Cell 176, 897-912 e820 (2019).
6. K. K. Farh et al., Genetic and epigenetic fine mapping of causal autoimmune disease variants. Nature 518, 337-343 (2015).
7. S. Hellberg et al., Dynamic Response Genes in CD4+ T Cells Reveal a Network of Interactive Proteins that Classifies Disease Activity in Multiple Sclerosis. Cell Rep 16, 2928-2939 (2016).
8. I. S. Brorson et al., No differential gene expression for CD4(+) T cells of MS patients and healthy controls. Mult Scler JExp Transl Clin 5, 2055217319856903 (2019).
9. G. L. Cvetanovich, D. A. Hafler, Human regulatory T cells in autoimmune diseases. Curr Opin Immunol 22, 753-760 (2010).
10. M. Miyara et al., Human FoxP3+ regulatory T cells in systemic autoimmune diseases. Autoimmun Rev 10, 744-755 (2011).
11. M. Dominguez-Villar, D. A. Hafler, Regulatory T cells in autoimmune disease. Nat Immunol 19, 665-673 (2018).
12. V. Viglietta, C. Baecher-Allan, H. L. Weiner, D. A. Hafler, Loss of functional suppression by CD4+CD25+ regulatory T cells in patients with multiple sclerosis. J Exp Med 199, 971-979 (2004).
13. M. Dominguez-Villar, C. M. Baecher-Allan, D. A. Hafler, Identification of T helper type 1-like, Foxp3+ regulatory T cells in human autoimmune disease. Nat Med 17, 673-675 (2011).
14. S. A. McClymont et al., Plasticity of human regulatory T cells in healthy subjects and patients with type 1 diabetes. J Immunol 186, 3918-3926 (2011).
15. M. F. Farez, M. P. Fiol, M. I. Gaitan, F. J. Quintana, J. Correale, Sodium intake is associated with increased disease activity in multiple sclerosis. J Neurol Neurosurg Psychiatry 86, 26-31 (2015).
16. M. Kleinewietfeld et al., Sodium chloride drives autoimmune disease by the induction of pathogenic TH17 cells. Nature 496, 518-522 (2013).
17. A. L. Hernandez et al., Sodium chloride inhibits the suppressive function of FOXP3+ regulatory T cells. J Clin Invest 125, 4212-4222 (2015).
18. T. Sumida et al., Activated beta-catenin in Foxp3(+) regulatory T cells links inflammatory environments to autoimmunity. Nat Immunol 19, 1391-1402 (2018).
19. T. Lappalainen et al., Transcriptome and genome sequencing uncovers functional variation in humans. Nature 501, 506-511 (2013).
20. H. Wu et al., High salt promotes autoimmunity by TET2-induced DNA demethylation and driving the differentiation of Tfh cells. Sci Rep 6, 28065 (2016).
21. C. Cotsapas et al., Pervasive sharing of genetic effects in autoimmune disease. PLoS Genet 7, e1002254 (2011).
22. M. R. Lincoln et al., Joint analysis reveals shared autoimmune disease associations and identifies common mechanisms. medRxiv, 2021.2005.2013.21257044 (2021).
23. H. S. Panitch, R. L. Hirsch, A. S. Haley, K. P. Johnson, Exacerbations of multiple sclerosis in patients treated with gamma interferon. Lancet 1, 893-895 (1987).
24. Y. Lee et al., Induction and molecular signature of pathogenic TH17 cells. Nat Immunol 13, 991-999 (2012).
25. C. Wu et al., Induction of pathogenic TH17 cells by inducible salt-sensing kinase SGK1. Nature 496, 513-517 (2013).
26. Y. Cao et al., Functional inflammatory profiles distinguish myelin-reactive T cells from patients with multiple sclerosis. Sci Transl Med 7, 287ra274 (2015).
27. A. Schwarz et al., Fine-tuning of regulatory T cell function: the role of calcium signals and naïve regulatory T cells for regulatory T cell deficiency in multiple sclerosis. J Immunol 190, 4965-4970 (2013).
28. F. Ciccocioppo et al., The Characterization of Regulatory T-Cell Profiles in Alzheimer's Disease and Multiple Sclerosis. Sci Rep 9, 8788 (2019).
29. K. S. Rauch et al., Id3 Maintains Foxp3 Expression in Regulatory T Cells by Controlling a Transcriptional Network of E47, Spi-B, and SOCS3. Cell Rep 17, 2827-2836 (2016).
30. D. Grun, L. Kester, A. van Oudenaarden, Validation of noise models for single-cell transcriptomics. Nat Methods 11, 637-640 (2014).
31. A. A. Kolodziejczyk, J. K. Kim, V. Svensson, J. C. Marioni, S. A. Teichmann, The technology and biology of single-cell RNA sequencing. Mol Cell 58, 610-620 (2015).
32. M. Stoeckius et al., Simultaneous epitope and transcriptome measurement in single cells. Nat Methods 14, 865-868 (2017).
33. M. Stoeckius et al., Cell Hashing with barcoded antibodies enables multiplexing and doublet detection for single cell genomics. Genome Biol 19, 224 (2018).
34. C. International Multiple Sclerosis Genetics et al., Analysis of immune-related loci identifies 48 new susceptibility variants for multiple sclerosis. Nat Genet 45, 1353-1360 (2013).
35. C. Soneson, M. D. Robinson, Bias, robustness and scalability in single-cell differential expression analysis. Nat Methods 15, 255-261 (2018).
36. N. M. Chapman et al., mTOR coordinates transcriptional programs and mitochondrial metabolism of activated Treg subsets to protect tissue homeostasis. Nat Commun 9, 2095 (2018).
37. F. Zhang, G. Liu, D. Li, C. Wei, J. Hao, DDIT4 and Associated lncDDIT4 Modulate Th17 Differentiation through the DDIT4/TSC/mTOR Pathway. J Immunol 200, 1618-1626 (2018).
38. C. S. Loo et al., A Genome-wide CRISPR Screen Reveals a Role for the Non-canonical Nucleosome-Remodeling BAF Complex in Foxp3 Expression and Regulatory T Cell Function. Immunity 53, 143-157 e148 (2020).
39. X. Yu et al., Metabolic control of regulatory T cell stability and function by TRAF3IP3 at the lysosome. J Exp Med 215, 2463-2476 (2018).
40. C. M. Paulos et al., The inducible costimulator (ICOS) is critical for the development of human T(H) 17 cells. Sci Transl Med 2, 55ra78 (2010).
41. Z. Hasan et al., Jun B is essential for IL-23-dependent pathogenicity of Th17 cells. Nat Commun 8, 15628 (2017).
42. C. Ma et al., Critical Role of CD6highCD4+ T Cells in Driving Th1/Th17 Cell Immune Responses and Mucosal Inflammation in IBD. J Crohns Colitis 13, 510-524 (2019).
43. N. Wang et al., CD226 Attenuates Treg Proliferation via Akt and Erk Signaling in an EAE Model. Front Immunol 11, 1883 (2020).
44. A. D. Keller, T. Maniatis, Identification and characterization of a novel repressor of beta-interferon gene expression. Genes Dev 5, 868-879 (1991).
45. G. A. Martins et al., Transcriptional repressor Blimp-1 regulates T cell homeostasis and function. Nat Immunol 7, 457-465 (2006).
46. M. H. Lin et al., T cell-specific BLIMP-1 deficiency exacerbates experimental autoimmune encephalomyelitis in nonobese diabetic mice by increasing Th1 and Th17 cells. Clin Immunol 151, 101-113 (2014).
47. R. Jain et al., Interleukin-23-Induced Transcription Factor Blimp-1 Promotes Pathogenicity of T Helper 17 Cells. Immunity 44, 131-142 (2016).
48. R. Bankoti et al., Differential regulation of Effector and Regulatory T cell function by Blimp1. Sci Rep 7, 12078 (2017).
49. C. Ogawa et al., Blimp-1 Functions as a Molecular Switch to Prevent Inflammatory Activity in Foxp3(+)RORgammat(+) Regulatory T Cells. Cell Rep 25, 19-28 e15 (2018).
50. G. Garg et al., Blimp1 Prevents Methylation of Foxp3 and Loss of Regulatory T Cell Identity at Sites of Inflammation. Cell Rep 26, 1854-1868 e1855 (2019).
51. I. Gyory, G. Fejer, N. Ghosh, E. Seto, K. L. Wright, Identification of a functionally impaired positive regulatory domain I binding factor 1 transcription repressor in myeloma cell lines. J Immunol 170, 3125-3133 (2003).
52. M. Boi, E. Zucca, G. Inghirami, F. Bertoni, PRDM1/BLIMPl: a tumor suppressor gene in B and T cell lymphomas. Leuk Lymphoma 56, 1223-1228 (2015).
53. X. Wang et al., High-resolution genome-wide functional dissection of transcriptional regulatory regions and nucleotides in human. Nat Commun 9, 5380 (2018).
54. E. P. Consortium, An integrated encyclopedia of DNA elements in the human genome. Nature 489, 57-74 (2012).
55. C. Roadmap Epigenomics et al., Integrative analysis of 111 reference human epigenomes. Nature 518, 317-330 (2015).
56. L. He et al., NEBULA is a fast negative binomial mixed model for differential or co-expression analysis of large-scale multi-subject single-cell data. Commun Biol 4, 629 (2021).
57. J. A. Hill et al., Foxp3 transcription-factor-dependent and -independent regulation of the regulatory T cell transcriptional signature. Immunity 27, 786-800 (2007).
58. A. Ferraro et al., Interindividual variation in human T regulatory cells. Proceedings of the National Academy of Sciences 111, E 1111-E1120 (2014).
59. A. Fragale et al., IFN regulatory factor-1 negatively regulates CD4+CD25+ regulatory T cell differentiation by repressing Foxp3 expression. J Immunol 181, 1673-1682 (2008).
60. N. Hayatsu et al., Analyses of a Mutant Foxp3 Allele Reveal BATF as a Critical Transcription Factor in the Differentiation and Accumulation of Tissue Regulatory T Cells. Immunity 47, 268-283 e269 (2017).
61. F. Renoux et al., The AP1 Transcription Factor Fos12 Promotes Systemic Autoimmunity and Inflammation by Repressing Treg Development. Cell Rep 31, 107826 (2020).
62. C. Wu et al., SGK1 Governs the Reciprocal Development of Th17 and Regulatory T Cells. Cell Rep 22, 653-665 (2018).
63. Z. Li et al., Identification of transcription factor binding sites using ATAC-seq. Genome Biology 20, 45 (2019).
64. M. Bentsen et al., ATAC-seq footprinting unravels kinetics of transcription factor binding during zygotic genome activation. Nat Commun 11, 4267 (2020).
65. P. Li et al., BATF-JUN is critical for IRF4-mediated transcription in T cells. Nature 490, 543-546 (2012).
66. T. L. Murphy, R. Tussiwand, K. M. Murphy, Specificity through cooperation: BATF-IRF interactions control immune-regulatory networks. Nat Rev Immunol 13, 499-509 (2013).
67. R. E. Gate et al., Genetic determinants of co-accessible chromatin regions in activated T cells across humans. Nat Genet 50, 1140-1150 (2018).
68. D. Pham et al., Batf Pioneers the Reorganization of Chromatin in Developing Effector T Cells via Ets1-Dependent Recruitment of Ctcf Cell Rep 29, 1203-1220 e1207 (2019).
69. S. M. Lee, B. Gao, D. Fang, FoxP3 maintains Treg unresponsiveness by selectively inhibiting the promoter DNA-binding activity of AP-1. Blood 111, 3599-3606 (2008).
70. R. M. Samstein et al., Foxp3 exploits a pre-existent enhancer landscape for regulatory T cell lineage specification. Cell 151, 153-166 (2012).
71. A. J. Lam et al., Optimized CRISPR-mediated gene knockin reveals FOXP3-independent maintenance of human Treg identity. Cell Rep 36, 109494 (2021).
72. C. M. Costantino, C. Baecher-Allan, D. A. Hafler, Multiple sclerosis and regulatory T cells. J Clin Immunol 28, 697-706 (2008).
73. M. Sambucci et al., FoxP3 isoforms and PD-1 expression by T regulatory cells in multiple sclerosis. Scientific Reports 8, 3674 (2018).
74. M. Rincon, R. A. Flavell, AP-1 transcriptional activity requires both T-cell receptor-mediated and co-stimulatory signals in primary T lymphocytes. EMBO J 13, 4370-4381 (1994).
75. M. Yukawa et al., AP-1 activity induced by co-stimulation is required for chromatin opening during T cell activation. J Exp Med 217, (2020).
76. L. Bossini-Castillo et al., Immune disease variants modulate gene expression in regulatory CD4+ T cells and inform drug targets. bioRxiv, 654632 (2019).
77. C. International Multiple Sclerosis Genetics, Multiple sclerosis genomic map implicates peripheral immune cells and microglia in susceptibility. Science 365, (2019).
78. B. J. Schmiedel et al., Impact of Genetic Polymorphisms on Human Immune Cell Gene Expression. Cell 175, 1701-1715 e1716 (2018).
79. H. Xi, G. Blanck, The IRF-2 DNA binding domain facilitates the activation of the class II transactivator (CIITA) type IV promoter by IRF-1. Mol Immunol 39, 677-684 (2003).
80. A. W. Mould, M. A. Morgan, A. C. Nelson, E. K. Bikoff, E. J. Robertson, Blimp1/Prdm1 Functions in Opposition to Irf1 to Maintain Neonatal Tolerance during Postnatal Intestinal Maturation. PLoS Genet 11, e1005375 (2015).
81. G. Lal et al., Distinct inflammatory signals have physiologically divergent effects on epigenetic regulation of Foxp3 expression and Treg function. Am J Transplant 11, 203-214 (2011).
82. E. Cretney et al., The transcription factors Blimp-1 and IRF4 jointly control the differentiation and function of effector regulatory T cells. Nat Immunol 12, 304-311 (2011).
83. S. Konermann et al., Genome-scale transcriptional activation by an engineered CRISPR-Cas9 complex. Nature 517, 583-588 (2015).
84. S. Fishilevich et al., GeneHancer: genome-wide integration of enhancers and target genes in GeneCards. Database (Oxford) 2017, (2017).
85. P. van Galen et al., A Multiplexed System for Quantitative Comparisons of Chromatin Landscapes. Mol Cell 61, 170-180 (2016).
86. E. Glasmacher et al., A genomic regulatory element that directs assembly and function of immune-specific AP-1-IRF complexes. Science 338, 975-980 (2012).
87. M. Ciofani et al., A validated regulatory network for Th17 cell specification. Cell 151, 289-303 (2012).
88. J. Li, H. Y. Comeau, Z. Zhang, X. Ren, Landscape of transcript isoforms in single T cells infiltrating in non-small-cell lung cancer. J Genet Genomics 47, 373-388 (2020).
89. K. Huhn et al., Skin sodium is increased in male patients with multiple sclerosis and related animal models. Proc Natl Acad Sci USA 118, (2021).
90. J. D. Buenrostro, B. Wu, H. Y. Chang, W. J. Greenleaf, ATAC-seq: A Method for Assaying Chromatin Accessibility Genome-Wide. Curr Protoc Mol Biol 109, 21 29 21-21 29 29 (2015).
91. J. D. Buenrostro et al., Single-cell chromatin accessibility reveals principles of regulatory variation. Nature 523, 486-490 (2015).
92. A. Dobin et al., STAR: ultrafast universal RNA-seq aligner. Bioinformatics 29, 15-21 (2013).
93. B. Li, C. N. Dewey, RSEM: accurate transcript quantification from RNA-Seq data with or without a reference genome. BMC Bioinformatics 12, 323 (2011).
94. J. Harrow et al., GENCODE: the reference human genome annotation for The ENCODE Project. Genome Res 22, 1760-1774 (2012).
95. M. D. Robinson, D. J. McCarthy, G. K. Smyth, edgeR: a Bioconductor package for differential expression analysis of digital gene expression data. Bioinformatics 26, 139-140 (2010).
96. M. E. Ritchie et al., limma powers differential expression analyses for RNA-sequencing and microarray studies. Nucleic Acids Res 43, e47 (2015).
97. C. W. Law, Y. Chen, W. Shi, G. K. Smyth, voom: precision weights unlock linear model analysis tools for RNA-seq read counts. Genome Biology 15, R29 (2014).
98. D. Risso, J. Ngai, T. P. Speed, S. Dudoit, Normalization of RNA-seq data using factor analysis of control genes or samples. Nat Biotechnol 32, 896-902 (2014).
99. M. Martin, Cutadapt removes adapter sequences from high-throughput sequencing reads. 2011 17, 3 (2011).
100. B. Langmead, S. L. Salzberg, Fast gapped-read alignment with Bowtie 2. Nat Methods 9, 357-359 (2012).
101. Y. Zhang et al., Model-based analysis of ChIP-Seq (MACS). Genome Biol 9, R137 (2008).
102. A. R. Quinlan, I. M. Hall, BEDTools: a flexible suite of utilities for comparing genomic features. Bioinformatics 26, 841-842 (2010).
103. E. G. Gusmao, M. Allhoff, M. Zenke, I. G. Costa, Analysis of computational footprinting methods for DNase sequencing experiments. Nat Methods 13, 303-309 (2016).
104. R. A. Fisher, Dispersion on a sphere. Proceedings of the Royal Society of London. Series A. Mathematical and Physical Sciences 217, 295-305 (1953).
105. A. Banerjee, I. S. Dhillon, J. Ghosh, S. Sra, Clustering on the Unit Hypersphere using von Mises-Fisher Distributions. J. Mach. Learn. Res. 6, 1345-1382 (2005).
106. K. Polanski et al., BBKNN: fast batch alignment of single cell transcriptomes. Bioinformatics 36, 964-965 (2019).
107. B. Hie, B. Bryson, B. Berger, Efficient integration of heterogeneous single-cell transcriptomes using Scanorama. Nature Biotechnology 37, 685-691 (2019)
108. A. Unterman et al., Single-cell multi-omics reveals dyssynchrony of the innate and adaptive immune system in progressive COVID-19. Nat Commun 13, 440 (2022).
109. M. A. Smith et al. PRDM1/Blimp-1 controls effector cytokine production in human NK cells. J Immunol 185, 6058-6067 (2010).
110. Lowther, D. E. et al. PD-1 marks dysfunctional regulatory T cells in malignant gliomas. JCI Insight 1 (2016).
111. Lucca, L. E. et al. TIGIT signaling restores suppressor function of Th1 Tregs. JCI Insight 4 (2019).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of restoring a regulatory T cell (Treg) function comprising contacting the Treg with an effective amount of an agent capable of reducing expression and/or function of a short isoform of PRDM1 (PRDM1-S).

2. The method of claim 1, wherein the agent inhibits expression and/or function of Blimp1-S, the protein encoded by a PRDM1-S gene.

3. The method of claim 1, wherein the agent inhibits expression of an mRNA transcribed from a PRDM1-S promoter.

4. The method of claim 3, wherein the agent blocks at least one regulatory element that controls transcription of the PRDM1-S mRNA.

5. The method of claim 4, wherein the at least one regulatory element is a promoter, an enhancer, or an insulator.

6. The method of claim 1, wherein the agent is a small molecule, an antibody or antigen-binding fragment thereof, or an aptamer.

7. The method of claim 1, wherein the agent is a gene regulation system.

8. The method of claim 7, wherein the agent comprises is-a CRISPR interference (CRISPRi) or CRISPRoff molecule.

9. A method of restoring a regulatory T cell (Treg) function comprising contacting the Treg with an effective amount of an agent capable of increasing expression and/or function of a long isoform of PRDM1 (PRDM1-L).

10-13. (canceled)

14. The method of claim 1, wherein the contacting occurs in vivo.

15. The method of claim 1, wherein the contacting occurs ex vivo.

16. The method of claim 1, wherein the Treg is in a subject and the agent is administered to the subject.

17. A method of treating a disease in a subject in need thereof comprising administering to the subject an effective amount of an agent capable of reducing expression and/or function of a short isoform of PRDM1 (PRDM1-S).

18-24. (canceled)

25. A method of treating a disease in a subject in need thereof comprising administering to the subject an effective amount of an agent capable of increasing expression and/or function of a long isoform of PRDM1 (PRDM1-L).

26-32. (canceled)

33. The method of claim 16, wherein the subject is a dry-nosed mammal.

34. The method of claim 33, wherein the subject is a human.

35. A method of screening for a candidate compound that restores a regulatory T cell (Treg) function, comprising:
(a) determining expression or function of a short isoform of PRDM1 (PRDM1-S) and/or a long form of PRDM1 (PRDM1-L) in the Treg before and after contacting said Treg with a test compound; and
(b) selecting a compound that i) reduces expression and/or function of PRDM1-S in comparison with expression and/or function of PRDM1-S determined in the absence of the test compound; and/or ii) increases expression or function of long form of PRDM1 (PRDM1-L) in comparison with expression and/or function of PRDM1-L determined in the absence of the test compound.
